SEQUENCE LISTING (14 sequences)

SEQ ID NO: 1 - protein, 188 aa, Homo sapiens [sequence omitted]

SEQ ID NO: 2 - protein, 188 aa, Homo sapiens [sequence omitted]

SEQ ID NO: 3 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 4 - protein, 188 aa, Homo sapiens [sequence omitted]

SEQ ID NO: 5 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 6 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 7 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 8 - protein, 189 aa, Homo sapiens [sequence omitted]

SEQ ID NO: 9 - protein, 188 aa, Homo sapiens
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 10 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSED

SEQ ID NO: 11 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDD

SEQ ID NO: 12 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDD

SEQ ID NO: 13 - protein, 119 aa, Homo sapiens
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDEN

SEQ ID NO: 14 - protein, 120 aa, Homo sapiens
MAANKSKGQSSLALHKVIMVGSGGVGKSALTLQFMYDEFVEDYEPTKADSYRKKVVLDGEEVQIDILDTAGQEDYAAIRDNYFRSGEGFLLVFSITEHESFTATAEFREQILRVKAEEDK

(19) United States
(12) Patent Application Publication
(10) Pub. No.: US 2026/0274821 A1
(43) Pub. Date: Sep. 17, 2026

(54) RAS INHIBITORS

(71) Applicant: Kumquat Biosciences Inc., San Diego, CA (US)

(72) Inventors: Pingda Ren, San Diego, CA (US); Xiaoming Li, San Diego, CA (US); Xiaohui He, San Diego, CA (US); Rasmus Hansen, San Diego, CA (US); Liansheng Li, San Diego, CA (US); Baogen Wu, San Diego, CA (US); Zhiyong Chen, San Diego, CA (US); Yuan Liu, San Diego, CA (US)

(21) Appl. No.: 19/564,596

(22) Filed: Mar. 12, 2026

Related U.S. Application Data
(63) Continuation of application No. PCT/US2024/046476, filed on Sep. 12, 2024.
(60) Provisional application No. 63/582,318, filed on Sep. 13, 2023.
(60) Provisional application No. 63/582,327, filed on Sep. 13, 2023.
(60) Provisional application No. 63/582,343, filed on Sep. 13, 2023.
(60) Provisional application No. 63/582,351, filed on Sep. 13, 2023.
(60) Provisional application No. 63/582,374, filed on Sep. 13, 2023.
(60) Provisional application No. 63/582,377, filed on Sep. 13, 2023.
(60) Provisional application No. 63/582,420, filed on Sep. 13, 2023.
(60) Provisional application No. 63/582,435, filed on Sep. 13, 2023.
(60) Provisional application No. 63/582,445, filed on Sep. 13, 2023.
(60) Provisional application No. 63/582,391, filed on Sep. 13, 2023.

(51) Int. Cl. C07D 401/14; A61K 31/517; A61K 31/5377; A61K 45/06; C07D 471/04; C07D 487/04

(52) U.S. Cl. CPC C07D 401/14; A61K 31/517; A61K 31/5377; A61K 45/06; C07D 471/04; C07D 487/04

(57) ABSTRACT

The present disclosure provides compounds and pharmaceutically acceptable salts thereof, and methods of using the same. The compounds and methods have a range of utilities as therapeutics, diagnostics, and research tools. In particular, the subject compositions and methods are useful for reducing signaling output of oncogenic protein.

### CROSS-REFERENCE

This application is a continuation of International Application No. PCT/US2024/046476, filed Sep. 12, 2024, which claims the benefit of U.S. Provisional Application No. 63/582,318, filed Sep. 13, 2023, U.S. Provisional Application No. 63/582,327, filed Sep. 13, 2023, U.S. Provisional Application No. 63/582,343, filed Sep. 13, 2023, U.S. Provisional Application No. 63/582,351, filed Sep. 13, 2023, U.S. Provisional Application No. 63/582,374, filed Sep. 13, 2023, U.S. Provisional Application No. 63/582,377, filed Sep. 13, 2023, U.S. Provisional Application No. 63/582,420, filed Sep. 13, 2023, U.S. Provisional Application No. 63/582,435, filed Sep. 13, 2023, U.S. Provisional Application No. 63/582,445, filed Sep. 13, 2023, and U.S. Provisional Application No. 63/582,391, filed Sep. 13, 2023, each incorporated herein by reference in its entirety.

### SEQUENCE LISTING

The instant application contains a Sequence Listing which has been submitted electronically in XML format and is hereby incorporated by reference in its entirety. Said XML copy, created on Mar. 11, 2026, is named 56690_795_301_SL.xml and is 15,007 bytes in size.

### BACKGROUND

A majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. Some may have been misled into believing that a small molecule modulator could be discovered for most proteins provided a reasonable amount of resources, time, and effort, given the fact that many such drug/target interacting pairs are known. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward small molecule drug discovery and are commonly referred to as “undruggable”. These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy, especially since mutations in Ras proteins account for approximately 30% of all human cancers in the United States, many of which are fatal. Dysregulation of Ras proteins by overexpression, upstream activation, or activating mutations is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state, leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by dysregulation of Ras proteins, including by the various Ras mutations.

### SUMMARY

In view of the foregoing, there remains a considerable need for a new design of therapeutics and diagnostics that can specifically target Ras, including wildtype Ras, mutants and/or associated proteins of Ras to reduce Ras signaling output. Of particular interest are Ras inhibitors, including pan Ras inhibitors capable of inhibiting two or more Ras mutants and/or wildtype Ras, as well as mutant-selective inhibitors targeting mutant Ras proteins such as Ras G12D, G12C, G12S, G13D, and/or G12V, for the treatment of Ras-associated diseases (e.g., cancer). Such compositions and methods can be particularly useful for treating a variety of diseases including, but not limited to, cancers and neoplasia conditions. The present disclosure addresses these needs, and provides additional advantages applicable for diagnosis, prognosis, and/or treatment for a wide diversity of diseases.

In certain aspects, the present disclosure provides a compound of Formula (I):

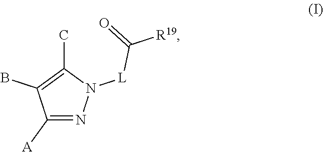

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A and B are independently selected from C₃₋₁₂ carbocycle and 3- to 12-membered heterocycle, each of which is optionally substituted;
C is selected from hydrogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

In some embodiments, for a compound of Formula (I), C is selected from hydrogen, halogen, —CN, C₁₋₃ alkyl, C₃₋₅ cycloalkyl, C₁₋₃ haloalkyl, —CH₂CN, —CH(CN)CH₃, —CH₂OH, and —CH(OH)CH₃. In some embodiments, A and B are independently selected from optionally substituted indazolyl.

In some embodiments, the compound of Formula (I) is a compound of Formula (II):

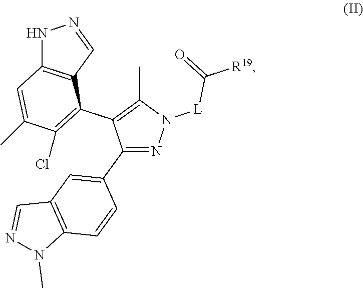

or a pharmaceutically acceptable salt or solvate thereof.

In some embodiments, for a compound of Formula (I) or (II), L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is selected from a bond and C₁₋₃ alkyl. In some embodiments, L² is optionally substituted 3- to 12-membered heterocycle; and L³ is a bond. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In some embodiments, for a compound of Formula (I) or (II), -L²-L³-C(O)R¹⁹ is selected from:

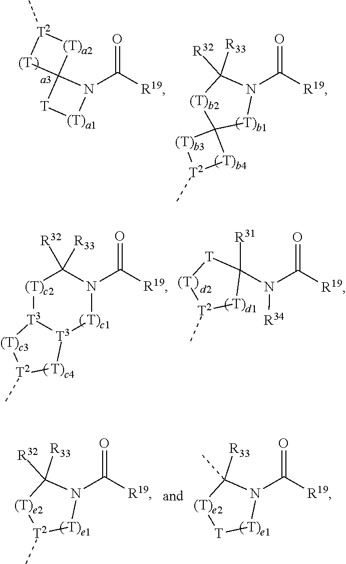

wherein:
a1, b1, b3, and b4 are independently 1, 2, 3, 4, or 5;
a2, a3, and b2 are independently 0, 1, 2, 3, 4, or 5;
c1, c2, c3, c4, d1, d2, e1, and e2 are independently 0, 1, 2, 3, or 4;
wherein the sum of a1, a2, and a3 is less than 9; the sum of b1, b2, b3, and b4 is less than 9; the sum of c1, c2, c3, and c4 is less than 8; the sum of d1 and d2 is less than 6; and the sum of e1 and e2 is less than 6;
T is independently selected at each occurrence from N(R³⁵), C(R³⁶)₂, C(O), O, S(O), and S(O)₂;
T² and T³ are independently selected at each occurrence from N and C(R³⁶);
R³¹, R³², R³³, and R³⁶ are independently selected at each occurrence from hydrogen and R⁴⁰;
R³⁴ and R³⁵ are independently selected at each occurrence from hydrogen and R⁴¹;
R⁴⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R⁴⁰ attached to the same carbon atom optionally join to form ═NR¹², ═C(R¹⁴)₂, or ═O; wherein two R⁴⁰ and the atom(s) to which they are attached optionally form C₃₋₁₂ carbocycle or 3- to 12-membered heterocycle; wherein R⁴⁰ and R⁴¹ and the atoms to which they are attached optionally form 3- to 12-membered heterocycle; and wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁴¹ is independently selected at each occurrence from —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, R⁴⁰ is independently selected at each occurrence from halogen, —CN, C₁₋₆ alkyl, and C₃₋₆ cycloalkyl, or two R⁴⁰ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R⁴¹ is independently selected at each occurrence from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, the compound of Formula (I) is:

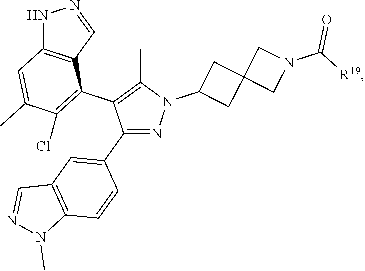

or a pharmaceutically acceptable salt or solvate thereof.

In some embodiments, for a compound of Formula (I) or (II), R¹⁹ is selected from 1,2,3-triazol-1-yl, 1,2,3-triazol-2-yl, 1,2,4-triazol-1-yl, and 1,2,4-triazol-4-yl, each of which is optionally substituted. In some embodiments, R¹⁹ is optionally substituted imidazol-1-yl. In some embodiments, R¹⁹ is optionally substituted with one or two substituents independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents independently selected from halogen, —CN, C₁₋₆ alkyl, —O(C₁₋₆ alkyl), and —O(C₁₋₆ haloalkyl).

In certain aspects, the present disclosure provides a pharmaceutical composition comprising a compound disclosed herein, or a pharmaceutically acceptable salt or solvate thereof, and a pharmaceutically acceptable excipient.

In certain aspects, the present disclosure provides a method of modifying a Ras mutant protein, comprising contacting the Ras mutant protein with an effective amount of a compound disclosed herein, or a salt or solvate thereof. In some embodiments, the modified Ras mutant protein exhibits a reduced Ras signaling output. In some embodiments, the reduced Ras signaling output is evidenced by one or more output selected from (i) an increase in steady state level of GDP-bound modified protein; (ii) a reduction in steady state level of GTP-bound modified protein; (iii) a reduction of phosphorylated AKTs473; (iv) a reduction of phosphorylated ERK T202/Y204; (v) a reduction of phosphorylated S6 S235/236; (vi) a reduction of cell growth of a tumor cell expressing a Ras G12S mutant protein; and (vii) a reduction in Ras interaction with a Ras-pathway signaling protein. In some embodiments, the Ras mutant protein comprises an amino acid sequence in SEQ ID No. 4 having a serine residue corresponding to position 12 of SEQ ID No. 1. In some embodiments, the Ras mutant protein comprises an amino acid sequence of SEQ ID No. 4. In some embodiments, the modified Ras mutant protein comprises an amino acid sequence of SEQ ID No. 1, or a fragment thereof that comprises the serine residue corresponding to position 12 of SEQ ID No. 1, and wherein the compound selectively labels the serine residue as compared to (i) an aspartate residue of a K-Ras G12D mutant protein, said aspartate corresponding to position 12 of SEQ ID No. 2; (ii) a valine residue of a K-Ras G12V mutant protein, said valine corresponding to position 12 of SEQ ID No. 3; and/or (iii) a glycine residue of a K-Ras wildtype protein, said glycine corresponding to position 12 of SEQ ID No. 1. In some embodiments, the compound selectively labels the serine residue by at least 2-fold when assayed under comparable conditions. In some embodiments, the compound selectively labels the serine residue by at least 5-fold when assayed under comparable conditions. In some embodiments, the contacting occurs in vivo.

In certain aspects, the present disclosure provides a method of treating cancer in a subject in need thereof, comprising administering to the subject a therapeutically effective amount of a compound disclosed herein, or a pharmaceutically acceptable salt or solvate thereof. In certain aspects, the present disclosure provides a method of treating cancer in a subject comprising a Ras mutant protein, the method comprising: inhibiting the Ras mutant protein of said subject by administering to said subject a compound disclosed herein, wherein the compound is characterized in that upon contacting the Ras mutant protein, said Ras mutant protein exhibits reduced Ras signaling output. In certain aspects, the present disclosure provides a method of modulating signaling output of a Ras protein, comprising contacting a Ras protein with an effective amount of a compound disclosed herein, or a pharmaceutically acceptable salt or solvate thereof, thereby modulating the signaling output of the Ras protein. In certain aspects, the present disclosure provides a method of inhibiting cell growth, comprising administering an effective amount of a compound disclosed herein, or a pharmaceutically acceptable salt or solvate thereof, to a cell expressing a Ras protein, thereby inhibiting growth of said cells.

In practicing any of the subject methods, the cancer may be a solid tumor or a hematological cancer. In some embodiments, the cancer comprises a K-Ras G12S mutant protein. A method of the present disclosure may further comprise administering an additional agent.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a sequence alignment of various wild type Ras proteins including K-Ras, H-Ras, N-Ras, RalA, and RalB, from top to bottom.

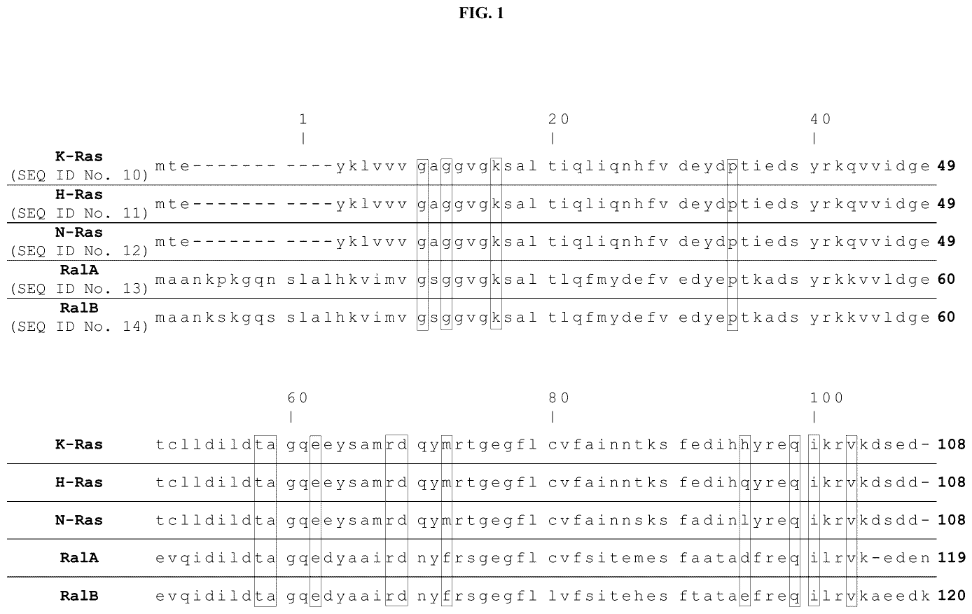

### DETAILED DESCRIPTION

Unless defined otherwise, all technical and scientific terms used herein have the same meaning as is commonly understood by one of skill in the art to which this disclosure belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Chemical structures are named herein according to IUPAC conventions as implemented in ChemDraw® software (Perkin Elmer, Inc., Cambridge, MA). The section headings used herein are for organizational purposes only and are not to be construed as limiting the subject matter described. As used in the specification and claims, the singular forms “a”, “an” and “the” include plural references unless the context clearly dictates otherwise. Furthermore, use of the term “including” as well as other forms, such as “include”, “includes”, and “included”, is not limiting. The section headings used herein are for organizational purposes only and are not to be construed as limiting the subject matter described.

The term “C_{x-y}” or “Cₓ-C_{y}” when used in conjunction with a chemical moiety, such as alkyl, alkenyl, or alkynyl, is meant to include groups that contain from x to y carbons in the chain. For example, the term “C_{x-y} alkyl” refers to substituted or unsubstituted saturated hydrocarbon groups, including straight-chain alkyl and branched-chain alkyl groups, that contain from x to y carbons in the chain.

“Alkyl” refers to substituted or unsubstituted saturated hydrocarbon groups, including linear and branched alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including linear and branched alkenyl groups, containing at least one double bond. An alkenyl group may contain from two to twelve carbon atoms (e.g., C₂₋₁₂ alkenyl), such as two to eight carbon atoms (C₂₋₈ alkenyl) or two to six carbon atoms (C₂₋₆ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including linear and branched alkynyl groups, containing at least one triple bond. An alkynyl group may contain from two to twelve carbon atoms (e.g., C₂₋₁₂ alkynyl), such as two to eight carbon atoms (C₂₋₈ alkynyl) or two to six carbon atoms (C₂₋₆ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including linear alkylene and branched alkylene groups, that contain from one to twelve carbon atoms (e.g., C₁₋₁₂ alkylene), such as one to eight carbon atoms (C₁₋₈ alkylene) or one to six carbon atoms (C₁₋₆ alkylene). Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons of the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkyl group has a chain length of 3 to 8 atoms. Connection to the rest of the molecule may be through either a heteroatom or a carbon in the heteroalkyl, heteroalkenyl, or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkylene group has a chain length of 3 to 8 atoms. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkylene, heteroalkenylene or heteroalkynylene chain. Unless stated otherwise specifically in the specification, a heteroalkylene, heteroalkenylene, or heteroalkynylene group is optionally substituted by one or more substituents such as those substituents described herein.

“Nitrene” refers to an unsubstituted divalent group of the general formula —NH— and its R-substituted derivatives of the general formula —N(R)—. Unless stated otherwise specifically in the specification, a nitrene group is optionally substituted, such as by a substituent described herein. Exemplary nitrene groups include —NH—, —N(CH₃)—, —N(CH₂CH₃)—, —N(CH₂CH₂CH₃), —N(CH(CH₃)₂)—, —N(CH₂CH(CH₃)₂)—, —N(C(CH₃)₃), —N(CH₂F)—, —N(CHF₂)—, —N(CF₃)—, —N(cyclopropyl)-, —N(cyclobutyl)-, and —N(cyclopentyl)-.

“Carbocycle” refers to a saturated, unsaturated or aromatic ring in which each atom of the ring is a carbon atom. Carbocycle may include C₃₋₁₀ monocyclic rings, C₅₋₁₂ bicyclic rings, C₅₋₁₈ polycyclic rings, C₅₋₁₂ spirocyclic rings, and C₅₋₁₂ bridged rings. Each ring of a bicyclic or polycyclic carbocycle may be selected from saturated, unsaturated, and aromatic rings. A polycyclic carbocycle contains a number or rings equal to the minimum number of scissions required to convert the carbocycle into an acyclic skeleton (e.g., bicyclic, tricyclic, tetracyclic, etc.). In some embodiments, the carbocycle is a C₆₋₁₂ aryl group, such as C₆₋₁₀ aryl. In some embodiments, the carbocycle is a C₃₋₁₂ cycloalkyl group. In some embodiments, the carbocycle is a C₅₋₁₂ cycloalkenyl group. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic rings, as valence permits, are included in the definition of carbocycle. A carbocycle may comprise a fused ring, a bridged ring, a spirocyclic ring, a saturated ring, an unsaturated ring, an aromatic ring, or any combination thereof. Exemplary carbocycles include cyclopentyl, cyclohexyl, cyclohexenyl, adamantly, phenyl, indanyl, and naphthyl. Unless state otherwise specifically in the specification, a carbocycle is optionally substituted by one or more substituents such as those substituents described herein.

“Heterocycle” refers to a saturated, unsaturated or aromatic ring comprising one or more heteroatoms, for example 1, 2, 3, or 4 heteroatoms selected from O, S, P, and N. Heterocycle may include 3- to 10-membered monocyclic rings, 5- to 12-membered bicyclic rings, 5- to 18-membered polycyclic rings, 5- to 12-membered spirocyclic rings, and 5- to 12-membered bridged rings. Each ring of a bicyclic or polycyclic heterocycle may be selected from saturated, unsaturated, and aromatic rings. A polycyclic heterocycle contains a number or rings equal to the minimum number of scissions required to convert the heterocycle into an acyclic skeleton (e.g., bicyclic, tricyclic, tetracyclic, etc.). The heterocycle may be attached to the rest of the molecule through any atom of the heterocycle, valence permitting, such as a carbon or nitrogen atom of the heterocycle. In some embodiments, the heterocycle is a 5- to 10-membered heteroaryl group, such as 5- or 6-membered heteroaryl. In some embodiments, the heterocycle is a 3- to 12-membered heterocycloalkyl group. A heterocycle may comprise a fused ring, a bridged ring, a spirocyclic ring, a saturated ring, an unsaturated ring, an aromatic ring, or any combination thereof. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, benzothienyl, benzoxazolyl, and quinolinyl. Unless stated otherwise specifically in the specification, a heterocycle is optionally substituted by one or more substituents such as those substituents described herein.

“Heteroaryl” refers to an aromatic ring that comprises at least one heteroatom, for example 1, 2, 3, or 4 heteroatoms selected from O, S and N. Heteroaryl may include 5- to 10-membered monocyclic rings, 6- to 12-membered bicyclic rings, 6- to 18-membered polycyclic rings, 5- to 12-membered spirocyclic rings, and 6- to 12-membered bridged rings. As used herein, the heteroaryl ring may be selected from monocyclic, bicyclic, or polycyclic—including fused, spirocyclic and bridged ring systems—wherein at least one of the rings in the ring system is aromatic and comprises at least one heteroatom. A polycyclic heteroaryl contains a number or rings equal to the minimum number of scissions required to convert the heteroaryl into an acyclic skeleton (e.g., bicyclic, tricyclic, tetracyclic, etc.). The heteroatom(s) in the heteroaryl may optionally be oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryl groups include, but are not limited to, azepinyl, benzimidazolyl, benzisothiazolyl, benzisoxazolyl, benzofuranyl, benzothiazolyl, benzothiophenyl, benzoxazolyl, furanyl, imidazolyl, indazolyl, indolyl, isoquinolinyl, isothiazolyl, isoxazolyl, oxadiazolyl, oxazolyl, purinyl, pyrazinyl, pyrazolidinyl, pyrazolyl, pyridazinyl, pyridazolyl, pyridyl, pyrimidinyl, pyrrolyl, quinazolinyl, quinolinyl, quinoxalinyl, tetrahydroquinolinyl, thiadiazolyl, thiazolyl, and thienyl groups. Unless stated otherwise specifically in the specification, a heteroaryl is optionally substituted by one or more substituents such as those substituents described herein.

Unless stated otherwise, hydrogen atoms are implied in structures depicted herein as necessary to satisfy the valence requirement.

A waved line “ [symbol] ” drawn across or at the end of a bond or a dashed bond “ [symbol] ” are used interchangeably herein to denote where a bond disconnection or attachment occurs. For example, in the structure

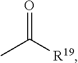

if R¹⁹ is 1,2,4-triazol-1-yl as in

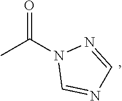

then R¹⁹ may be depicted as

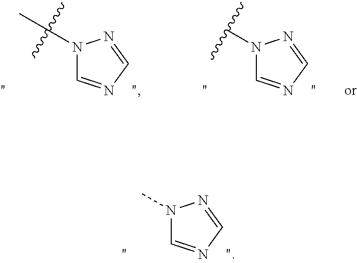

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, heteroatoms such as nitrogen may have any permissible substituents of organic compounds described herein which satisfy the valences of the heteroatoms.

A compound disclosed herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is optionally substituted by one or more—such as 1, 2 or 3—substituents selected from:
halogen, oxo, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, and —S(═O)(═NR²²)N(R²²)(R²³); wherein two substituents attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3 to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH; R²² is independently selected at each occurrence from hydrogen, C₁₋₆ alkyl, C₁₋₆ haloalkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein —C₀₋₆ alkyl-(C₃₋₁₂ carbocycle) and —C₀₋₆ alkyl-(3- to 12-membered heterocycle) are optionally substituted with one, two, or three groups independently selected from halogen and C₁₋₆ alkyl; and
R²³ is independently selected at each occurrence from hydrogen and C₁₋₆ alkyl; or R²² and R²³ attached to the same nitrogen atom form 3- to 10 membered heterocycle.

In some embodiments, a compound disclosed herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is optionally substituted by one or more—such as 1, 2 or 3—substituents selected from:
halogen, oxo, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —OC(O)R²², —C(O)N(R²²)(R²³), —C(O)C(O)N(R²²)(R²³), —N(R²²)C(O)R²², —S(O)₂R²², —S(O)(NR²²)R²², and —S(O)₂N(R²²)(R²³)—, wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², and ═C(R²¹)₂;
R²¹ is independently selected at each occurrence from hydrogen, halogen, C₁₋₆ alkyl, and C₁₋₆ haloalkyl;
R²² is independently selected at each occurrence from hydrogen, C₁₋₆ alkyl, C₁₋₆ haloalkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein —C₀₋₆ alkyl-(C₃₋₁₂ carbocycle) and —C₀₋₆ alkyl-(3- to 12-membered heterocycle) are optionally substituted with one, two, or three groups independently selected from halogen and C₁₋₆ alkyl;
R²³ is independently selected at each occurrence from hydrogen and C₁₋₆ alkyl; or R²² and R²³ attached to the same nitrogen atom form 3- to 10 membered heterocycle.

In some embodiments, a compound disclosed herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is optionally substituted by one or more-such as 1, 2 or 3-substituents selected from halogen, oxo, ═NH, —CN, —NO₂, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OH, —OCH₃, —OCH₂CH₃, —NH₂, —NHCH₃, and —NHCH₂CH₃, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH(CH₃)₂, —C(CH₃)₃, —OH, —OCH₃, —OCH₂CH₃, —NH₂, —NHCH₃, and —NHCH₂CH₃.

It will be understood by those skilled in the art that substituents can themselves be substituted, if appropriate. Unless specifically stated as “unsubstituted”, references to chemical moieties herein are understood to include substituted variants. For example, reference to a “heteroaryl” group or moiety implicitly includes both substituted and unsubstituted variants.

Where bivalent substituent groups are specified herein by their conventional chemical formulae, written from left to right, they are intended to encompass the isomer that would result from writing the structure from right to left, e.g., —CH₂O— is also intended to encompass —OCH₂—.

“Optional” or “optionally” means that the subsequently described event or circumstances may or may not occur, and that the description includes instances where the event or circumstance occurs and instances in which it does not. For example, an “optionally substituted” group may be either unsubstituted or substituted.

Compounds of the present disclosure also include crystalline and amorphous forms of those compounds, pharmaceutically acceptable salts, and active metabolites having the same type of activity, including, for example, polymorphs, pseudopolymorphs, solvates, hydrates, unsolvated polymorphs (including anhydrates), conformational polymorphs, amorphous forms of the compounds, and mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Examples of isotopes that may be incorporated into compounds of the present disclosure include, but are not limited to, ²H, ³H, ¹³C, ¹⁴C, ¹⁵N, ¹⁸O, ¹⁷O, ³⁵S, ³⁶Cl, and ¹⁸F. Of particular interest are compounds of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV) enriched in tritium or carbon-14, which can be used, for example, in tissue distribution studies; compounds of the disclosure enriched in deuterium—especially at a site of metabolism—resulting, for example, in compounds having greater metabolic stability; and compounds of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV) enriched in a positron emitting isotope, such as ¹¹C, ¹⁸F, ¹⁵O and ¹³N, which can be used, for example, in Positron Emission Topography (PET) studies. Isotopically-enriched compounds may be prepared by conventional techniques well known to those skilled in the art.

As used herein, the phrase “of the formula”, “having the formula” or “having the structure” is not intended to be limiting and is used in the same way that the term “comprising” is commonly used. For example, if one structure is depicted, it is understood that all stereoisomer and tautomer forms are encompassed, unless stated otherwise.

Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. In some embodiments, in order to optimize the therapeutic activity of the compounds of the disclosure, e.g., to treat cancer, it may be desirable that the carbon atoms have a particular configuration (e.g., (R,R), (S,S), (S,R), or (R,S)) or are enriched in a stereoisomeric form having such configuration. The compounds of the disclosure may be provided as racemic mixtures. Accordingly, the disclosure relates to racemic mixtures, pure stereoisomers (e.g., enantiomers and diastereomers), stereoisomer-enriched mixtures, and the like, unless otherwise indicated. When a chemical structure is depicted herein without any stereochemistry, it is understood that all possible stereoisomers are encompassed by such structure. Similarly, when a particular stereoisomer is shown or named herein, it will be understood by those skilled in the art that minor amounts of other stereoisomers may be present in the compositions of the disclosure unless otherwise indicated, provided that the utility of the composition as a whole is not eliminated by the presence of such other isomers. Individual stereoisomers may be obtained by numerous methods that are known in the art, including preparation using chiral synthons or chiral reagents, resolution using chiral chromatography using a suitable chiral stationary phase or support, or by chemically converting them into diastereomers, separating the diastereoisomers by conventional means such as chromatography or recrystallization, then regenerating the original stereoisomer.

Additionally, where applicable, all cis-trans or E/Z isomers (geometric isomers), tautomeric forms and topoisomeric forms of the compounds described herein are included with the scope of the disclosure unless otherwise specified.

The term “pharmaceutically acceptable” refers to a material that is not biologically or otherwise unacceptable when used in the subject compositions and methods. For example, the term “pharmaceutically acceptable carrier” refers to a material—such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc., and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, “treating” or “treatment” refers to an approach for obtaining beneficial or desired results with respect to a disease, disorder, or medical condition (such as cancer) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating cancer” would include preventing cancer from occurring, ameliorating cancer, suppressing cancer, and alleviating the symptoms of cancer. Also, a therapeutic benefit is achieved with the eradication or amelioration of one or more of the physiological symptoms associated with the underlying disorder such that an improvement is observed in the subject, notwithstanding that the subject may still be afflicted with the underlying disorder.

A “therapeutic effect”, as that term is used herein, encompasses a therapeutic benefit and/or prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “antagonist” and “inhibitor” are used interchangeably, and they refer to a compound having the ability to inhibit a biological function (e.g., activity, expression, binding, protein-protein interaction) of a target protein (e.g., K-Ras). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

The terms “subject” and “patient” refer to an animal, such as a mammal, for example a human. The methods described herein can be useful in both human therapeutics and veterinary applications. In some embodiments, the subject is a mammal, such as a human. “Mammal” includes humans and both domestic animals such as laboratory animals and household pets (e.g., cats, dogs, swine, cattle, sheep, goats, horses, rabbits), and non-domestic animals such as wildlife and the like.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

An “antigen” is a moiety or molecule that contains an epitope, and, as such, also specifically binds to an antibody. An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or V_{H}H), a heavy chain found in sharks (V-NAR domain), a single domain antibody (sdAb, e.g., “nanobody”) that comprises a single antigen-binding domain, Fv, Fd, Fab, Fab′, F(ab′)2, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

“Prodrug” is meant to indicate a compound that may be converted under physiological conditions or by solvolysis to a biologically active compound described herein (e.g., a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV)). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press each of which is incorporated in full by reference herein). The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

The term “in vivo” refers to an event that takes place in a subject's body. The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body. The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound disclosed herein to a mammal for a period of time sufficient to yield a metabolic product thereof. Such products are typically identified by administering a radiolabeled compound of the disclosure in a detectable dose to an animal, such as rat, mouse, guinea pig, monkey, or to a human, allowing sufficient time for metabolism to occur, and isolating its conversion products from the urine, blood or other biological samples.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (also used interchangeably herein as K-Ras, K-ras, or Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof.

The terms “mutant Ras” and “Ras mutant”, as used interchangeably herein, refer to a Ras protein with one or more amino acid mutations, such as with respect to a common reference sequence such as a wild-type (WT) sequence. In some embodiments, a mutant Ras is selected from a mutant KRAS, mutant HRAS, mutant NRAS, mutant MRAS, mutant ERAS, mutant RRAS2, mutant RALA, mutant RALB, mutant RIT1, and any combination thereof, such as from a mutant KRAS, mutant HRAS, mutant NRAS, mutant RALA, mutant RALB, and any combination thereof. In some embodiments, a mutation can be an introduced mutation, a naturally occurring mutation, or a non-naturally occurring mutation. In some embodiments, a mutation can be a substitution (e.g., a substituted amino acid), insertion (e.g., addition of one or more amino acids), or deletion (e.g., removal of one or more amino acids). In some embodiments, two or more mutations can be consecutive, non-consecutive, or a combination thereof. In some embodiments, a mutation can be present at any position of Ras. In some embodiments, a mutation can be present at position 12, 13, 62, 92, 95, 96 (e.g., Y96D), or any combination thereof of Ras relative to SEQ ID No. 1 when optimally aligned. In some embodiments, a mutant Ras may comprise about or at least about 1, 2, 3, 4, 5, 6, 7, 8, 9, 10, 11, 12, 13, 14, 15, 16, 17, 18, 19, 20, 25, 30, 35, 40, 45, 50, or more than 50 mutations. In some embodiments, a mutant Ras may comprise up to about 1, 2, 3, 4, 5, 6, 7, 8, 9, 10, 11, 12, 13, 14, 15, 16, 17, 18, 19, 20, 25, 30, 35, 40, 45, or 50 mutations. In some embodiments, the mutant Ras is about or up to about 500, 400, 300, 250, 240, 233, 230, 220, 219, 210, 208, 206, 204, 200, 195, 190, 189, 188, 187, 186, 185, 180, 175, 174, 173, 172, 171, 170, 169,168, 167, 166, 165, 160, 155, 150, 125, 100, 90, 80, 70, 60, 50, or fewer than 50 amino acids in length. In some embodiments, an amino acid of a mutation is a proteinogenic, natural, standard, non-standard, non-canonical, essential, non-essential, or non-natural amino acid. In some embodiments, an amino acid of a mutation has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, a mutation comprises a reactive moiety. In some embodiments, a substituted amino acid comprises a reactive moiety. In some embodiments, a mutant Ras can be further modified, such as by conjugation with a detectable label. In some embodiments, a mutant Ras is a full-length or truncated polypeptide. For example, a mutant Ras can be a truncated polypeptide comprising residues 1-169 or residues 11-183 (e.g., residues 11-183 of a mutant RALA or mutant RALB).

As used herein, the term “corresponding to” or “corresponds to” as applied to an amino acid residue in a polypeptide sequence refers to the correspondence of such amino acid relative to a reference sequence when optimally aligned (e.g., taking into consideration of gaps, insertions and mismatches; wherein alignment may be primary sequence alignment or three-dimensional structural alignment of the folded proteins). For instance, the serine residue in a K-Ras G12S mutant refers to the serine corresponding to residue 12 of SEQ ID No. 4, which can serve as a reference sequence. For instance, the aspartate residue in a K-Ras G12D mutant refers to the aspartate corresponding to residue 12 of SEQ ID No. 2, which can serve as a reference sequence. When an amino acid of a mutant Ras protein corresponds to an amino acid position in the WT Ras protein, it will be understood that although the mutant Ras protein amino acid may be a different amino acid (e.g., G12D, wherein the wildtype G at position 12 is replaced by an aspartate at position 12 of SEQ ID. No. 1), the mutant amino acid is at the position corresponding to the wildtype amino acid (e.g., of SEQ ID No. 1). In some embodiments, a modified Ras mutant protein disclosed herein may comprise truncations at the C-terminus, or truncations at the N-terminal end preceding the serine residue. The serine residue in such N-terminal truncated modified mutant is still considered corresponding to position 12 of SEQ ID No. 1. In addition, an aspartate residue at position 12 of SEQ ID No. 2 finds a corresponding residue in SEQ ID Nos. 6 and 8.

As used herein, the term “leaving group” refers to an atom or group that becomes detached from an atom in the residual or main part of the substrate in a specified reaction. The residual or main part of the substrate is also referred to herein as the “staying group”.

### Compounds of Formula (I) and (II)

In certain aspects, the present disclosure provides a compound of Formula (I):

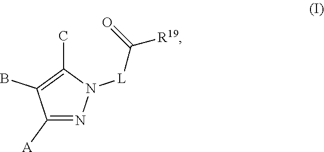

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A and B are independently selected from C₃₋₁₂ carbocycle and 3- to 12-membered heterocycle, each of which is optionally substituted;
C is selected from hydrogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

In some embodiments, for a compound of Formula (I), C is selected from hydrogen, halogen, —CN, C₁₋₃ alkyl, C₃₋₅ cycloalkyl, C₁₋₃ haloalkyl, —CH₂CN, —CH(CN)CH₃, —CH₂OH, and —CH(OH)CH₃. In some embodiments, A and B are independently selected from optionally substituted indazolyl.

In certain aspects, the present disclosure provides a compound of Formula (II):

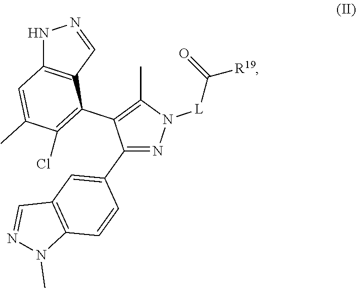

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted; and
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted.

In some embodiments, the compound of Formula (I) is:

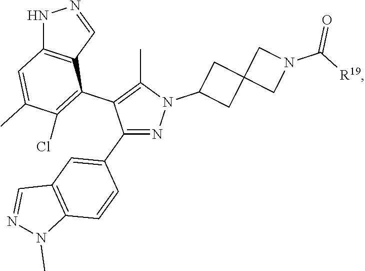

or a pharmaceutically acceptable salt or solvate thereof.

In some embodiments, for a compound of Formula (I) or (II), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea. In some embodiments, L is a bond.

In some embodiments, for a compound of Formula (I) or (II), L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH— or —CH₂N(CH₃)—. In some embodiments, L¹ is selected from a bond and 2- to 6-membered heteroalkyl. In some embodiments, L¹ is selected from a bond and C₁₋₃ alkyl. In some embodiments, L¹ is C₁₋₃ alkyl.

In some embodiments, for a compound of Formula (I) or (II), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (I) or (II), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (I) or (II), L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In certain aspects, the present disclosure provides a compound selected from:

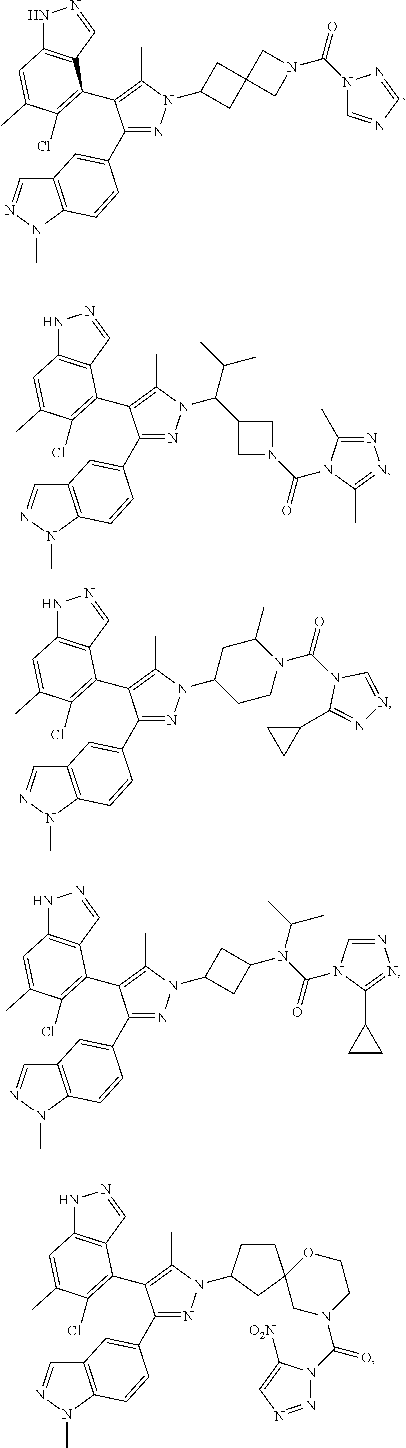

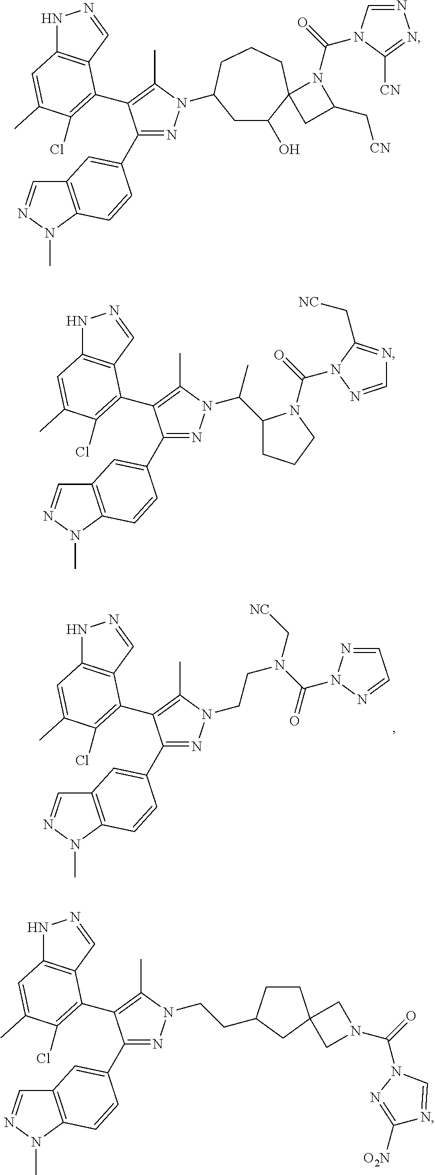

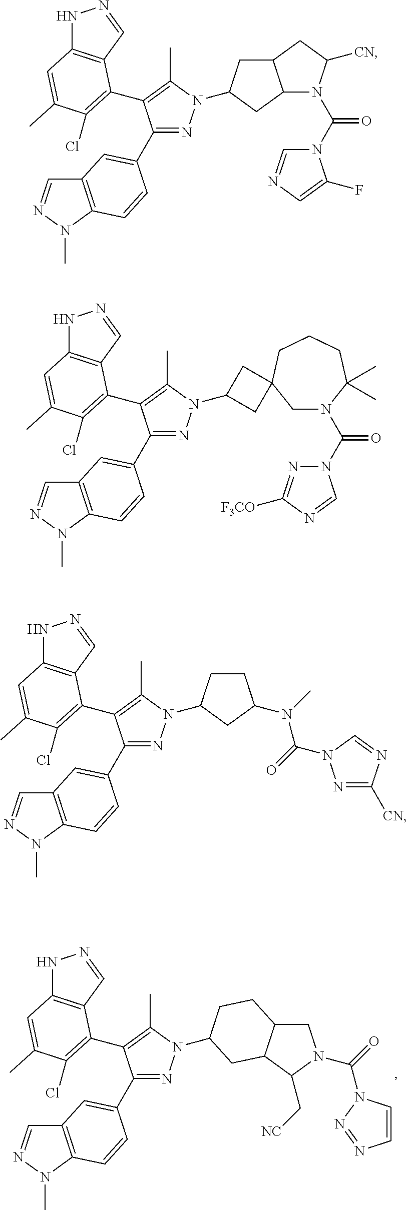 [SMILES: Cc1cc2[nH]ncc2c(-c2c(-c3ccc4c(cnn4C)c3)nn(C3CC4CC(C#N)N(C(=O)n5cncc5F)C4C3)c2C)c1Cl ; Cc1cc2[nH]ncc2c(-c2c(-c3ccc4c(cnn4C)c3)nn(C3CC4(CCCC(C)(C)N(C(=O)n5cnc(OC(F)(F)F)n5)C4)C3)c2C)c1Cl ; Cc1cc2[nH]ncc2c(-c2c(-c3ccc4c(cnn4C)c3)nn(C3CCC(N(C)C(=O)n4cnc(C#N)n4)C3)c2C)c1Cl ; Cc1cc2[nH]ncc2c(-c2c(-c3ccc4c(cnn4C)c3)nn(C3CCC4CN(C(=O)n5ccnn5)C(CC#N)C4C3)c2C)c1Cl]

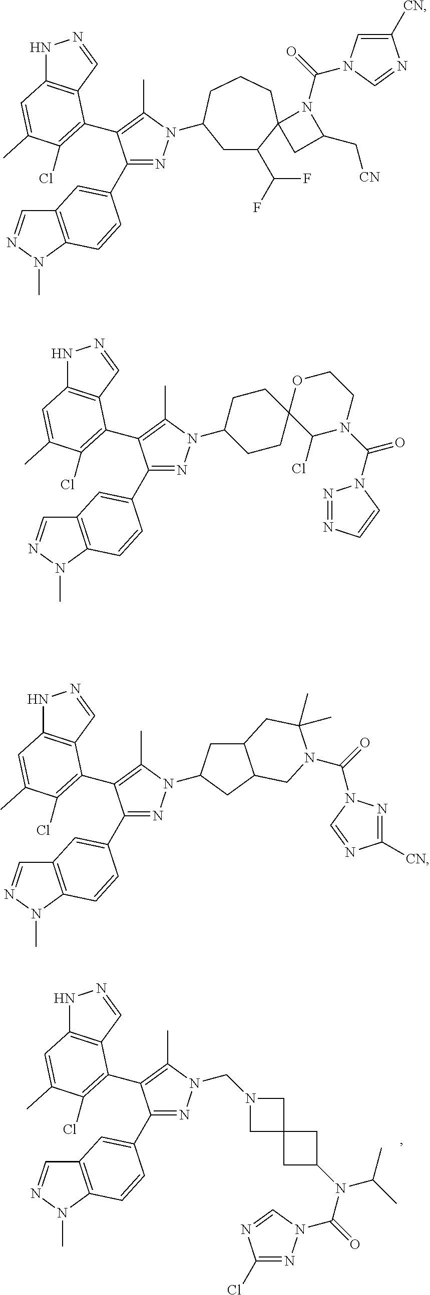 [SMILES: Cc1cc2[nH]ncc2c(-c2c(-c3ccc4c(cnn4C)c3)nn(C3CCCC4(CC(CC#N)N4C(=O)n4cnc(C#N)c4)C(C(F)F)C3)c2C)c1Cl ; Cc1cc2[nH]ncc2c(-c2c(-c3ccc4c(cnn4C)c3)nn(C3CCC4(CC3)OCCN(C(=O)n3ccnn3)C4Cl)c2C)c1Cl ; Cc1cc2[nH]ncc2c(-c2c(-c3ccc4c(cnn4C)c3)nn(C3CC4CN(C(=O)n5cnc(C#N)n5)C(C)(C)CC4C3)c2C)c1Cl ; Cc1cc2[nH]ncc2c(-c2c(-c3ccc4c(cnn4C)c3)nn(CN3CC4(CC(N(C(=O)n5cnc(Cl)n5)C(C)C)C4)C3)c2C)c1Cl]

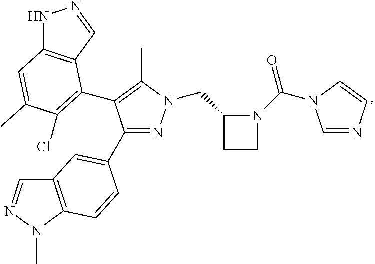 [SMILES: Cc1cc2[nH]ncc2c(-c2c(-c3ccc4c(cnn4C)c3)nn(C[C@H]3CCN3C(=O)n3ccnc3)c2C)c1Cl]

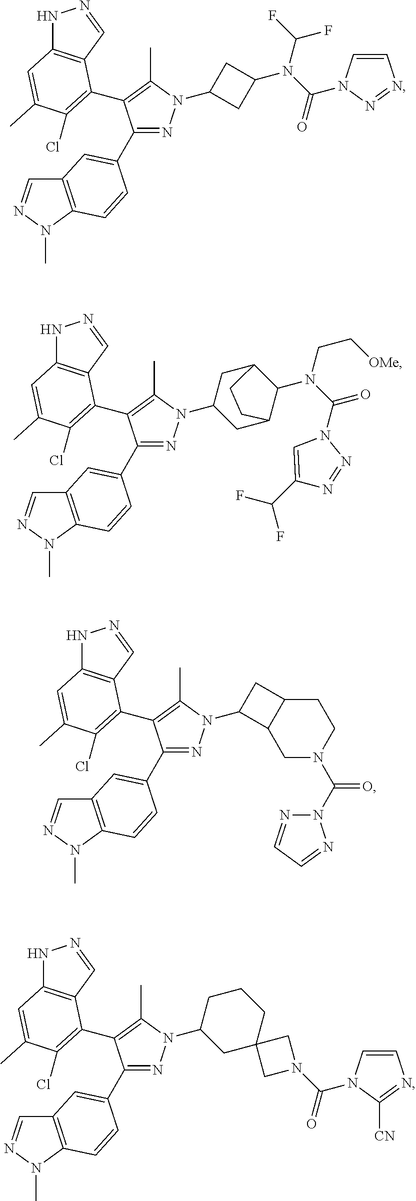 [SMILES: Cc1cc2[nH]ncc2c(-c2c(-c3ccc4c(cnn4C)c3)nn(C3CC(N(C(=O)n4ccnn4)C(F)F)C3)c2C)c1Cl ; COCCN(C(=O)n1cc(C(F)F)nn1)C1C2CCC1CC(n1nc(-c3ccc4c(cnn4C)c3)c(-c3c(Cl)c(C)cc4[nH]ncc34)c1C)C2 ; Cc1cc2[nH]ncc2c(-c2c(-c3ccc4c(cnn4C)c3)nn(C3CC4CCN(C(=O)n5nccn5)CC43)c2C)c1Cl ; Cc1cc2[nH]ncc2c(-c2c(-c3ccc4c(cnn4C)c3)nn(C3CCCC4(C3)CN(C(=O)n3ccnc3C#N)C4)c2C)c1Cl]

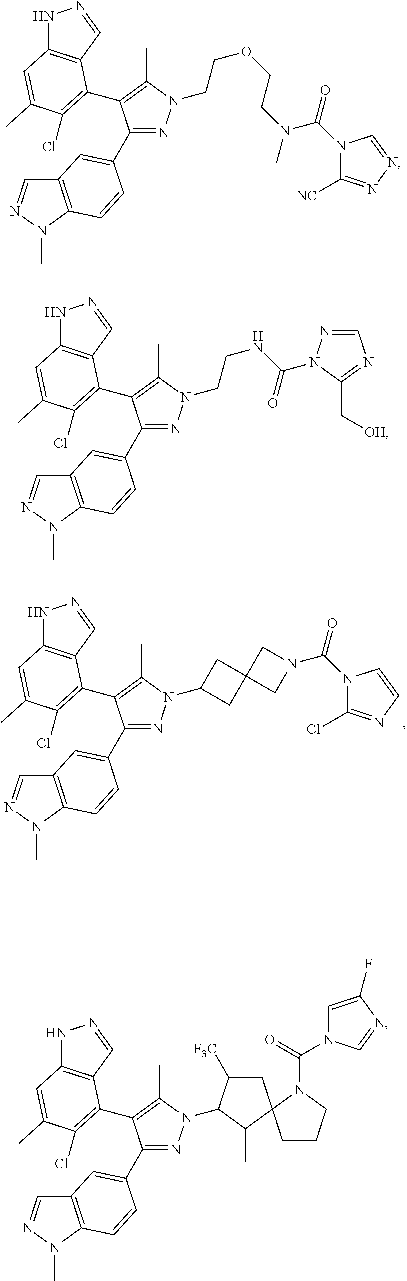 [SMILES: Cc1cc2[nH]ncc2c(-c2c(-c3ccc4c(cnn4C)c3)nn(CCOCCN(C)C(=O)n3cnnc3C#N)c2C)c1Cl ; Cc1cc2[nH]ncc2c(-c2c(-c3ccc4c(cnn4C)c3)nn(CCNC(=O)n3ncnc3CO)c2C)c1Cl ; Cc1cc2[nH]ncc2c(-c2c(-c3ccc4c(cnn4C)c3)nn(C3CC4(C3)CN(C(=O)n3ccnc3Cl)C4)c2C)c1Cl ; Cc1cc2[nH]ncc2c(-c2c(-c3ccc4c(cnn4C)c3)nn(C3C(C(F)(F)F)CC4(CCCN4C(=O)n4cnc(F)c4)C3C)c2C)c1Cl]

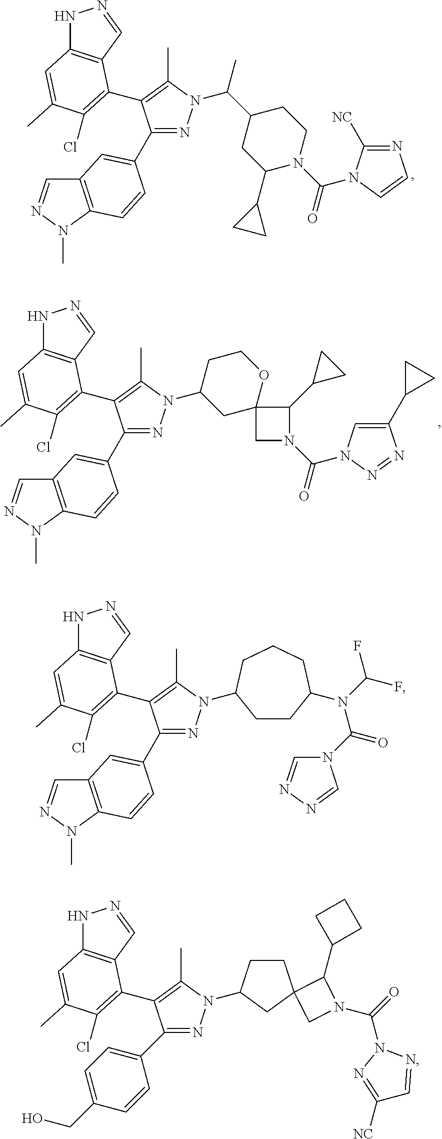

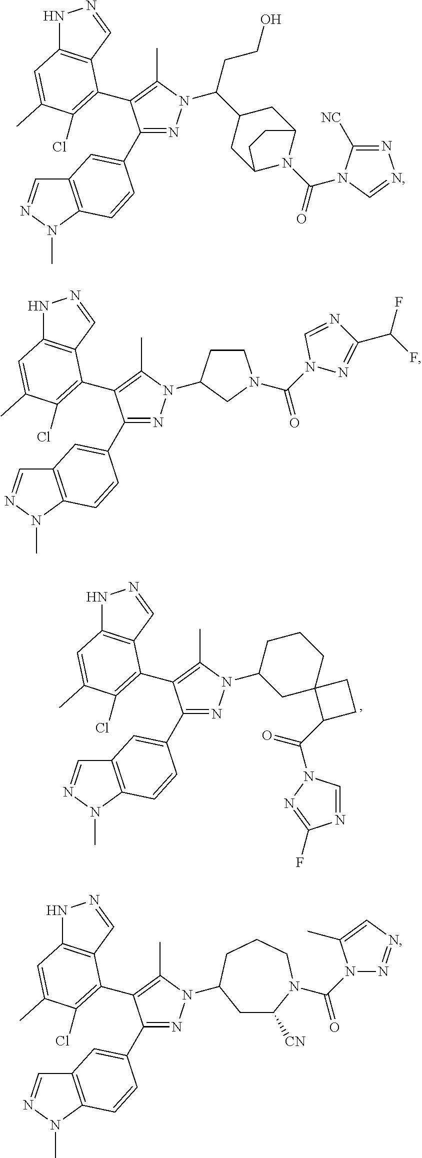

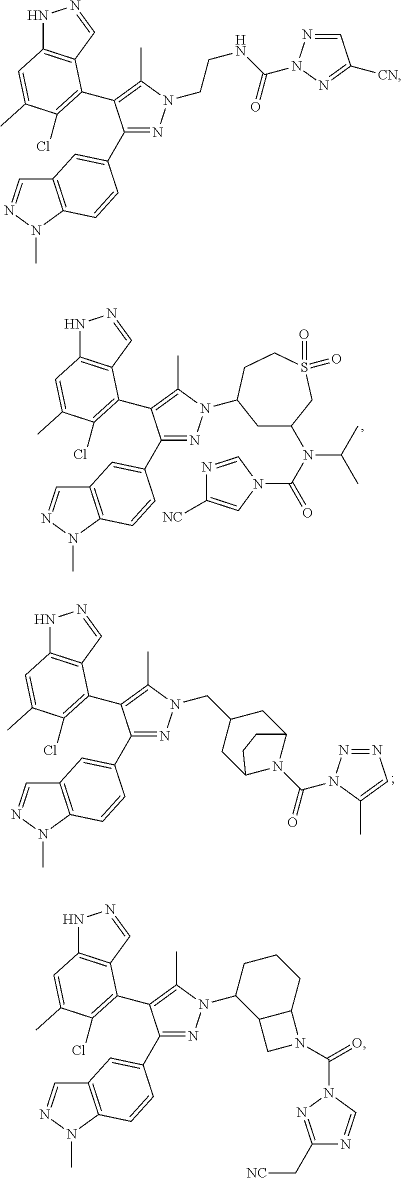

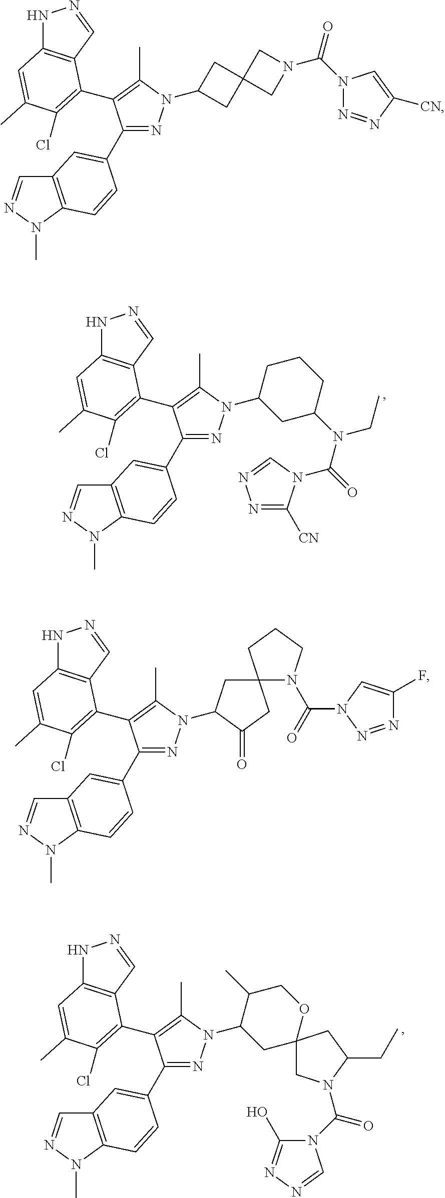

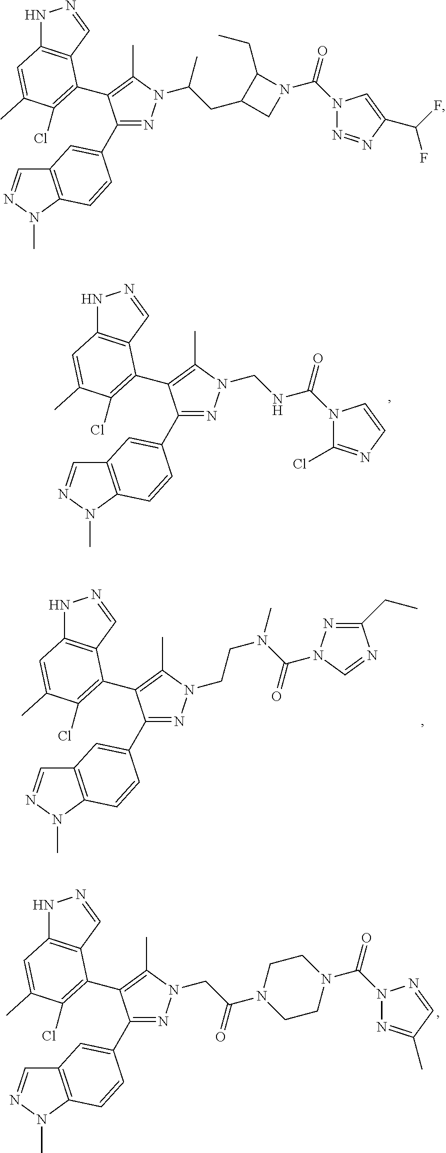

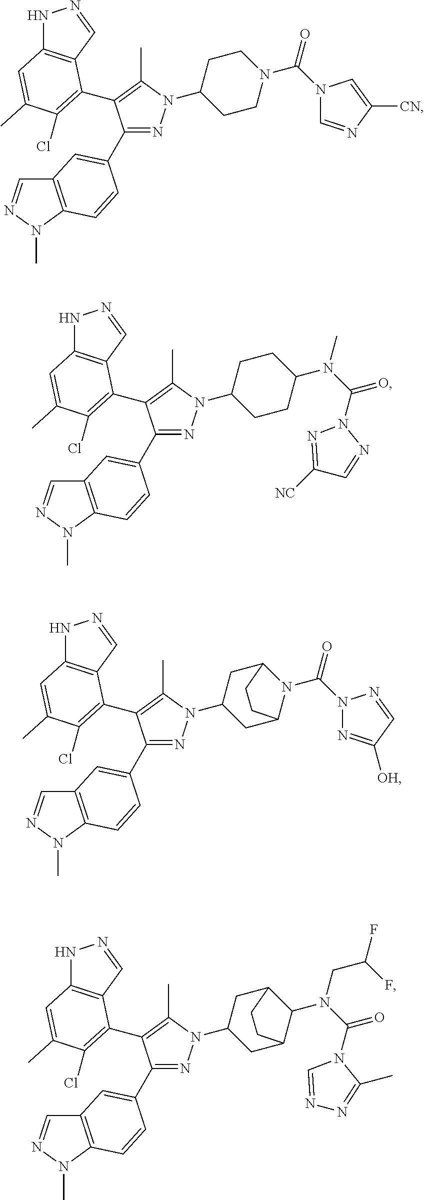

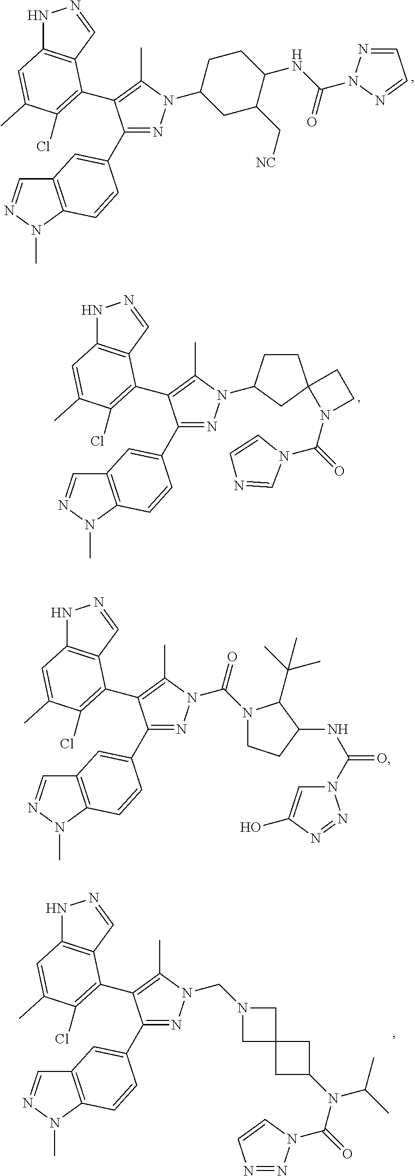

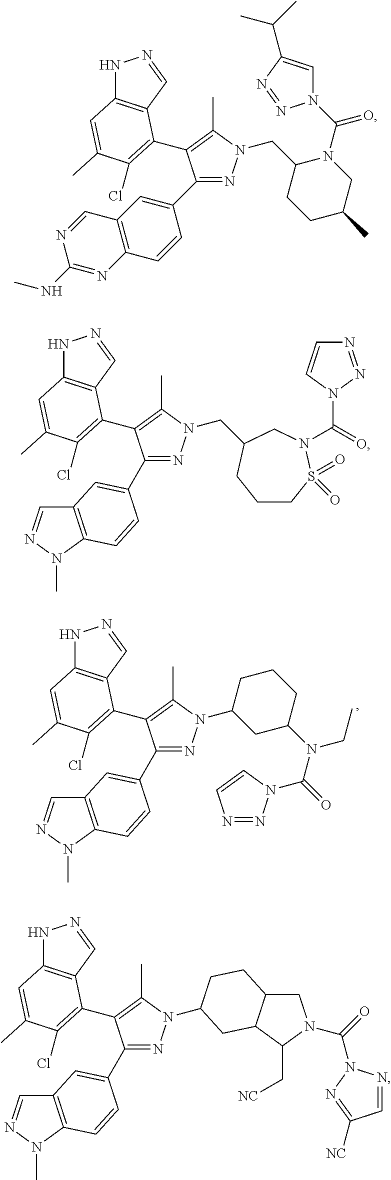

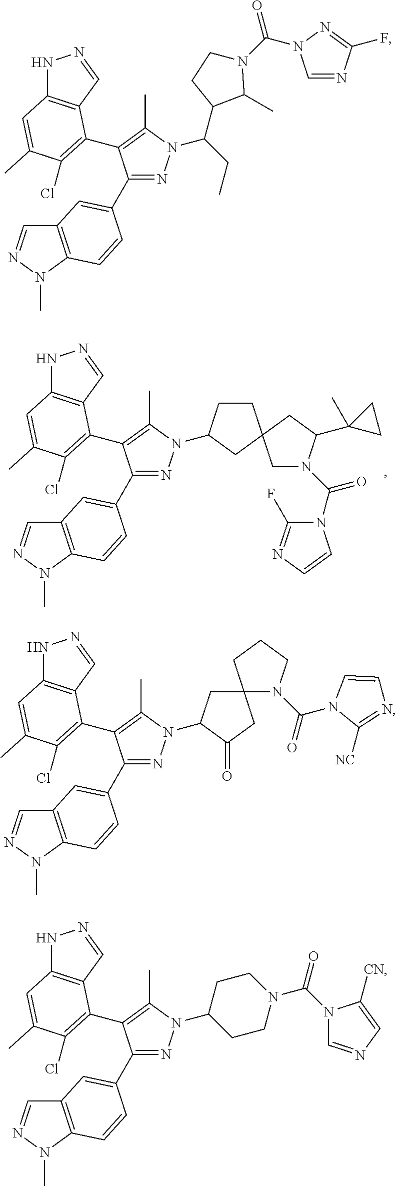

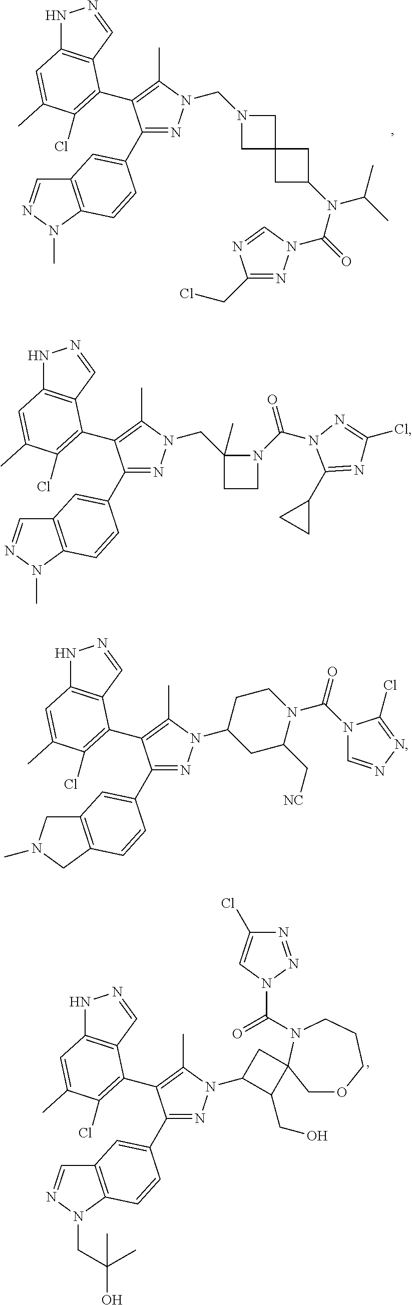

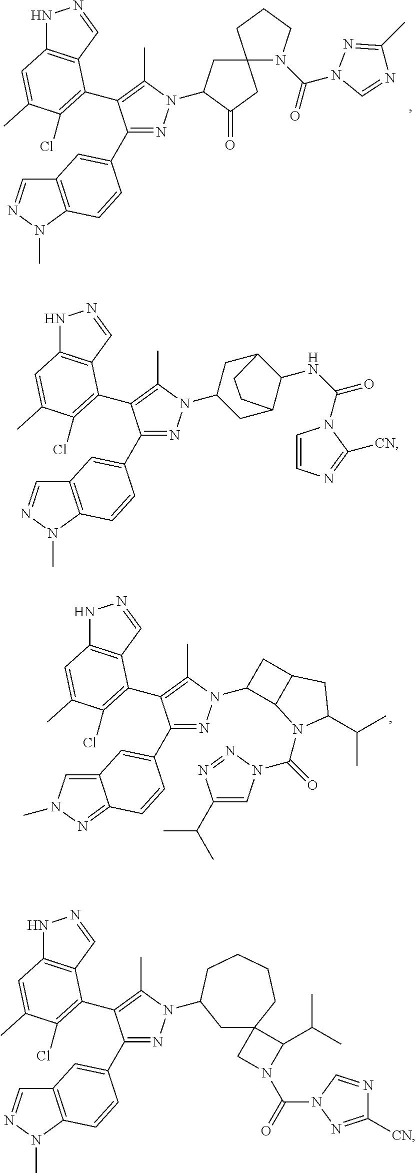

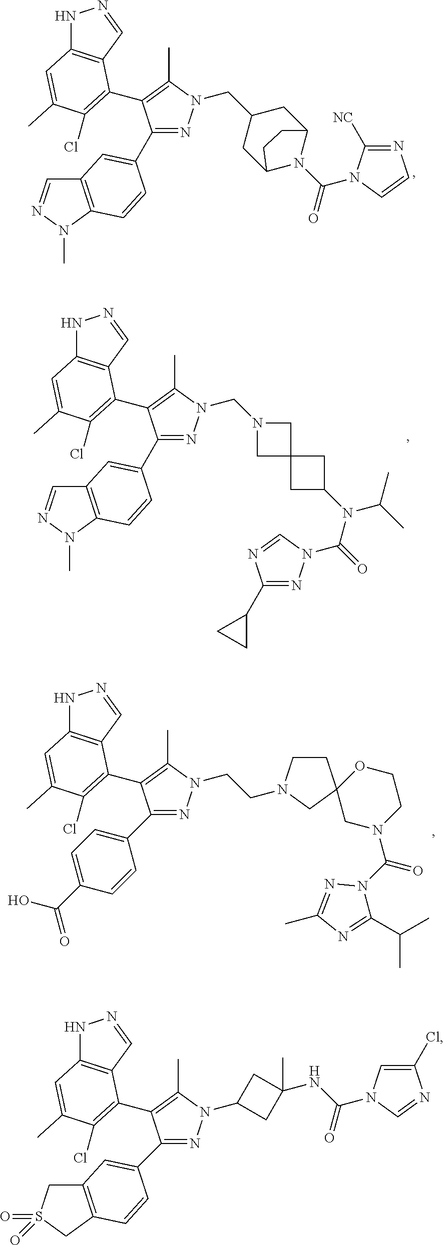 [SMILES: Cc1cc2[nH]ncc2c(-c2c(-c3ccc4c(cnn4C)c3)nn(CC3CC4CCC(C3)N4C(=O)n3ccnc3C#N)c2C)c1Cl ; Cc1cc2[nH]ncc2c(-c2c(-c3ccc4c(cnn4C)c3)nn(CN3CC4(CC(N(C(=O)n5cnc(C6CC6)n5)C(C)C)C4)C3)c2C)c1Cl ; Cc1nc(C(C)C)n(C(=O)N2CCOC3(CCN(CCn4nc(-c5ccc(C(=O)O)cc5)c(-c5c(Cl)c(C)cc6[nH]ncc56)c4C)C3)C2)n1 ; Cc1cc2[nH]ncc2c(-c2c(-c3ccc4c(c3)CS(=O)(=O)C4)nn(C3CC(C)(NC(=O)n4cnc(Cl)c4)C3)c2C)c1Cl]

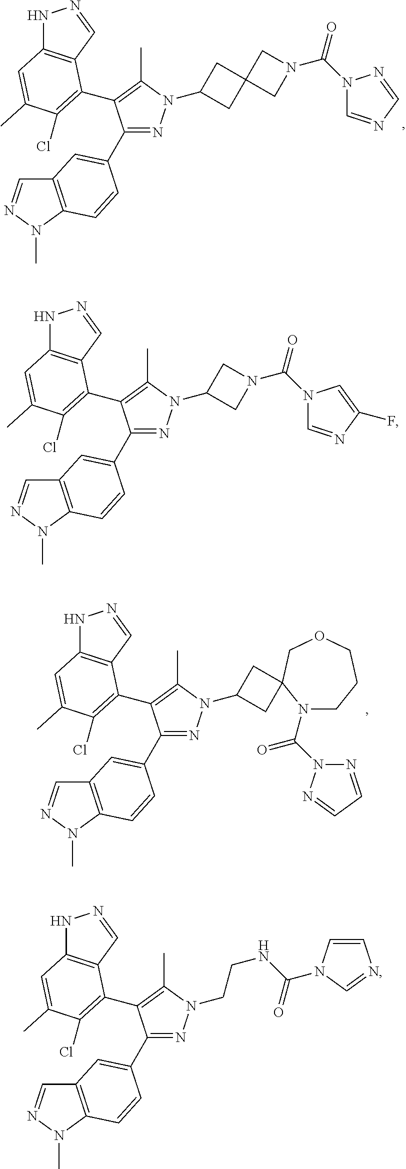 [SMILES: Cc1cc2[nH]ncc2c(-c2c(-c3ccc4c(cnn4C)c3)nn(C3CC4(C3)CN(C(=O)n3cncn3)C4)c2C)c1Cl ; Cc1cc2[nH]ncc2c(-c2c(-c3ccc4c(cnn4C)c3)nn(C3CN(C(=O)n4cnc(F)c4)C3)c2C)c1Cl ; Cc1cc2[nH]ncc2c(-c2c(-c3ccc4c(cnn4C)c3)nn(C3CC4(COCCCN4C(=O)n4nccn4)C3)c2C)c1Cl ; Cc1cc2[nH]ncc2c(-c2c(-c3ccc4c(cnn4C)c3)nn(CCNC(=O)n3ccnc3)c2C)c1Cl]

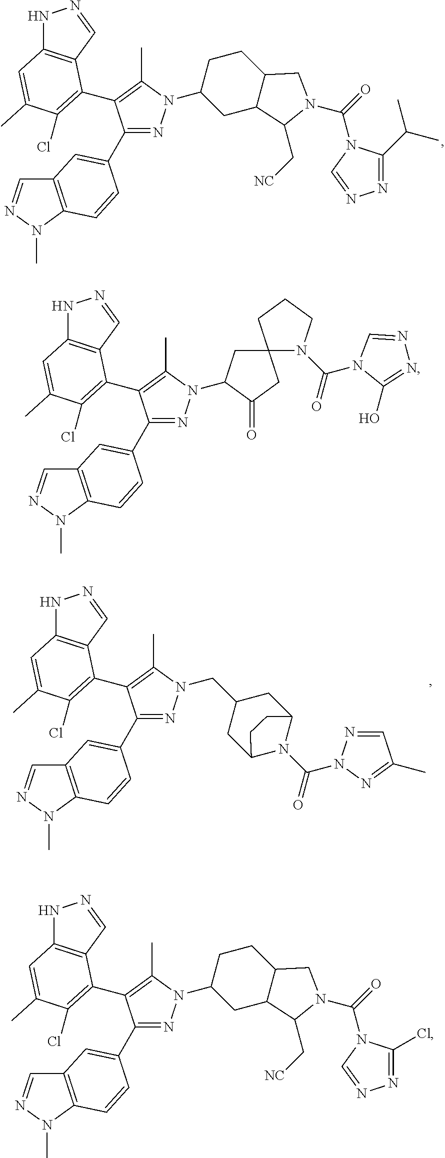

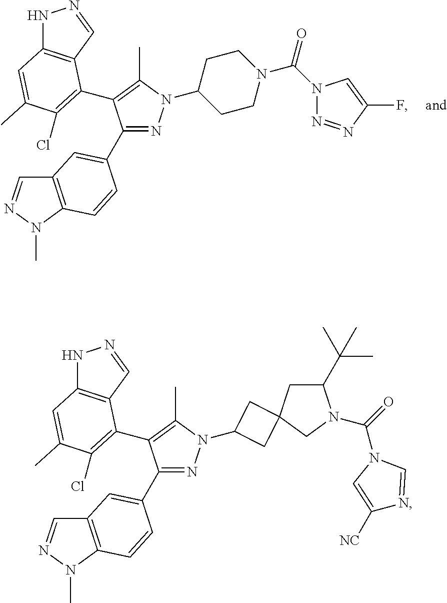 [SMILES: Cc1cc2[nH]ncc2c(-c2c(-c3ccc4c(cnn4C)c3)nn(C3CCN(C(=O)n4cc(F)nn4)CC3)c2C)c1Cl ; Cc1cc2[nH]ncc2c(-c2c(-c3ccc4c(cnn4C)c3)nn(C3CC4(C3)CC(C(C)(C)C)N(C(=O)n3cnc(C#N)c3)C4)c2C)c1Cl]

or a pharmaceutically acceptable salt, solvate, stereoisomer, or prodrug thereof.

In general, compounds of the disclosure may be prepared by the following reaction scheme:

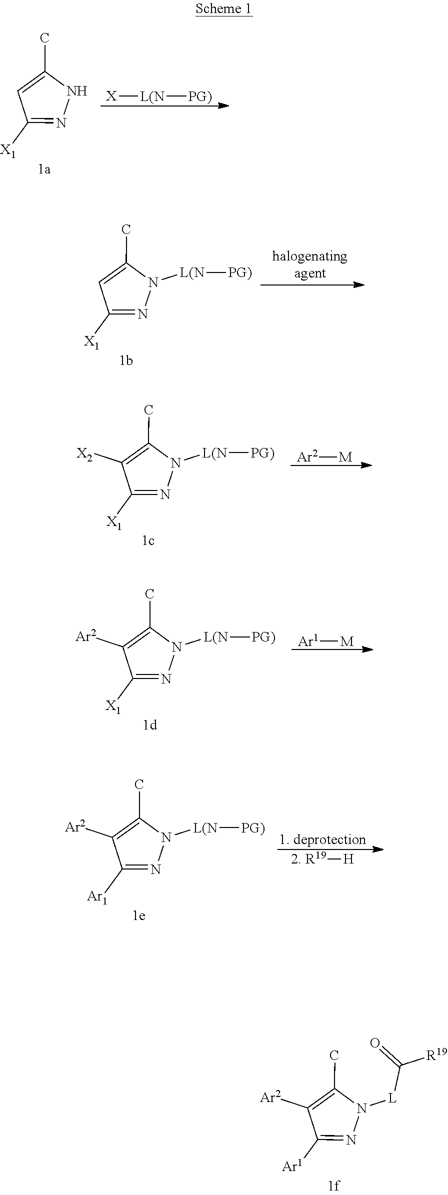

In some embodiments, a compound of Formula 1f may be prepared according to Scheme 1. For example, an appropriate halogenated heteroaromatic compound (1a) such as 3-iodo-5-methyl-1H-pyrazole may be alkylated with X-L(N-PG), wherein L comprises a suitably protected nitrogen atom (e.g., wherein PG is Boc, Cbz, or Fmoc), in the presence of a base to provide 1b. Addition of a halogenating agent can provide di-halogenated pyrazole 1c, which can undergo a cross-coupling reaction—such as a Stille or Suzuki reaction with a suitable aryl or heteroaryl coupling partner (Ar²-M)—to provide Ar²-substituted pyrazole 1d. A second cross-coupling reaction with Ar¹-M can similarly provide corresponding Ar¹-substituted derivative 1e. Removal of the N-protecting group can be followed by reaction with R¹⁹—H in the presence of triphosgene bis(trichloromethyl) carbonate (BTC) and pyridine to provide a compound of Formula 1f.

Synthetic procedures for certain intermediates, such as a compound of Formula 1a, may be found in WO 2021/124222 and WO 2021/120890, each of which is incorporated herein by reference in its entirety, including any synthetic methods, recitations of compound variables, combination therapies, and methods of use disclosed therein.

### Compounds of Formula (I) and (IV)

In certain aspects, the present disclosure provides a compound of Formula (III):

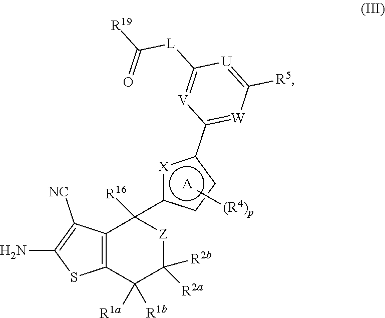

or a pharmaceutically acceptable salt or solvate thereof, wherein:
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
Z is —(CR^{6a}R^{6b})ₙ—;
X is ═C(R¹⁵)— or —N(R¹⁵)—;
R¹⁵ and R¹⁶, together with the atoms to which they are attached, form C₅₋₇ cycloalkyl or 5- to 7-membered heterocycloalkyl, each of which is optionally substituted with one or more substituents independently selected from C₁₋₄ alkyl, C₁₋₄ haloalkyl, and halogen;
W is —N═ or —CH═;
V is —N═ or —CH═;
U is —N═ or —C(R¹¹)═;
ring A is selected from pyrrole, furan, thiophene, imidazole, pyrazole, oxazole, isoxazole, thiazole, isothiazole, and triazole;
n is 0, 1, or 2;
p is 0, 1, 2, or 3;
R^{1a}, R^{1b}, R^{2a}, R^{2b}, R^{6a}, and R^{6b} are independently selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; optionally wherein one of R^{1a} or R^{1b} and one of R^{2a} or R^{2b}, together with the carbon atoms to which they are attached form a cyclopropane ring; and optionally wherein R^{6a} and R^{6b}, together with the carbon atom to which they are attached, form a cyclopropane ring;
R⁴ is selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁵ is selected from halogen, —OC₁₋₆ alkyl substituted with optionally substituted 3- to 12-membered heterocycle, and optionally substituted 3- to 12-membered heterocycle;
R¹¹ is selected from hydrogen, halogen, and C₁₋₄ alkoxy;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

In certain aspects, the present disclosure provides a compound of Formula (IV):

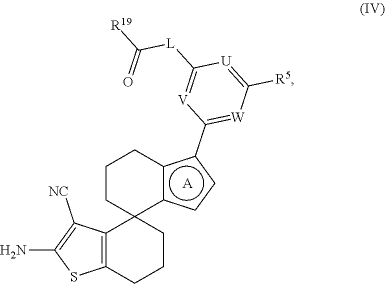

or a pharmaceutically acceptable salt or solvate thereof, wherein:
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
W is —N═ or —CH═;
V is —N═ or —CH═;
U is —N═ or —C(R¹¹)═;
ring A is selected from pyrrole, furan, thiophene, imidazole, pyrazole, oxazole, isoxazole, thiazole, isothiazole, and triazole;
R⁵ is selected from halogen, —OC₁₋₆ alkyl substituted with optionally substituted 3- to 12-membered heterocycle, and optionally substituted 3- to 12-membered heterocycle;
R¹¹ is selected from hydrogen, halogen, and C₁₋₄ alkoxy; and
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted.

In some embodiments, for a compound of Formula (III) or (IV), ring A is selected from pyrazole, isoxazole, and 1,2,3-triazole. In some embodiments, ring A is pyrazole. In some embodiments, ring A is isoxazole. In some embodiments, ring A is 1,2,3-triazole.

In some embodiments, the compound of Formula (III) is:

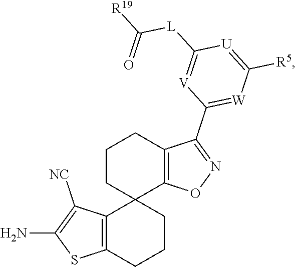

or a pharmaceutically acceptable salt or solvate thereof.

In some embodiments, for a compound of Formula (III) or (IV), W is —N═, V is —CH═, and U is —N═. In some embodiments, W is —N═, V is —N═, and U is —CH═. In some embodiments, W is —N═, V is —N═, and U is —N═. In some embodiments, R⁵ is selected from —OC₁₋₃ alkyl substituted with optionally substituted 5- to 10-membered heterocycle, and optionally substituted 5- to 10-membered heterocycle. In some embodiments, R⁵ is —OC₁₋₃ alkyl substituted with optionally substituted 5- to 10-membered heterocycle. In some embodiments, R⁵ is optionally substituted 5- to 10-membered heterocycle. In some embodiments, R⁵ is selected from

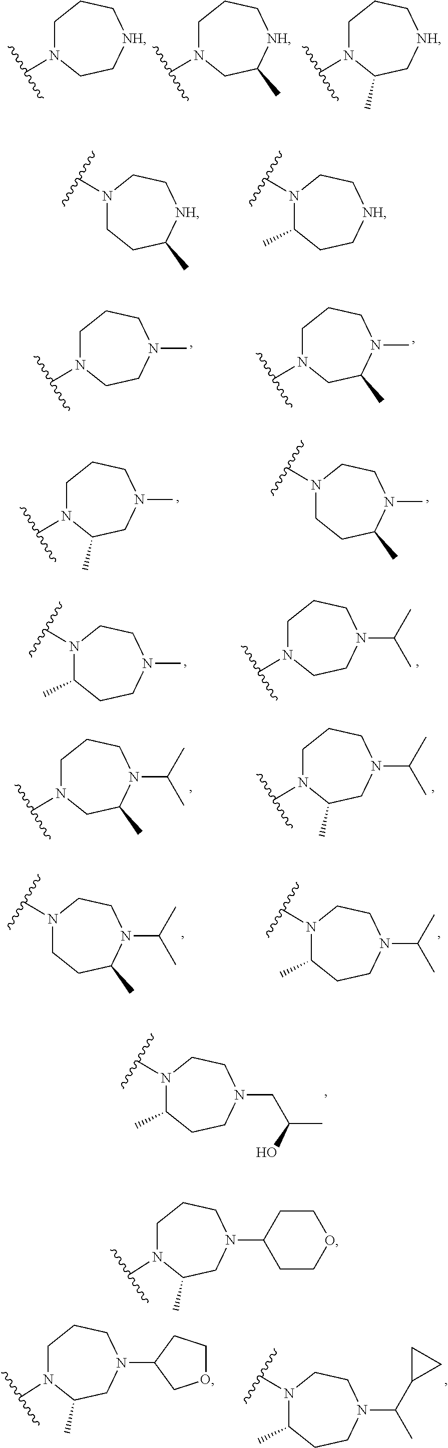

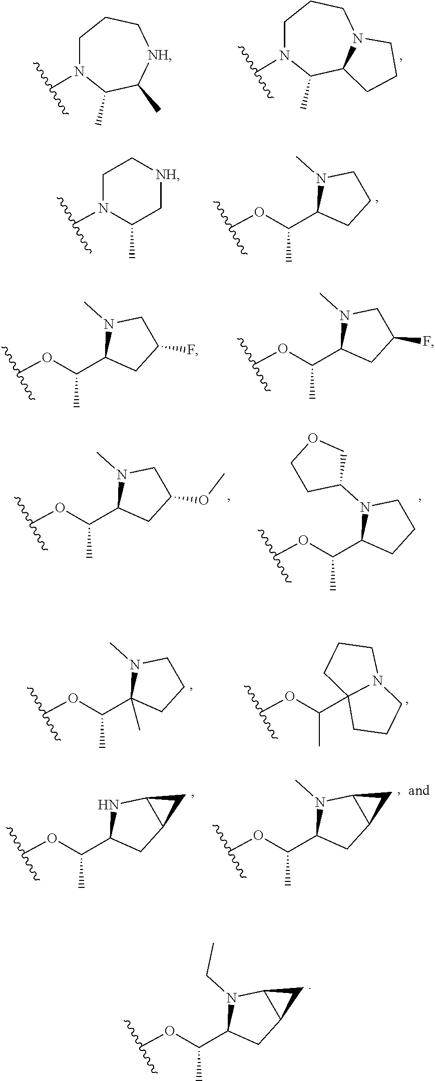

In some embodiments, the compound of Formula (III) is:

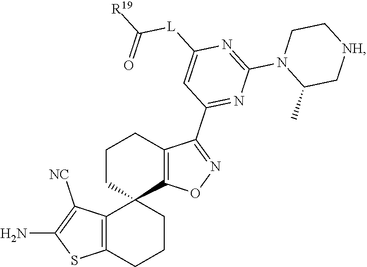

or a pharmaceutically acceptable salt or solvate thereof.

In some embodiments, for a compound of Formula (III) or (IV), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L³ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea.

In some embodiments, for a compound of Formula (III) or (IV), L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH—, —CH₂N(CH₃)—, —NHCH₂—, or —N(CH₃)CH₂—. In some embodiments, L¹ is selected from a bond, 2- to 6-membered heteroalkyl, and —N(R¹²)—.

In some embodiments, for a compound of Formula (III) or (IV), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (III) or (IV), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (III) or (IV), L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In certain aspects, the present disclosure provides a compound selected from:

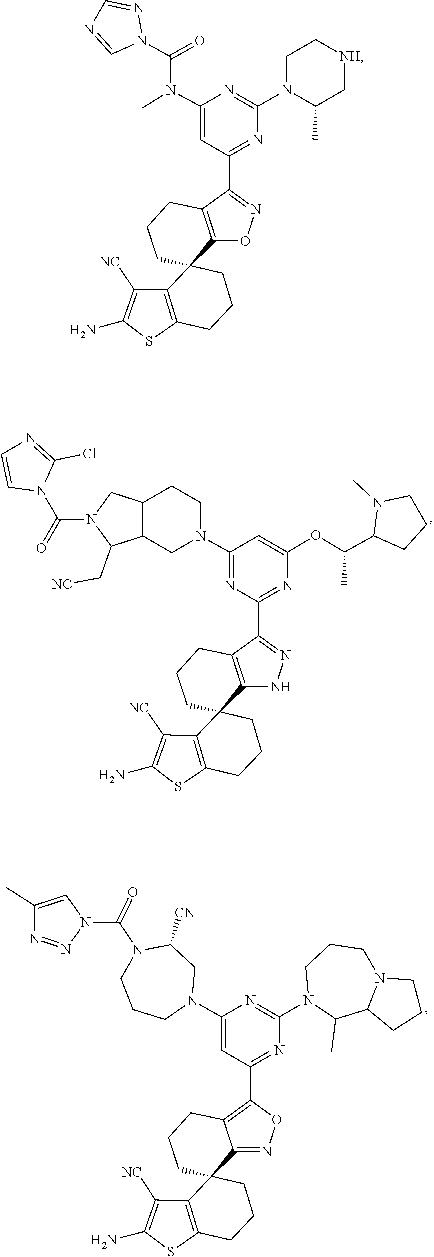 [SMILES: C[C@H]1CNCCN1c1nc(-c2noc3c2CCC[C@@]32CCCc3sc(N)c(C#N)c32)cc(N(C)C(=O)n2cncn2)n1 ; C[C@H](Oc1cc(N2CCC3CN(C(=O)n4ccnc4Cl)C(CC#N)C3C2)nc(-c2n[nH]c3c2CCC[C@@]32CCCc3sc(N)c(C#N)c32)n1)C1CCCN1C ; Cc1cn(C(=O)N2CCCN(c3cc(-c4onc5c4CCC[C@@]54CCCc5sc(N)c(C#N)c54)nc(N4CCCN5CCCC5C4C)n3)C[C@H]2C#N)nn1]

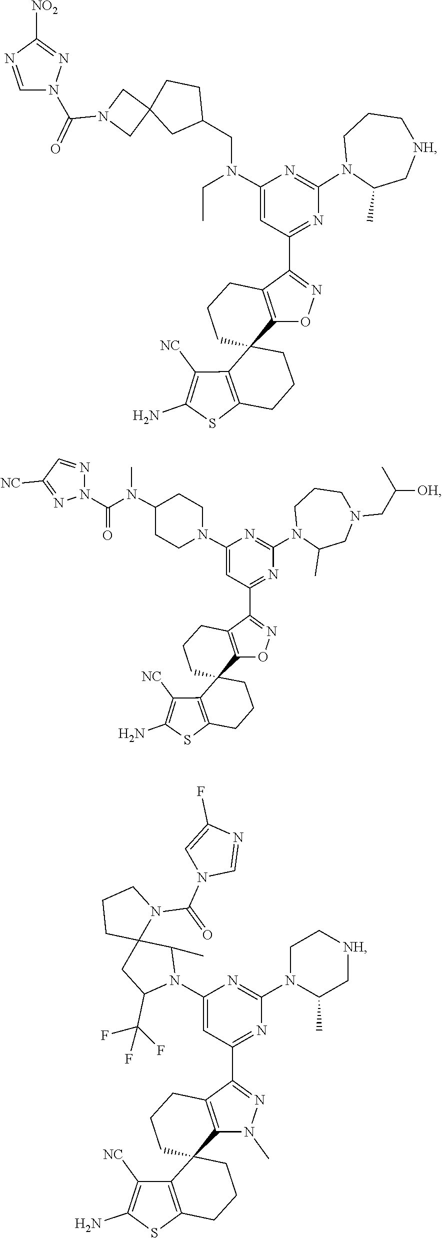 [SMILES: CCN(CC1CCC2(C1)CN(C(=O)n1cnc([N+](=O)[O-])n1)C2)c1cc(-c2noc3c2CCC[C@@]32CCCc3sc(N)c(C#N)c32)nc(N2CCCNC[C@@H]2C)n1 ; CC(O)CN1CCCN(c2nc(-c3noc4c3CCC[C@@]43CCCc4sc(N)c(C#N)c43)cc(N3CCC(N(C)C(=O)n4ncc(C#N)n4)CC3)n2)C(C)C1 ; CC1N(c2cc(-c3nn(C)c4c3CCC[C@@]43CCCc4sc(N)c(C#N)c43)nc(N3CCNC[C@@H]3C)n2)C(C(F)(F)F)CC12CCCN2C(=O)n1cnc(F)c1]

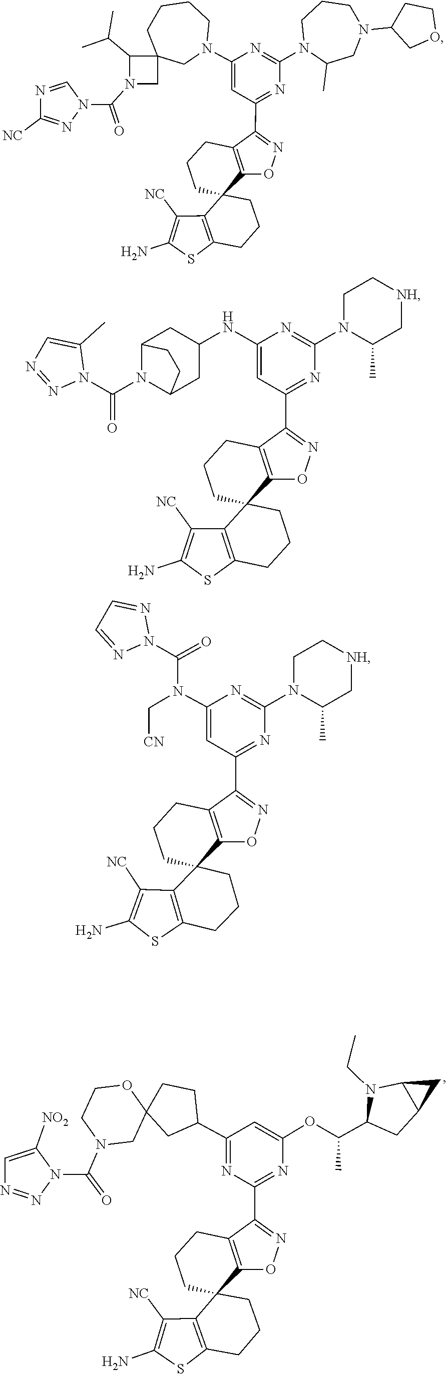

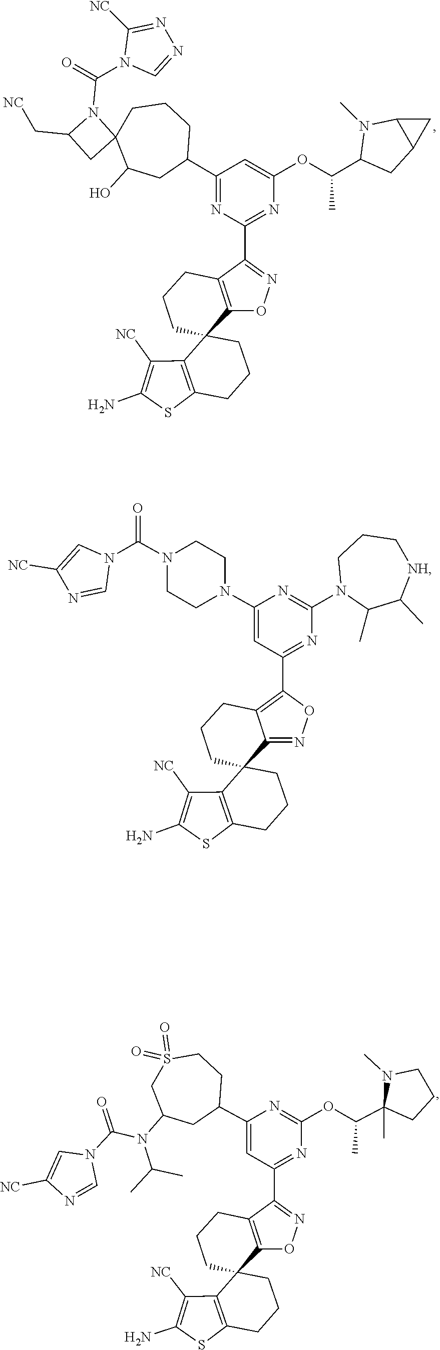 [SMILES: C[C@H](Oc1cc(C2CCCC3(CC(CC#N)N3C(=O)n3cnnc3C#N)C(O)C2)nc(-c2noc3c2CCC[C@@]32CCCc3sc(N)c(C#N)c32)n1)C1CC2CC2N1C ; CC1NCCCN(c2nc(-c3onc4c3CCC[C@@]43CCCc4sc(N)c(C#N)c43)cc(N3CCN(C(=O)n4cnc(C#N)c4)CC3)n2)C1C ; CC(C)N(C(=O)n1cnc(C#N)c1)C1CC(c2cc(-c3noc4c3CCC[C@@]43CCCc4sc(N)c(C#N)c43)nc(O[C@@H](C)[C@@]3(C)CCCN3C)n2)CCS(=O)(=O)C1]

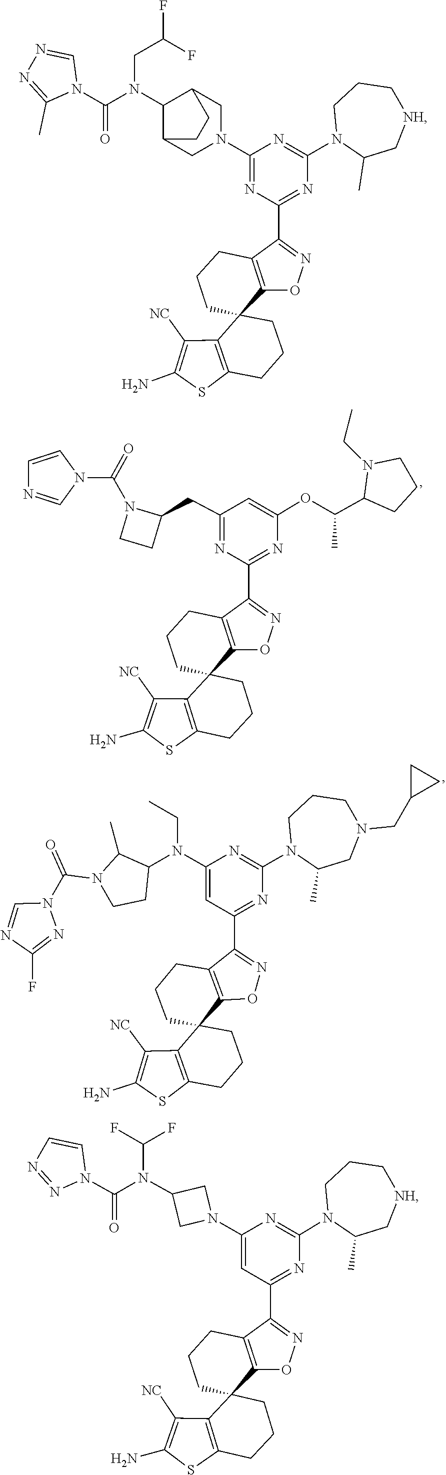

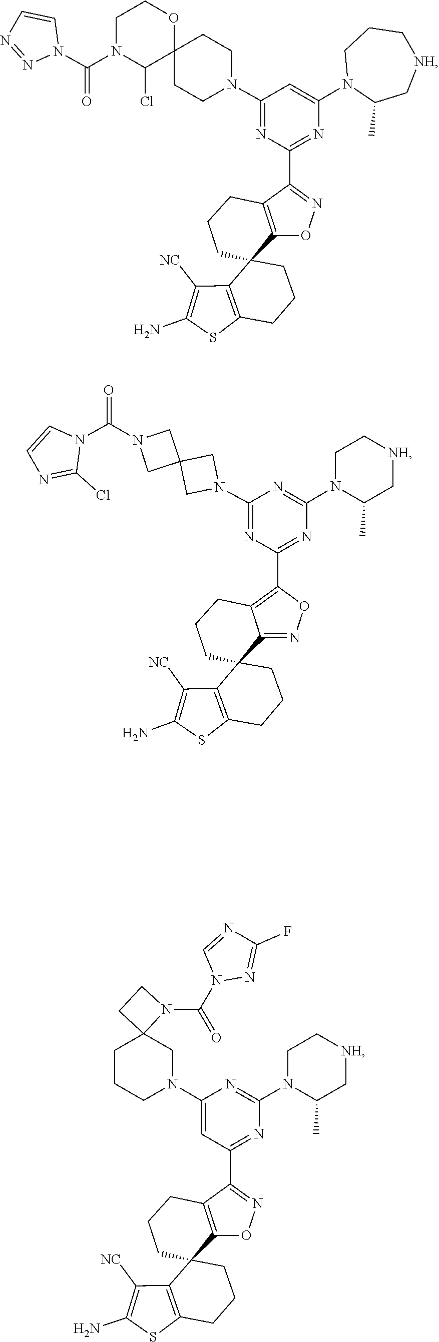 [SMILES: C[C@H]1CNCCCN1c1cc(N2CCC3(CC2)OCCN(C(=O)n2ccnn2)C3Cl)nc(-c2noc3c2CCC[C@@]32CCCc3sc(N)c(C#N)c32)n1 ; C[C@H]1CNCCN1c1nc(-c2onc3c2CCC[C@@]32CCCc3sc(N)c(C#N)c32)nc(N2CC3(CN(C(=O)n4ccnc4Cl)C3)C2)n1 ; C[C@H]1CNCCN1c1nc(-c2noc3c2CCC[C@@]32CCCc3sc(N)c(C#N)c32)cc(N2CCCC3(CCN3C(=O)n3cnc(F)n3)C2)n1]

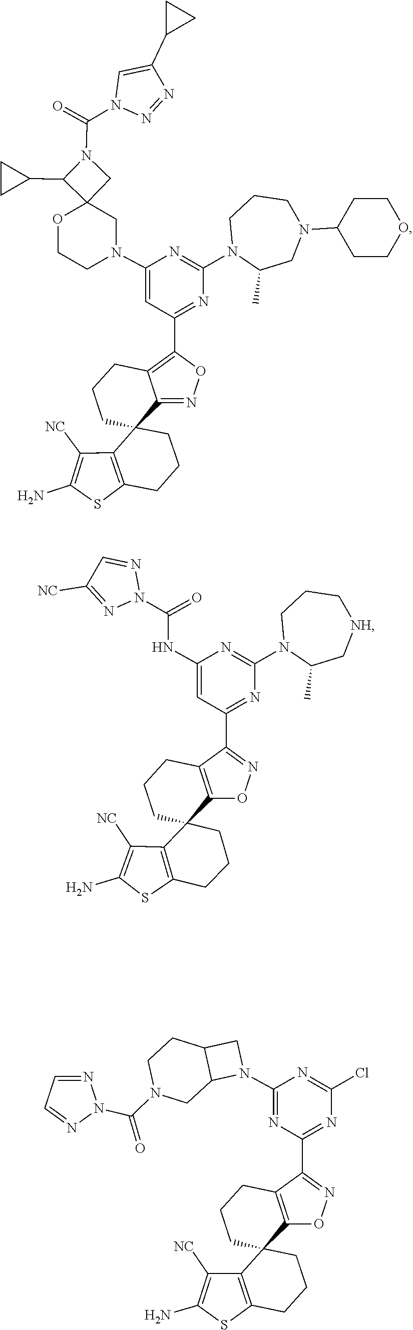 [SMILES: C[C@H]1CN(C2CCOCC2)CCCN1c1nc(-c2onc3c2CCC[C@@]32CCCc3sc(N)c(C#N)c32)cc(N2CCOC3(C2)CN(C(=O)n2cc(C4CC4)nn2)C3C2CC2)n1 ; C[C@H]1CNCCCN1c1nc(NC(=O)n2ncc(C#N)n2)cc(-c2noc3c2CCC[C@@]32CCCc3sc(N)c(C#N)c32)n1 ; N#Cc1c(N)sc2c1[C@@]1(CCC2)CCCc2c(-c3nc(Cl)nc(N4CC5CCN(C(=O)n6nccn6)CC54)n3)noc21]

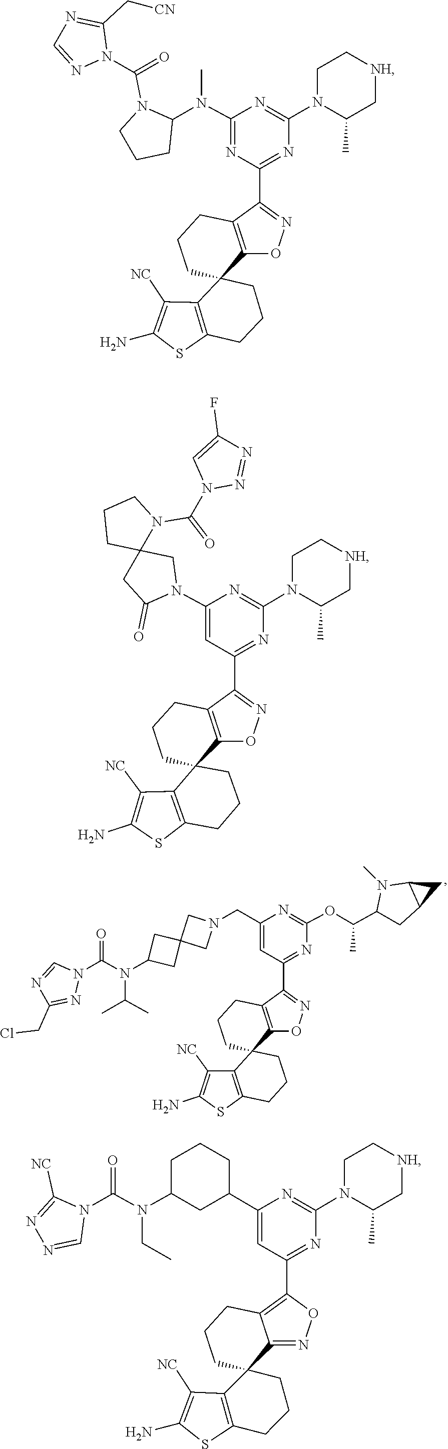

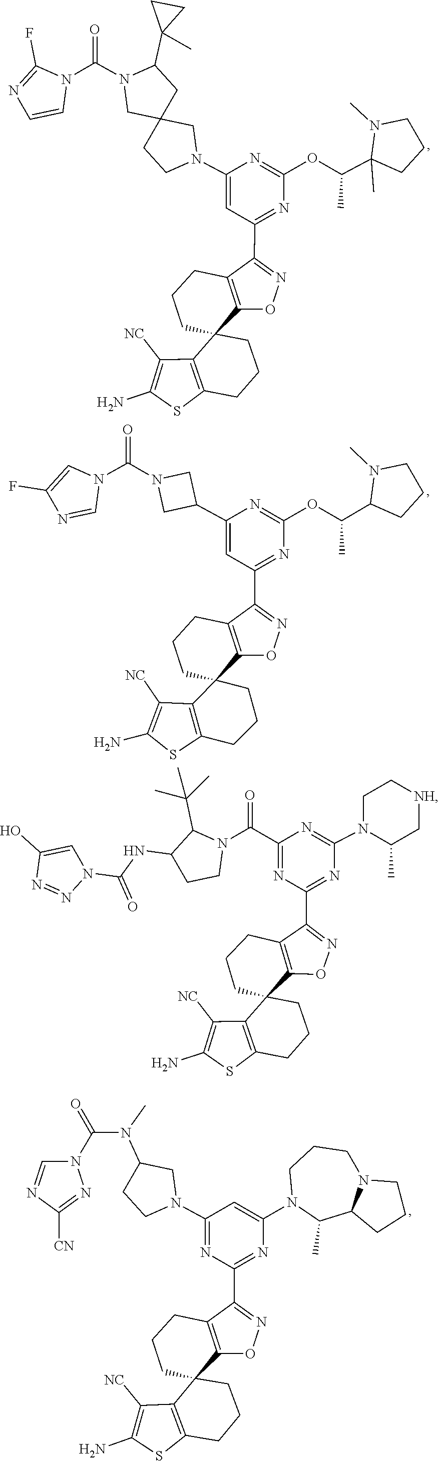

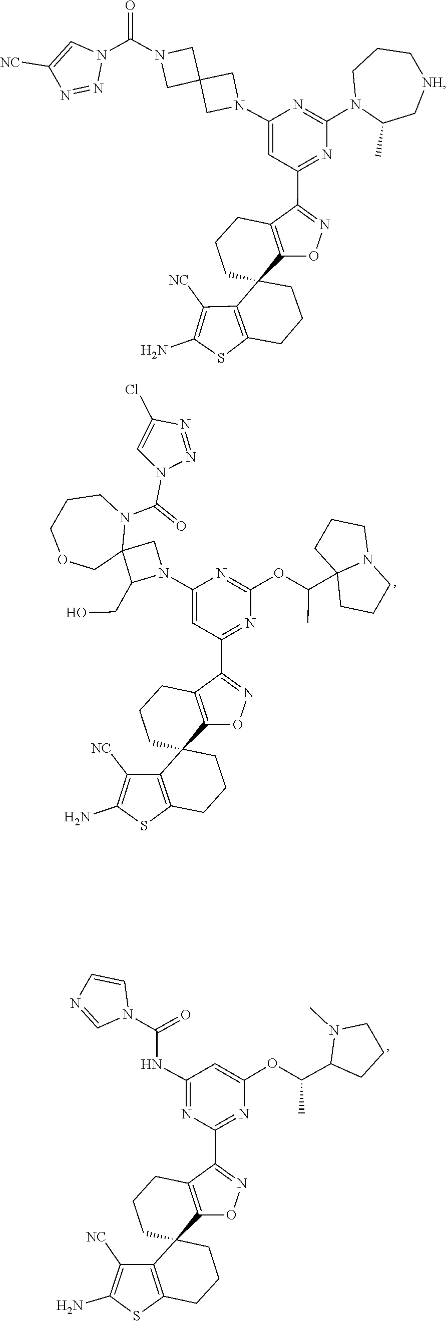 [SMILES: C[C@H]1CNCCCN1c1nc(-c2noc3c2CCC[C@@]32CCCc3sc(N)c(C#N)c32)cc(N2CC3(CN(C(=O)n4cc(C#N)nn4)C3)C2)n1 ; CC(Oc1nc(-c2noc3c2CCC[C@@]32CCCc3sc(N)c(C#N)c32)cc(N2CC3(COCCCN3C(=O)n3cc(Cl)nn3)C2CO)n1)C12CCCN1CCC2 ; C[C@H](Oc1cc(NC(=O)n2ccnc2)nc(-c2noc3c2CCC[C@@]32CCCc3sc(N)c(C#N)c32)n1)C1CCCN1C]

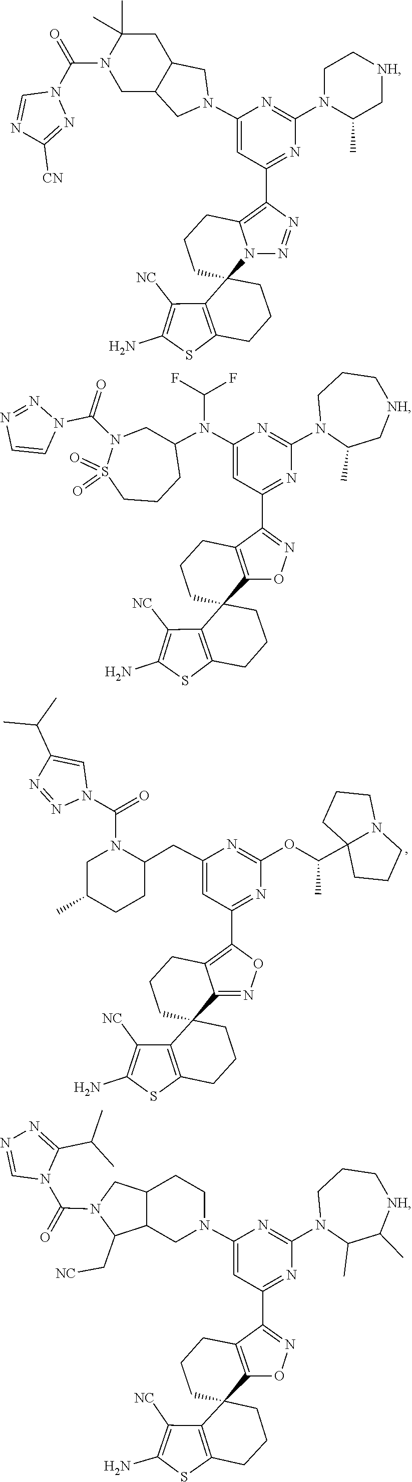

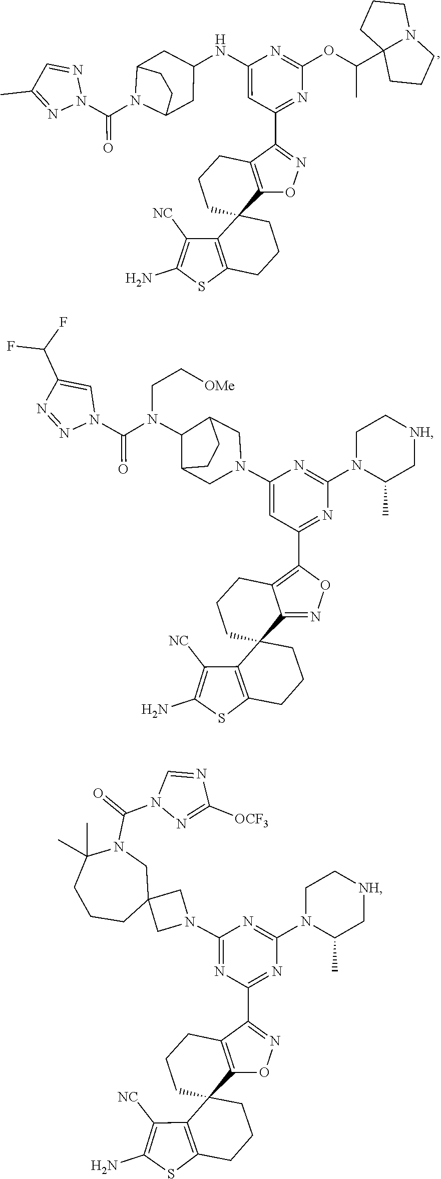 [SMILES: Cc1cnn(C(=O)N2C3CCC2CC(Nc2cc(-c4noc5c4CCC[C@@]54CCCc5sc(N)c(C#N)c54)nc(OC(C)C45CCCN4CCC5)n2)C3)n1 ; COCCN(C(=O)n1cc(C(F)F)nn1)C1C2CCC1CN(c1cc(-c3onc4c3CCC[C@@]43CCCc4sc(N)c(C#N)c43)nc(N3CCNC[C@@H]3C)n1)C2 ; C[C@H]1CNCCN1c1nc(-c2noc3c2CCC[C@@]32CCCc3sc(N)c(C#N)c32)nc(N2CC3(CCCC(C)(C)N(C(=O)n4cnc(OC(F)(F)F)n4)C3)C2)n1]

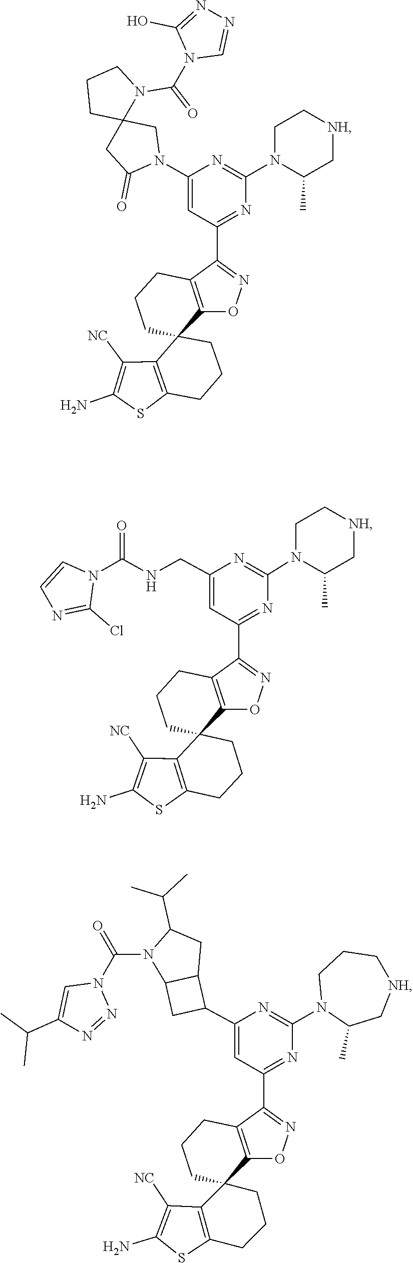 [SMILES: C[C@H]1CNCCN1c1nc(-c2noc3c2CCC[C@@]32CCCc3sc(N)c(C#N)c32)cc(N2CC3(CCCN3C(=O)n3cnnc3O)CC2=O)n1 ; C[C@H]1CNCCN1c1nc(CNC(=O)n2ccnc2Cl)cc(-c2noc3c2CCC[C@@]32CCCc3sc(N)c(C#N)c32)n1 ; CC(C)c1cn(C(=O)N2C(C(C)C)CC3C(c4cc(-c5noc6c5CCC[C@@]65CCCc6sc(N)c(C#N)c65)nc(N5CCCNC[C@@H]5C)n4)CC32)nn1]

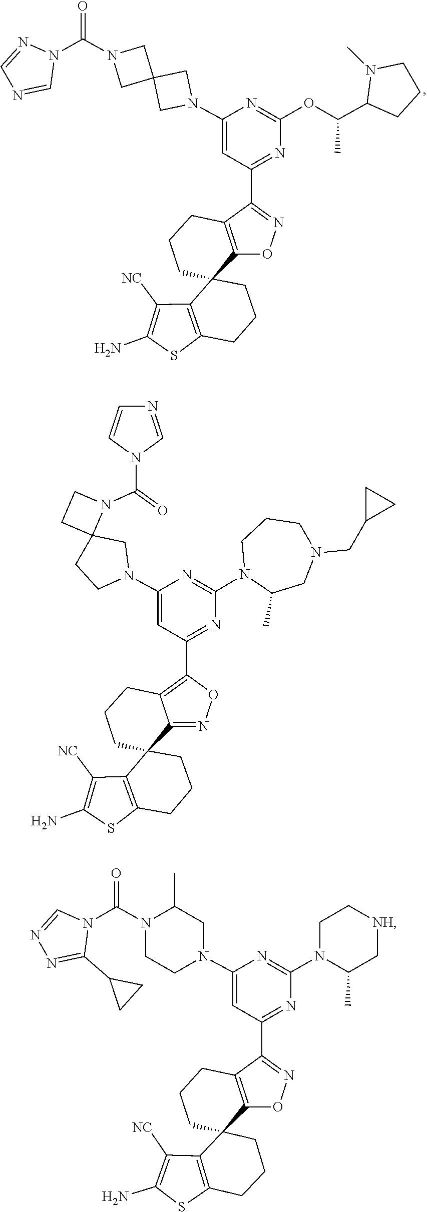

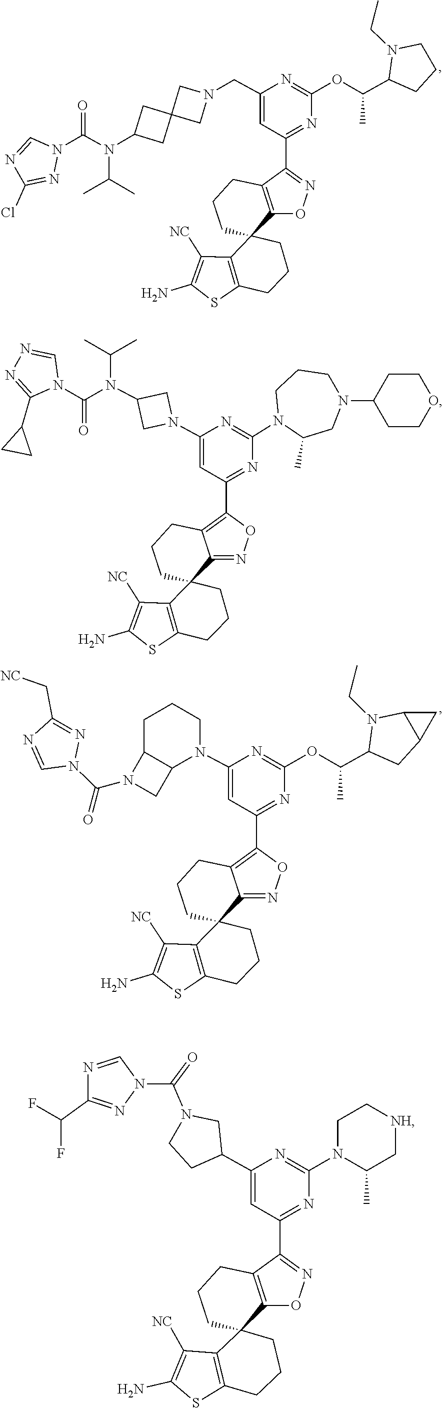 [SMILES: CCN1CCCC1[C@H](C)Oc1nc(CN2CC3(CC(N(C(=O)n4cnc(Cl)n4)C(C)C)C3)C2)cc(-c2noc3c2CCC[C@@]32CCCc3sc(N)c(C#N)c32)n1 ; CC(C)N(C(=O)n1cnnc1C1CC1)C1CN(c2cc(-c3onc4c3CCC[C@@]43CCCc4sc(N)c(C#N)c43)nc(N3CCCN(C4CCOCC4)C[C@@H]3C)n2)C1 ; CCN1C2CC2CC1[C@H](C)Oc1nc(-c2onc3c2CCC[C@@]32CCCc3sc(N)c(C#N)c32)cc(N2CCCC3C2CN3C(=O)n2cnc(CC#N)n2)n1 ; C[C@H]1CNCCN1c1nc(-c2noc3c2CCC[C@@]32CCCc3sc(N)c(C#N)c32)cc(C2CCN(C(=O)n3cnc(C(F)F)n3)C2)n1]

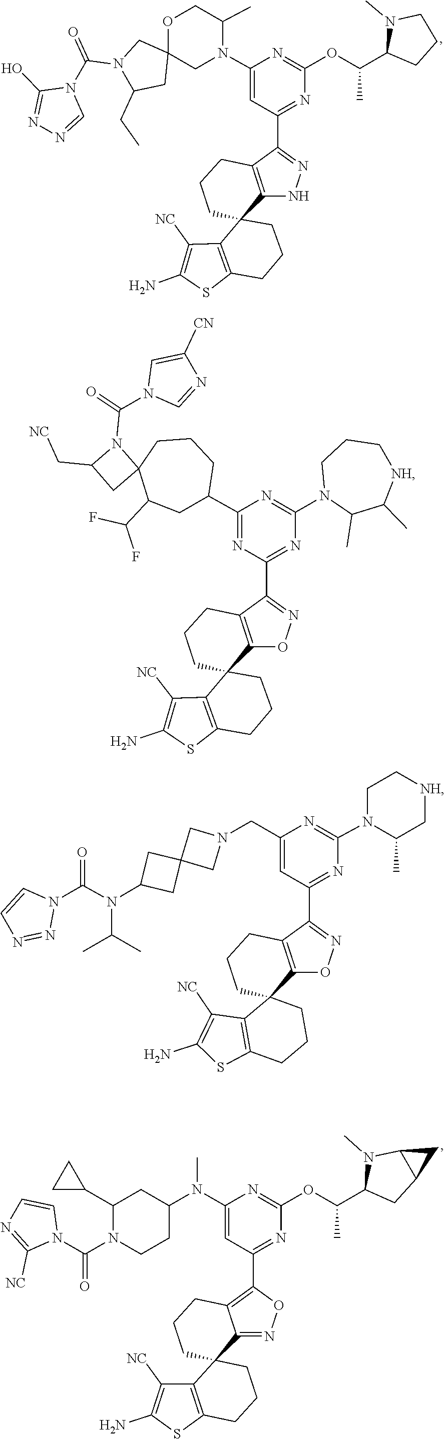

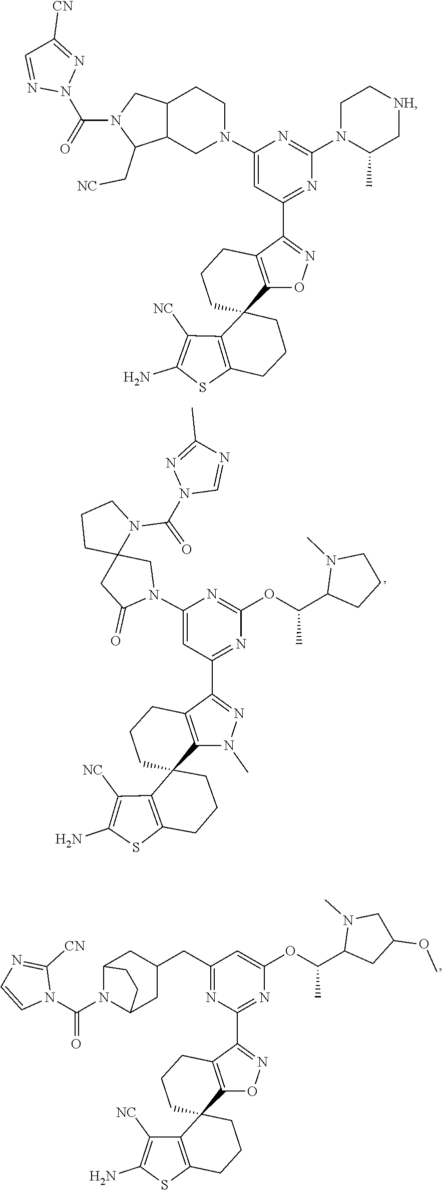

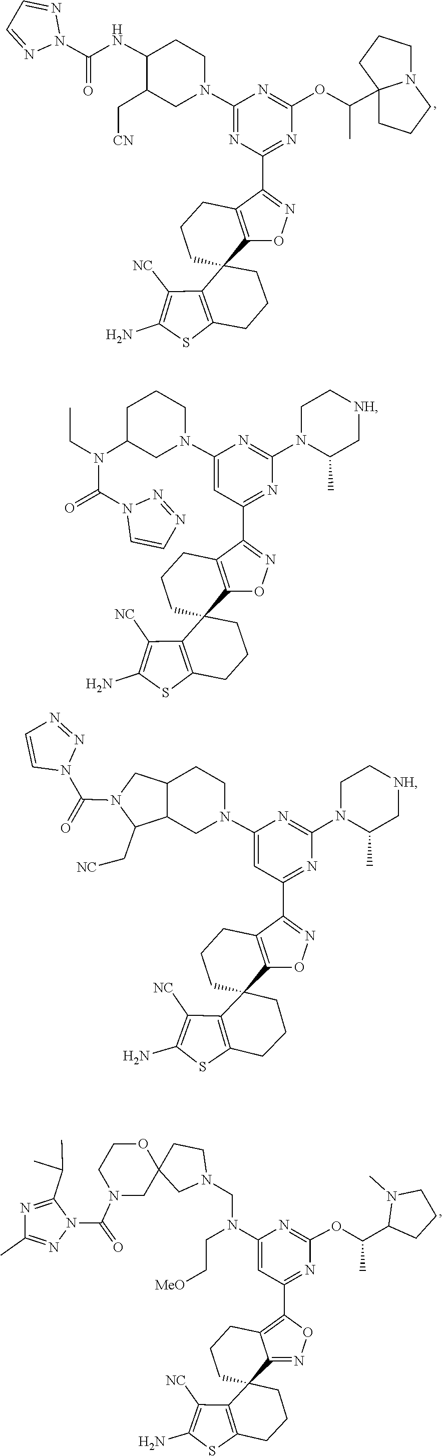 [SMILES: CC(Oc1nc(-c2noc3c2CCC[C@@]32CCCc3sc(N)c(C#N)c32)nc(N2CCC(NC(=O)n3nccn3)C(CC#N)C2)n1)C12CCCN1CCC2 ; CCN(C(=O)n1ccnn1)C1CCCN(c2cc(-c3noc4c3CCC[C@@]43CCCc4sc(N)c(C#N)c43)nc(N3CCNC[C@@H]3C)n2)C1 ; C[C@H]1CNCCN1c1nc(-c2noc3c2CCC[C@@]32CCCc3sc(N)c(C#N)c32)cc(N2CCC3CN(C(=O)n4ccnn4)C(CC#N)C3C2)n1 ; COCCN(CN1CCC2(C1)CN(C(=O)n1nc(C)nc1C(C)C)CCO2)c1cc(-c2onc3c2CCC[C@@]32CCCc3sc(N)c(C#N)c32)nc(O[C@@H](C)C2CCCN2C)n1]

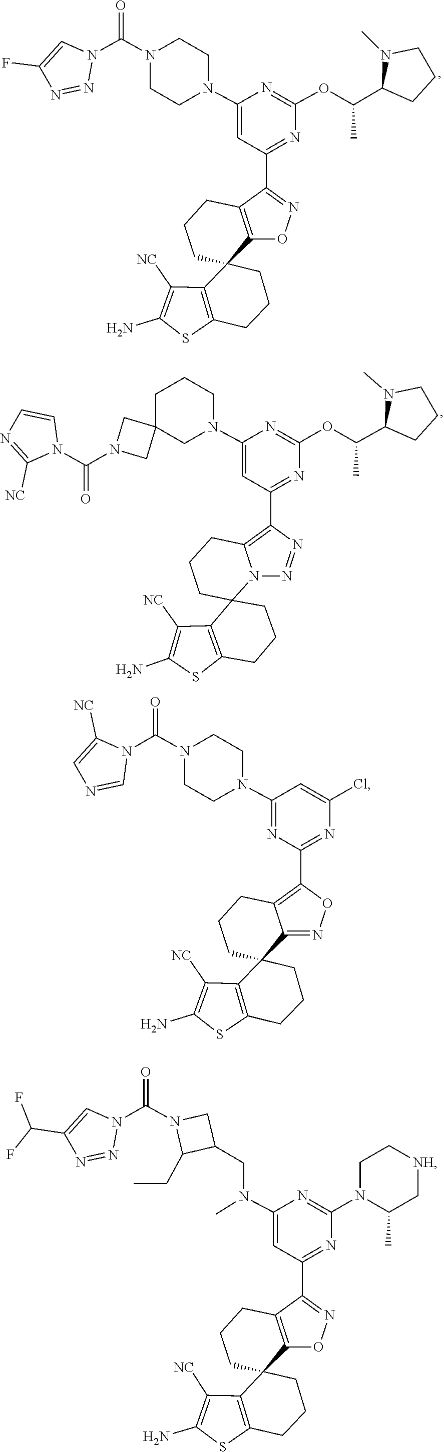

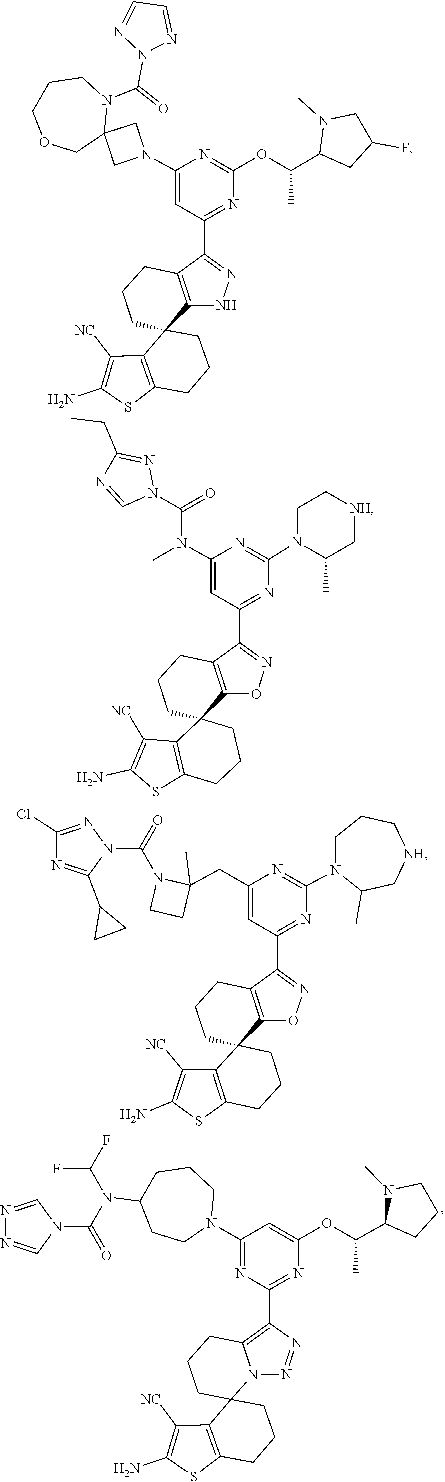

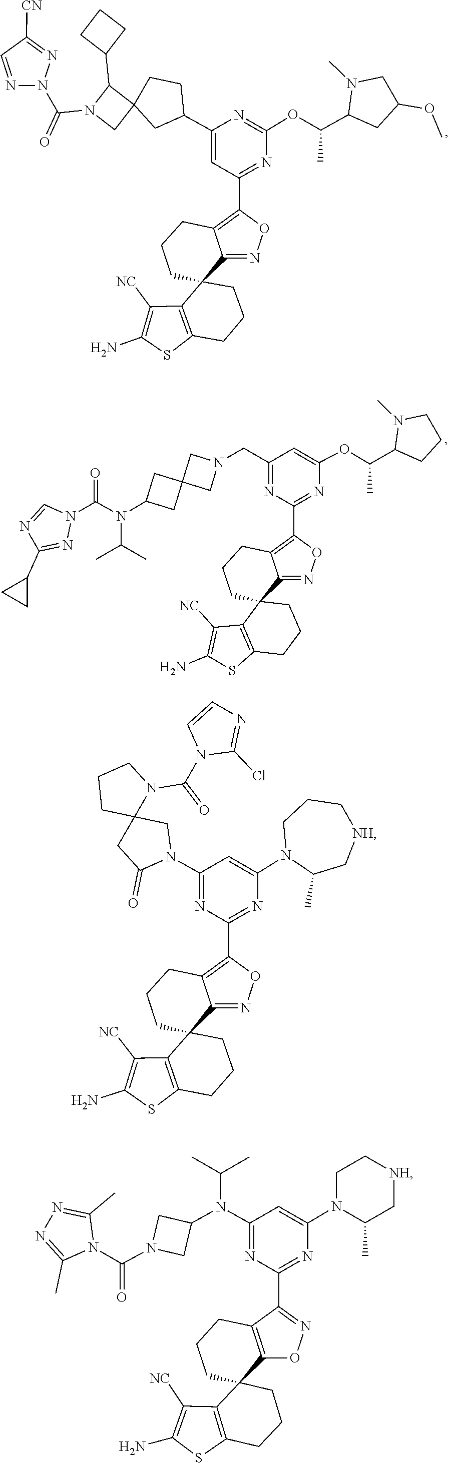

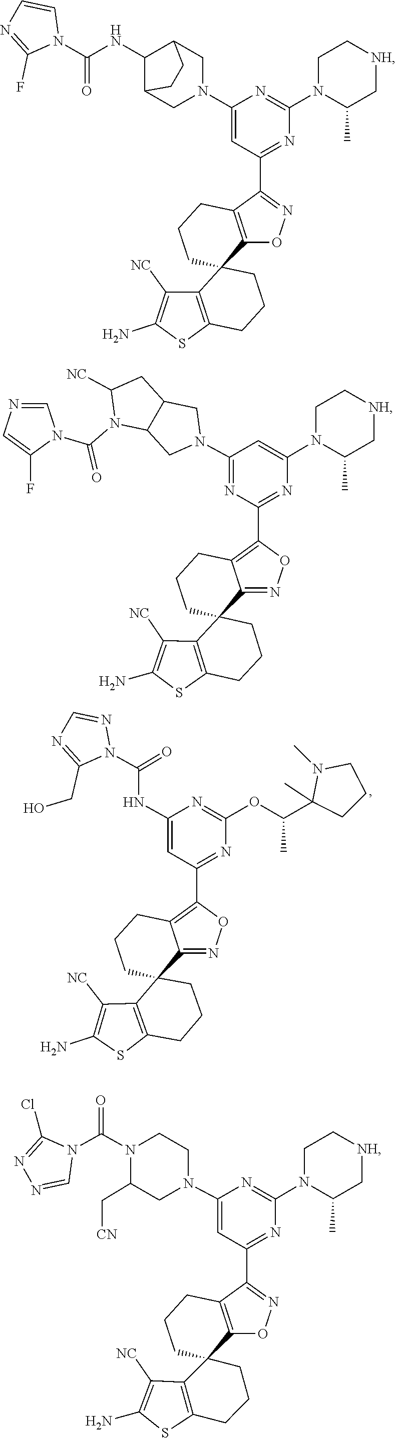

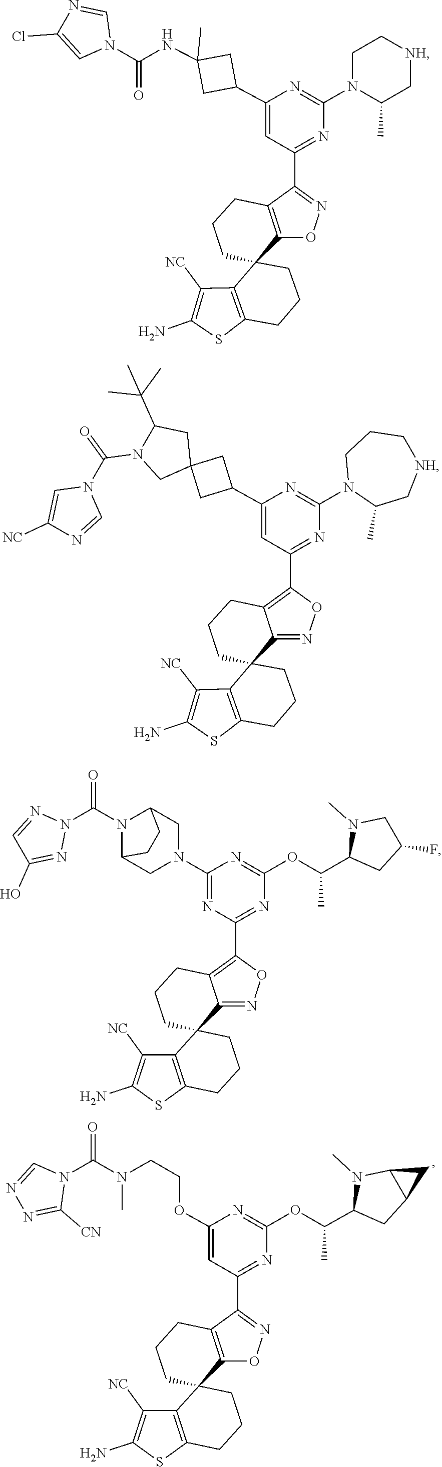

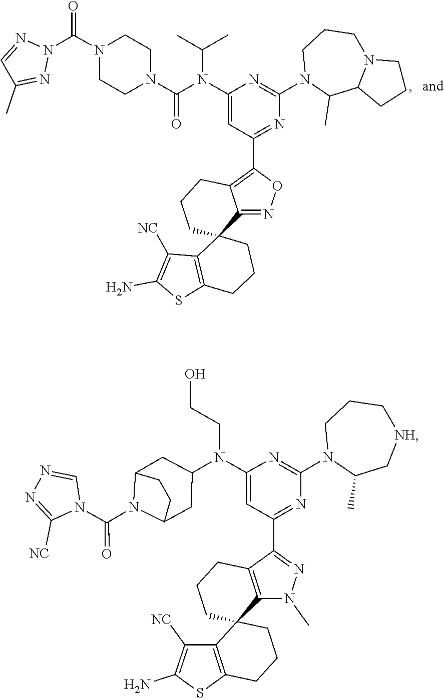 [SMILES: Cc1cnn(C(=O)N2CCN(C(=O)N(c3cc(-c4onc5c4CCC[C@@]54CCCc5sc(N)c(C#N)c54)nc(N4CCCN5CCCC5C4C)n3)C(C)C)CC2)n1 ; C[C@H]1CNCCCN1c1nc(-c2nn(C)c3c2CCC[C@@]32CCCc3sc(N)c(C#N)c32)cc(N(CCO)C2CC3CCC(C2)N3C(=O)n2cnnc2C#N)n1]

or a pharmaceutically acceptable salt, solvate, stereoisomer, or prodrug thereof.

In general, compounds of the disclosure may be prepared by the following reaction scheme:

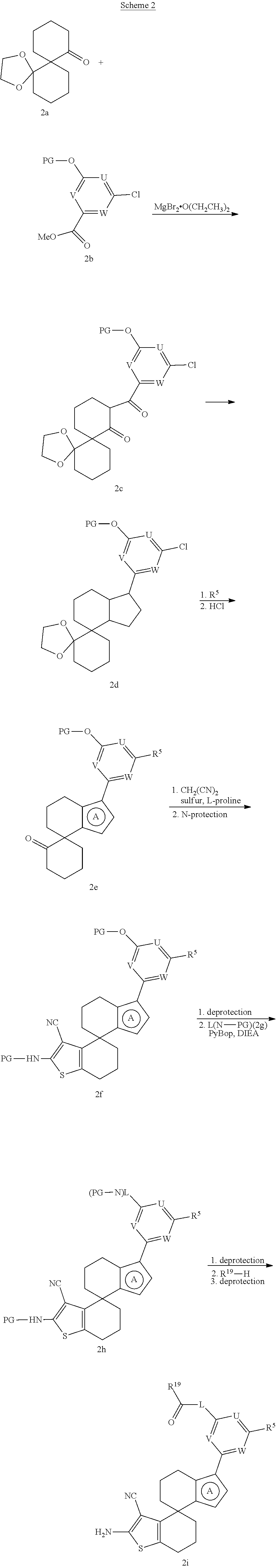

In some embodiments, a compound of Formula 2i may be prepared according to Scheme 2. For example, ketone 2a can be treated with magnesium bromide ethyl etherate, then methyl ester 2b added with a suitable base to form 2c. Following cyclization of ring A to give 2d, R⁵ substitution and deprotection of the ketone can provide 2e. The 2-amino-3-cyano thiophene can be formed and the amino group protected to provide 2f. Following a deprotection step, the resulting alcohol can be treated with 2g—wherein L comprises an unprotected primary or secondary amine, and further wherein L comprises a suitably protected nitrogen atom (e.g., wherein PG is Boc, Cbz, or Fmoc)—in the presence of PyBop and a suitable base to provide the aminated product (2h). Deprotection can be followed by reaction with R¹⁹—H in the presence of triphosgene bis(trichloromethyl) carbonate (BTC) and pyridine and a second deprotection step to provide a compound of Formula 2i.

Synthetic procedures for certain intermediates may be found in WO 2023/099623, which is incorporated herein by reference in its entirety, including any synthetic methods, recitations of compound variables, combination therapies, and methods of use disclosed therein.

### Compounds of Formula (V) and (VI)

In certain aspects, the present disclosure provides a compound of Formula (V):

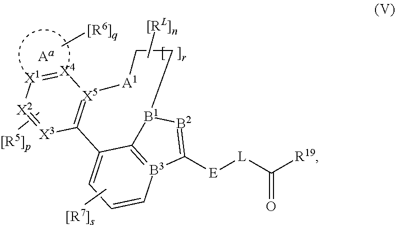

or a pharmaceutically acceptable salt or solvate thereof, wherein:
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted; E is selected from a bond and optionally substituted —C(O)-phen-1,4-diyl-;
one of B¹ and B³ is C and the other is N;
B² is N or C(R^{B}), wherein R^{B} is selected from hydrogen, C₁₋₆ alkyl, C₂₋₆ alkenyl, C₂₋₆ alkynyl, and C₃₋₆ cycloalkyl;
ring A^{a} is absent or 5- to 6-membered heteroaryl containing 1 to 3 heteroatoms selected from N, O, and S;
X¹, X², X³, X⁴, and X⁵ are independently selected from N and C, with the proviso that no more than two of X¹, X², X³, X⁴, and X⁵ are N;
R⁵, R⁶, R⁷, and R^{L} are independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and optionally wherein two geminally substituted R^{L} substituents together with the carbon atom to which they are attached form an optionally substituted 3- to 6-membered spirocyclic ring containing 0 to 1 heteroatoms selected from N, O, and S;
A¹ is selected from —CH₂—, —CH₂N(R^{L})—, —C(O)N(R^{L})—, —CH₂O—, —CH(R^{L})—, —NH—, —N(R^{L})—, —S—, —S(O)—, and —O—;
n, q, r, and s are independently 0, 1, 2, or 3;
p is 0, 1, 2, 3, or 4;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In certain aspects, the present disclosure provides a compound of Formula (VI):

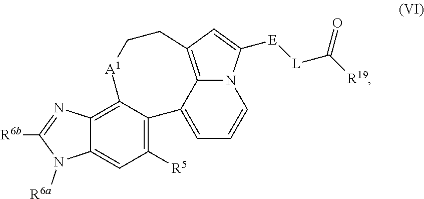

L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
E is selected from a bond and optionally substituted —C(O)-phen-1,4-diyl-;
R^{6a} and R^{6b} are independently selected from hydrogen and R⁶;
R⁵, R⁶, and R^{L} are independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
A¹ is selected from —CH₂—, —CH₂N(R^{L})—, —C(O)N(R^{L})—, —CH₂O—, —CH(R^{L})—, —NH—, —N(R^{L})—, —S—, —S(O)—, and —O—;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, the compound of Formula (V) or (VI) is selected from:

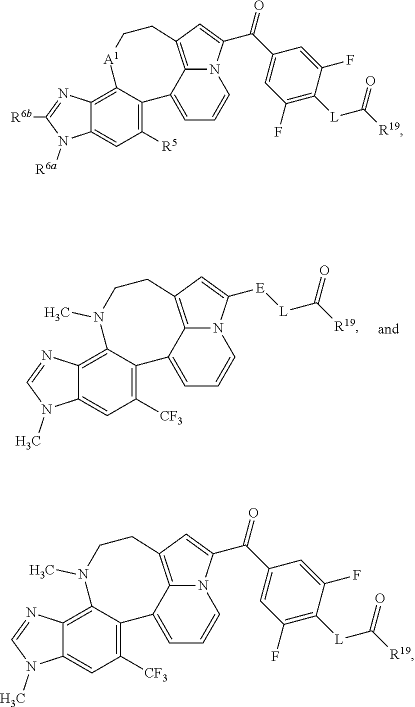

or a pharmaceutically acceptable salt or solvate thereof.

In some embodiments, for a compound of Formula (V) or (VI), A¹ is selected from —N(R^{L})—, —S—, —S(O)—, and —O—. In some embodiments, A¹ is selected from —N(CH₃)—, —S—, and —O—. In some embodiments, A¹ is —N(CH₃)—. In some embodiments, A¹ is —S—. In some embodiments, A¹ is —O—.

In some embodiments, for a compound of Formula (V) or (VI), R⁵ is selected from —CH₃, —CF₃, —OCH₃, and —OCHF₂. In some embodiments, R⁵ is selected from —CF₃ and —OCH₃. In some embodiments, R⁵ is —CH₃. In some embodiments, R⁵ is —CF₃. In some embodiments, R⁵ is —OCH₃. In some embodiments, R⁵ is —OCHF₂.

In some embodiments, for a compound of Formula (V) or (VI), R⁶ is selected from —CH₃, —CH₂OCH₃, and —CH₂CH₂OCH₃. In some embodiments, R⁶ is —CH₃. In some embodiments, R⁶ is —CH₂OCH₃. In some embodiments, R⁶ is —CH₂CH₂OCH₃. In some embodiments, q is 0, 1, or 2. In some embodiments, q is 1 or 2. In some embodiments, q is 0. In some embodiments, q is 1. In some embodiments, q is 2.

In some embodiments, for a compound of Formula (VI), R^{6a} and R^{6b} are independently selected from hydrogen, —CH₃, —CH₂OCH₃, and —CH₂CH₂OCH₃. In some embodiments, R^{6a} is selected from hydrogen, —CH₃, and —CH₂CH₂OCH₃; and R^{6b} is selected from hydrogen, —CH₃, and —CH₂OCH₃. In some embodiments, R^{6a} is —CH₃ and R¹ is hydrogen. In some embodiments, R^{6a} is —CH₂CH₂OCH₃ and R^{6b} is hydrogen. In some embodiments, R^{6a} is —CH₂CH₂OCH₃ and R^{6b} is —CH₃. In some embodiments, R^{6a} is —CH₃ and R^{6b} is —CH₃. In some embodiments, R^{6a} is —CH₃ and R^{6b} is —CH₂OCH₃.

In some embodiments, for a compound of Formula (V) or (VI), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea. In some embodiments, L is a bond.

In some embodiments, for a compound of Formula (V) or (VI), L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH—, —CH₂N(CH₃)—, —NHCH₂—, or —N(CH₃)CH₂—. In some embodiments, L¹ is selected from a bond, 2- to 6-membered heteroalkyl, and —N(R¹²)—.

In some embodiments, for a compound of Formula (V) or (VI), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (V) or (VI), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (V) or (VI), L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In certain aspects, the present disclosure provides a compound selected from:

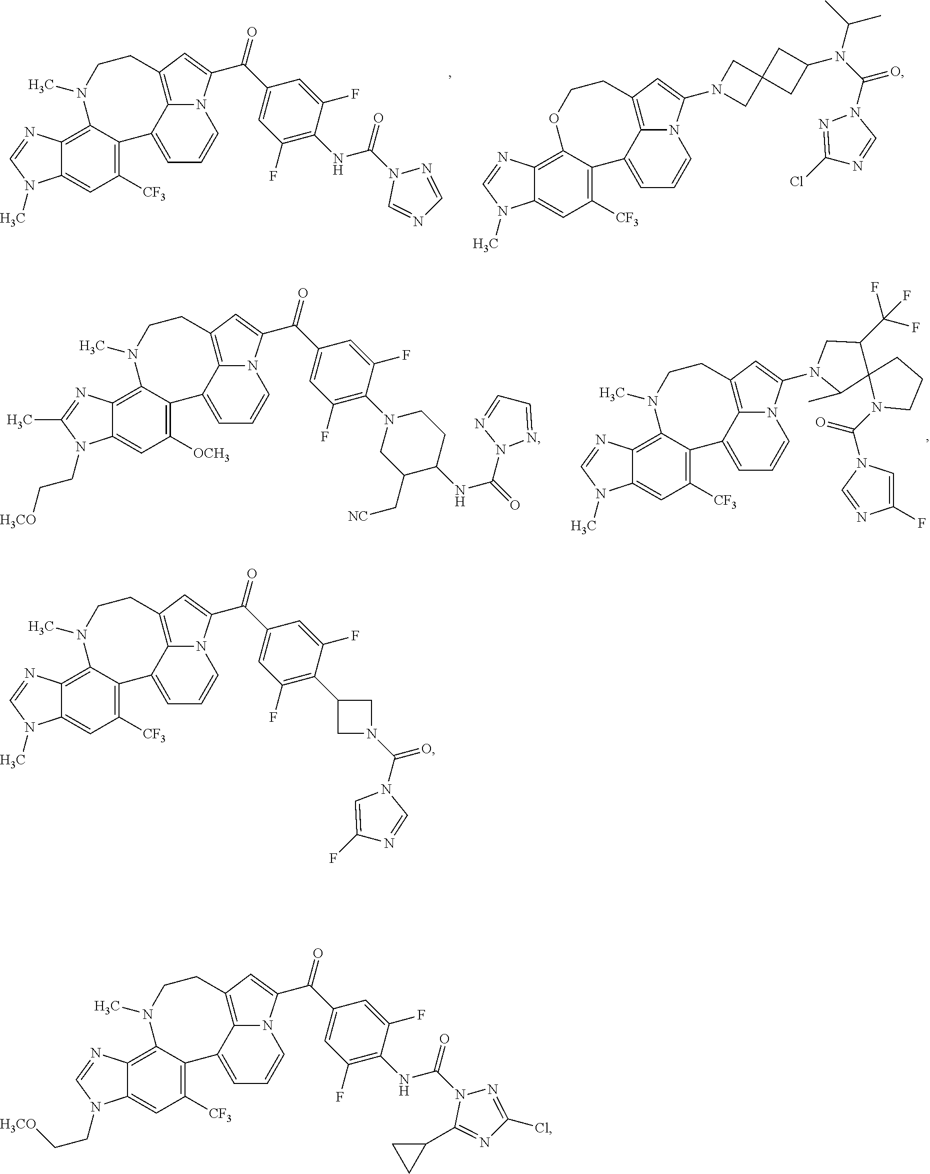

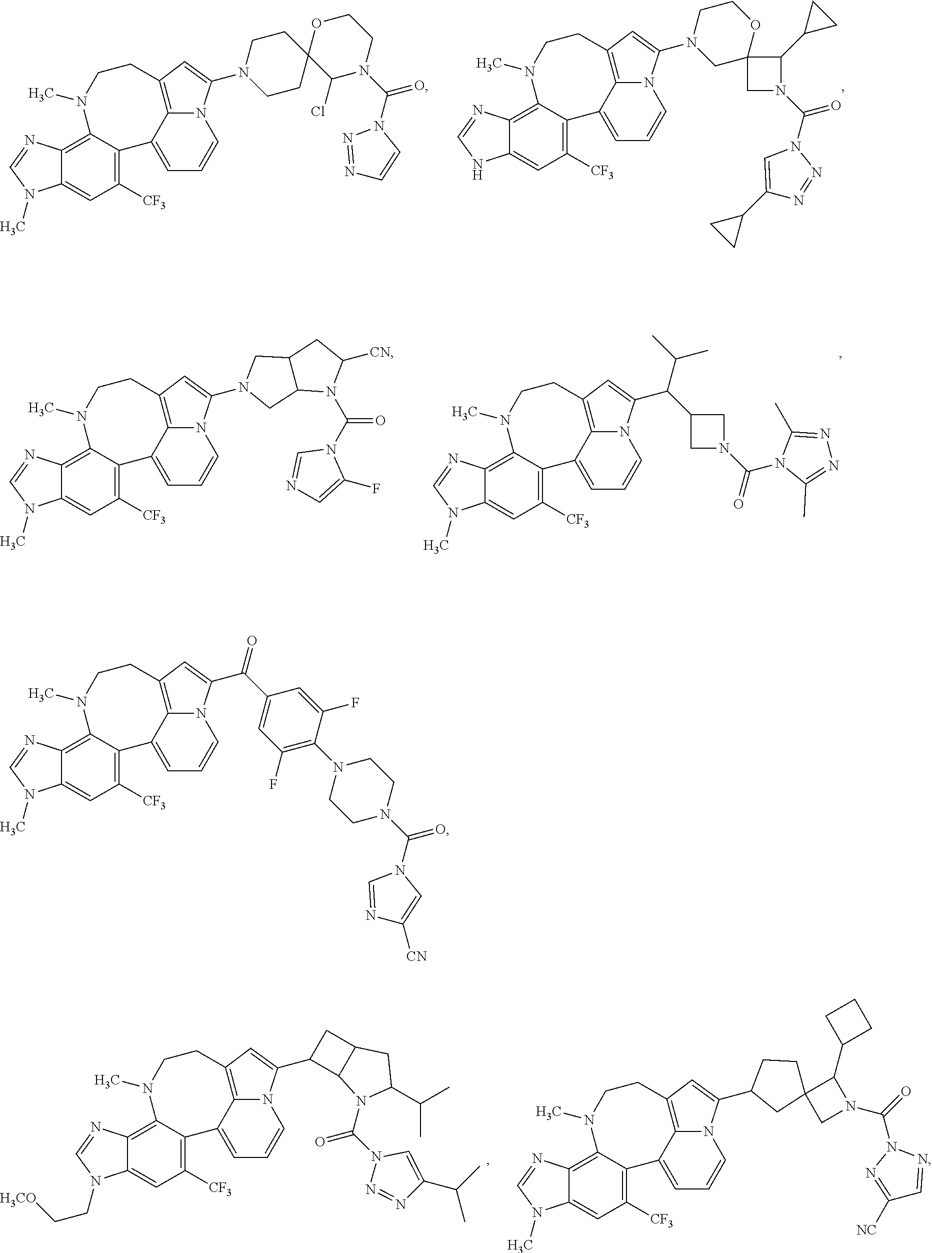

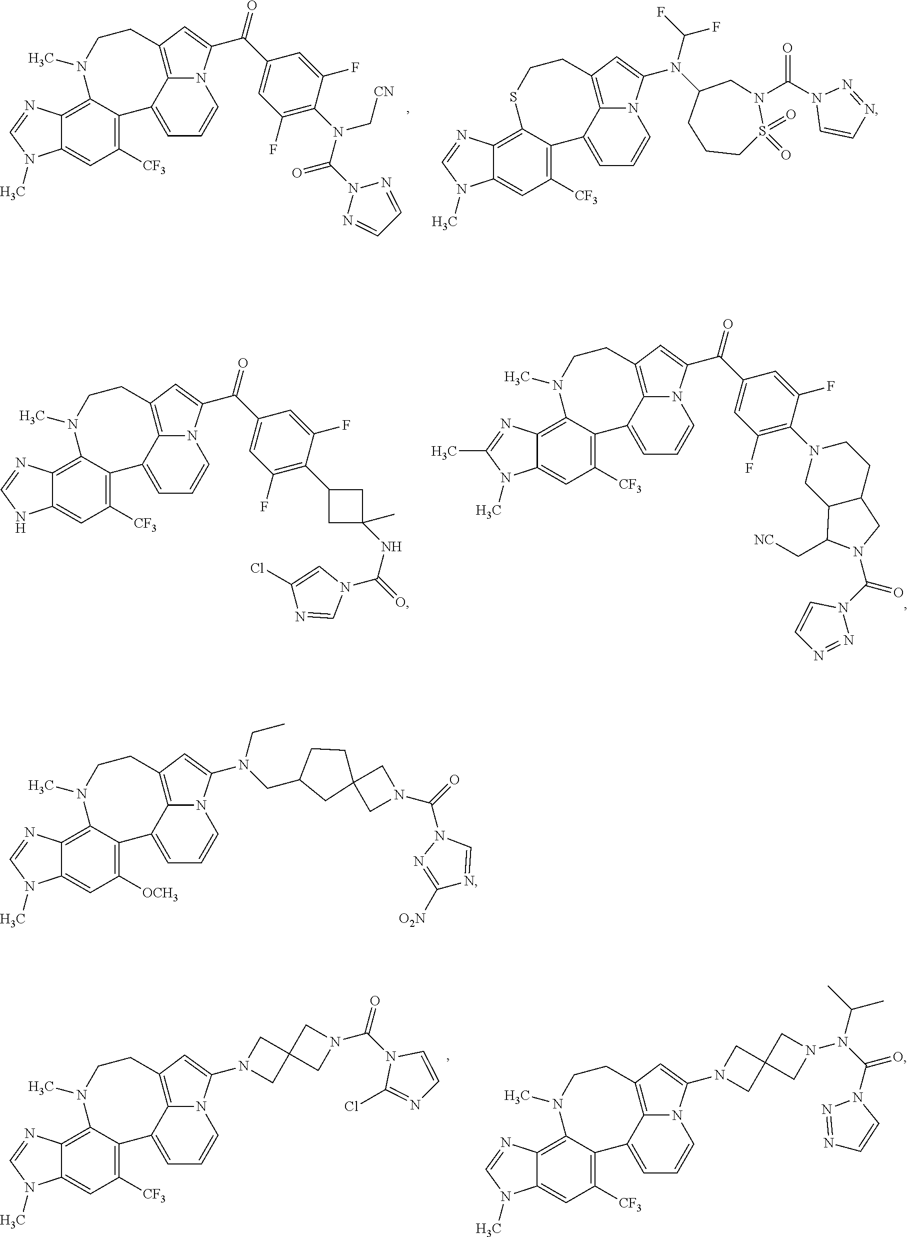

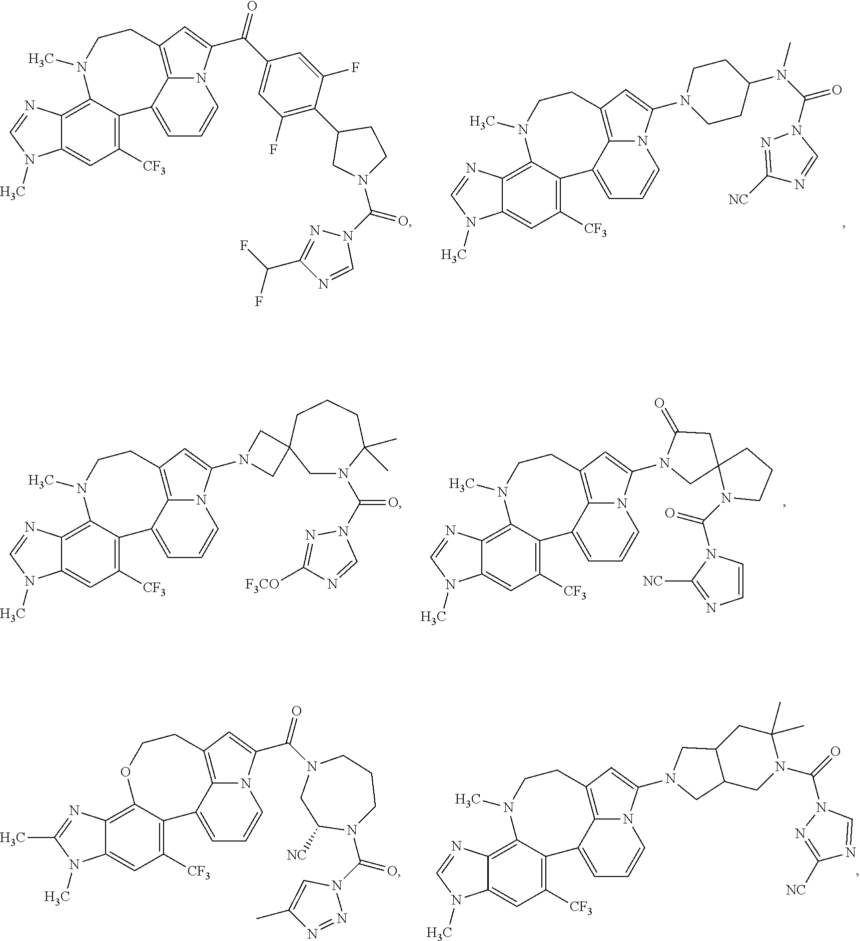

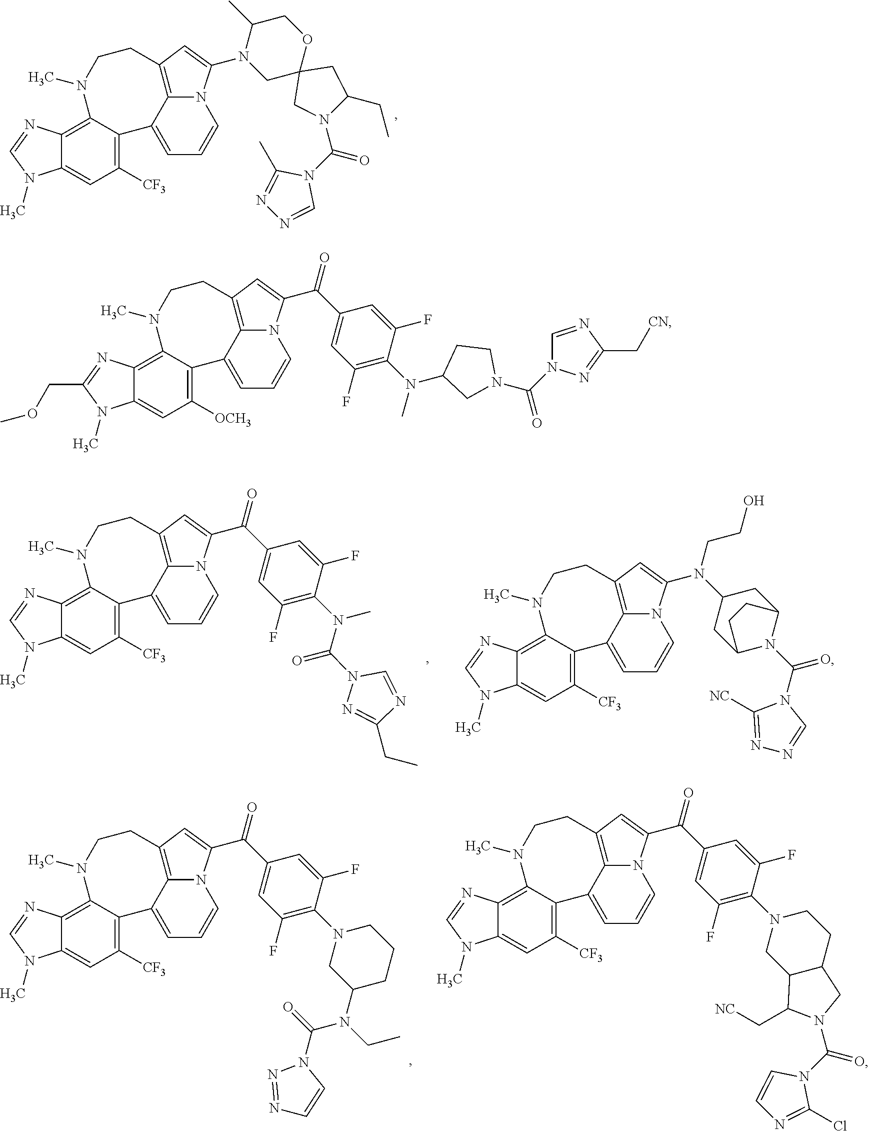

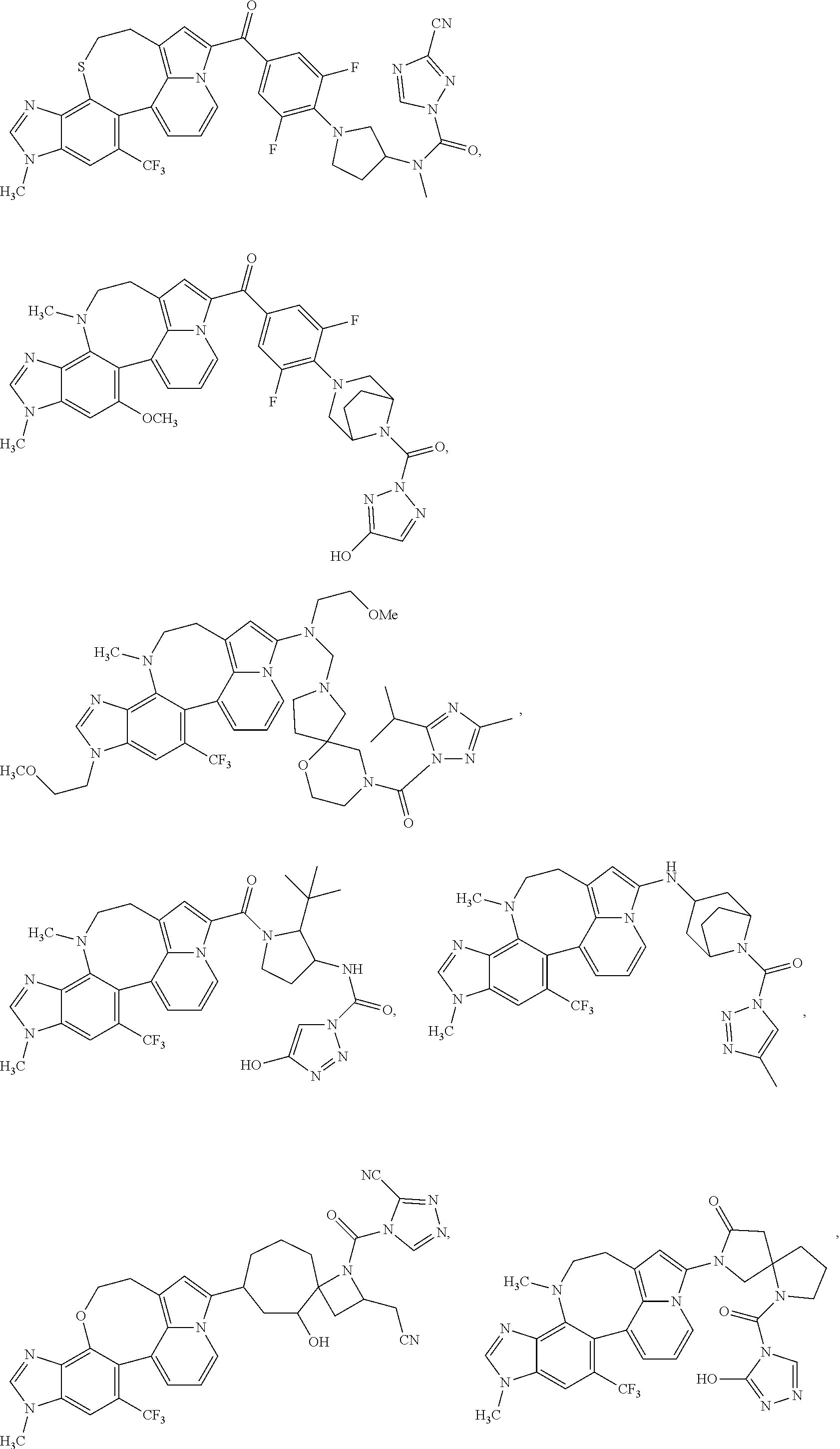

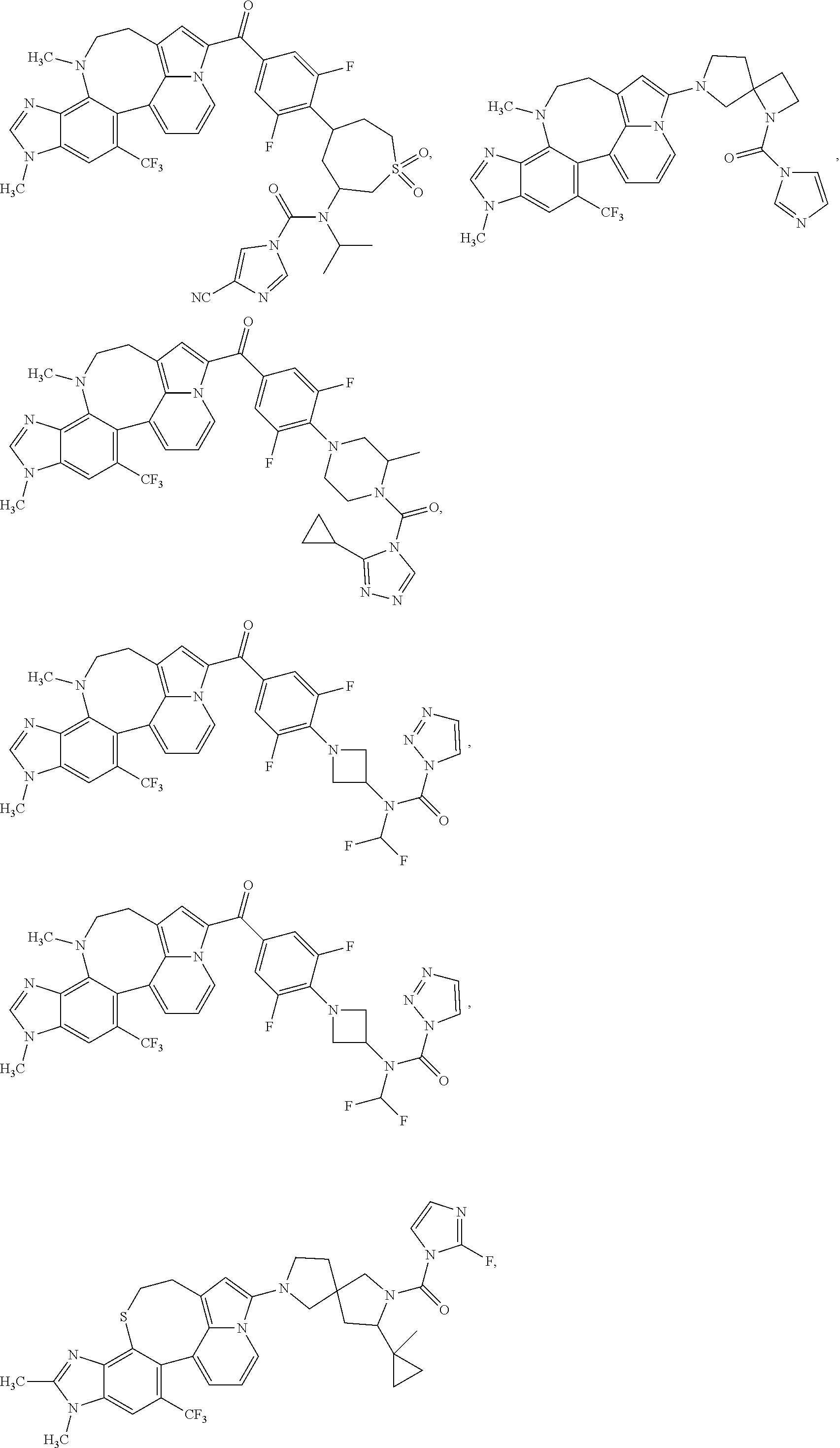

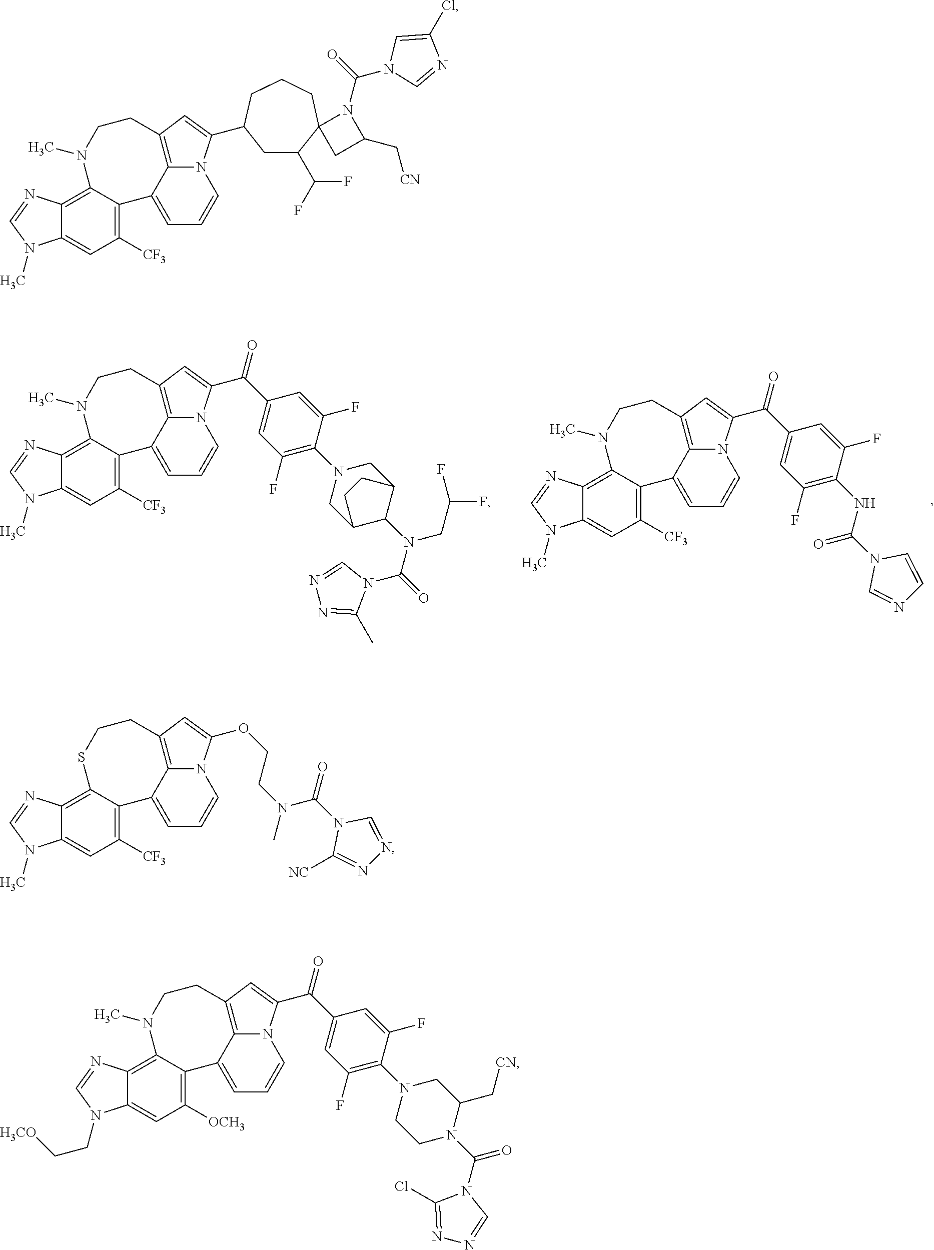 [SMILES: CN1CCc2cc(C3CCCC4(CC(CC#N)N4C(=O)n4cnc(Cl)c4)C(C(F)F)C3)n3cccc(c23)-c2c(C(F)(F)F)cc3c(ncn3C)c21 ; Cc1nncn1C(=O)N(CC(F)F)C1C2CCC1CN(c1c(F)cc(C(=O)c3cc4c5c(cccn35)-c3c(C(F)(F)F)cc5c(ncn5C)c3N(C)CC4)cc1F)C2 ; CN1CCc2cc(C(=O)c3cc(F)c(NC(=O)n4ccnc4)c(F)c3)n3cccc(c23)-c2c(C(F)(F)F)cc3c(ncn3C)c21 ; CN(CCOc1cc2c3c(cccn13)-c1c(C(F)(F)F)cc3c(ncn3C)c1SCC2)C(=O)n1cnnc1C#N ; COCCn1cnc2c3c(c(OC)cc21)-c1cccn2c(C(=O)c4cc(F)c(N5CCN(C(=O)n6cnnc6Cl)C(CC#N)C5)c(F)c4)cc(c12)CCN3C]

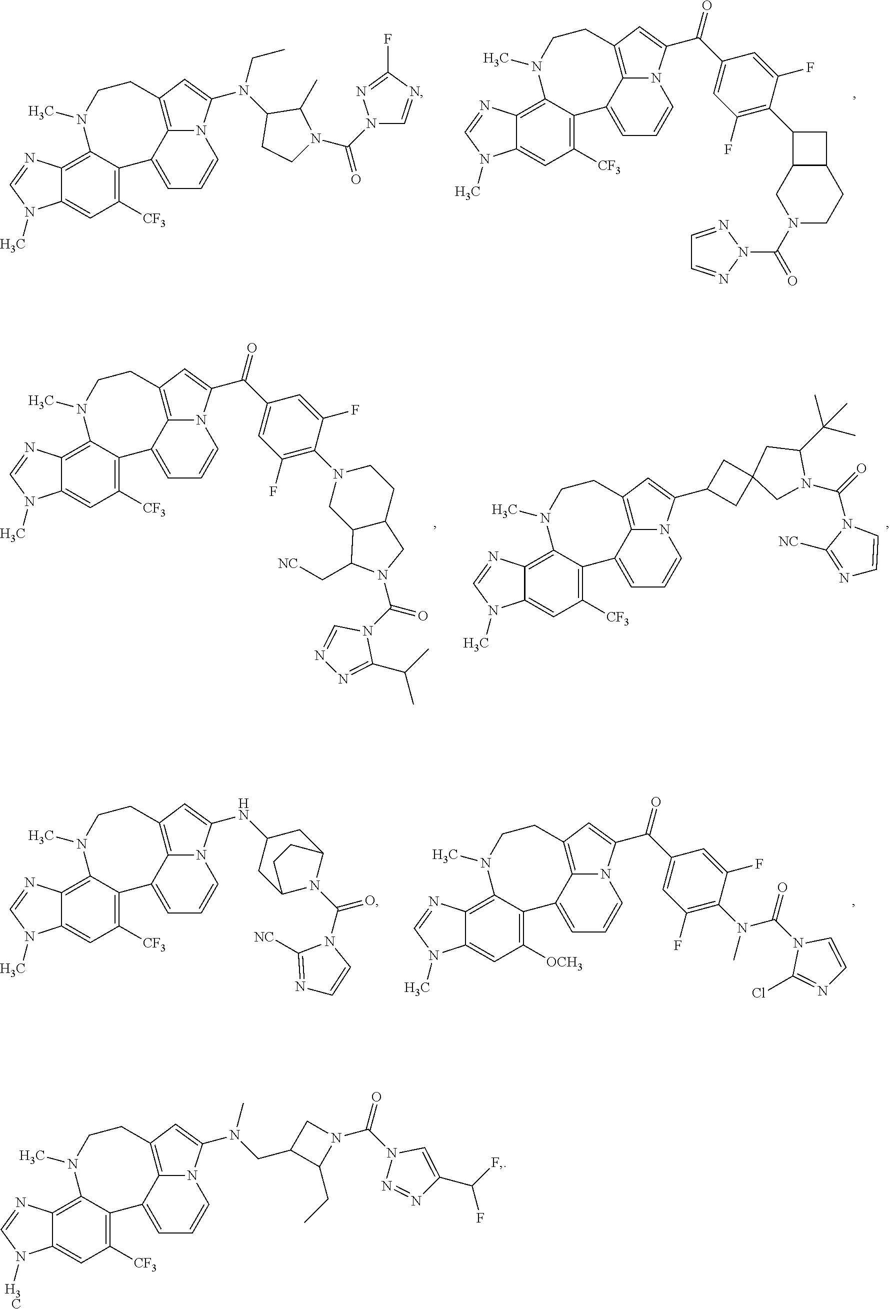

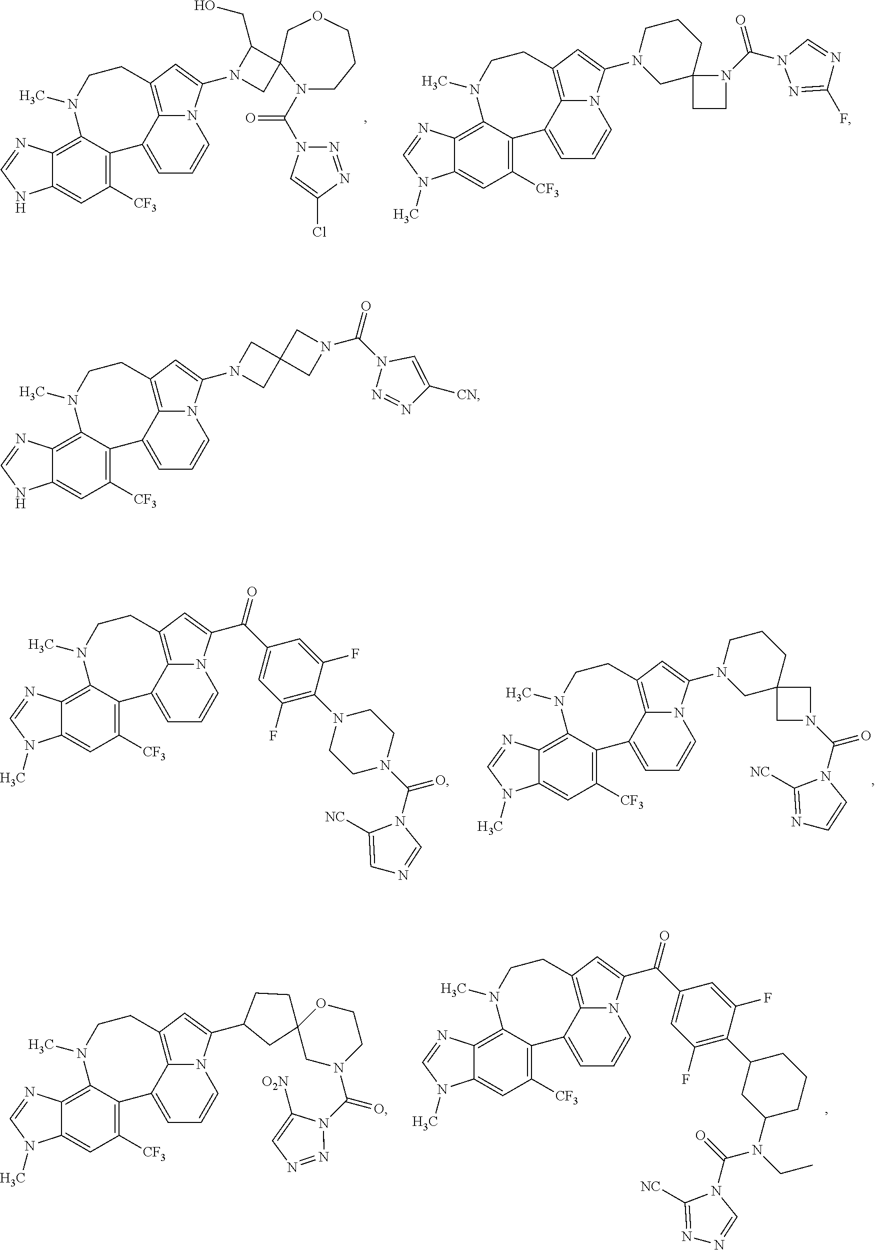

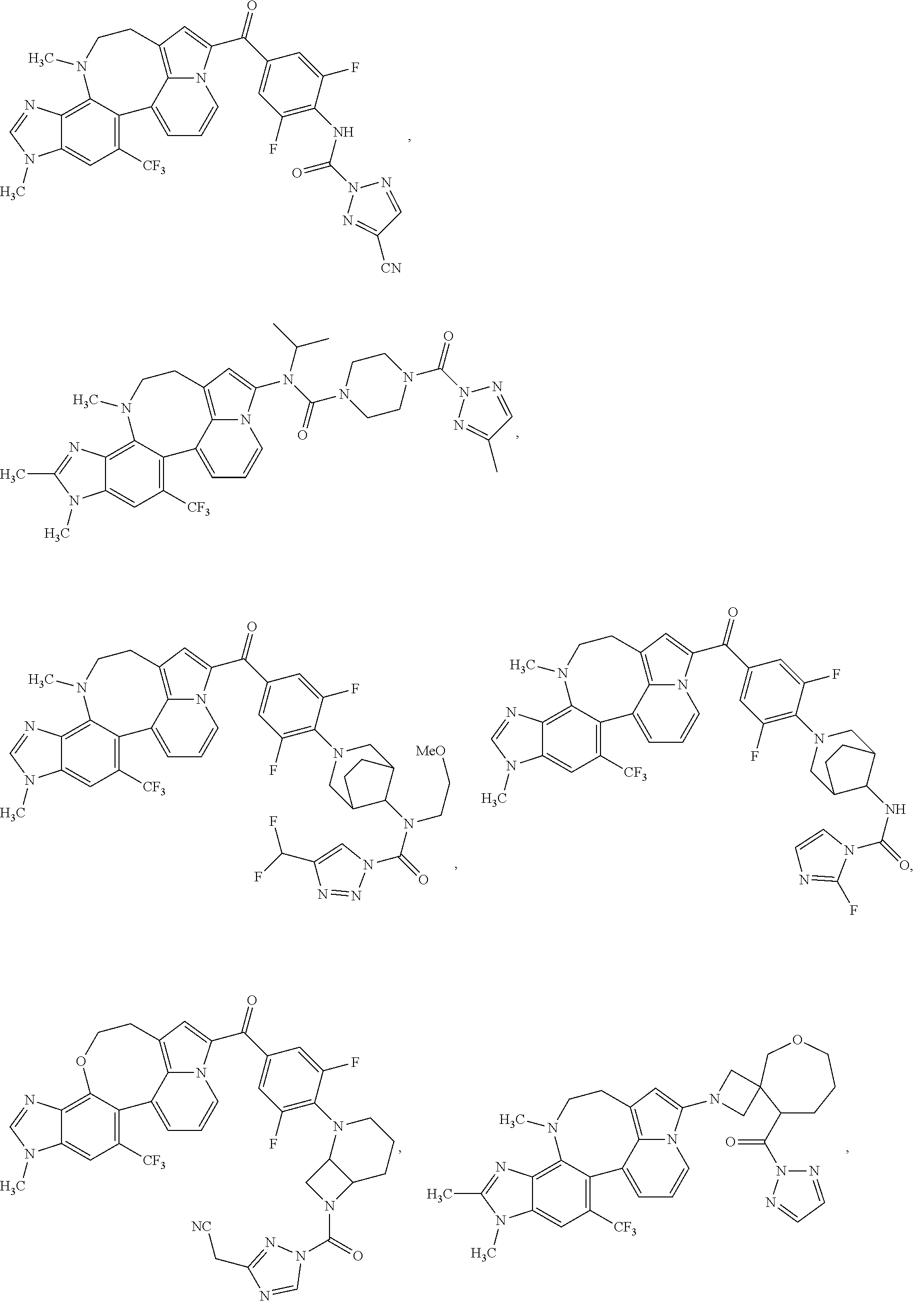

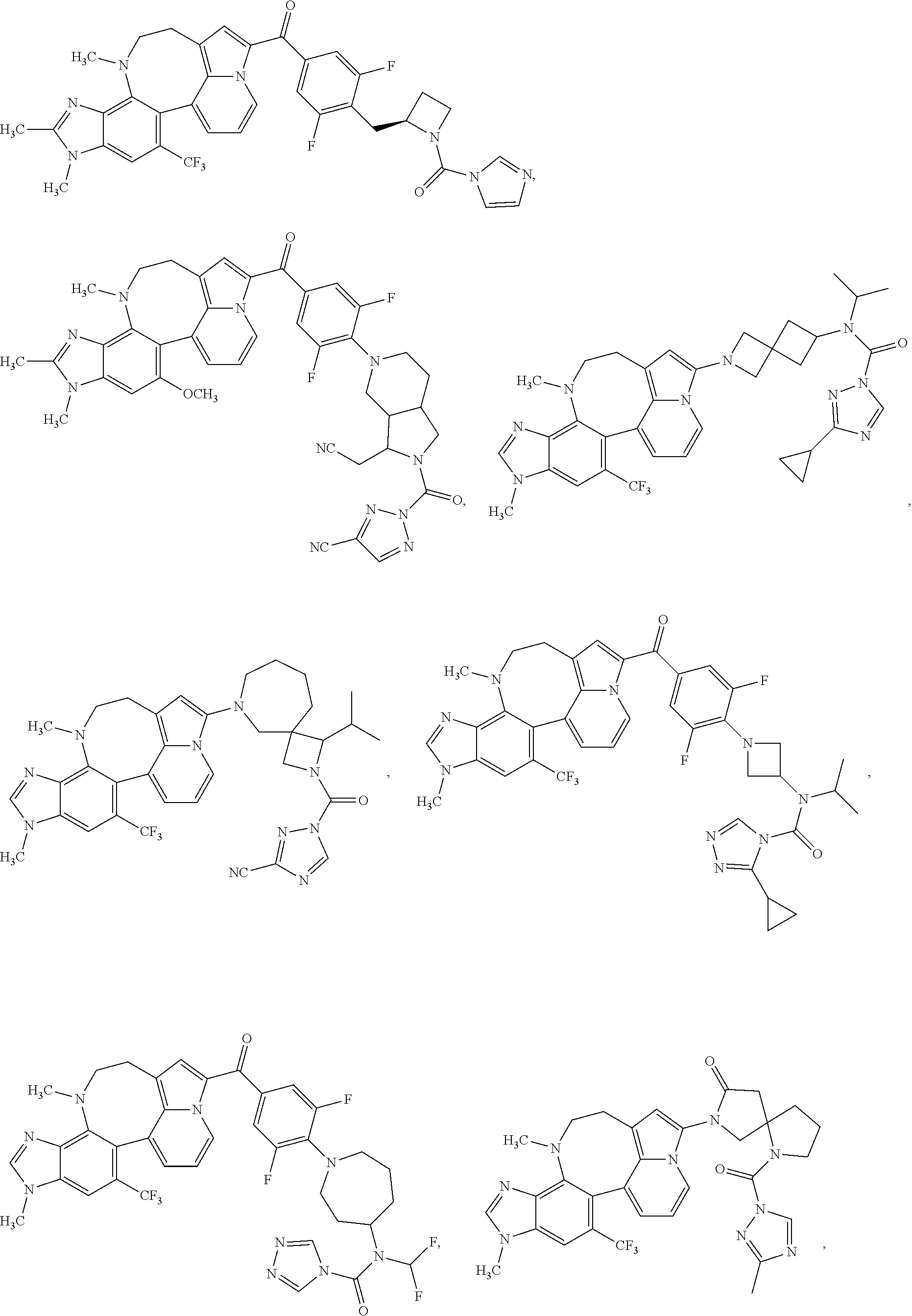

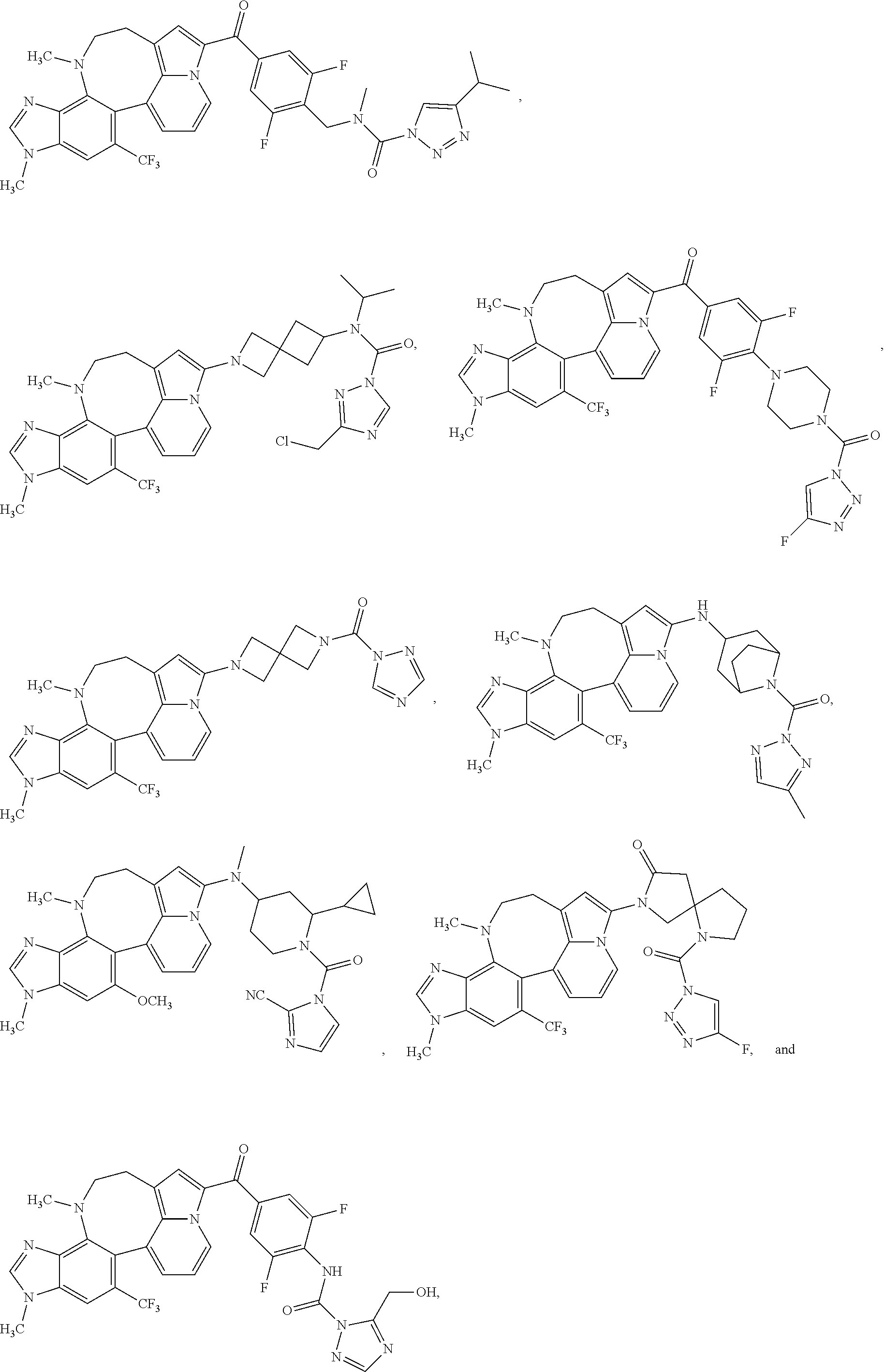

or a pharmaceutically acceptable salt, solvate, stereoisomer, or prodrug thereof.

In general, compounds of the disclosure may be prepared by the following reaction schemes:

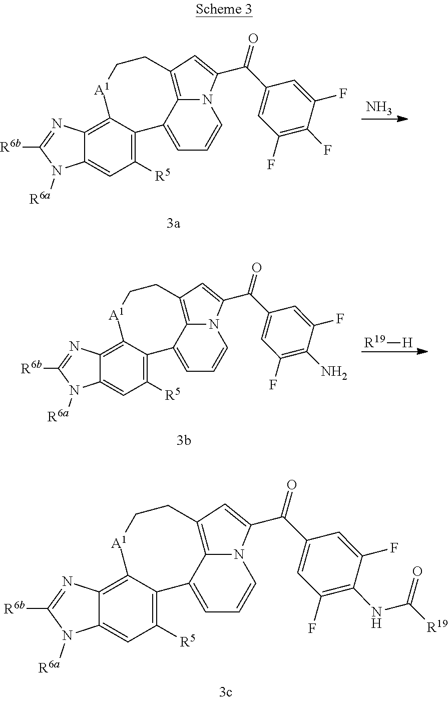

In some embodiments, a compound of Formula 3c may be prepared according to Scheme 3. For example, ammonolysis of aryl fluoride 3a can provide aniline 3b. Addition of R¹⁹—H to 3b in the presence of triphosgene bis(trichloromethyl) carbonate (BTC) and pyridine can provide a compound of Formula 3c.

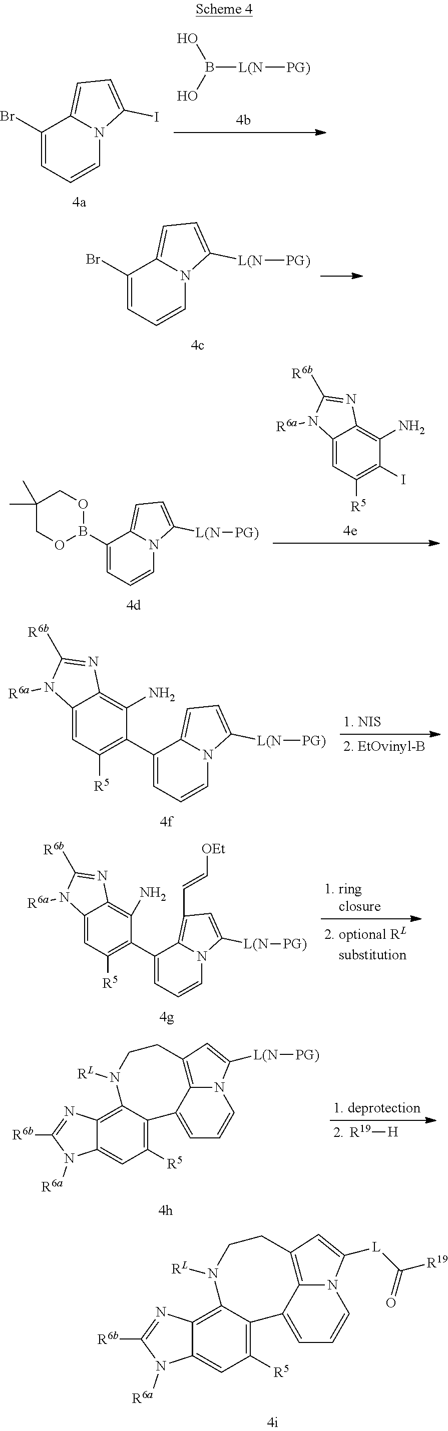

In some embodiments, a compound of Formula 4i may be prepared according to Scheme 4. For example, a cross-coupling reaction, such as a Suzuki reaction between 4a and 4b, can provide compound 4c (wherein L of compound 4c comprises a suitably protected nitrogen atom (e.g., wherein PG is Boc, Cbz, or Fmoc)). Following conversion of 4c to dioxaborinane 4d, a second cross-coupling reaction, such as a Suzuki reaction with 4e, can provide 4f. Halogenation, optionally using NIS, can be followed by a third cross-coupling reaction, such as a Suzuki reaction with 2-[(E)-2-ethoxyvinyl]-4,4,5,5-tetramethyl-1,3,2-dioxaborolane, to provide 4g. Ring closure can be accomplished in the presence of a suitable reducing agent and acid (e.g., NaBH(OAc)₃ and TFA), then the resulting secondary amine can optionally be substituted with R^{L} (e.g., via a reductive amination with HCHO) to provide 4h. Deprotection can be followed by reaction with R¹⁹—H in the presence of triphosgene bis(trichloromethyl) carbonate (BTC) and pyridine to provide a compound of Formula 4i.

Synthetic procedures for certain intermediates may be found in WO 2022/232318, which is incorporated herein by reference in its entirety, including any synthetic methods, recitations of compound variables, combination therapies, and methods of use disclosed therein.

### Compounds of Formula (VII) and (VIII)

In certain aspects, the present disclosure provides a compound of Formula (VII):

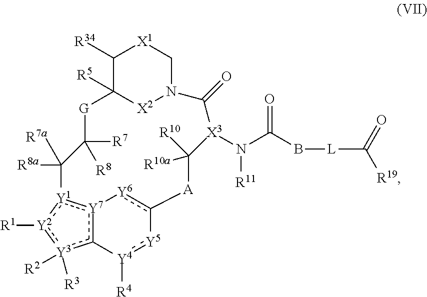

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from 2- to 6-membered heteroalkyl, C₃₋₁₂ carbocycle, and 3- to 12-membered heterocycle, each of which is optionally substituted;
B is selected from a bond, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted;
G is selected from C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)O—CH(R⁶)—, —C(O)NHCH(R⁶)—, C₃₋₁₂ carbocycle, and 3- to 12-membered heterocycle, wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, C₃-12 carbocycle, and 3- to 12-membered heterocycle are optionally substituted;
X¹ is selected from NR⁹, O, S(O), S(O)₂, and optionally substituted C₁₋₂ alkylene;
X² is selected from NH and O;
X³ is selected from N and CH;
Y¹ is selected from C, CH, and N;
Y², Y³, Y⁴, and Y⁷ are independently selected from C and N;
Y⁵ is selected from CH, CH₂, and N;
Y⁶ is selected from C(O), CH, CH₂, and N;
R¹ is selected from —CN, C₁₋₆ alkyl, 2- to 6-membered heteroalkyl, C₃₋₁₂ carbocycle, and 3- to 12-membered heterocycle, wherein C₁₋₆ alkyl, 2- to 6-membered heteroalkyl, C₃₋₁₂ carbocycle, and 3- to 12-membered heterocycle are optionally substituted; or R¹ and R², together with the atoms to which they are attached, form optionally substituted 3- to 12-membered heterocycle;
R² is absent or is selected from hydrogen, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; R³ is absent; or
R² and R³, together with the atom to which they are attached, form C₃₋₁₂ carbocycle or 3- to 12-membered heterocycle, each of which is optionally substituted;
R⁴ is absent or is selected from hydrogen, halogen, —CN, C₁₋₃ alkyl, and C₁₋₃ haloalkyl;
R⁵ is selected from hydrogen, C₁₋₆ alkyl, and C₃₋₆ carbocycle, wherein C₁₋₆ alkyl and C₃₋₆ carbocycle are optionally substituted;
R⁶ is selected from hydrogen and methyl; R⁷ is selected from hydrogen, halogen, and optionally substituted C₁₋₃ alkyl; or
R⁶ and R⁷, together with the carbon atoms to which they are attached, form C₃₋₆ carbocycle or 3- to 7-membered heterocycle, each of which is optionally substituted;
R⁸ is selected from hydrogen, halogen, —OR¹², —CN, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; or
R⁷ and R⁸, together with the carbon atom to which they are attached, form C═CR^{7′}R^{8′}, C═N(OH), C═N(O—C₁₋₃ alkyl), C═O, C═S, C═NH, C₃₋₆ carbocycle, or 3- to 7-membered heterocycle, wherein C₃₋₆ carbocycle and 3- to 7-membered heterocycle are optionally substituted;
R^{7a} and R^{8a} are independently selected from hydrogen, halogen, and optionally substituted C₁₋₆ alkyl; or R^{7a} and R^{8a}, together with the carbon atom to which they are attached, form C═O;
R^{7′} is selected from hydrogen, halogen, and optionally substituted C₁₋₆ alkyl; R^{8′} is selected from hydrogen, halogen, —OR¹², —CN, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; or
R^{7′} and R^{8′}, together with the carbon atom to which they are attached, form C₃₋₆ carbocycle or 3- to 7-membered heterocycle, each of which is optionally substituted;
R⁹ is selected from hydrogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹⁰ is selected from hydrogen, halogen, —OR¹², and optionally substituted C₁₋₃ alkyl;
R^{10a} is selected from hydrogen and halogen;
R¹¹ is selected from hydrogen and optionally substituted C₁₋₃ alkyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle;
R³⁴ is selected from hydrogen and optionally substituted C₁₋₃ alkyl; and
[symbol] indicates a single or double bond such that all valences are satisfied.

In certain aspects, the present disclosure provides a compound of Formula (VIII):

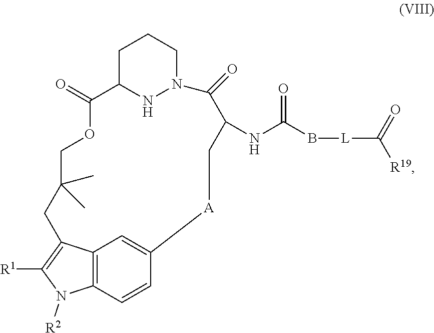

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from 2- to 6-membered heteroalkyl, C₃₋₁₂ carbocycle, and 3- to 12-membered heterocycle, each of which is optionally substituted;
B is selected from a bond, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted;
R¹ is selected from —CN, C₁₋₆ alkyl, 2- to 6-membered heteroalkyl, C₃₋₁₂ carbocycle, and 3- to 12-membered heterocycle, wherein C₁₋₆ alkyl, 2- to 6-membered heteroalkyl, C₃₋₁₂ carbocycle, and 3- to 12-membered heterocycle are optionally substituted;
R² is absent or is selected from hydrogen, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; and
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted.

In some embodiments, for a compound of Formula (VII) or (VIII), R¹ is selected from C₆₋₁₀ carbocycle and 6- to 10-membered heterocycle, each of which is optionally substituted. In some embodiments, R¹ is selected from C₆ carbocycle and 5- to 6-membered heterocycle, each of which is optionally substituted. In some embodiments, R¹ is selected from C₆ aryl and 6-membered heteroaryl, each of which is optionally substituted. In some embodiments, R¹ is substituted pyridyl, such as substituted pyrid-3-yl. In some embodiments, R¹ is optionally substituted pyrid-3-yl. In some embodiments, R¹ is 2-(1-methoxyethyl)pyridin-3-yl. In some embodiments, R¹ is 2-(1-methoxyethyl)pyridin-3-yl substituted with optionally substituted 3- to 8-membered heterocycle. In some embodiments, R¹ is selected from

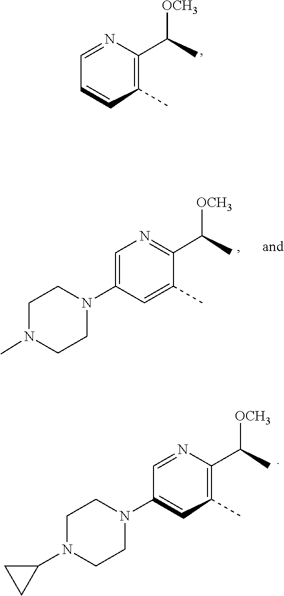

In some embodiments, for a compound of Formula (VII) or (VIII), R¹ is substituted with one or two substituents. In some embodiments, R¹ is substituted with one or more substituents independently selected from C₁₋₆ alkyl, 2- to 6-membered heteroalkyl, and optionally substituted 3- to 8-membered heterocycle. In some embodiments, R¹ is substituted with one or more substituents independently selected from —CH(CH₃)OCH₃, —CH₂OCH₃, 4-methylpiperazin-1-yl, and 4-cyclopropylpiperazin-1-yl. In some embodiments, R¹ is substituted with —CH(CH₃)OCH₃. In some embodiments, R¹ is substituted with —CH(CH₃)OCH₃ and optionally substituted piperazine. In some embodiments, R¹ is substituted with —CH(CH₃)OCH₃ and piperazine, wherein the piperazine is substituted at the 4-position with C₁₋₆ alkyl or C₃₋₆ carbocycle. In some embodiments, R¹ is substituted with —CH(CH₃)OCH₃ and either 4-methylpiperazin-1-yl or 4-cyclopropylpiperazin-1-yl.

In some embodiments, for a compound of Formula (VII) or (VIII), R² is selected from C₁₋₆ alkyl, C₁₋₆ fluoroalkyl, and C₃₋₆ cycloalkyl. In some embodiments, R² is selected from C₁₋₂ alkyl and C₁₋₂ haloalkyl. In some embodiments, R² is selected from —CH₂CH₃ and —CH₂CF₃. In some embodiments, R² is —CH₂CH₃. In some embodiments, R² is —CH₂CF₃.

In some embodiments, for a compound of Formula (VII) or (VIII), A is selected from C₅₋₈ carbocycle, and 5- to 8-membered heterocycle, each of which is optionally substituted. In some embodiments, A is optionally substituted phenyl. In some embodiments, A is selected from 6-membered heterocycloalkyl and 6-membered heterocycloalkenyl. In some embodiments, A is selected from 6-membered heteroaryl. In some embodiments, A is selected from phenyl, phenolyl, pyridyl, 1,2,3,6-tetrahydropyridyl, thiazolyl, morpholinyl, and oxazolyl. In some embodiments, A is selected from 1,2,3,6-tetrahydropyrid-1,5-diyl, thiazol-2,4-diyl, morpholin-2,4-diyl, 5-hydroxyphen-1,3-diyl, and oxazol-2,5-diyl.

In some embodiments, for a compound of Formula (VII) or (VIII), B is selected from a bond, C₁₋₆ alkyl, and 3- to 12-membered heterocycle, wherein C₁₋₆ alkyl and 3- to 12-membered heterocycle are optionally substituted. In some embodiments, B is selected from C₁₋₆ alkyl and 3- to 12-membered heterocycle, each of which is optionally substituted. In some embodiments, B is selected from a bond, C₁₋₃ alkyl, and C₂₋₃ alkenyl, wherein C₁₋₃ alkyl and C₂₋₃ alkenyl are optionally substituted. In some embodiments, B is selected from C₁₋₃ alkyl and C₂₋₃ alkenyl, each of which is optionally substituted. In some embodiments, B is a bond. In some embodiments, B is —CHR^{B}—, wherein R^{B} is selected from halogen, C₁₋₆ alkyl, C₁₋₆ haloalkyl, 2- to 6-membered heteroalkyl, C₃₋₆ cycloalkyl, and 3- to 7-membered heterocycloalkyl. In some embodiments, B is —CHR^{B}—, wherein R^{B} is selected from C₁₋₃ alkyl, C₁₋₃ haloalkyl, and C₃₋₆ cycloalkyl. In some embodiments, B is —CHR^{B}—, wherein R^{B} is selected from isopropyl and cyclopentyl. In some embodiments, B is selected from —CH(CH(CH₃)₂)— and —CH(cyclopentyl)-.

In some embodiments, the compound of Formula (VII) or (VIII) is selected from:

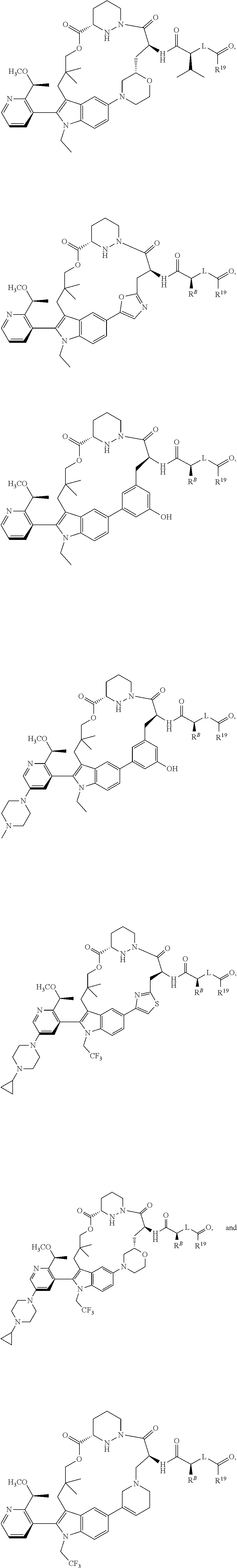

or a pharmaceutically acceptable salt or solvate thereof. In some embodiments, R^{B} is selected from halogen, C₁₋₆ alkyl, C₁₋₆ haloalkyl, 2- to 6-membered heteroalkyl, C₃₋₆ cycloalkyl, and 3- to 7-membered heterocycloalkyl. In some embodiments, R^{B} is selected from C₁₋₃ alkyl, C₁₋₃ haloalkyl, and C₃₋₆ cycloalkyl. In some embodiments, R^{B} is selected from —CH(CH₃)₂ and cyclopentyl.

In some embodiments, for a compound of Formula (VII) or (VIII), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea. In some embodiments, L is a bond.

In some embodiments, for a compound of Formula (VII) or (VIII), L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH—, —CH₂N(CH₃)—, —NHCH₂—, or —N(CH₃)CH₂—. In some embodiments, L¹ is selected from a bond, 2- to 6-membered heteroalkyl, and —N(R¹²)—.

In some embodiments, for a compound of Formula (VII) or (VIII), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (VII) or (VIII), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (VII) or (VIII), L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In certain aspects, the present disclosure provides a compound selected from:

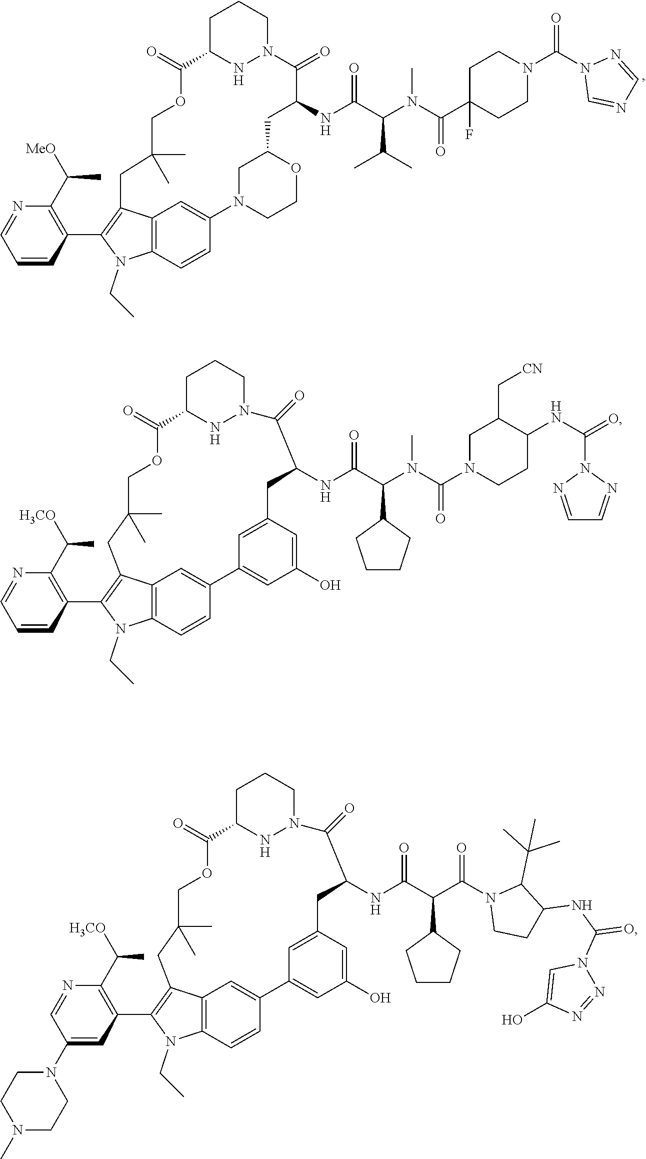

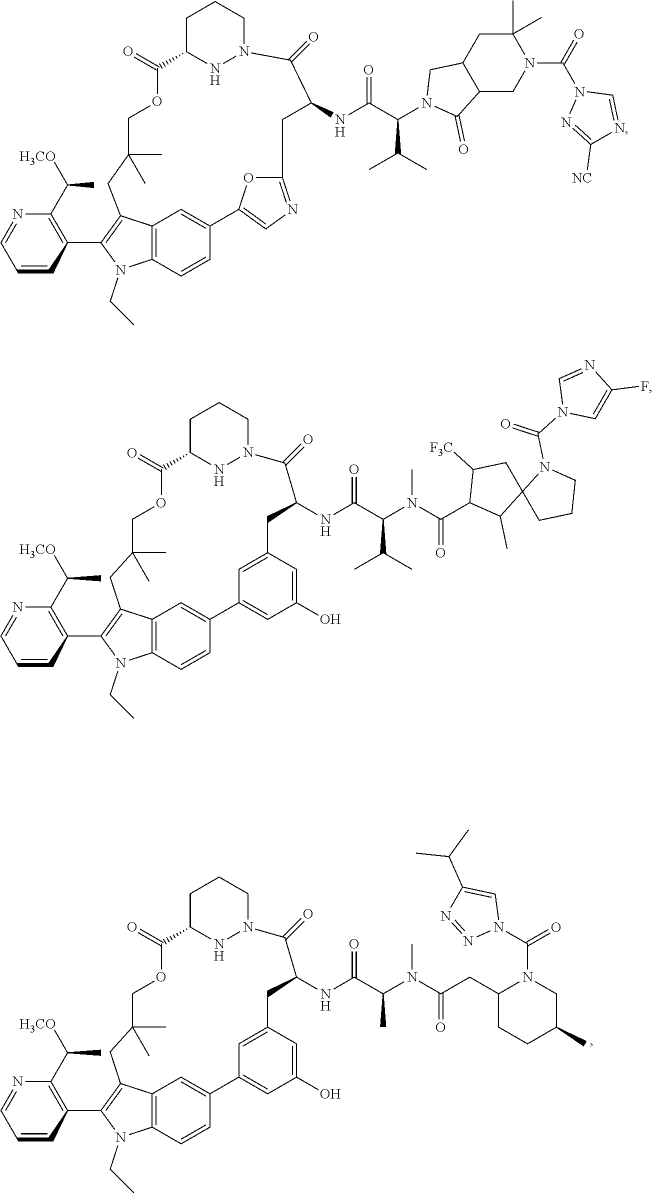

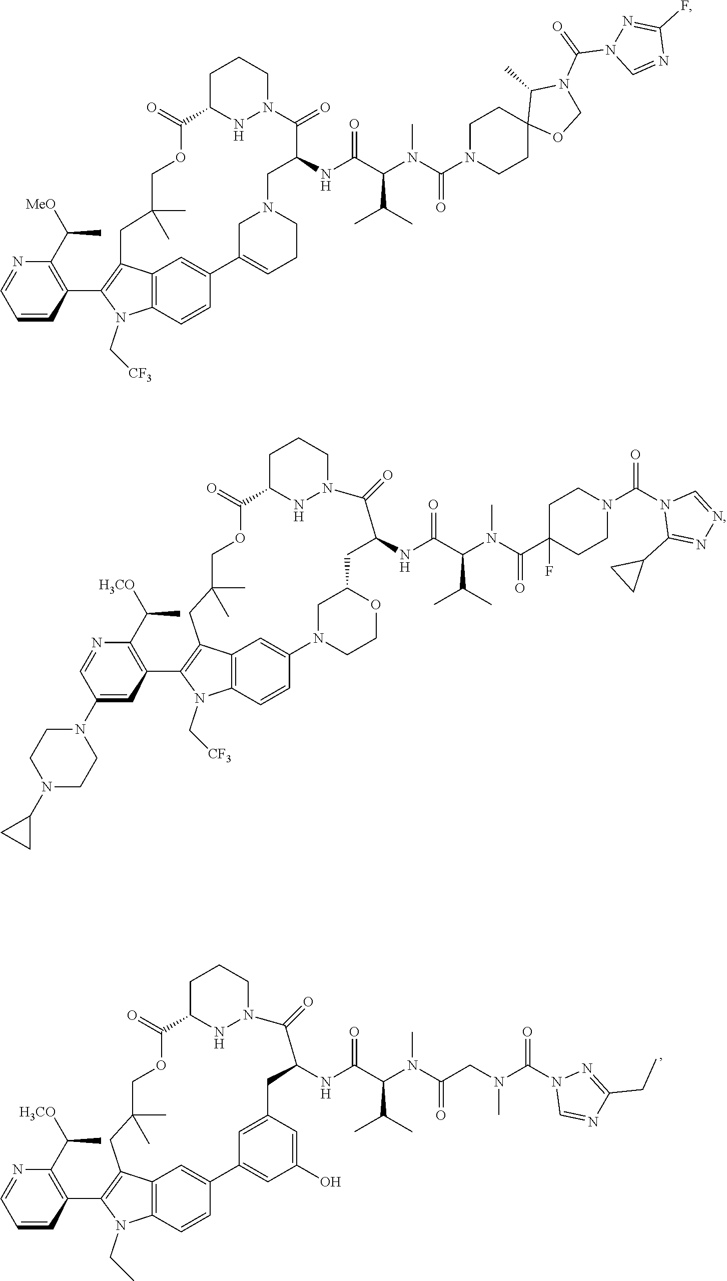

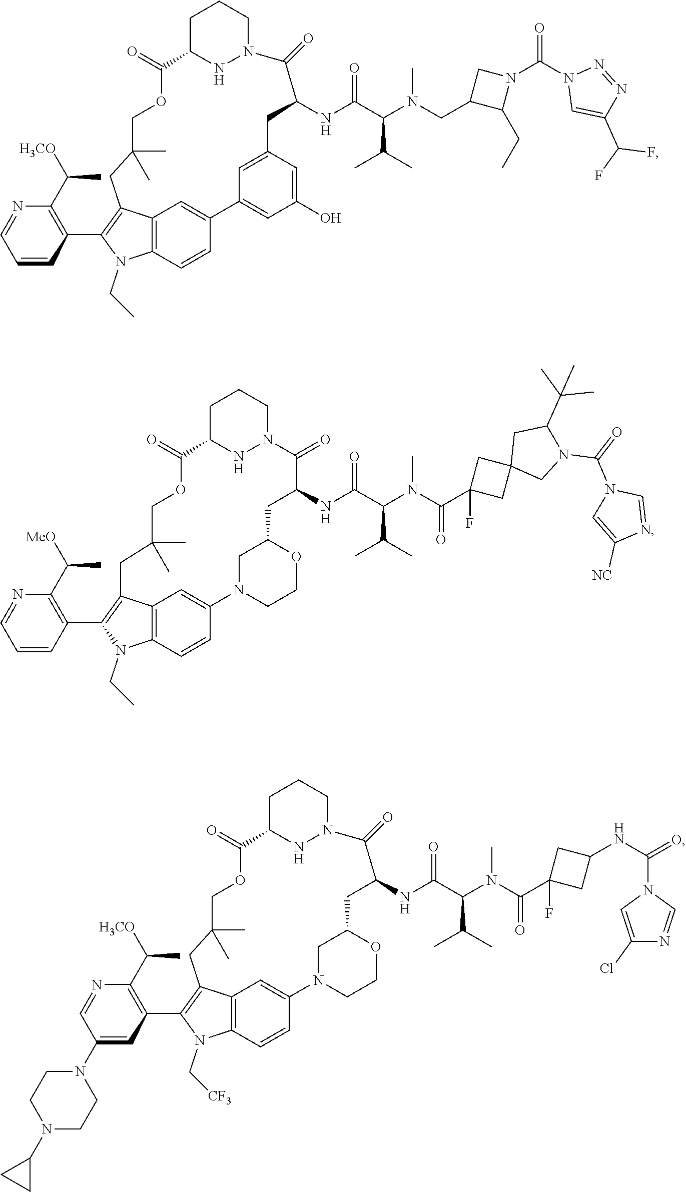

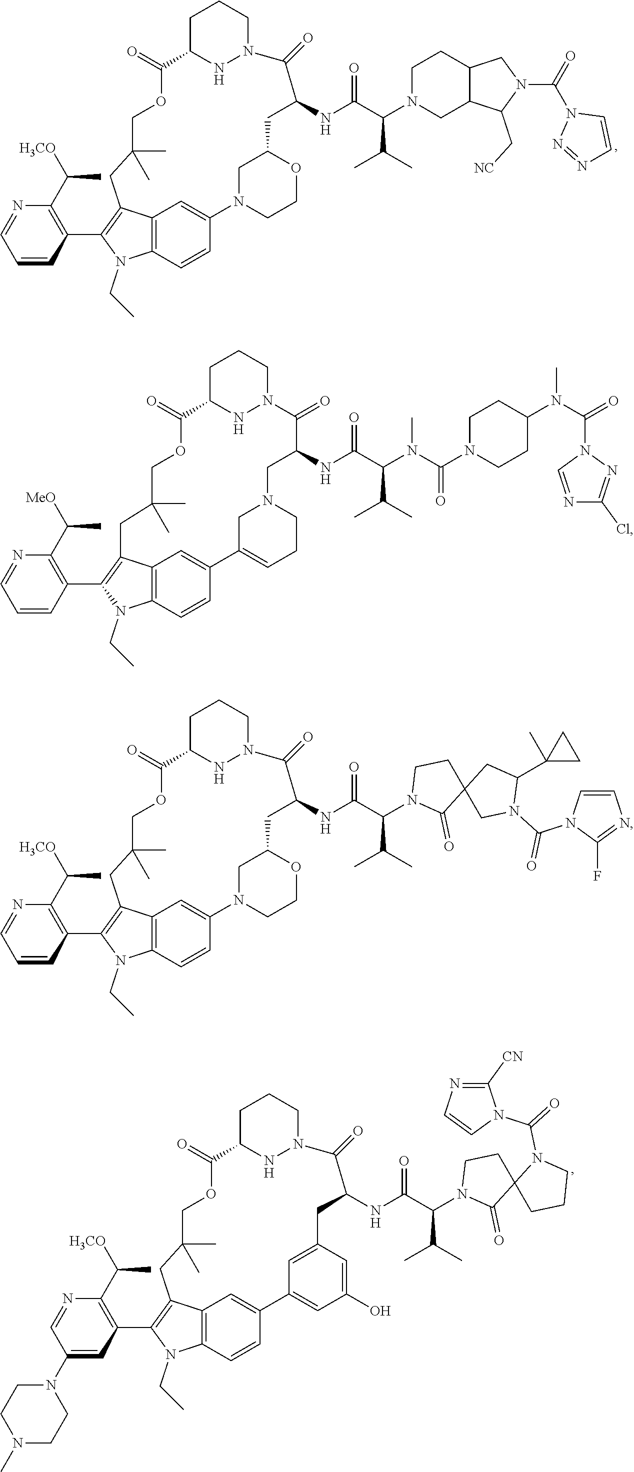

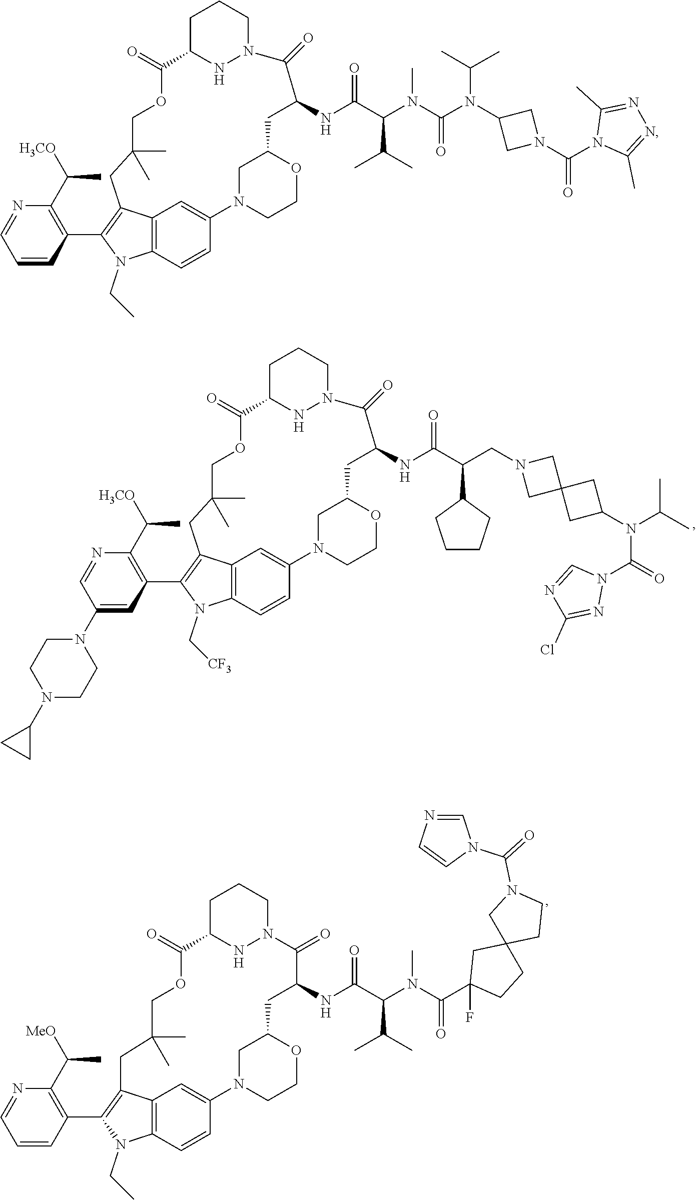

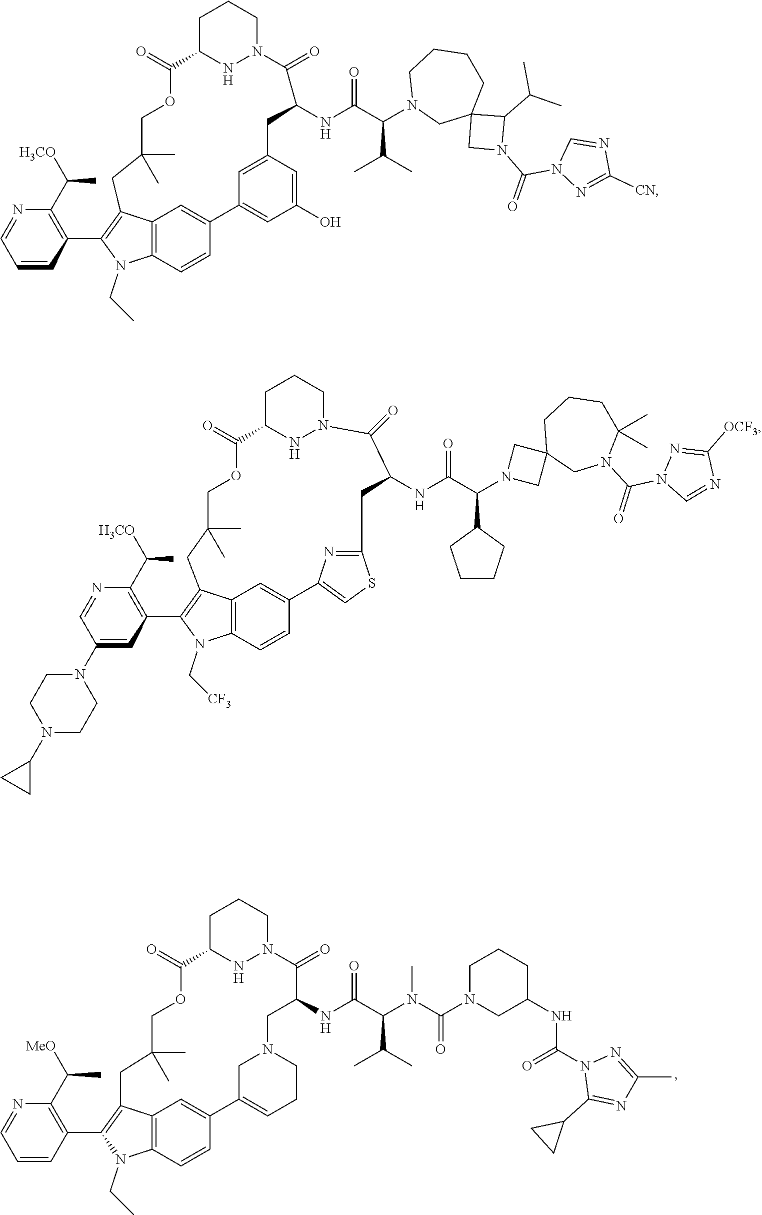

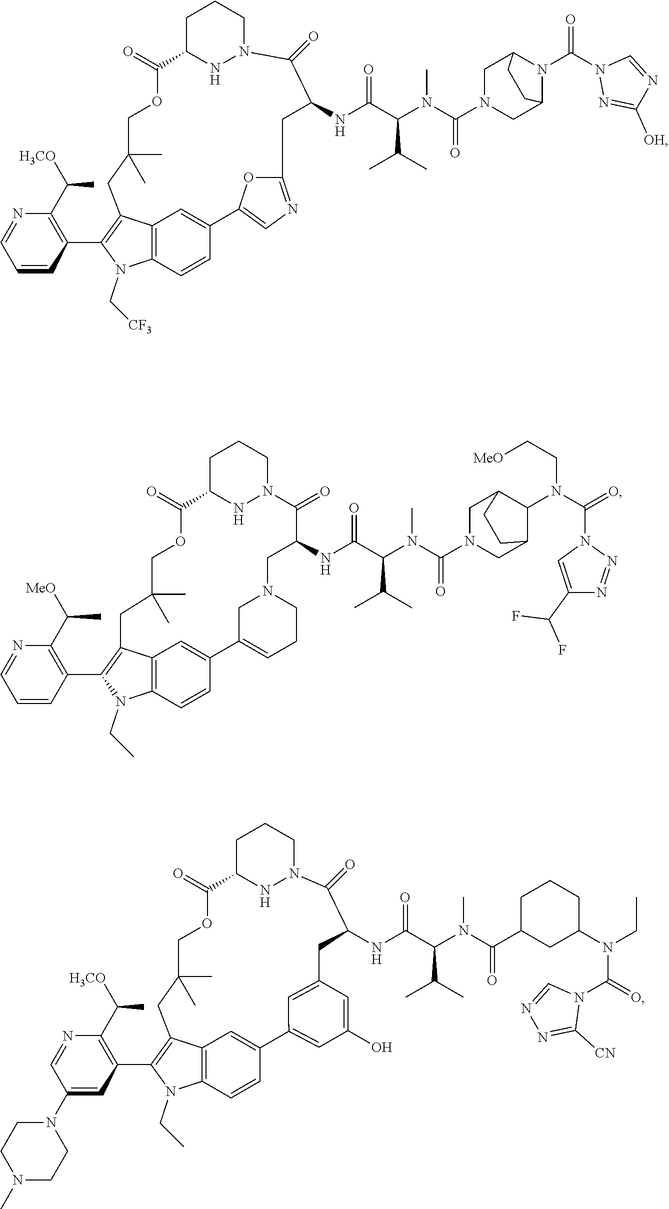

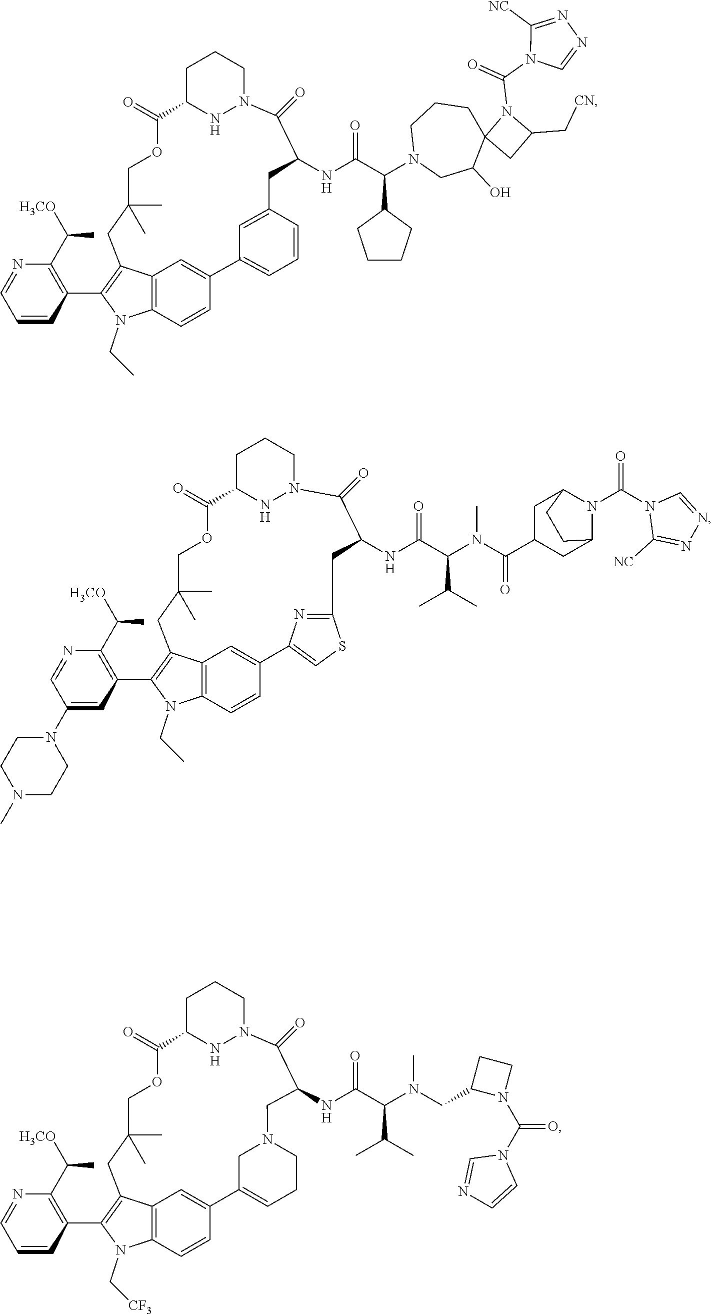

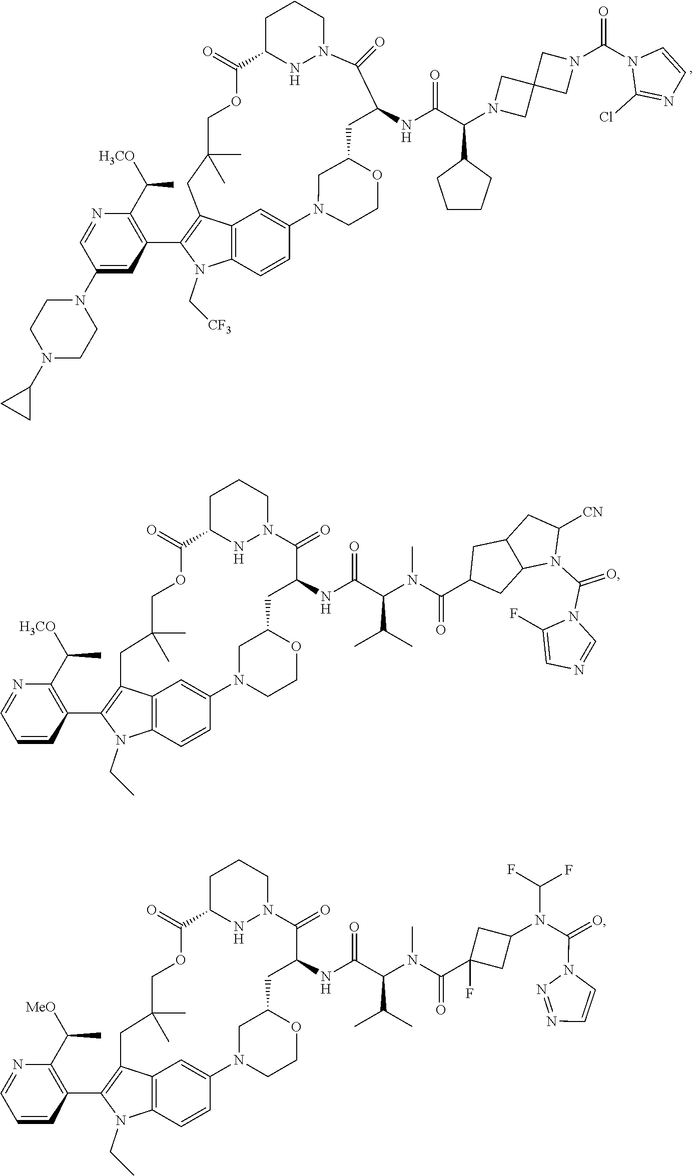

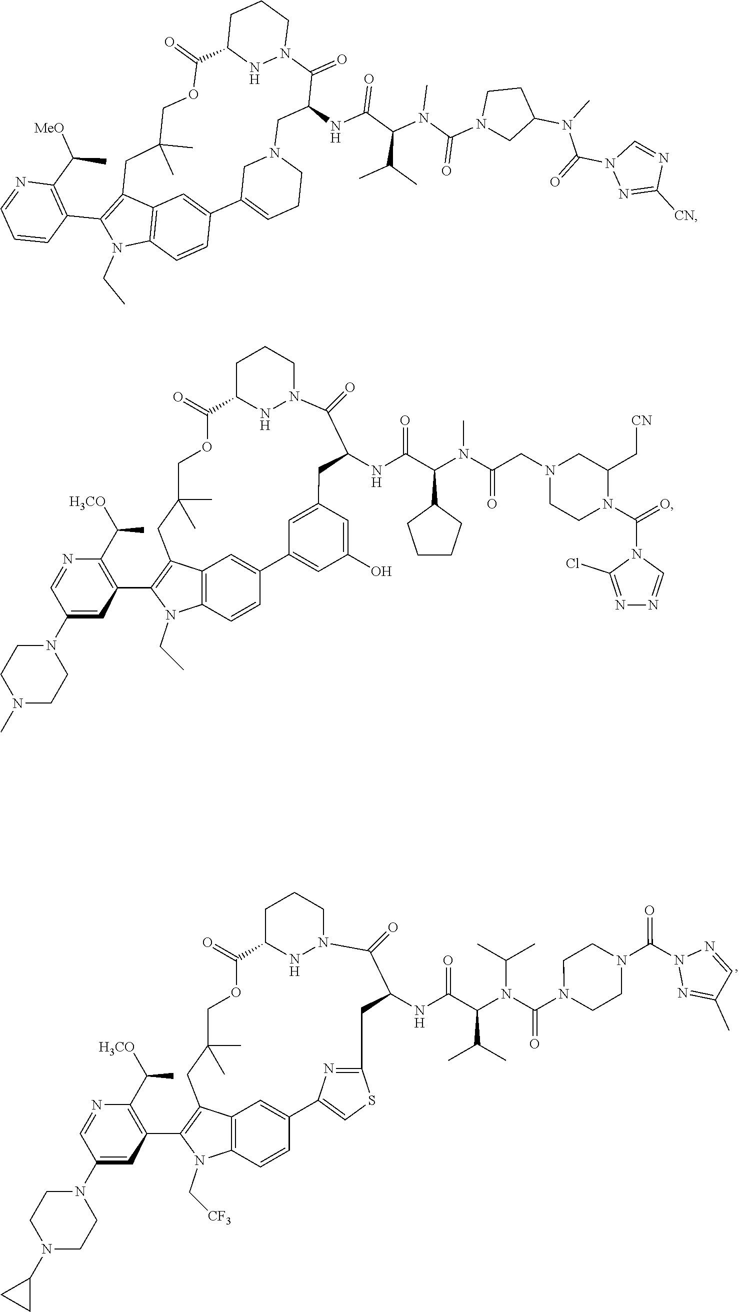

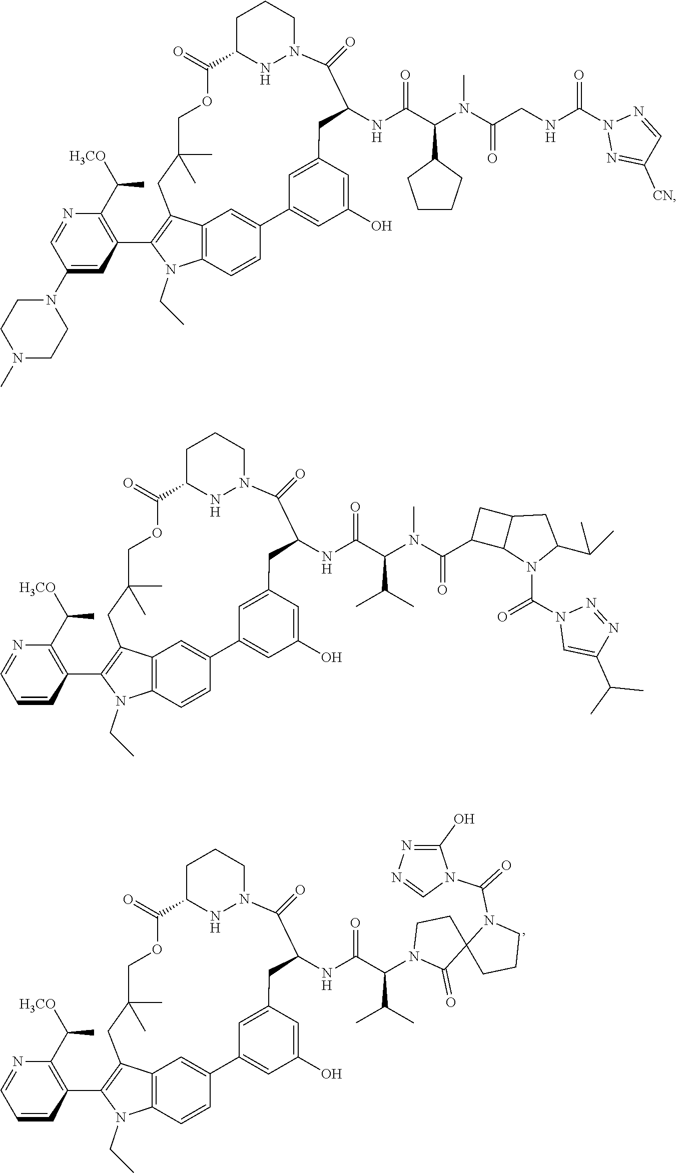

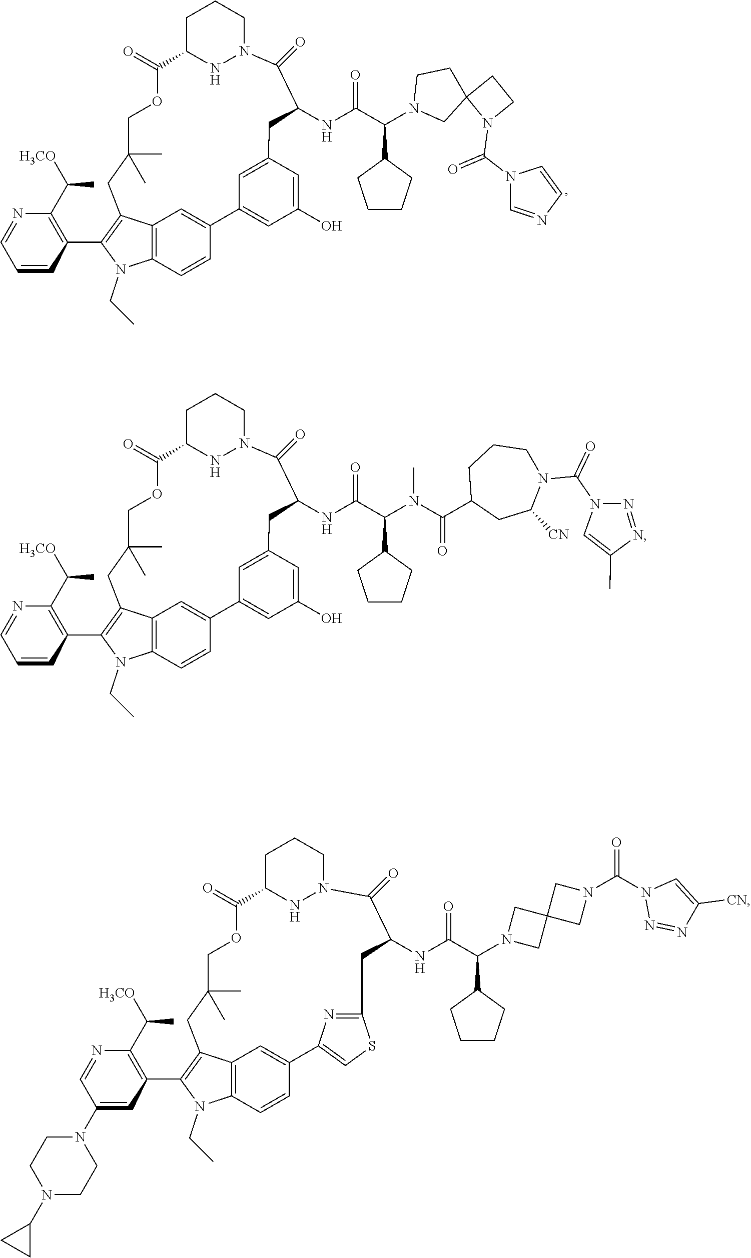

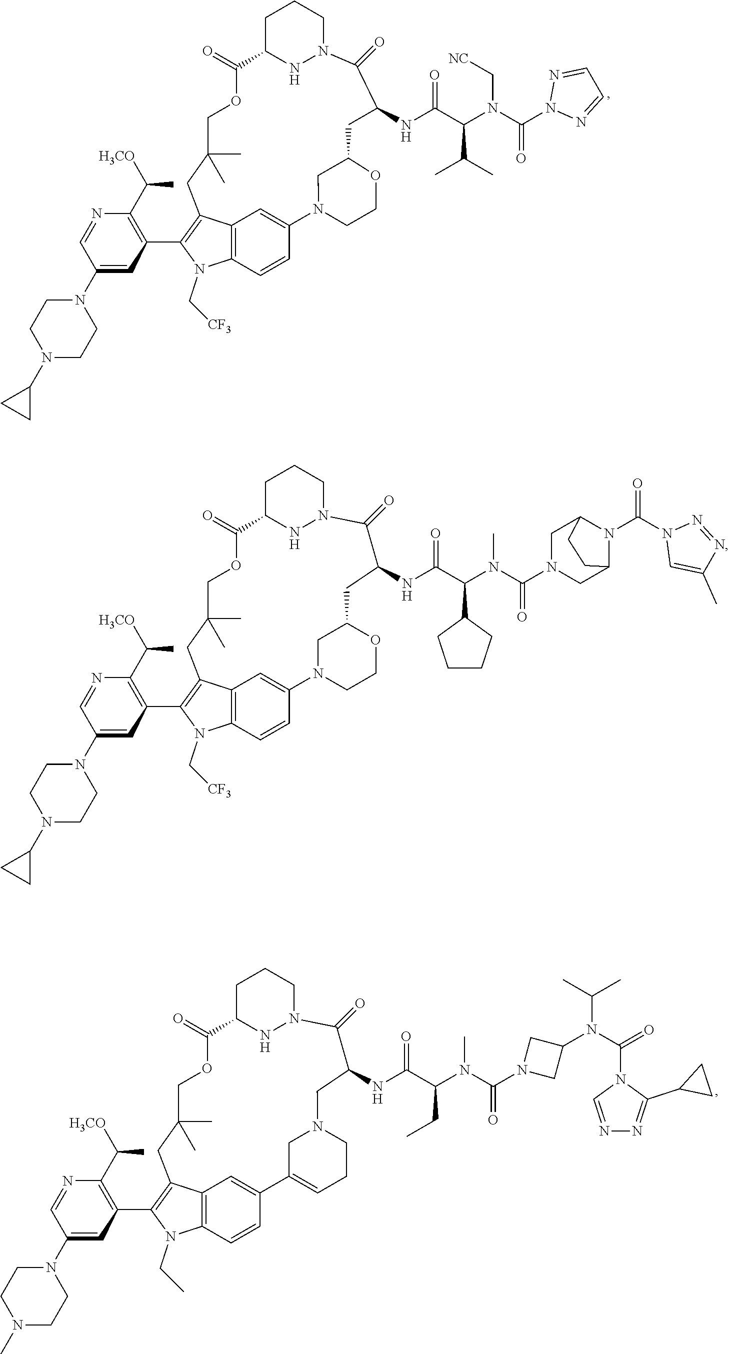

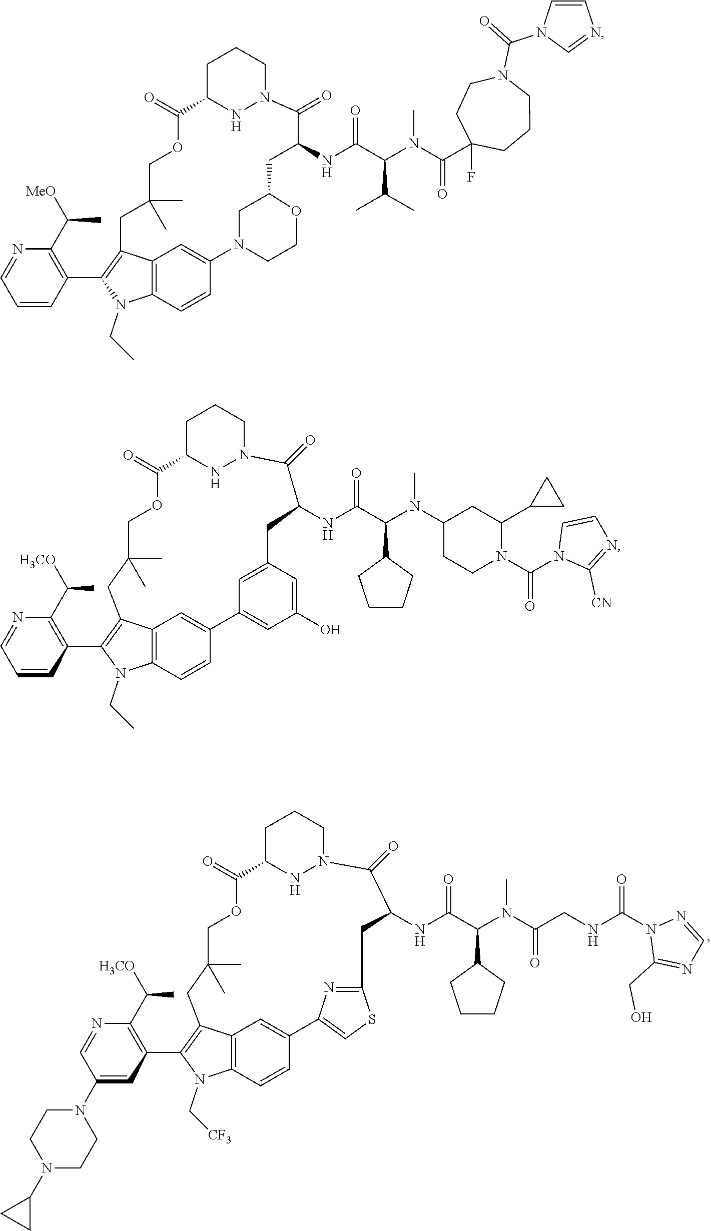

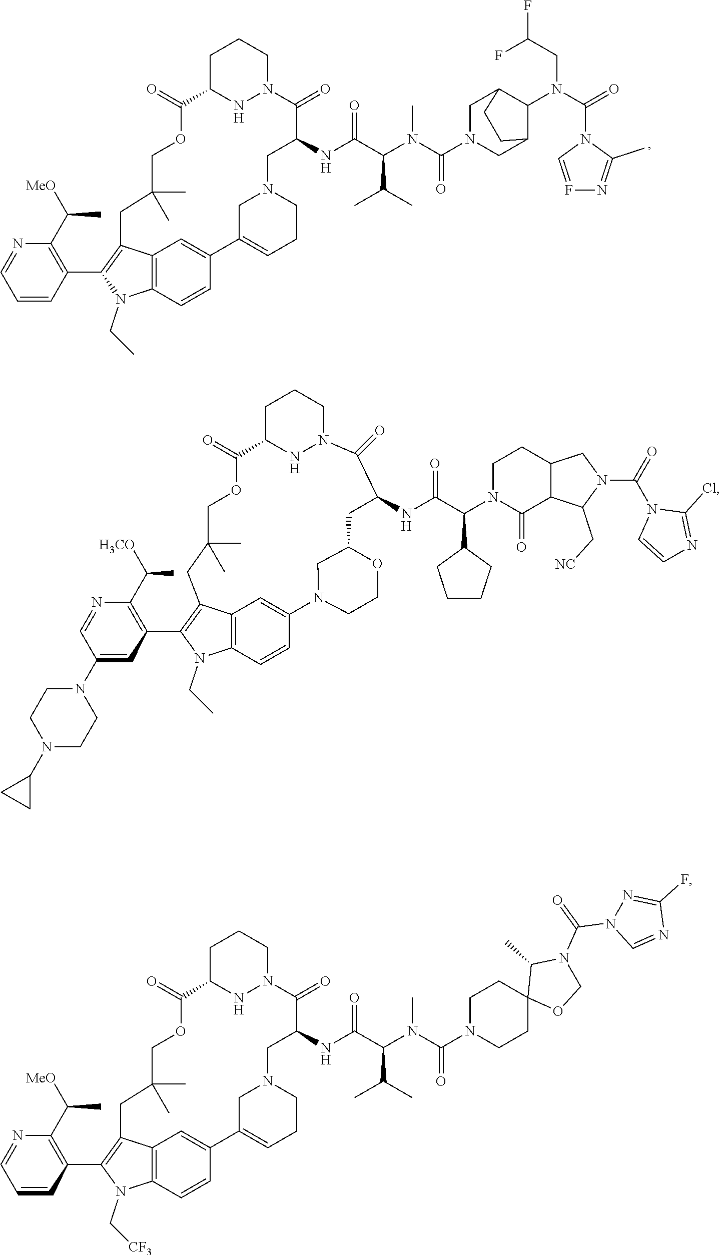

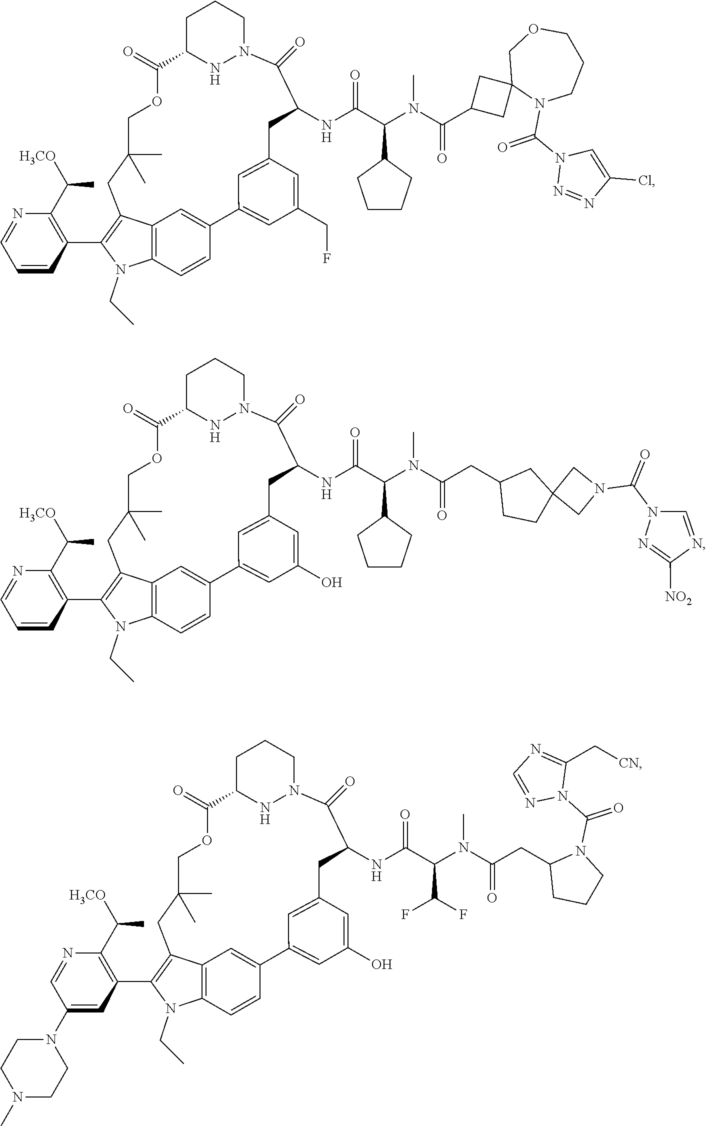

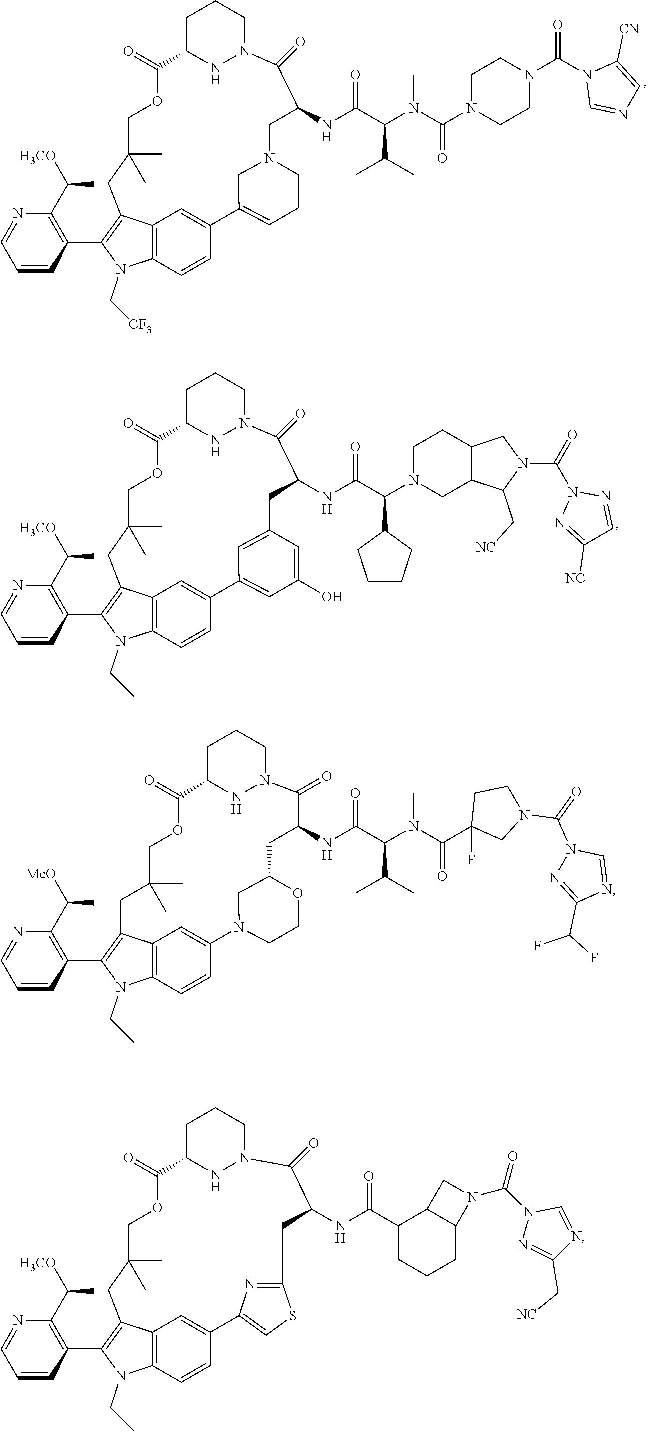

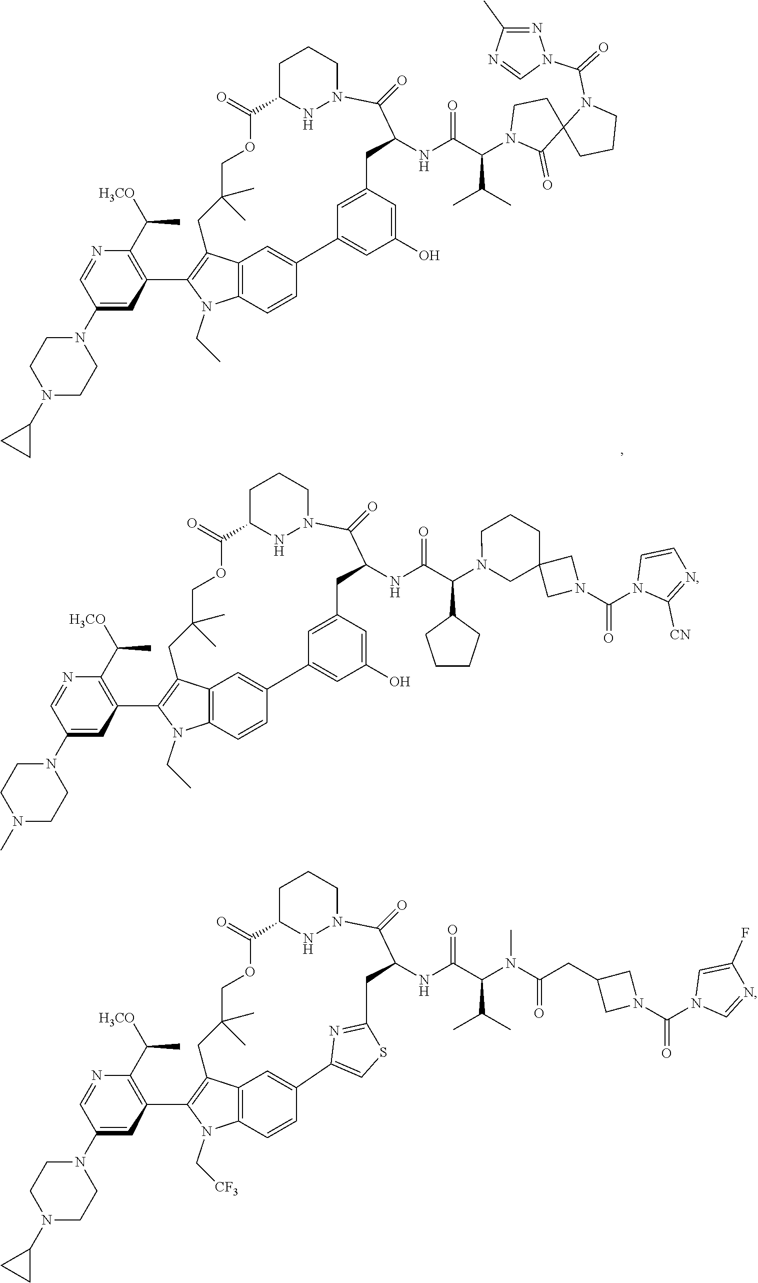

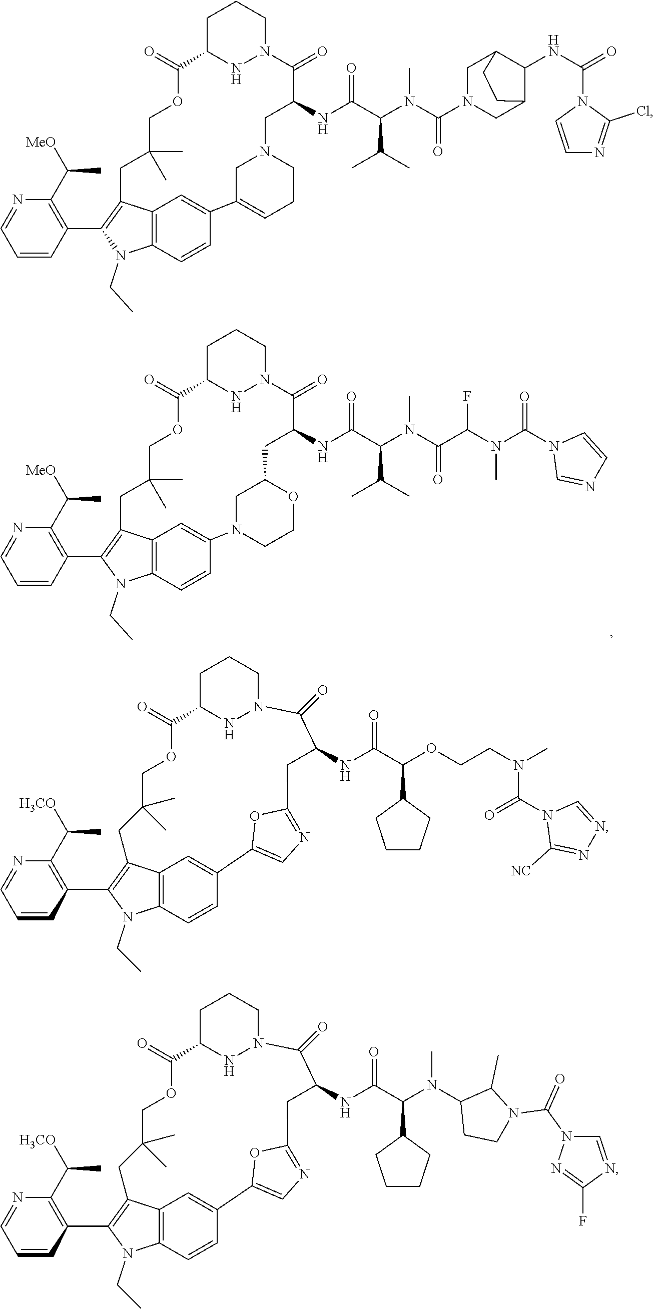

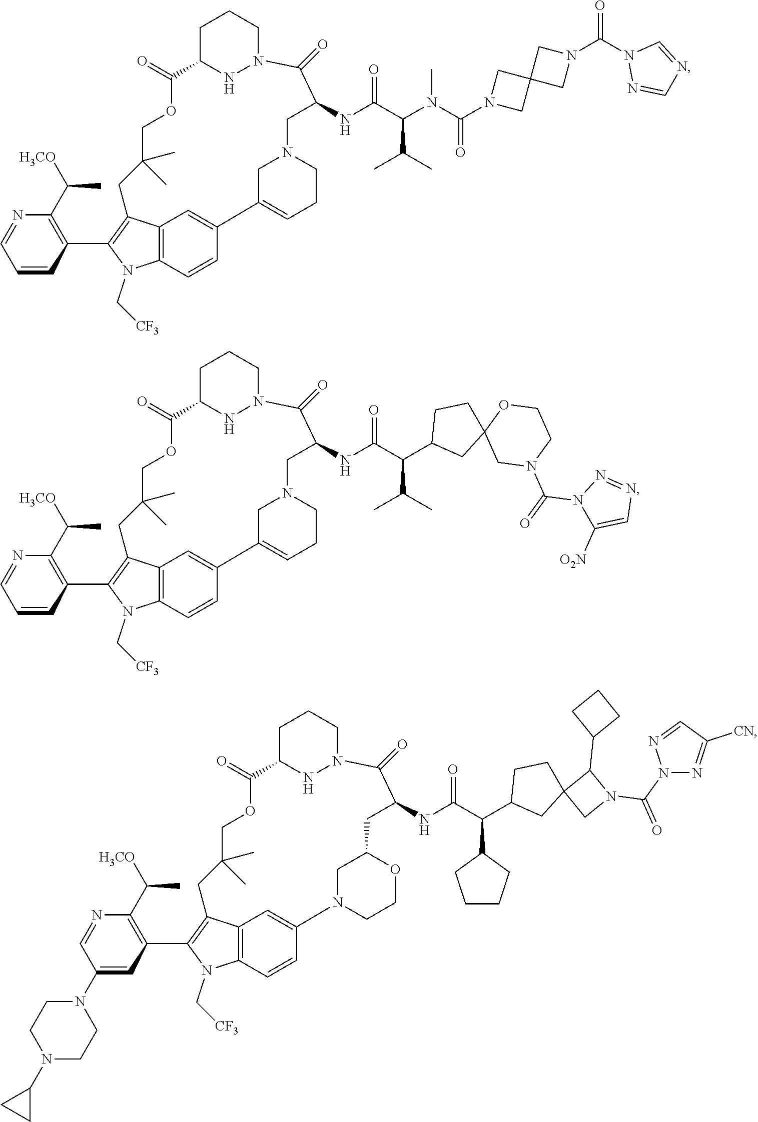

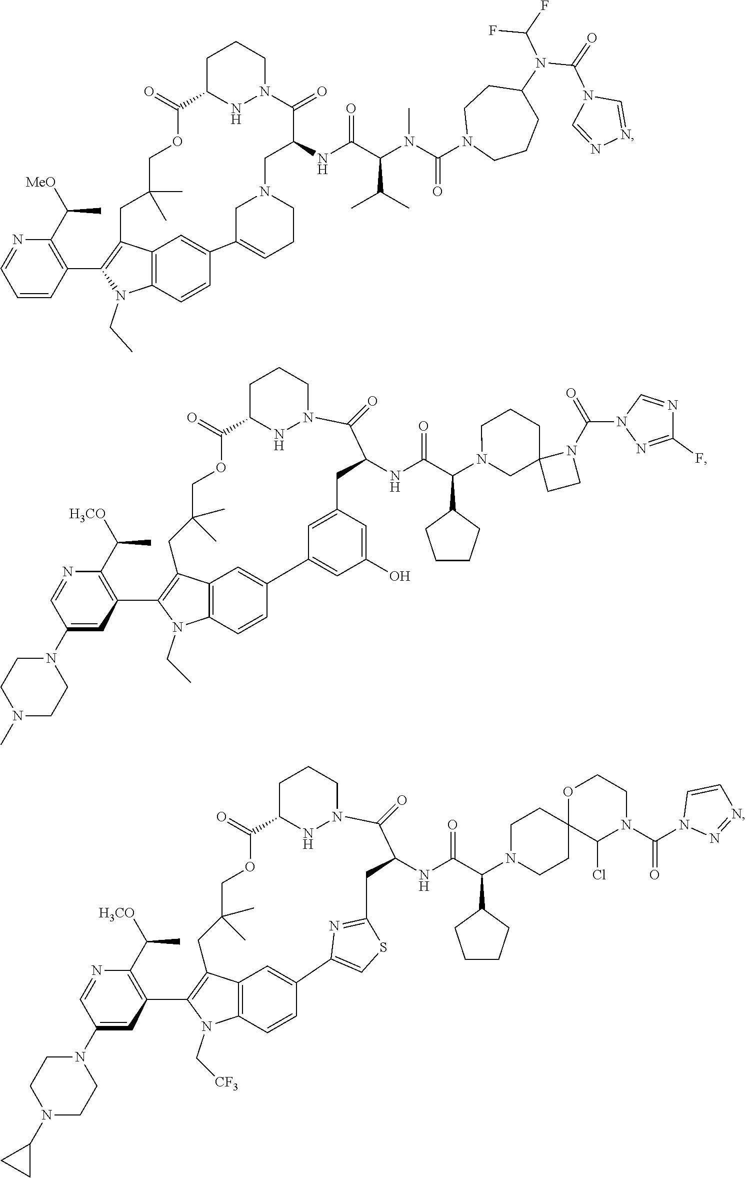

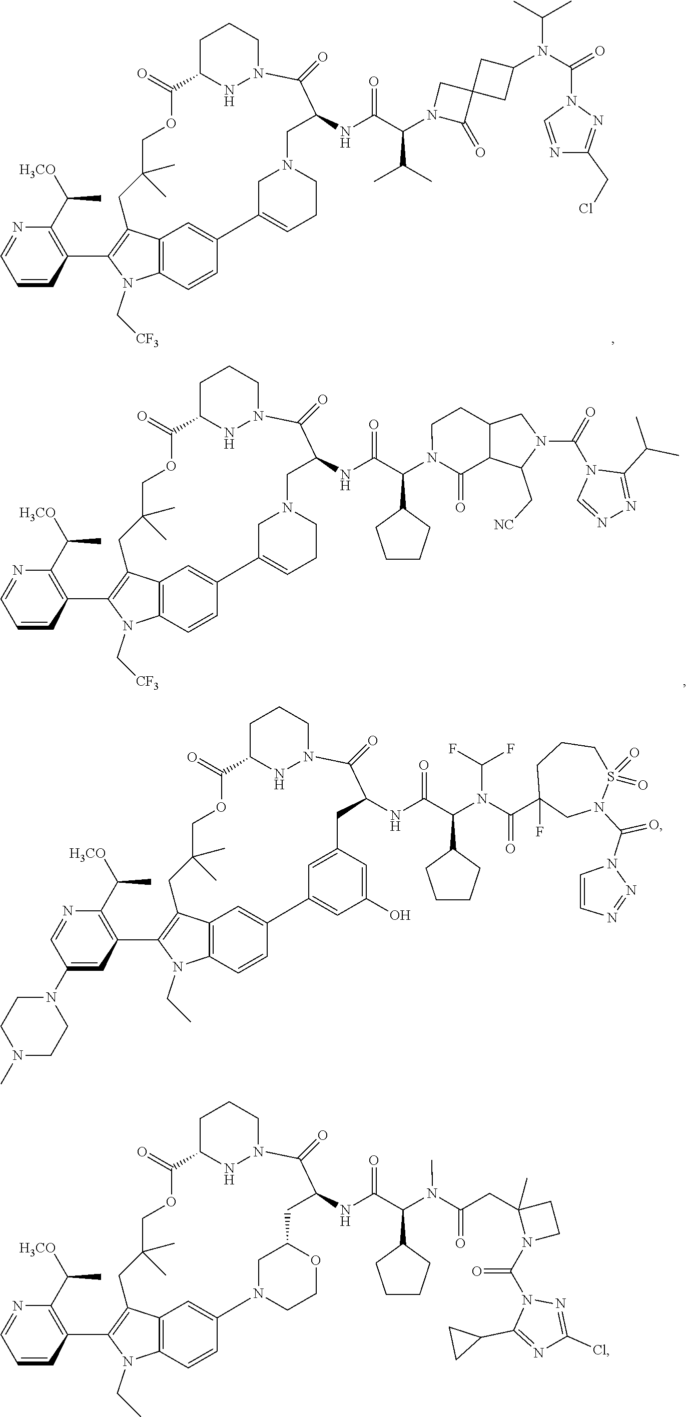

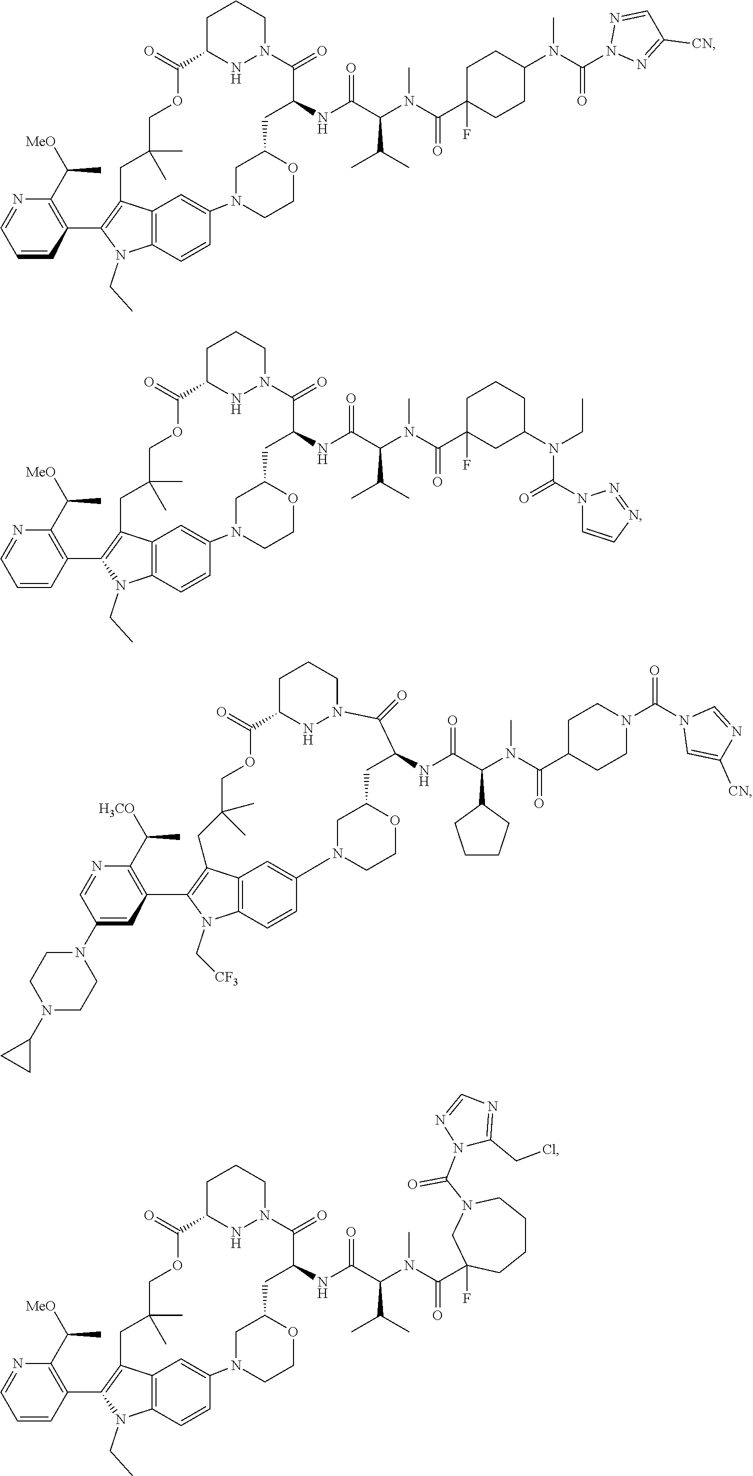

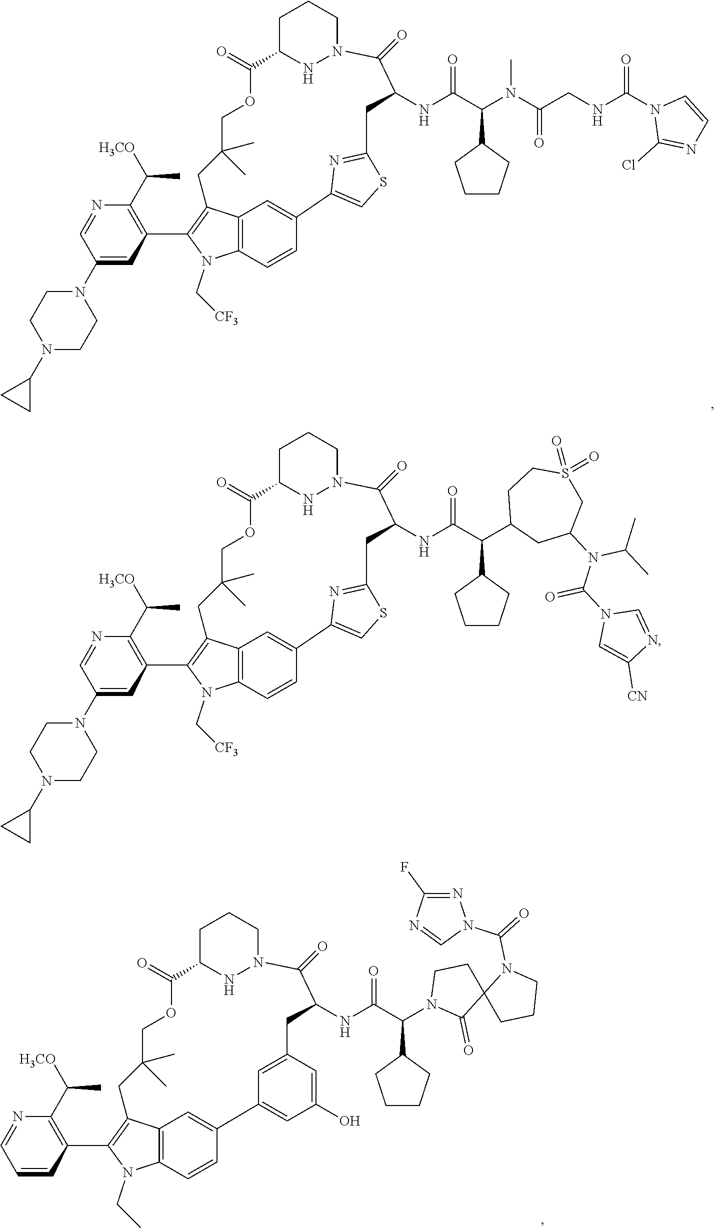

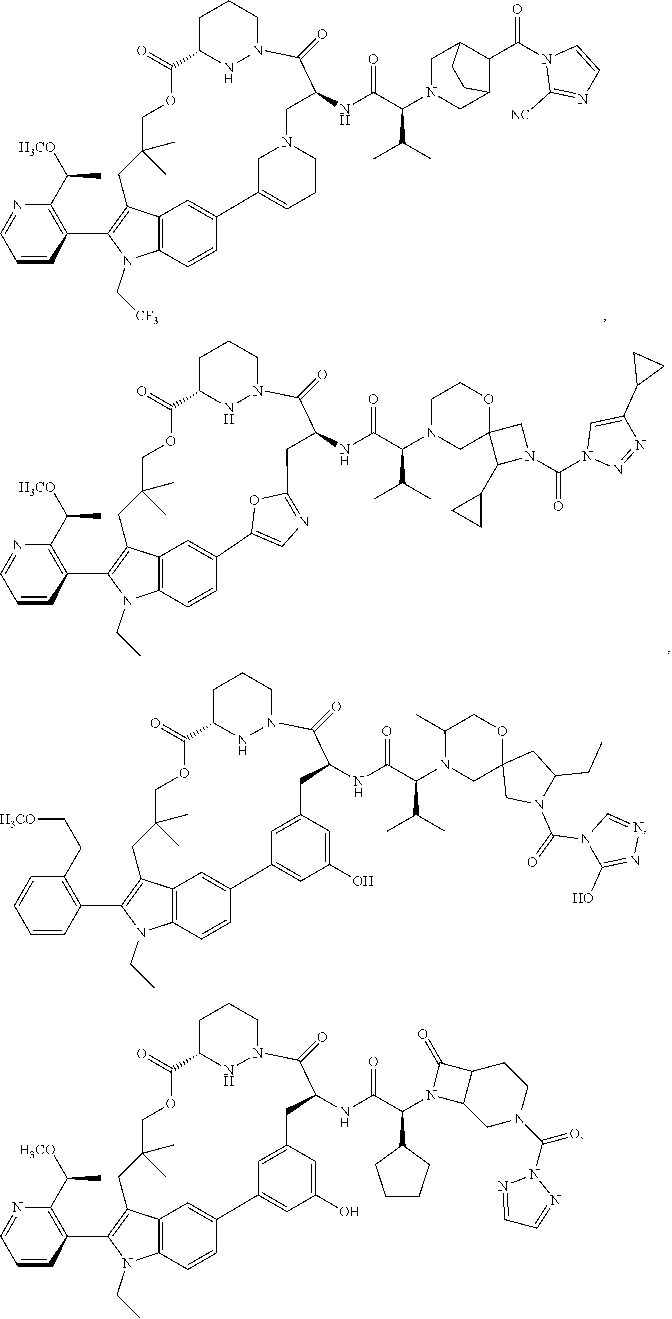

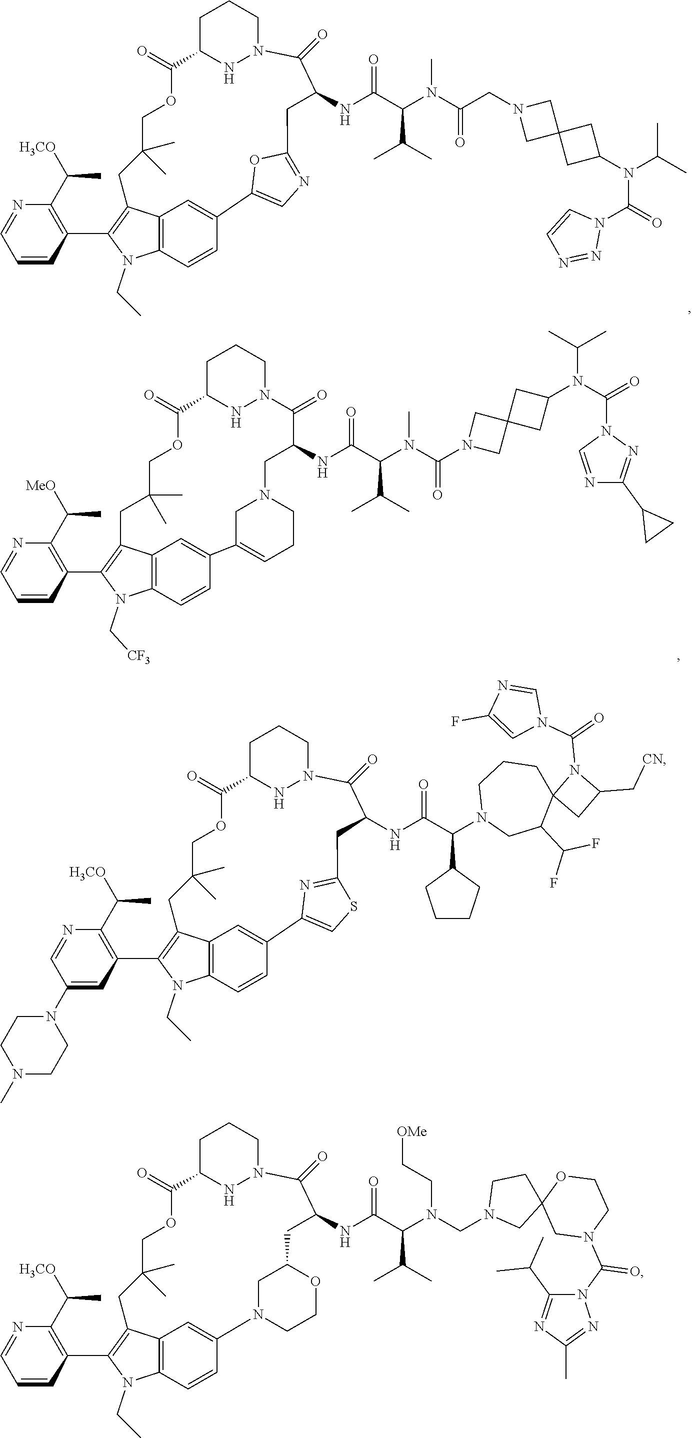

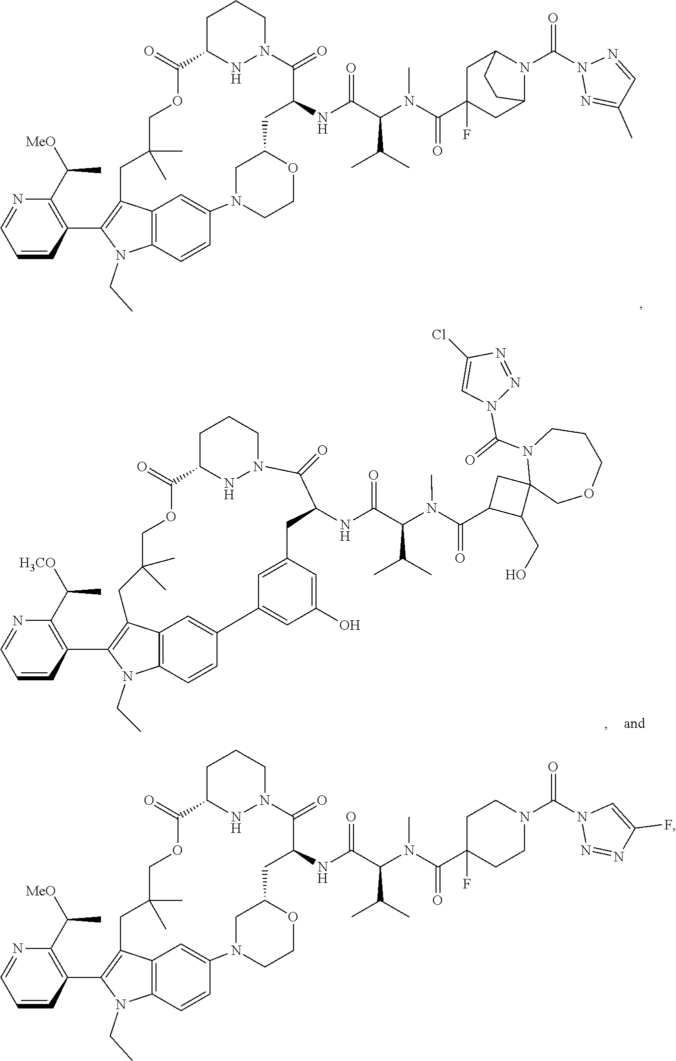

or a pharmaceutically acceptable salt, solvate, stereoisomer, or prodrug thereof.

In general, compounds of the disclosure may be prepared by the following reaction scheme:

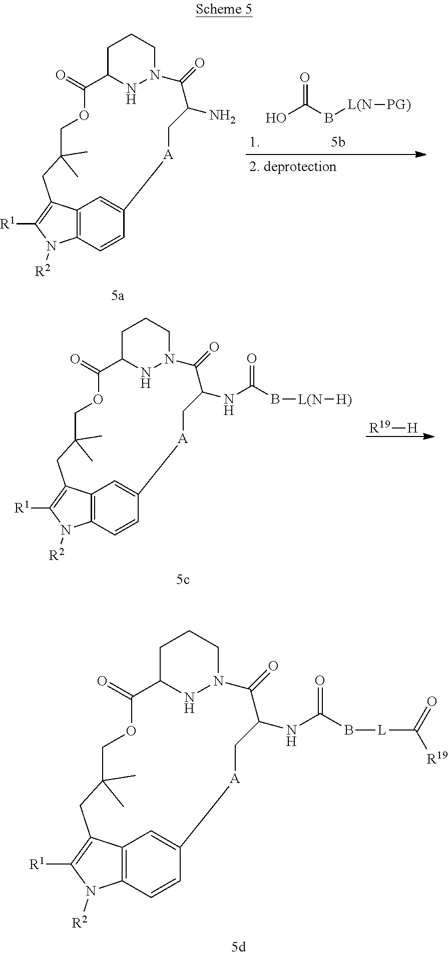

In some embodiments, a compound of Formula 5d may be prepared according to Scheme 5. For example, a peptide coupling reaction between amine 5a and carboxylic acid 5b, wherein L of compound 5b comprises a suitably protected nitrogen atom (e.g., wherein PG is Boc, Cbz, or Fmoc), can provide the corresponding amide. Removal of the N-protecting group to provide 5c can be followed by reaction with R¹⁹—H in the presence of triphosgene bis(trichloromethyl) carbonate (BTC) and pyridine to provide a compound of Formula 5d.

Synthetic procedures for certain intermediates, such as a compound of Formula 5a, may be found in WO 2020/132597, WO 2021/091956, WO 2021/091967, WO 2021/091982, WO 2022/060836, WO 2022/235864, WO 2022/235870, WO 2023/060253, and WO 2023/133543 each of which is incorporated herein by reference in its entirety, including any synthetic methods, recitations of compound variables, combination therapies, and methods of use disclosed therein.

### Compounds of Formula (IX) and (X)

In certain aspects, the present disclosure provides a compound of Formula (IX):

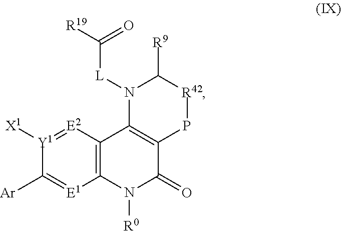

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
P is selected from O, NH, and NR^{m};
R⁴² is selected from —C(O)— and optionally substituted —C₁₋₃ alkyl-;
Y¹ is C and X¹ is selected from hydrogen and R¹⁰; or Y¹ is N and X¹ is absent;
E¹ is selected from N and CR⁵;
E² is selected from N and CR⁶;
provided that Y¹, E¹, and E² are not simultaneously N;
Ar is selected from C₃₋₁₂ carbocycle and 3- to 12-membered heterocycle, each of which is optionally substituted;
R⁰ is selected from C₃₋₁₂ carbocycle and 3- to 12-membered heterocycle, each of which is optionally substituted;
R^{m} is selected from C₁₋₆ alkyl and C₁₋₆ deuteroalkyl, each of which is optionally substituted;
R⁵ and R⁶ are independently selected from hydrogen and R¹⁰;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹⁰ is selected from halogen, —CN, —NO₂, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, for a compound of Formula (IX), P is NR^{m}; R⁴² is —C(O)—; Y¹ is C and X¹ is halogen; E¹ is N; E² is CH; Ar is 3-fluoro-1-hydroxy-phen-2-yl; R⁰ is selected from optionally substituted 5- to 6-membered heteroaryl; and R^{m} is selected from CH₃ and CD₃.

In certain aspects, the present disclosure provides a compound of Formula (X):

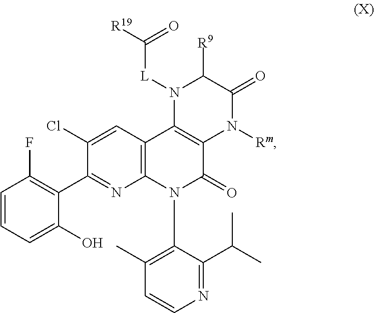

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R^{m} is selected from C₁₋₆ alkyl and C₁₋₆ deuteroalkyl, each of which is optionally substituted;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, the compound of Formula (IX) is selected from:

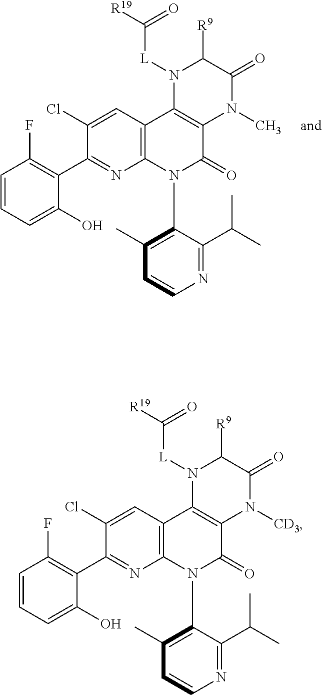

or a pharmaceutically acceptable salt or solvate thereof.

In some embodiments, for a compound of Formula (IX) or (X), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea.

In some embodiments, for a compound of Formula (IX) or (X), L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH— or —CH₂N(CH₃)—. In some embodiments, L¹ is selected from a bond and 2- to 6-membered heteroalkyl. In some embodiments, L¹ is selected from a bond and optionally substituted 2- to 6-membered heteroalkyl.

In some embodiments, for a compound of Formula (IX) or (X), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (IX) or (X), L², R⁹, and the atoms to which they are attached form optionally substituted 3- to 12-membered heterocycle. In some embodiments, L², R⁹, and the atoms to which they are attached form optionally substituted 3- to 7-membered monocyclic heterocycle, such as optionally substituted 3- to 7-membered monocyclic heterocycloalkyl. In some embodiments, L², R⁹, and the atoms to which they are attached form optionally substituted 5- to 8-membered monocyclic heterocycle, such as optionally substituted 5- to 8-membered monocyclic heterocycloalkyl. In some embodiments, L², R⁹, and the atoms to which they are attached form optionally substituted 6- to 12-membered spirocyclic heterocycle, such as optionally substituted 6- to 12-membered spirocyclic heterocycloalkyl. In some embodiments, L², R⁹, and the atoms to which they are attached form optionally substituted 7- to 12-membered fused bicyclic heterocycle, such as optionally substituted 7- to 12-membered fused bicyclic heterocycle.

In some embodiments, for a compound of Formula (IX) or (X), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (IX) or (X), L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle. In some embodiments, L¹ is a bond; L², R⁹, and the atoms to which they are attached form optionally substituted 3- to 12-membered heterocycle; and L³ is selected from a bond and optionally substituted nitrene.

In some embodiments, the compound of Formula (IX) is:

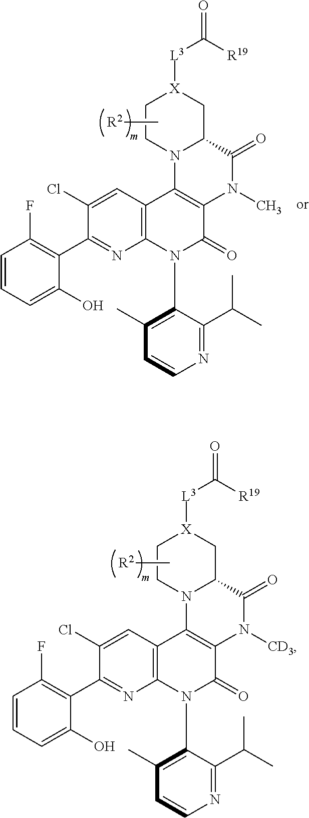

or a pharmaceutically acceptable salt or solvate thereof, wherein:
X is selected from CH and N;
R² is selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R² and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
m is 0, 1, 2, or 3.

In some embodiments, for a compound of Formula (IX), R² is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R² is selected from —CH₃, and —CH₂CN. In some embodiments, R² is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2. In some embodiments, R² is selected from —CH₃, and —CH₂CN; and m is 1 or 2. In some embodiments, m is 0. In some embodiments, X is N and L³ is a bond. In some embodiments, X is CH and L³ is optionally substituted nitrene.

In certain aspects, the present disclosure provides a compound selected from:

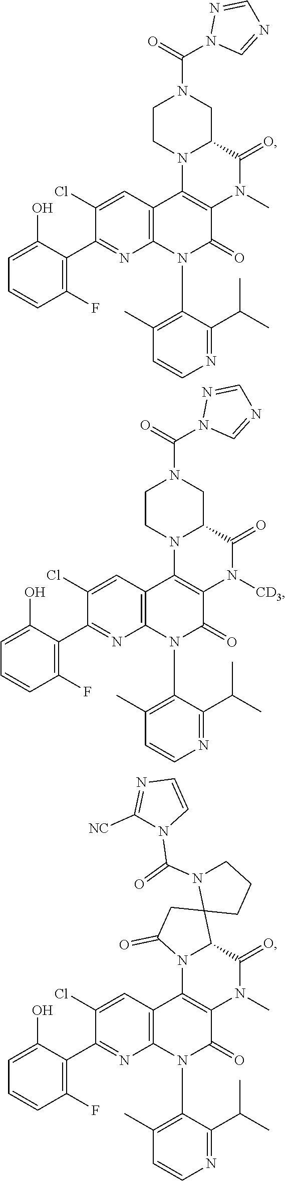

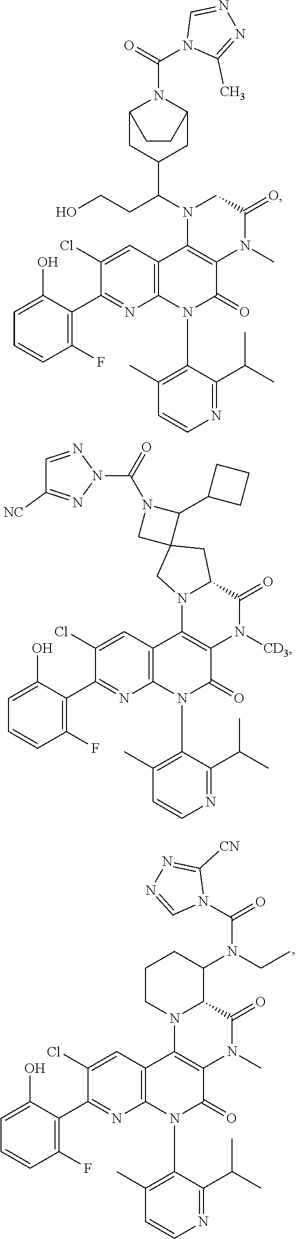

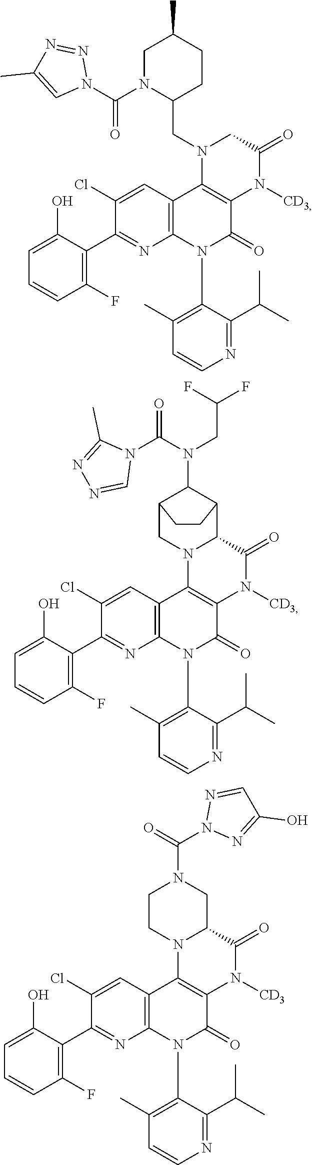

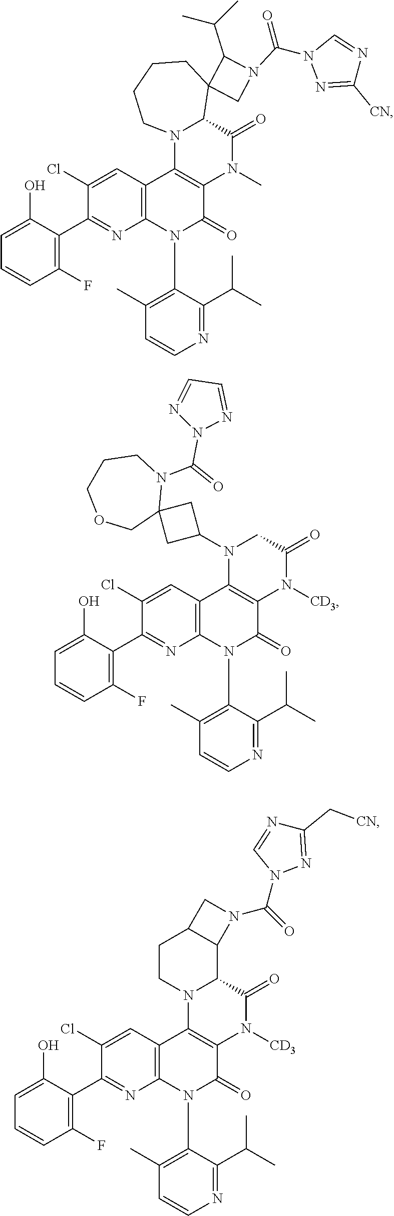

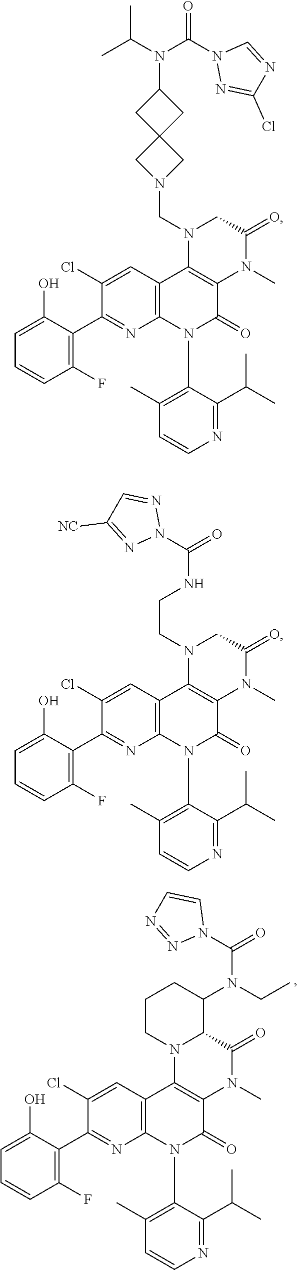

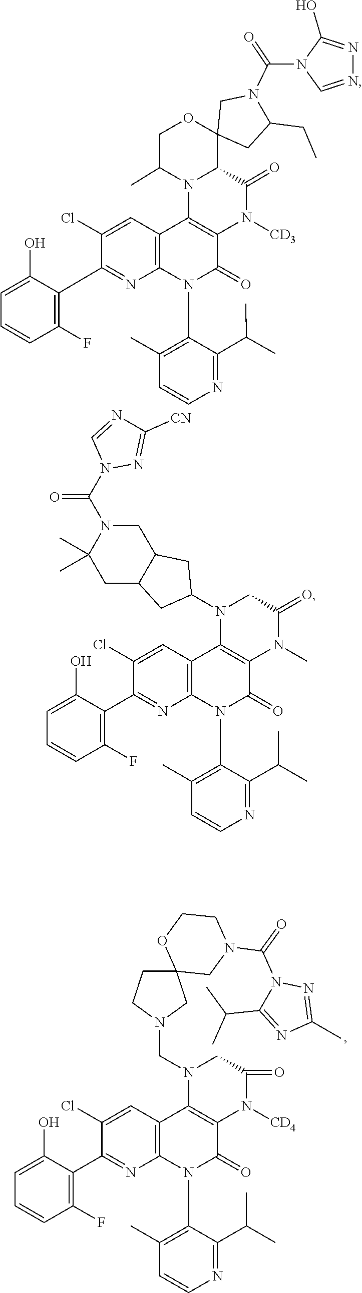

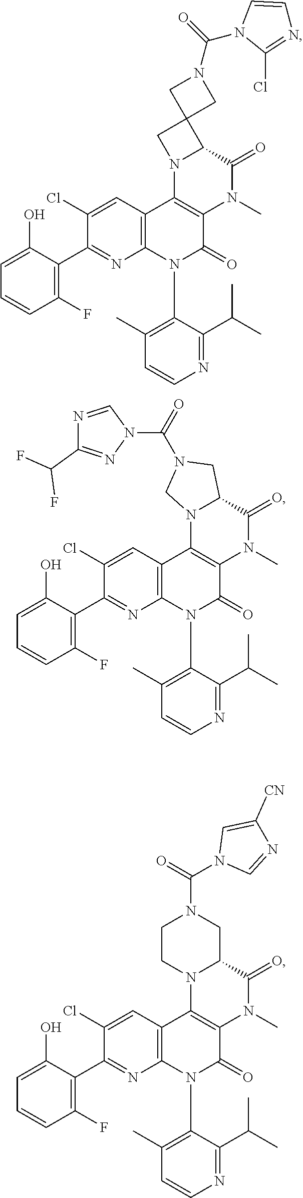

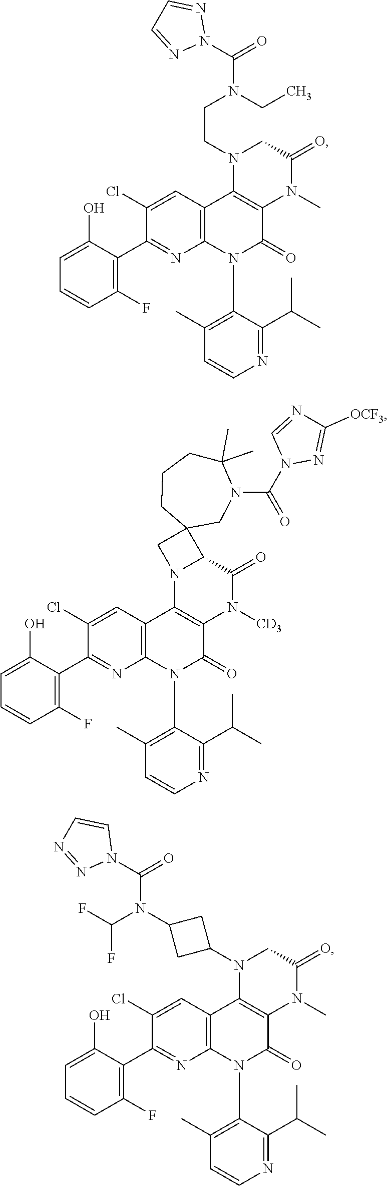

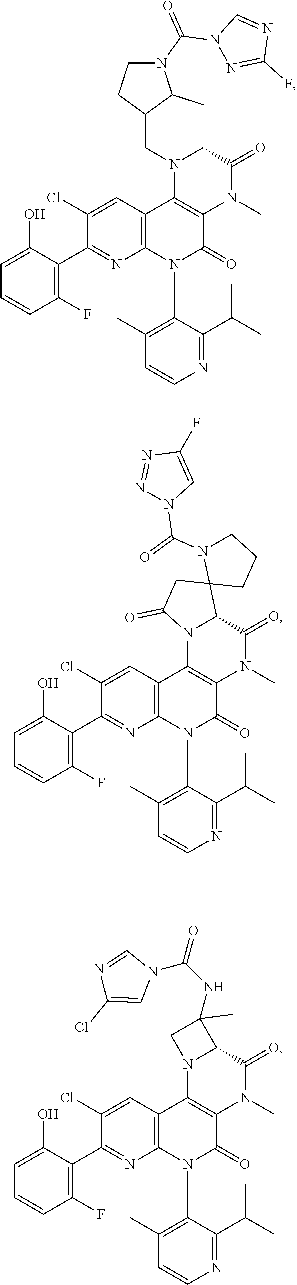

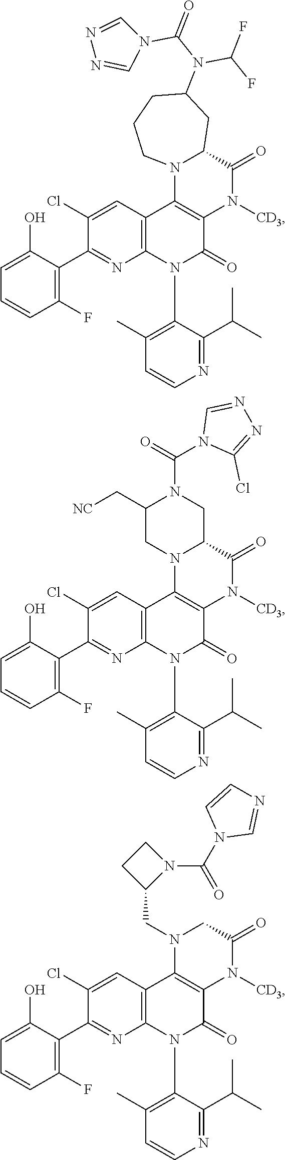

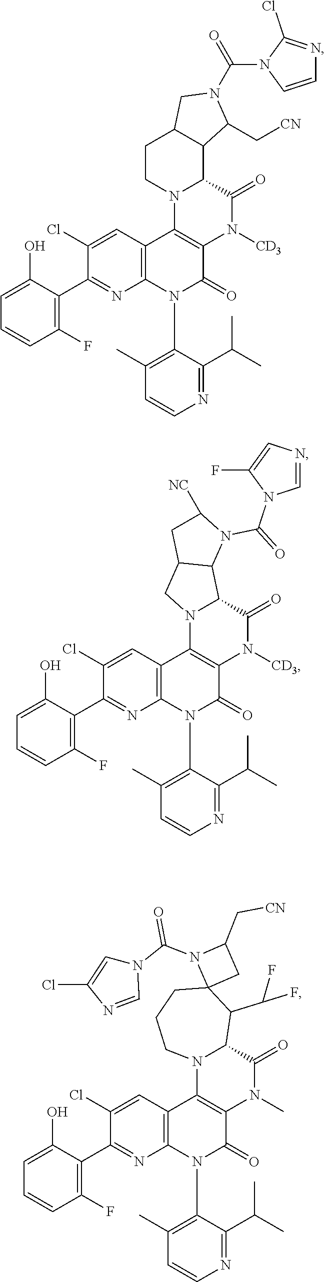 [SMILES: [2H]C([2H])([2H])N1C(=O)[C@H]2C3C(CCN2c2c1c(=O)n(-c1c(C)ccnc1C(C)C)c1nc(-c4c(O)cccc4F)c(Cl)cc21)CN(C(=O)n1ccnc1Cl)C3CC#N ; [2H]C([2H])([2H])N1C(=O)[C@H]2C3C(CC(C#N)N3C(=O)n3cncc3F)CN2c2c1c(=O)n(-c1c(C)ccnc1C(C)C)c1nc(-c3c(O)cccc3F)c(Cl)cc21 ; Cc1ccnc(C(C)C)c1-n1c(=O)c2c(c3cc(Cl)c(-c4c(O)cccc4F)nc31)N1CCCC3(CC(CC#N)N3C(=O)n3cnc(Cl)c3)C(C(F)F)[C@@H]1C(=O)N2C]

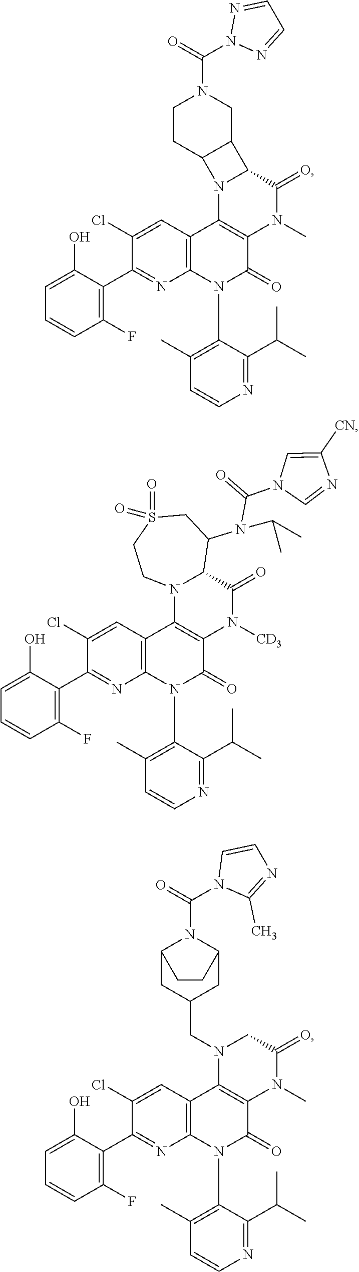

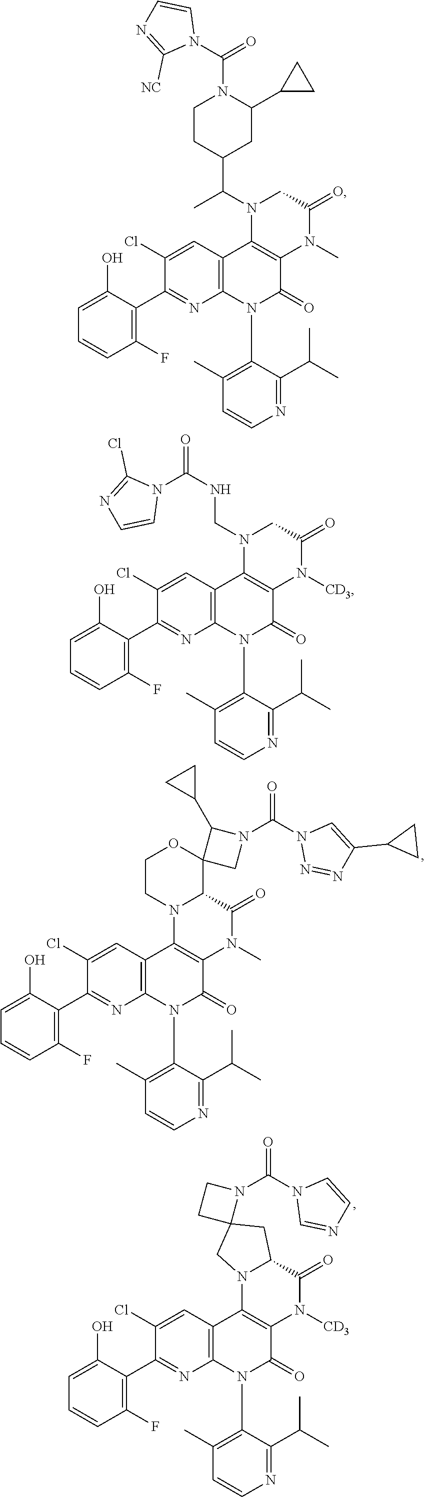

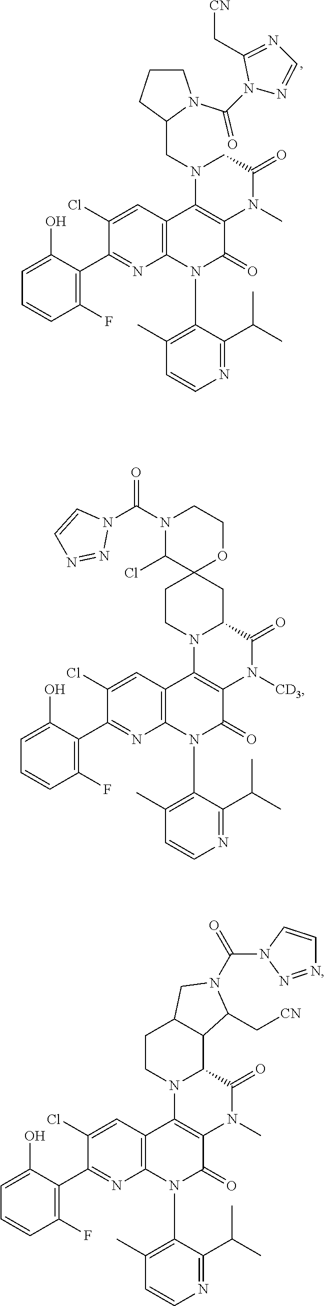

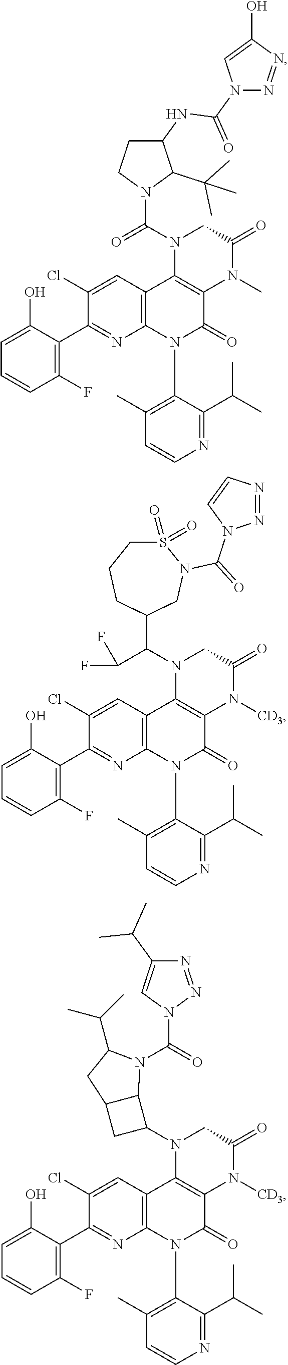

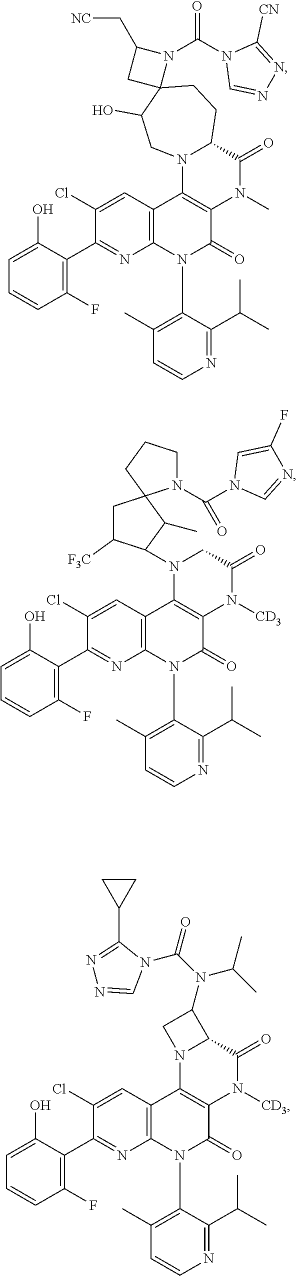

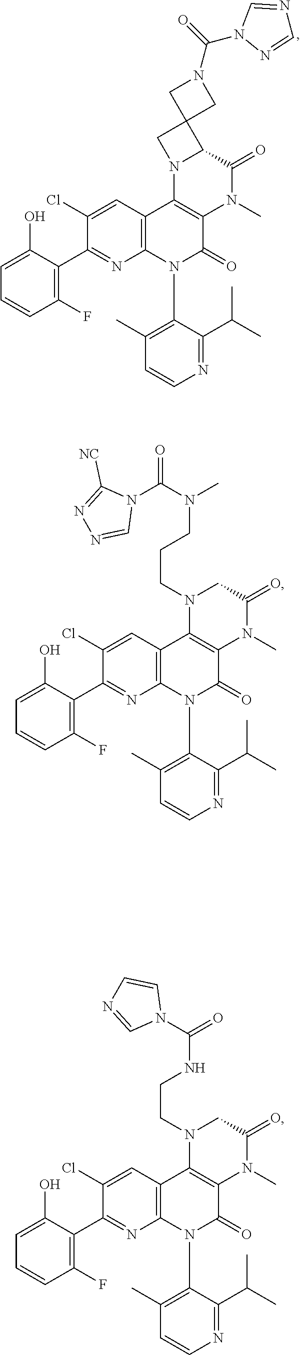

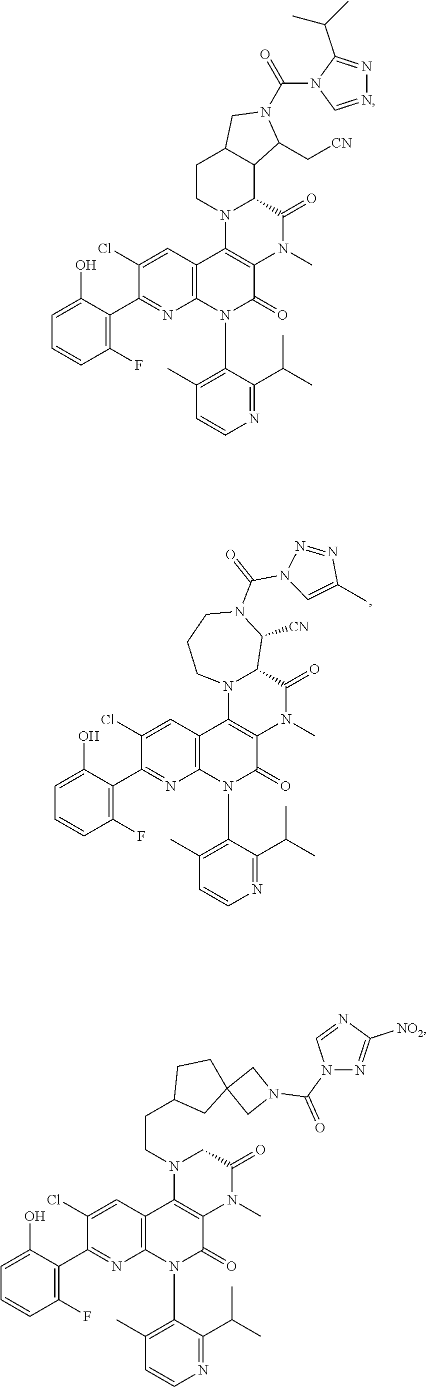

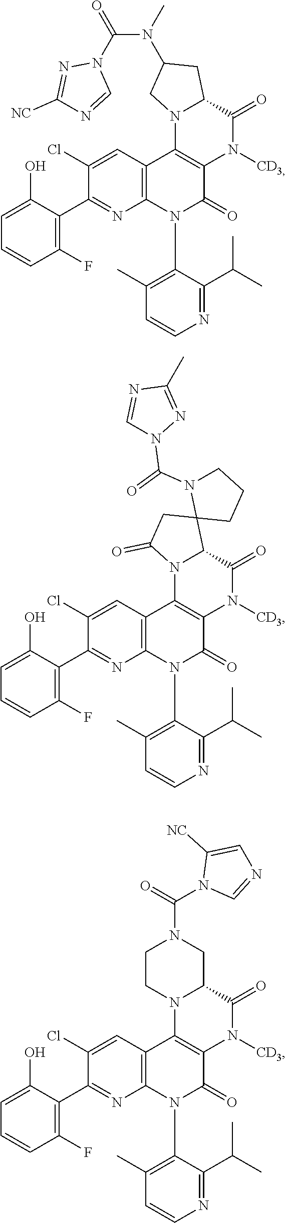

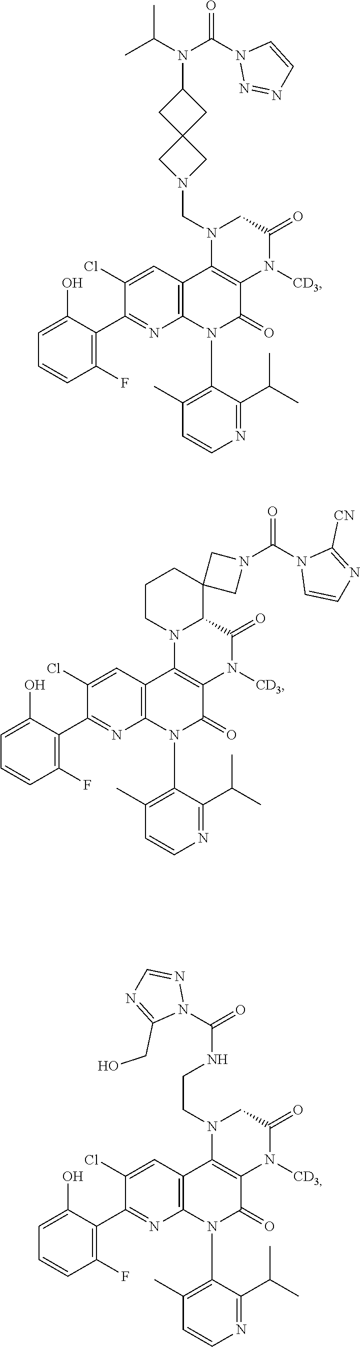

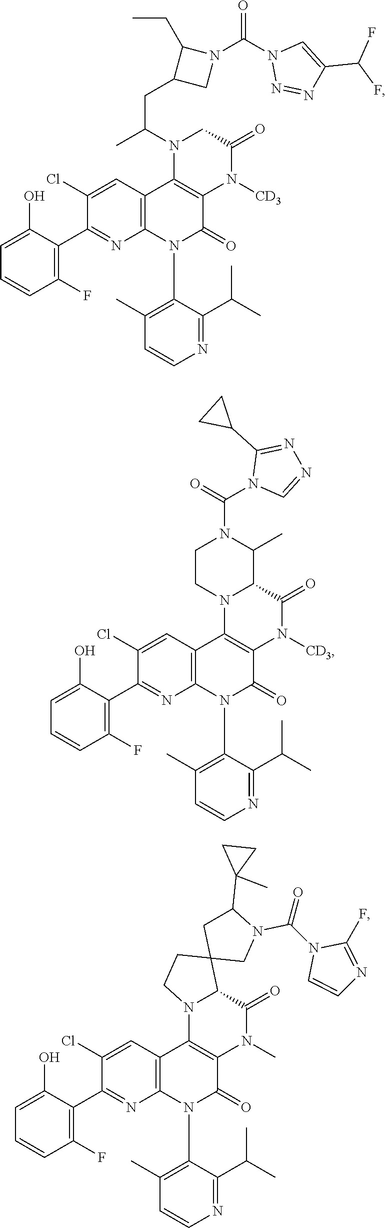

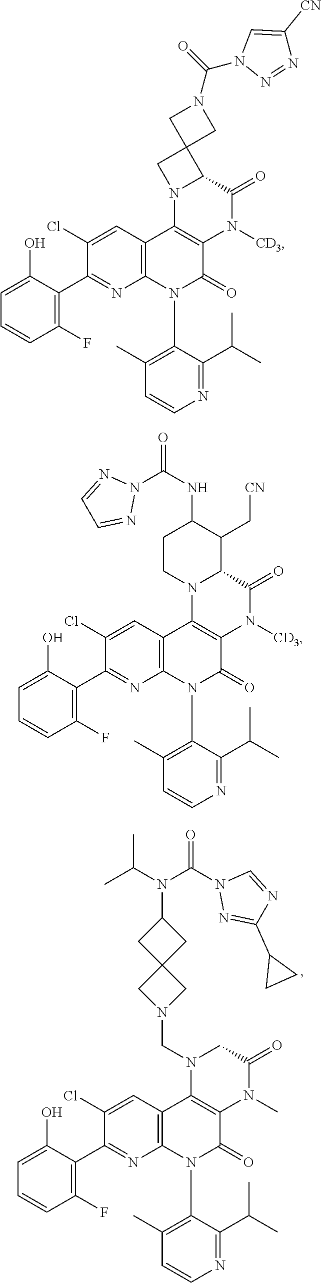

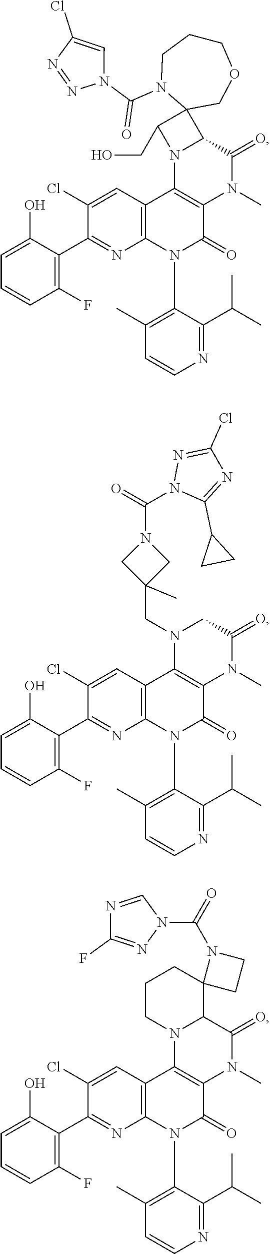

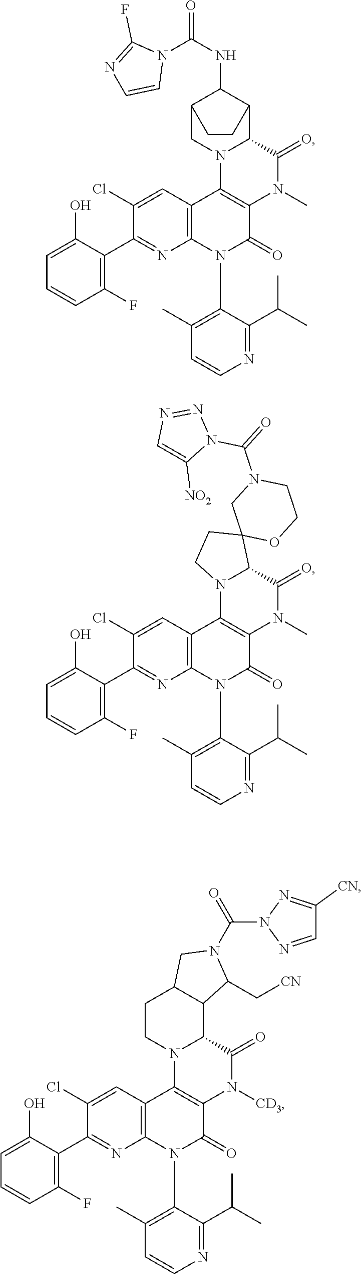

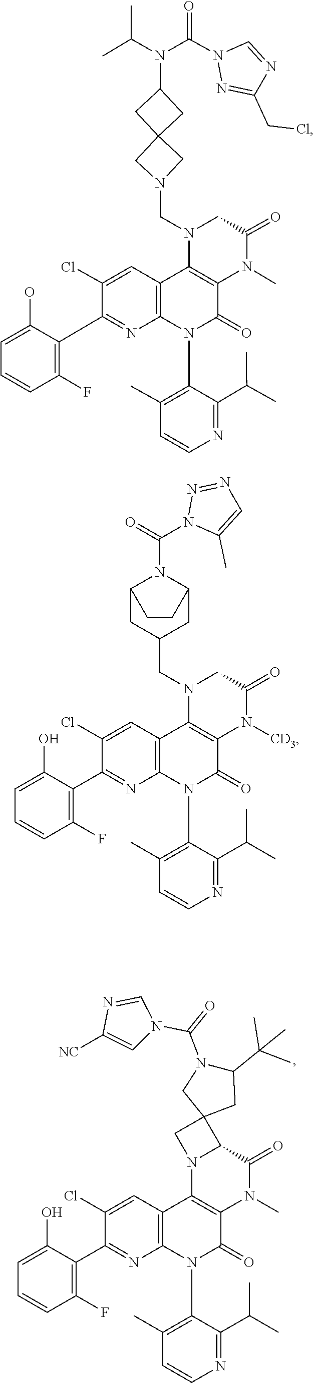

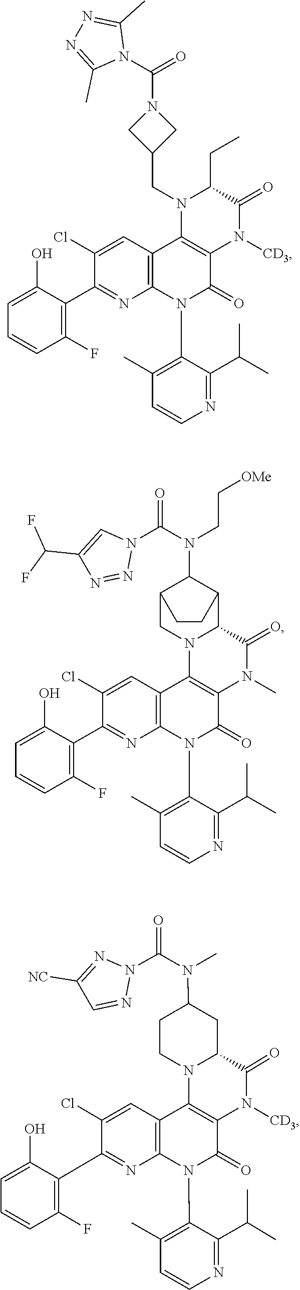 [SMILES: [2H]C([2H])([2H])N1C(=O)[C@@H](CC)N(CC2CN(C(=O)n3c(C)nnc3C)C2)c2c1c(=O)n(-c1c(C)ccnc1C(C)C)c1nc(-c3c(O)cccc3F)c(Cl)cc21 ; COCCN(C(=O)n1cc(C(F)F)nn1)C1C2CCC1[C@@H]1C(=O)N(C)c3c(c4cc(Cl)c(-c5c(O)cccc5F)nc4n(-c4c(C)ccnc4C(C)C)c3=O)N1C2 ; [2H]C([2H])([2H])N1C(=O)[C@H]2CC(N(C)C(=O)n3ncc(C#N)n3)CCN2c2c1c(=O)n(-c1c(C)ccnc1C(C)C)c1nc(-c3c(O)cccc3F)c(Cl)cc21]

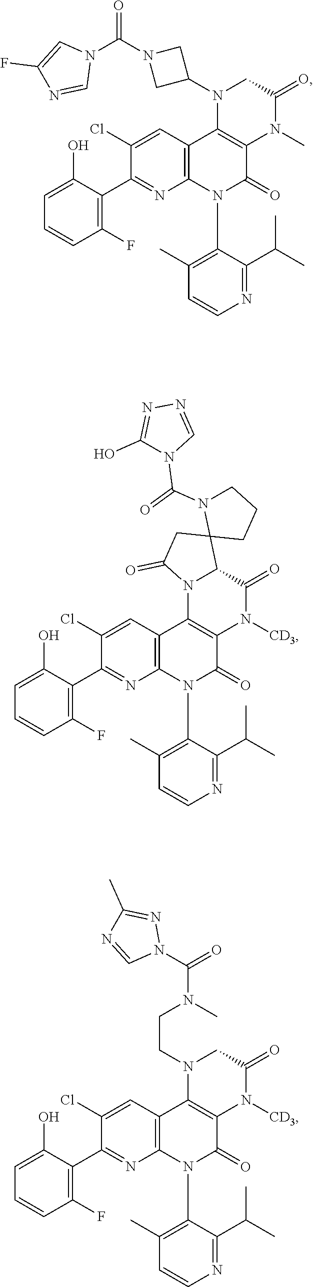

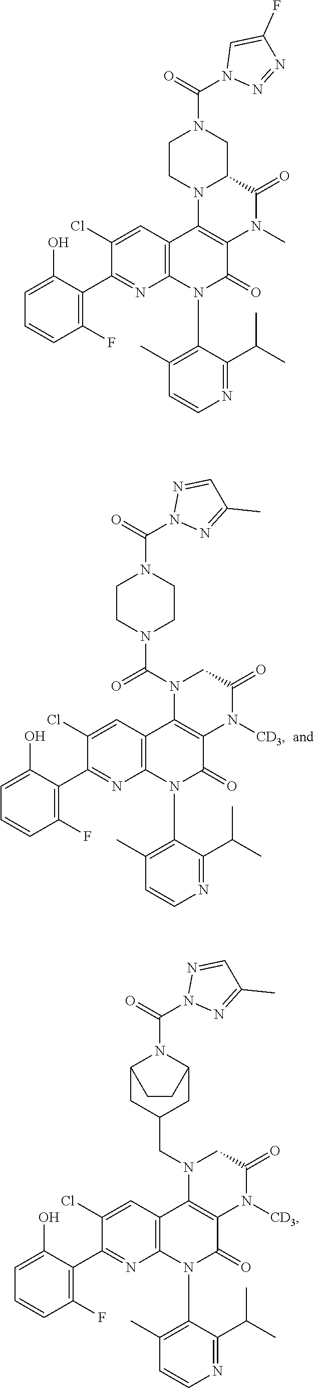

or a pharmaceutically acceptable salt, solvate, stereoisomer, or prodrug thereof.

In general, compounds of the disclosure may be prepared by the following reaction scheme:

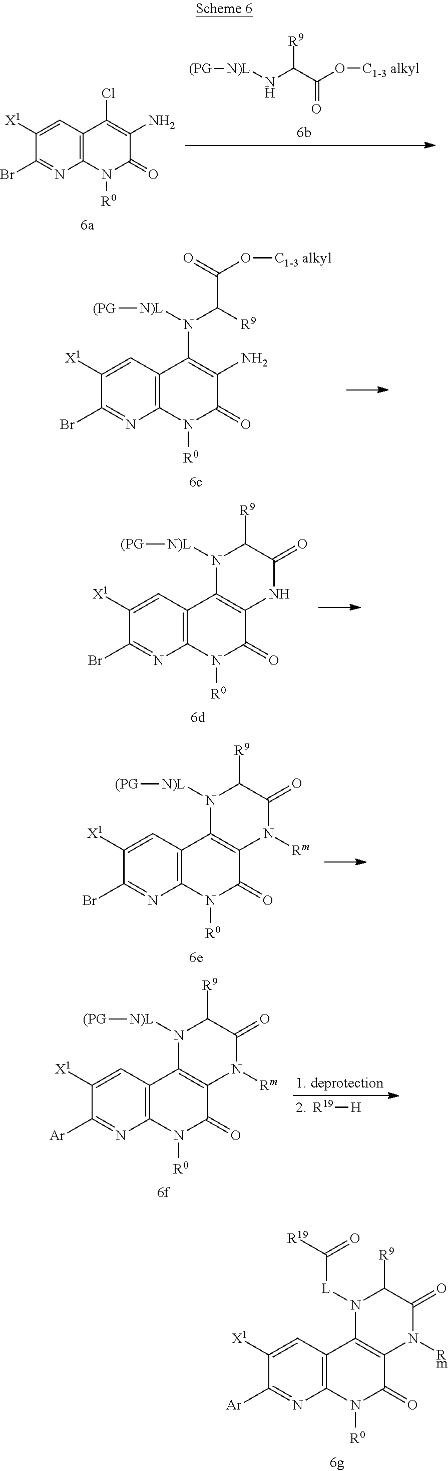

In some embodiments, a compound of Formula 6g may be prepared according to Scheme 6. For example, amine 6c can be formed from chloride 6a via a nucleophilic aromatic substitution reaction with amine 6b (wherein L of compound 6b comprises a suitably protected nitrogen atom (e.g., wherein PG is Boc, Cbz, or Fmoc)). Ring closure to 6d can be followed by an optional alkylation reaction of the amide with R^{m} to provide 6e. Substitution of the aryl bromide with a suitable boronic ester can provide the corresponding Ar-substituted compound (6. Deprotection can be followed by reaction with R¹⁹—H in the presence of triphosgene bis(trichloromethyl) carbonate (BTC) and pyridine to provide a compound of Formula 6g.

Synthetic procedures for certain intermediates may be found in WO 2021/083167 and US Pub. No. 2023/0084095, each of which is incorporated herein by reference in its entirety, including any synthetic methods, recitations of compound variables, combination therapies, and methods of use disclosed therein.

### Compounds of Formula (XI) and (XII)

In certain aspects, the present disclosure provides a compound of Formula (XI):

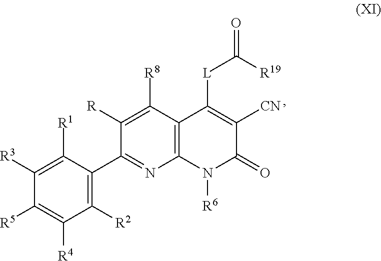

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R¹, R², R³, R⁴, R⁵, R⁷, and R⁸ are independently selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁶ is selected from C₁₋₁₄ alkyl, C₂₋₁₄ alkenyl, C₂₋₁₄ alkynyl, C₃₋₁₂ carbocycle, and 3- to 12-membered heterocycle, each of which is optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

In some embodiments, for a compound of Formula (XI), R¹, R², R³, R⁴, and R⁵ are independently selected from halogen, —NH₂, —OH, and —CF₃. In some embodiments, R¹ is —NH₂ and R², R³, R⁴, and R⁵ are each —F. In some embodiments, R⁷ is —Cl and R⁸ is hydrogen.

In certain aspects, the present disclosure provides a compound of Formula (XII):

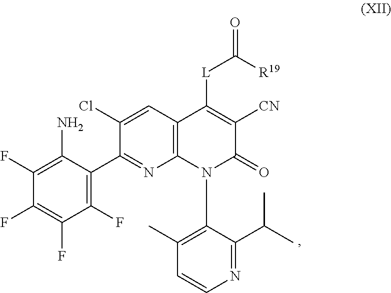

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted; and
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted.

In some embodiments, for a compound of Formula (XI) or (XII), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea.

In some embodiments, for a compound of Formula (XI) or (XII), L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH—, —CH₂N(CH₃)—, —NHCH₂—, or —N(CH₃)CH₂—. In some embodiments, L¹ is selected from a bond, 2- to 6-membered heteroalkyl, and —N(R¹²)—.

In some embodiments, for a compound of Formula (XI) or (XII), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (XI) or (XII), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (XI) or (XII), L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In some embodiments, the compound of Formula (XI) is:

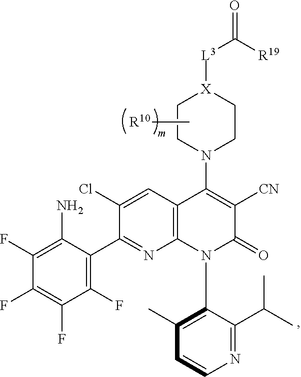

or a pharmaceutically acceptable salt or solvate thereof, wherein:
X is selected from CH and N;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
m is 0, 1, 2, or 3.

In some embodiments, for a compound of Formula (XI), R¹⁰ is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R¹⁰ is selected from —CH₃, and —CH₂CN. In some embodiments, R¹⁰ is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2. In some embodiments, R¹⁰ is selected from —CH₃, and —CH₂CN; and m is 1 or 2. In some embodiments, m is 0. In some embodiments, X is N and L³ is a bond. In some embodiments, X is CH and L³ is optionally substituted nitrene.

In certain aspects, the present disclosure provides a compound selected from:

[chemical structure]

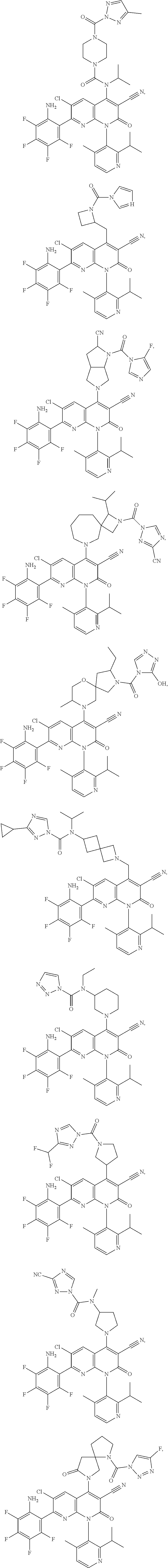

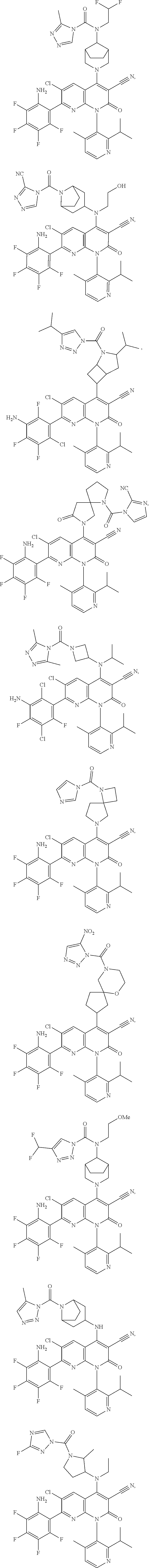

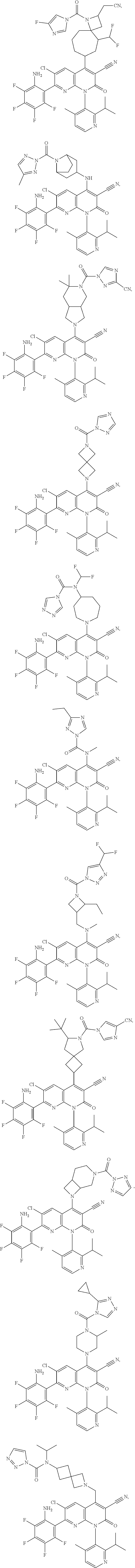

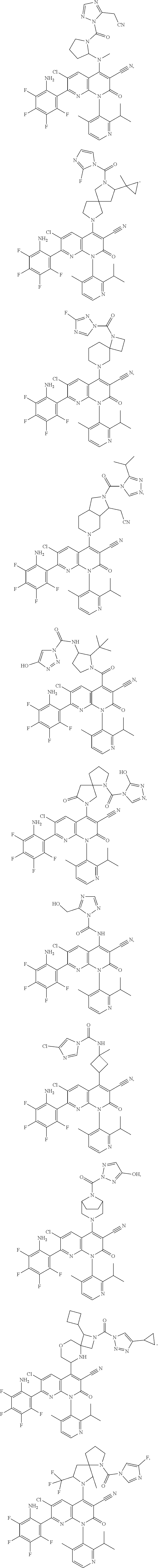

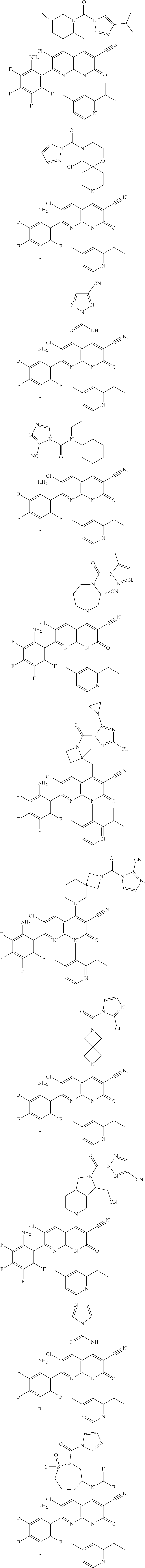

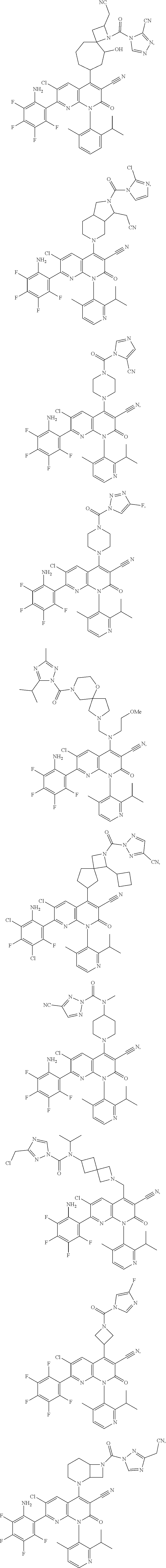

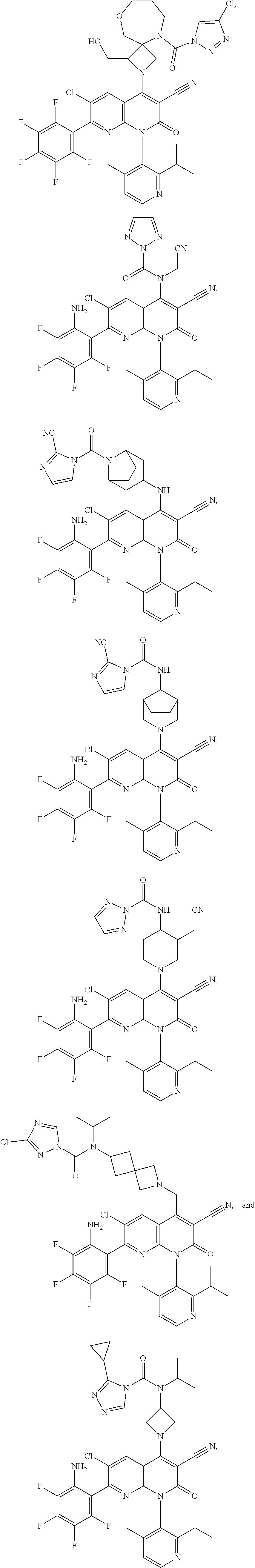

or a pharmaceutically acceptable salt, solvate, stereoisomer, or prodrug thereof.

In general, compounds of the disclosure may be prepared by the following reaction scheme:

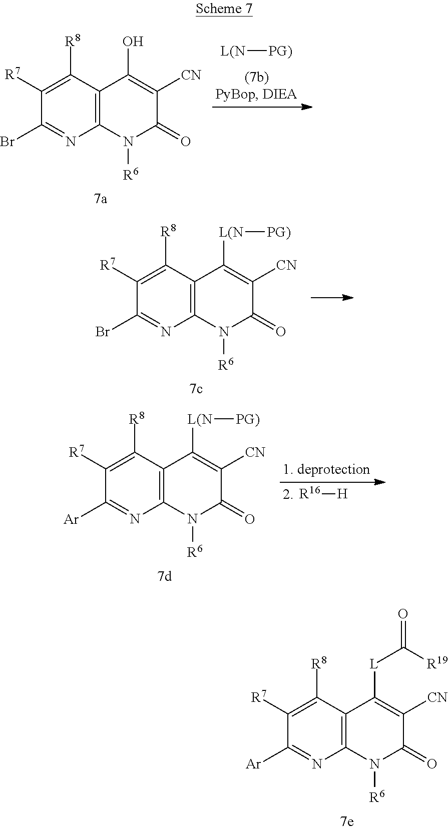

In some embodiments, a compound of Formula 7d may be prepared according to Scheme 7. For example, alcohol 7a can be treated with 7b—wherein L comprises an unprotected primary or secondary amine, and further wherein L comprises a suitably protected nitrogen atom (e.g., wherein PG is Boc, Cbz, or Fmoc)—in the presence of PyBop and a suitable base to provide the aminated product (7c). Substitution of the aryl bromide with a suitable boronic ester can provide the corresponding Ar-substituted compound (7d). Deprotection can be followed by reaction with R¹⁹—H in the presence of triphosgene bis(trichloromethyl) carbonate (BTC) and pyridine to provide a compound of Formula 7e.

Synthetic procedures for certain intermediates may be found in WO 2021/121367 and WO 2022/237815, each of which is incorporated herein by reference in its entirety, including any synthetic methods, recitations of compound variables, combination therapies, and methods of use disclosed therein.

### Compounds of Formula (XIII), (XIV), and (XV)

In certain aspects, the present disclosure provides a compound of Formula (XIII):

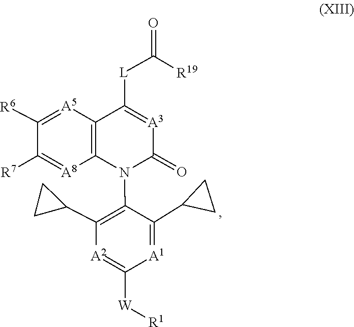

or a pharmaceutically acceptable salt or solvate thereof, wherein:
W is O, NR^{1a}, S, SO₂, or an optionally substituted 4- to 7-membered heterocycle;
R¹ is hydrogen, optionally substituted C₁₋₄ alkyl, or -L^{1b}-R^{1b},
L^{1b} is absent or an optionally substituted C₁₋₄ alkylene, optionally substituted 2- to 4-membered heteroalkylene, optionally substituted C₃₋₆ carbocycle, or optionally substituted 4- to 7-membered heterocycle,
R^{1b} is hydrogen, optionally substituted C₁₋₄ alkyl, optionally substituted C₂₋₄ alkenyl, optionally substituted C₂₋₄ alkynyl, —NR²¹R²², —OR²³, or optionally substituted 4- to 7 membered heterocycle, or
W—R¹ is hydrogen, —COOH, —COOR^{1e}, —CONR^{1c}R^{1d}, —CN, C₁₋₆ alkyl, C₂₋₆ alkenyl, C₂₋₆ alkynyl, or C₃₋₆ carbocycle, wherein the C₁₋₆ alkyl, C₂₋₆ alkenyl, C₂₋₆ alkynyl, or C₃₋₆ carbocycle is optionally substituted,
each of R^{1a}, R^{1c} and R^{1d} at each occurrence is independently hydrogen, optionally substituted C₁₋₄ alkyl, optionally substituted C₂₋₄ alkenyl, optionally substituted C₂₋₄ alkynyl, optionally substituted 2- to 4-membered heteroalkyl, optionally substituted C₃₋₆ carbocycle, optionally substituted 4- to 7-membered heterocycle, or a nitrogen protecting group;
R^{1e} at each occurrence is independently hydrogen, an optionally substituted C₁₋₄ alkyl, optionally substituted C₂₋₄ alkenyl, optionally substituted C₂₋₄ alkynyl, optionally substituted 2- to 4-membered heteroalkyl, optionally substituted C₃₋₆ carbocycle, optionally substituted 4- to 7-membered heterocycle, or an oxygen protecting group;
each of A¹, A², A³, A⁵, and A⁸ is independently CR³⁰ or N,
R³⁰ at each occurrence is independently hydrogen, —F, —Cl, C₁₋₄ alkyl, or C₁₋₄ alkoxyl; or
R¹, W, and A¹ together form an optionally substituted heterocycle;
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₅ carbocycle and 3- to 15-membered heterocycle, wherein C₃₋₁₅ carbocycle and 3- to 15-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R⁶ is hydrogen, halogen, —CN, C₃₋₄ carbocycle, optionally substituted C₁₋₄ alkyl, optionally substituted C₂₋₄ alkenyl, optionally substituted C₂₋₄ alkynyl, or optionally substituted C₁₋₄ alkoxyl;
R⁷ is selected from optionally substituted C₃₋₁₂ carbocycle and optionally substituted 3- to 12-membered heterocycle;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

In some embodiments, for a compound of Formula (XIII), W—R¹ is hydrogen, —COOH, —COOR^{1e}, —CONR^{1c}R^{1d}, —CN, C₁₋₆ alkyl, C₂₋₆ alkenyl, C₂₋₆ alkynyl, or C₃₋₆ carbocycle, wherein the C₁₋₆ alkyl, C₂₋₆ alkenyl, C₂₋₆ alkynyl, or C₃₋₆ carbocycle is optionally substituted with 1-3 groups independently selected from —F, —OH, protected —OH, and C₁₋₆ alkoxy.

In some embodiments, for a compound of Formula (XIII), L is a 4- to 15-membered heterocycle, wherein 4- to 15-membered heterocycle is optionally substituted with 1-3 groups independently selected from C₁₋₆ alkyl, C₂₋₆ alkenyl, C₂₋₆ alkynyl, C₃₋₄ carbocycle, 3- to 4-membered heterocycle, fluorine substituted C₁₋₄ alkyl, hydroxyl substituted C₁₋₄ alkyl, and cyano substituted C₁₋₄ alkyl.

In certain aspects, the present disclosure provides a compound of Formula (XIV):

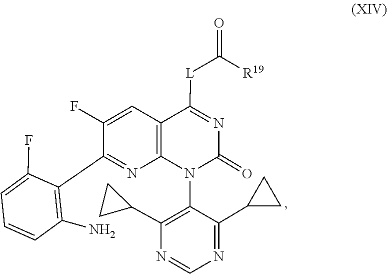

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₅ carbocycle and 3- to 15-membered heterocycle, wherein C₃₋₁₅ carbocycle and 3- to 15-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted; and
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted.

In some embodiments, for a compound of Formula (XIII) or (XIV), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea.

In some embodiments, for a compound of Formula (XIII) or (XIV), L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —O—, —N(R¹²)—, —C(O)—, —N(R¹²)C(O)—, —N(R¹²)S(O)—, —N(R¹²)S(O)₂—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH—, —CH₂N(CH₃)—, —NHCH₂—, or —N(CH₃)CH₂—. In some embodiments, L¹ is selected from a bond, 2- to 6-membered heteroalkyl, and —N(R¹²)—.

In some embodiments, for a compound of Formula (XIII) or (XIV), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (XIII) or (XIV), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (XIII) or (XIV), L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₁2 carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle.

In some embodiments, the compound of Formula (XIII) is:

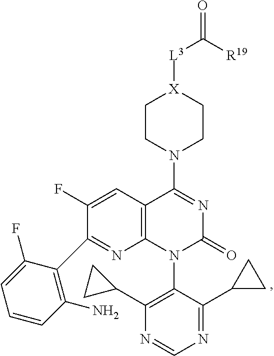

or a pharmaceutically acceptable salt or solvate thereof, wherein:
X is selected from CH and N;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, for a compound of Formula (XIII), R¹⁰ is independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2. In some embodiments, R¹⁰ is —CH₃. In some embodiments, m is 2.

In some embodiments, for a compound of Formula (XIII), X is N and L³ is a bond. In some embodiments, X is CH and L³ is optionally substituted nitrene.

In certain aspects, the present disclosure provides a compound selected from:

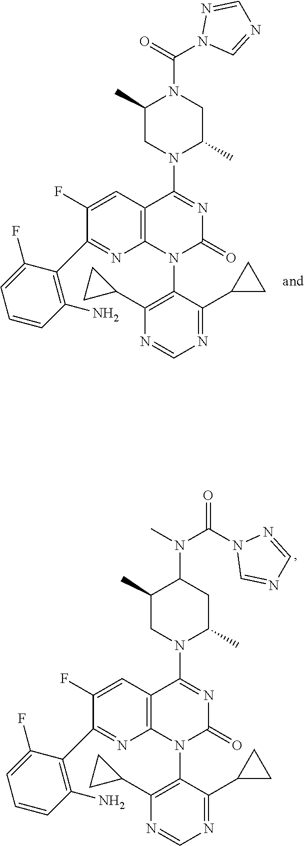 [SMILES: C[C@@H]1CN(c2nc(=O)n(-c3c(C4CC4)ncnc3C3CC3)c3nc(-c4c(N)cccc4F)c(F)cc23)[C@@H](C)CN1C(=O)n1cncn1 ; C[C@@H]1CN(c2nc(=O)n(-c3c(C4CC4)ncnc3C3CC3)c3nc(-c4c(N)cccc4F)c(F)cc23)[C@@H](C)CC1N(C)C(=O)n1cncn1]

or a pharmaceutically acceptable salt, solvate, stereoisomer, or prodrug thereof.

In certain aspects, the present disclosure provides a compound of Formula (XV):

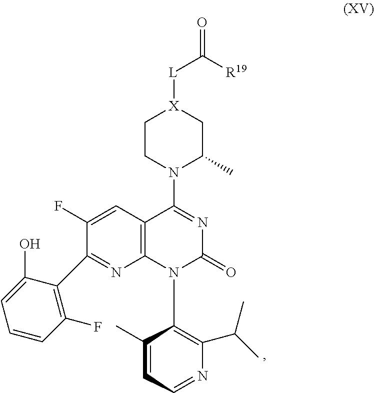

or a pharmaceutically acceptable salt or solvate thereof, wherein:
X is —CH— and L is —N(R¹²)—, or X is N and L is a bond;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted.

In some embodiments, for a compound of Formula (XV), X is —CH— and L is —N(R¹²)—. In some embodiments, X is N and L is a bond.

In certain aspects, the present disclosure provides a compound selected from:

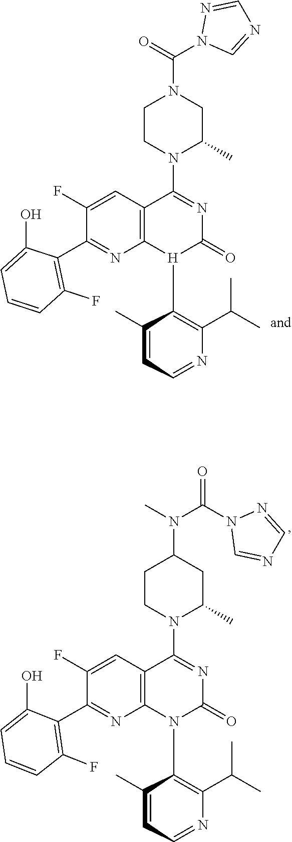

or a pharmaceutically acceptable salt, solvate, stereoisomer, or prodrug thereof.

In certain aspects, the present disclosure provides a compound selected from:

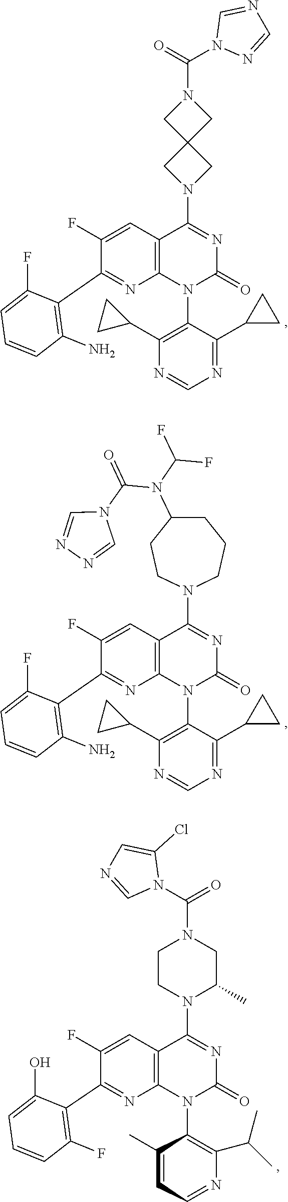

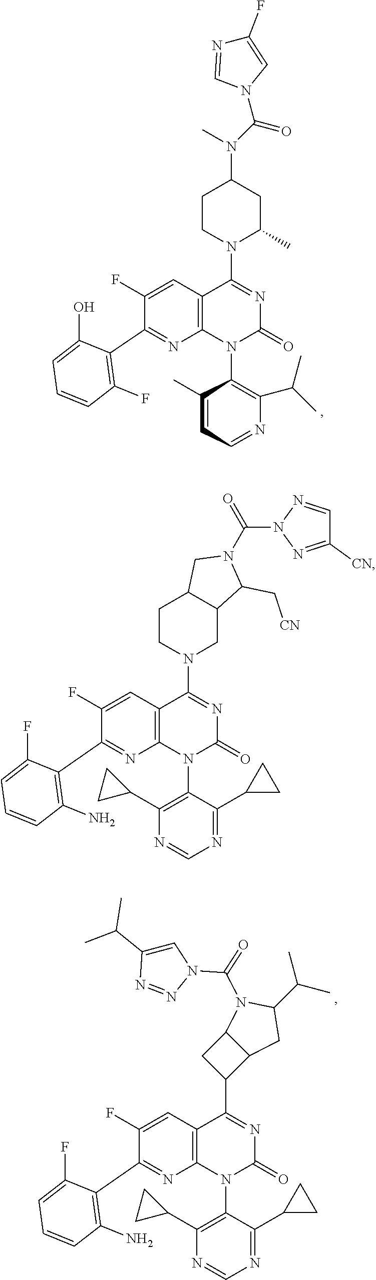

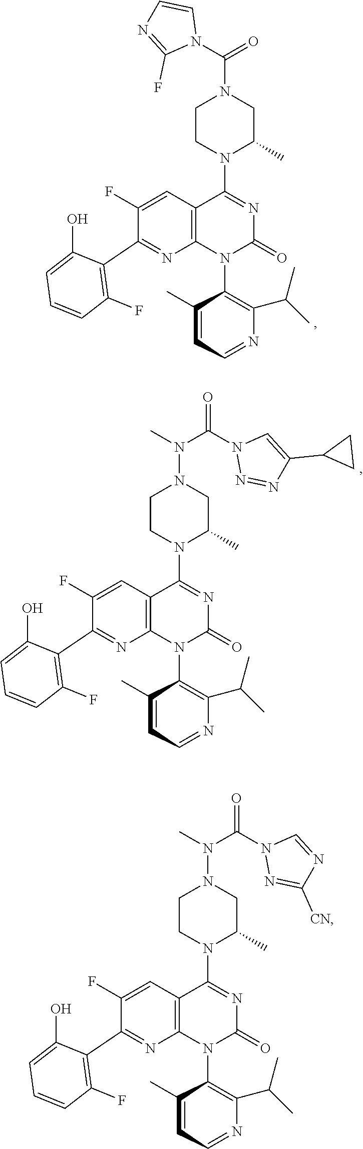

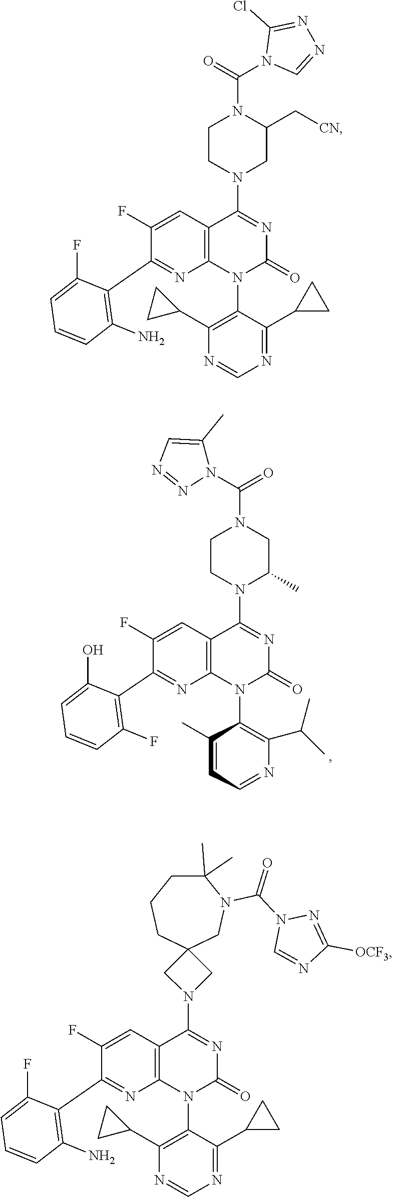

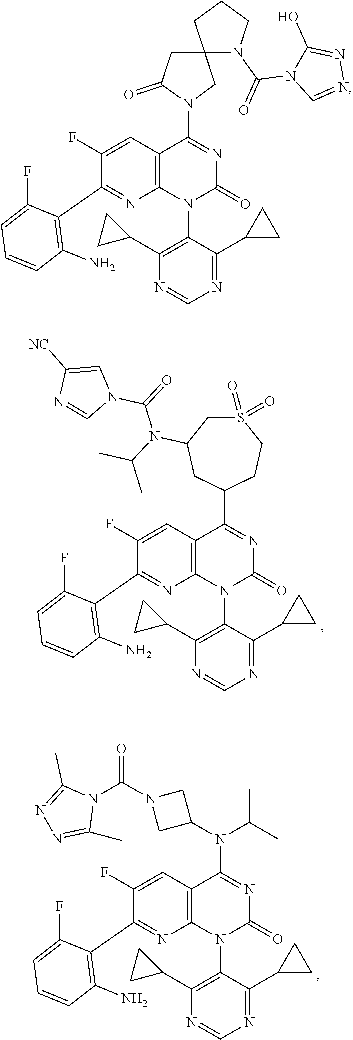 [SMILES: Nc1cccc(F)c1-c1nc2c(cc1F)c(N1CC3(CCCN3C(=O)n3cnnc3O)CC1=O)nc(=O)n2-c1c(C2CC2)ncnc1C1CC1 ; CC(C)N(C(=O)n1cnc(C#N)c1)C1CC(c2nc(=O)n(-c3c(C4CC4)ncnc3C3CC3)c3nc(-c4c(N)cccc4F)c(F)cc23)CCS(=O)(=O)C1 ; Cc1nnc(C)n1C(=O)N1CC(N(c2nc(=O)n(-c3c(C4CC4)ncnc3C3CC3)c3nc(-c4c(N)cccc4F)c(F)cc23)C(C)C)C1]

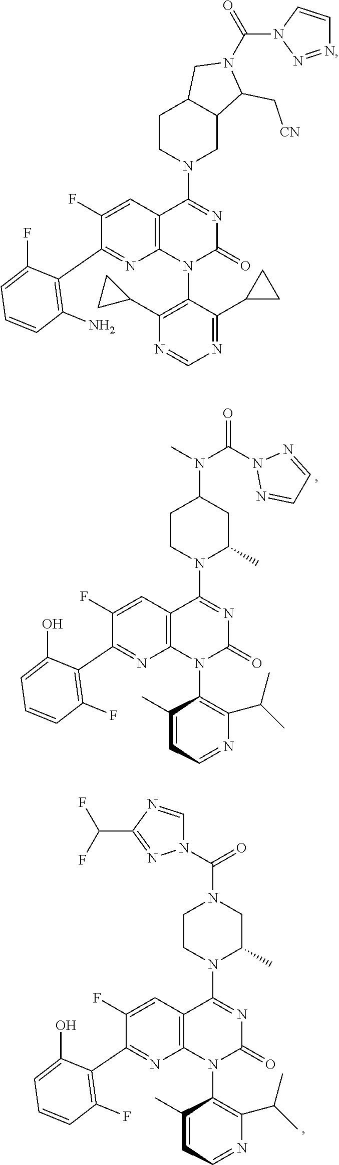

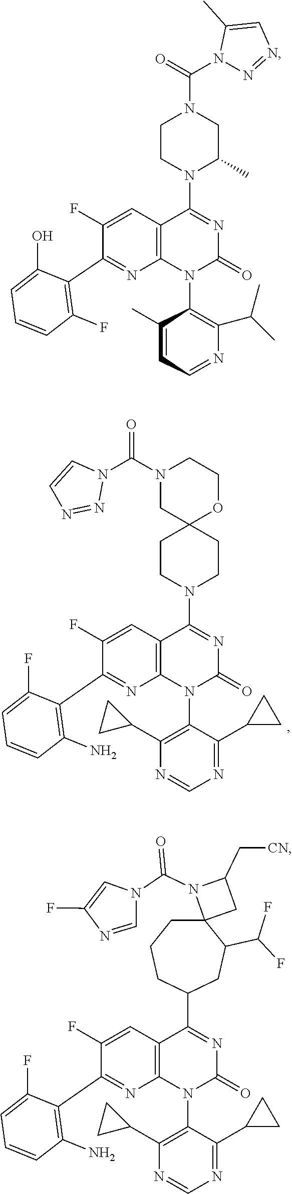

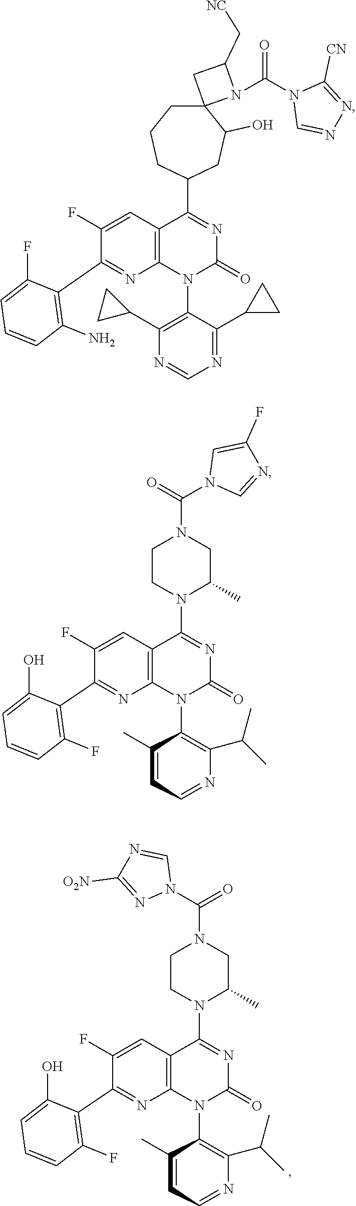

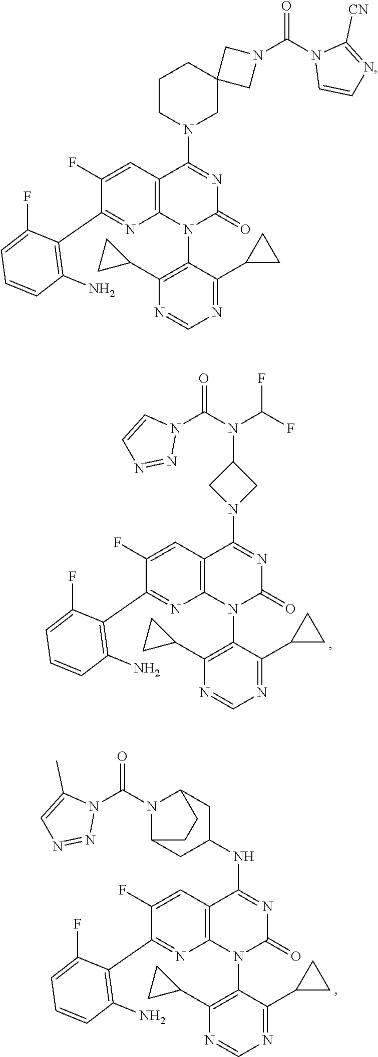 [SMILES: N#Cc1nccn1C(=O)N1CC2(CCCN(c3nc(=O)n(-c4c(C5CC5)ncnc4C4CC4)c4nc(-c5c(N)cccc5F)c(F)cc34)C2)C1 ; Nc1cccc(F)c1-c1nc2c(cc1F)c(N1CC(N(C(=O)n3ccnn3)C(F)F)C1)nc(=O)n2-c1c(C2CC2)ncnc1C1CC1 ; Cc1cnnn1C(=O)N1C2CCC1CC(Nc1nc(=O)n(-c3c(C4CC4)ncnc3C3CC3)c3nc(-c4c(N)cccc4F)c(F)cc13)C2]

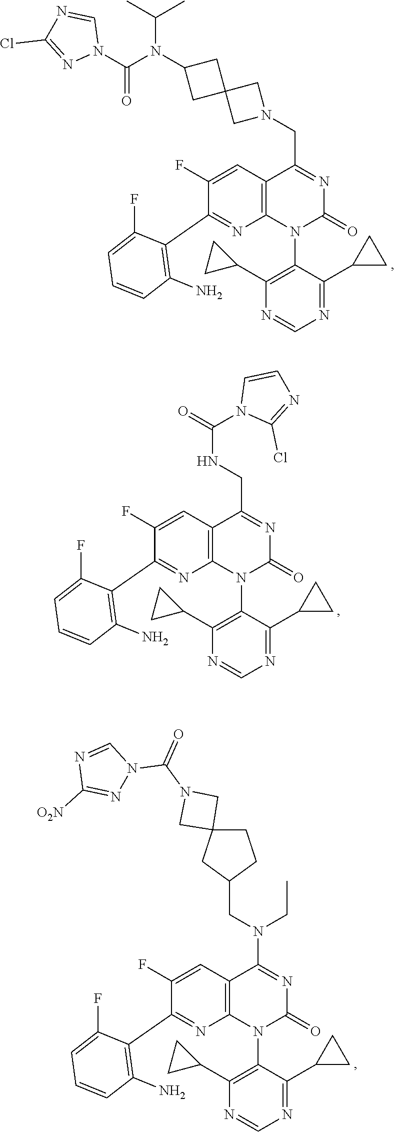

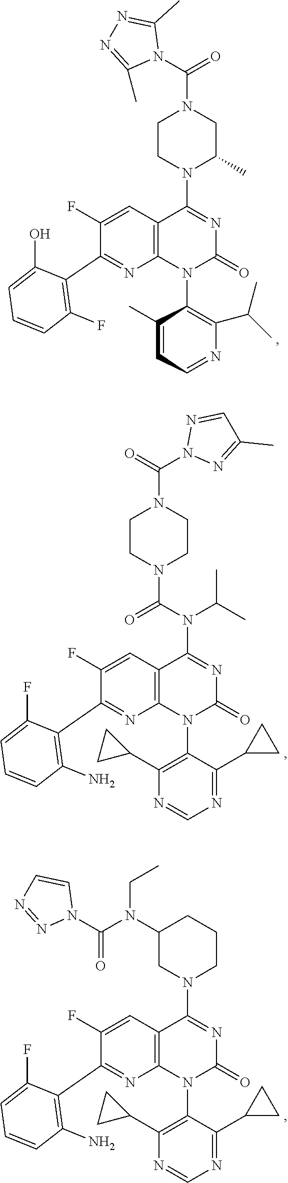

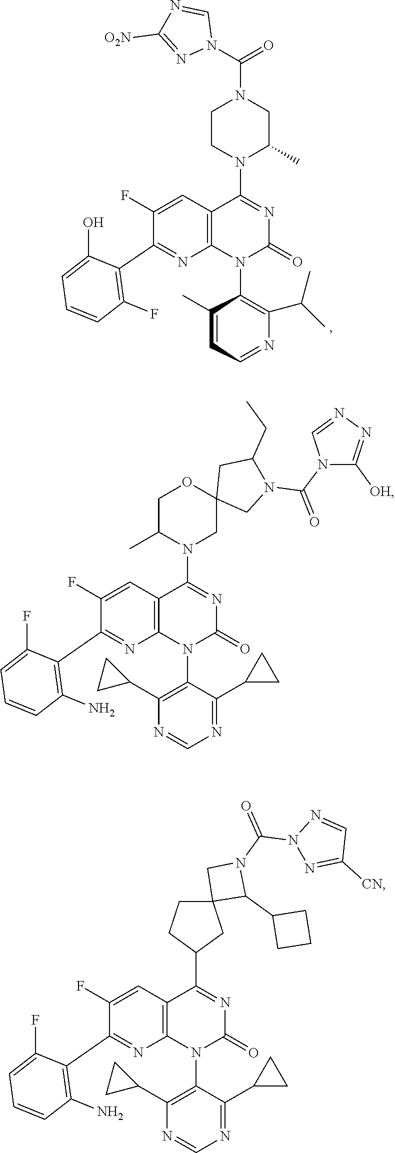

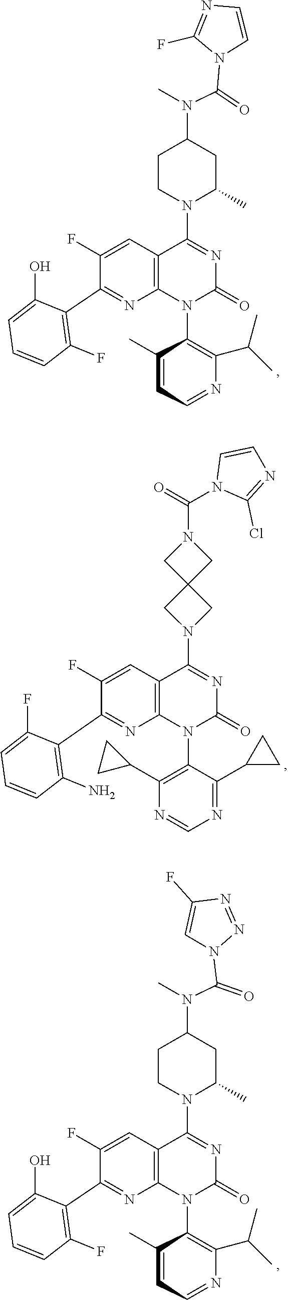

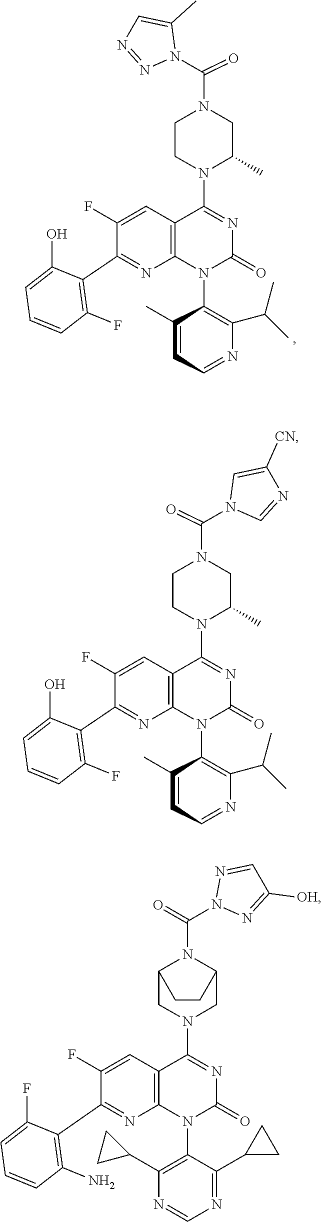

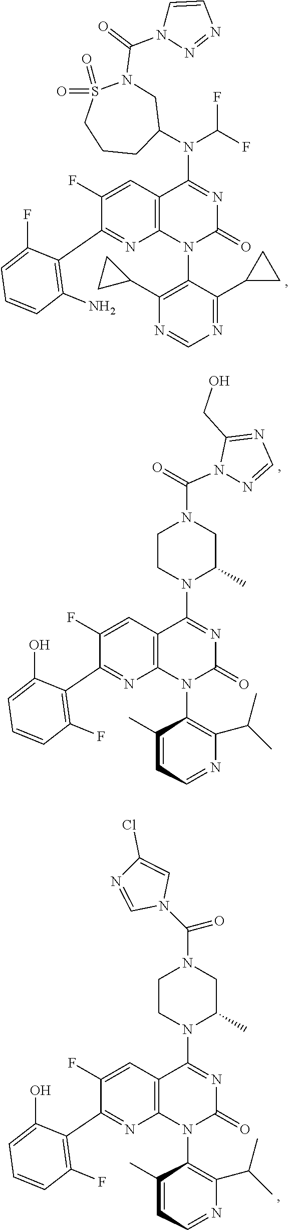

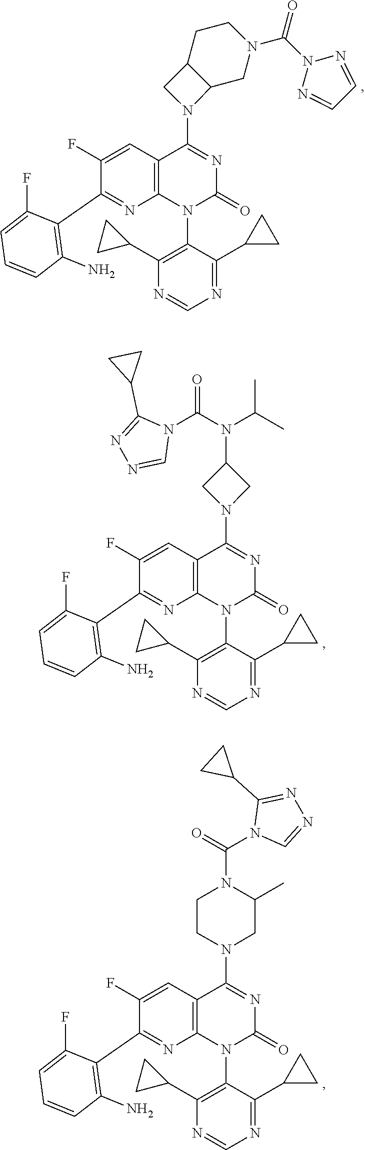

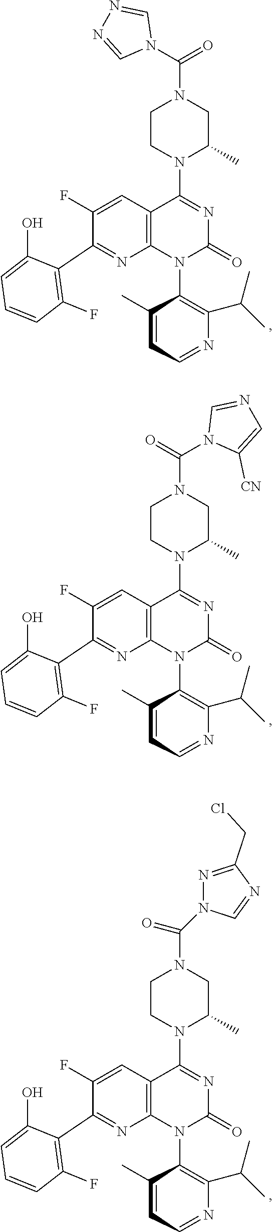

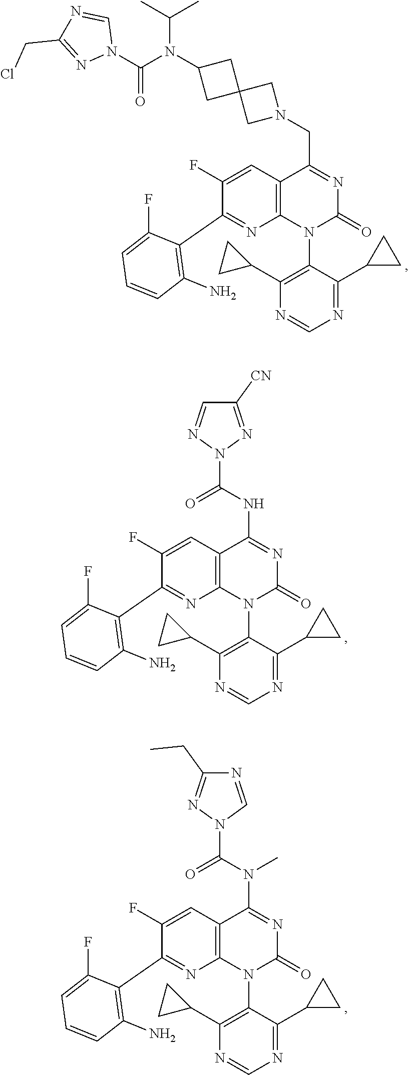 [SMILES: CC(C)N(C(=O)n1cnc(CCl)n1)C1CC2(C1)CN(Cc1nc(=O)n(-c3c(C4CC4)ncnc3C3CC3)c3nc(-c4c(N)cccc4F)c(F)cc13)C2 ; N#Cc1cnn(C(=O)Nc2nc(=O)n(-c3c(C4CC4)ncnc3C3CC3)c3nc(-c4c(N)cccc4F)c(F)cc23)n1 ; CCc1ncn(C(=O)N(C)c2nc(=O)n(-c3c(C4CC4)ncnc3C3CC3)c3nc(-c4c(N)cccc4F)c(F)cc23)n1]

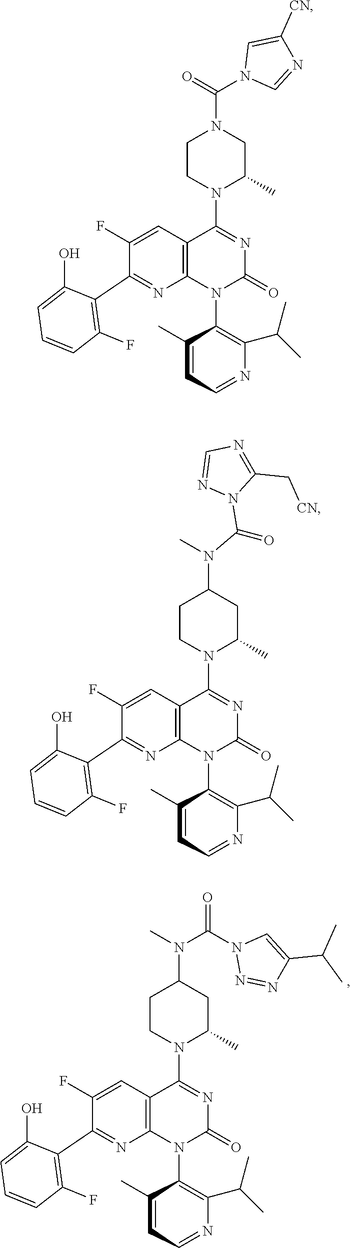

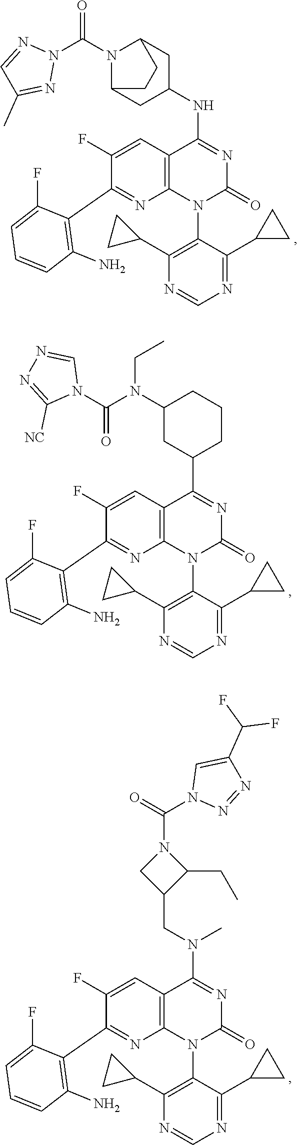 [SMILES: Cc1cnn(C(=O)N2C3CCC2CC(Nc2nc(=O)n(-c4c(C5CC5)ncnc4C4CC4)c4nc(-c5c(N)cccc5F)c(F)cc24)C3)n1 ; CCN(C(=O)n1cnnc1C#N)C1CCCC(c2nc(=O)n(-c3c(C4CC4)ncnc3C3CC3)c3nc(-c4c(N)cccc4F)c(F)cc23)C1 ; CCC1C(CN(C)c2nc(=O)n(-c3c(C4CC4)ncnc3C3CC3)c3nc(-c4c(N)cccc4F)c(F)cc23)CN1C(=O)n1cc(C(F)F)nn1]

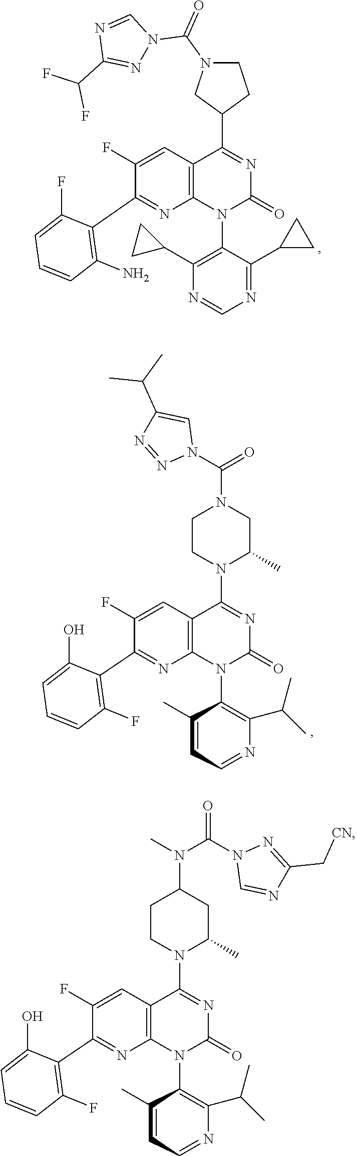

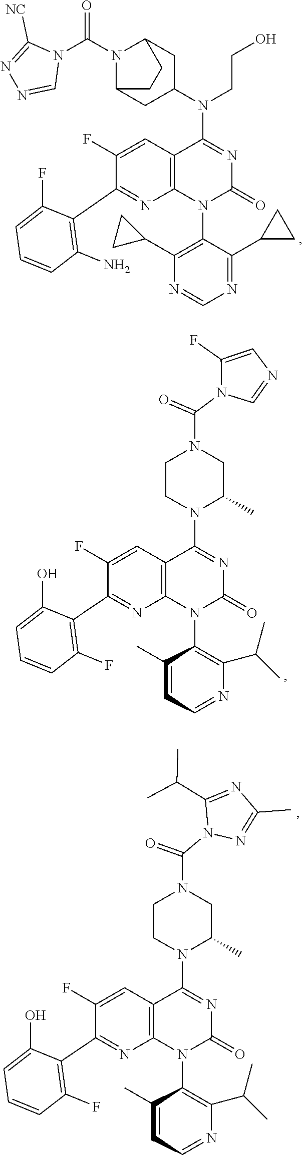

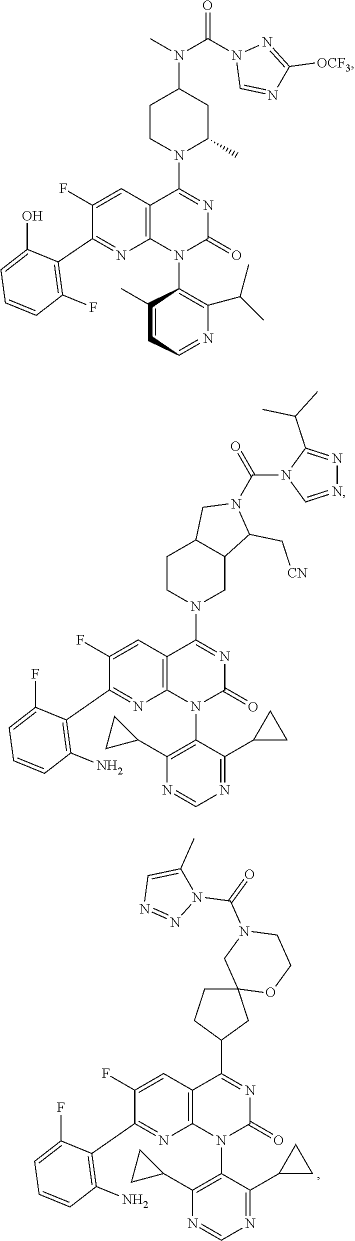

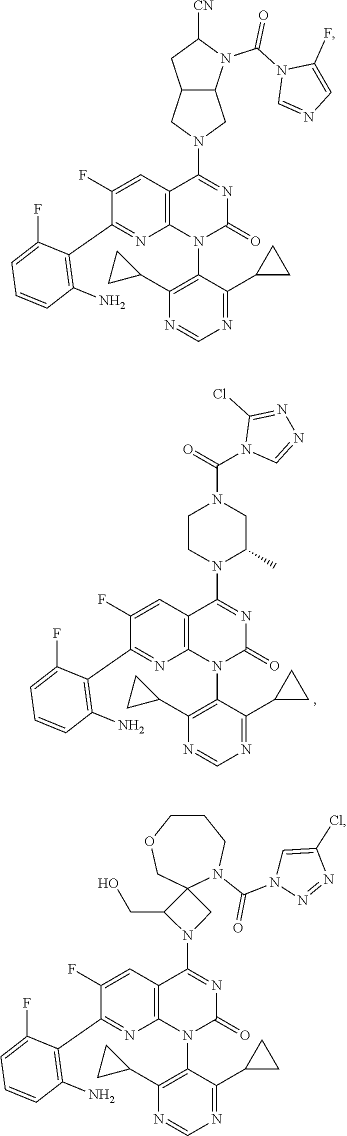 [SMILES: N#CC1CC2CN(c3nc(=O)n(-c4c(C5CC5)ncnc4C4CC4)c4nc(-c5c(N)cccc5F)c(F)cc34)CC2N1C(=O)n1cncc1F ; C[C@H]1CN(C(=O)n2cnnc2Cl)CCN1c1nc(=O)n(-c2c(C3CC3)ncnc2C2CC2)c2nc(-c3c(N)cccc3F)c(F)cc12 ; Nc1cccc(F)c1-c1nc2c(cc1F)c(N1CC3(COCCCN3C(=O)n3cc(Cl)nn3)C1CO)nc(=O)n2-c1c(C2CC2)ncnc1C1CC1]

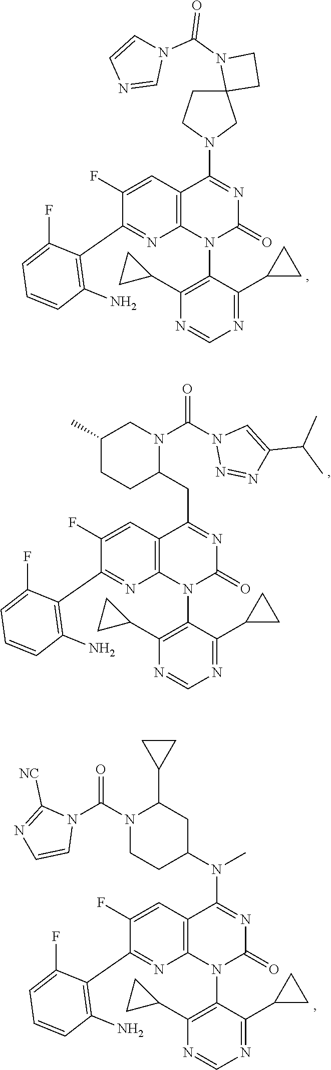 [SMILES: Nc1cccc(F)c1-c1nc2c(cc1F)c(N1CCC3(CCN3C(=O)n3ccnc3)C1)nc(=O)n2-c1c(C2CC2)ncnc1C1CC1 ; CC(C)c1cn(C(=O)N2C[C@@H](C)CCC2Cc2nc(=O)n(-c3c(C4CC4)ncnc3C3CC3)c3nc(-c4c(N)cccc4F)c(F)cc23)nn1 ; CN(c1nc(=O)n(-c2c(C3CC3)ncnc2C2CC2)c2nc(-c3c(N)cccc3F)c(F)cc12)C1CCN(C(=O)n2ccnc2C#N)C(C2CC2)C1]

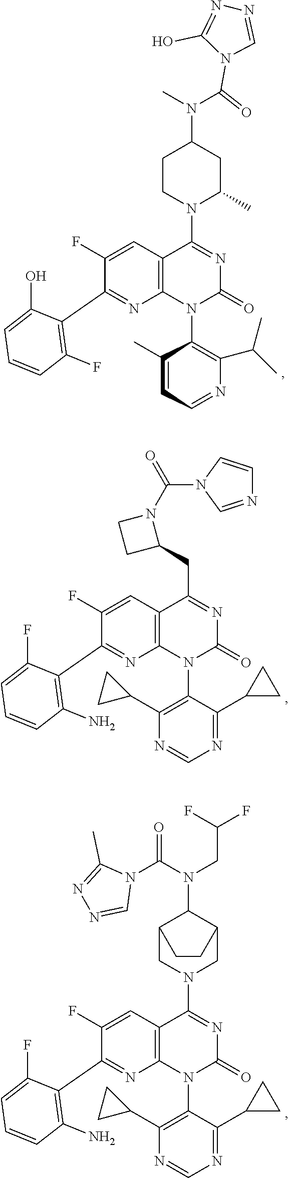

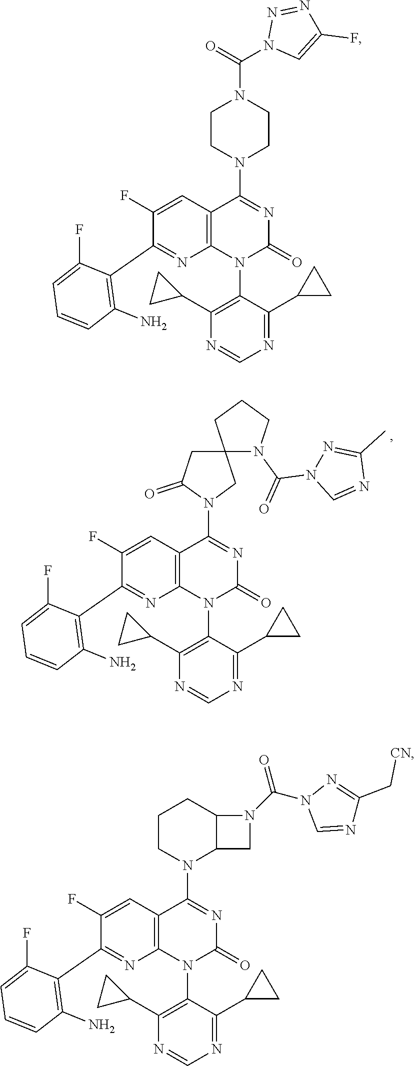

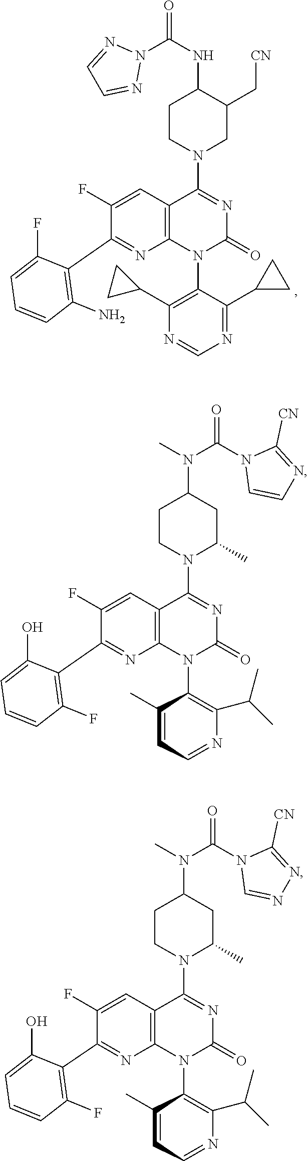

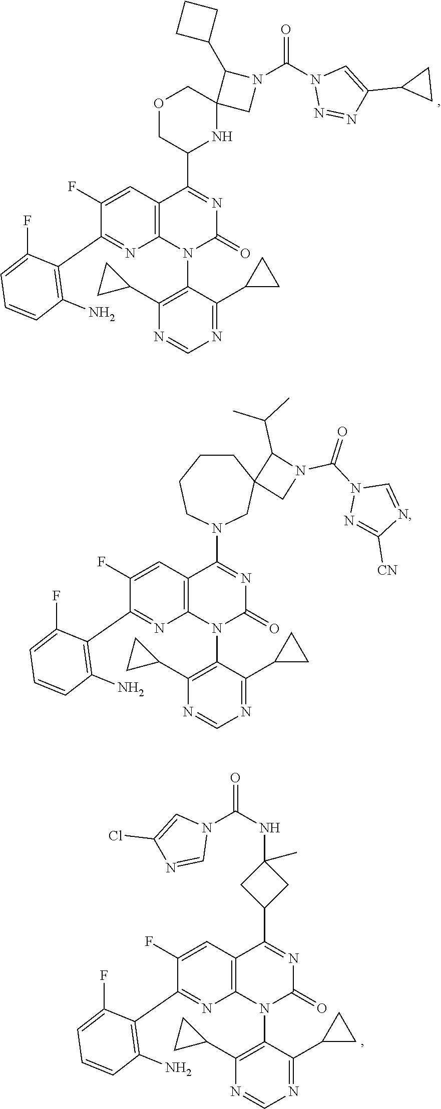 [SMILES: Nc1cccc(F)c1-c1nc2c(cc1F)c(C1COCC3(CN(C(=O)n4cc(C5CC5)nn4)C3C3CCC3)N1)nc(=O)n2-c1c(C2CC2)ncnc1C1CC1 ; CC(C)C1N(C(=O)n2cnc(C#N)n2)CC12CCCCN(c1nc(=O)n(-c3c(C4CC4)ncnc3C3CC3)c3nc(-c4c(N)cccc4F)c(F)cc13)C2 ; CC1(NC(=O)n2cnc(Cl)c2)CC(c2nc(=O)n(-c3c(C4CC4)ncnc3C3CC3)c3nc(-c4c(N)cccc4F)c(F)cc23)C1]

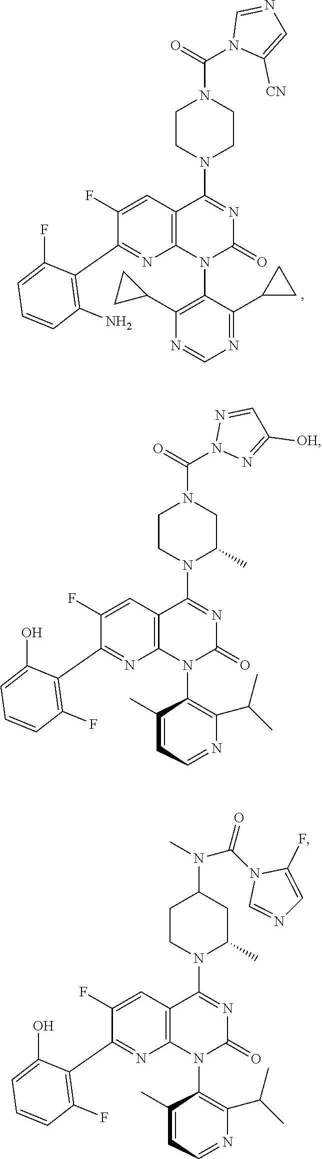

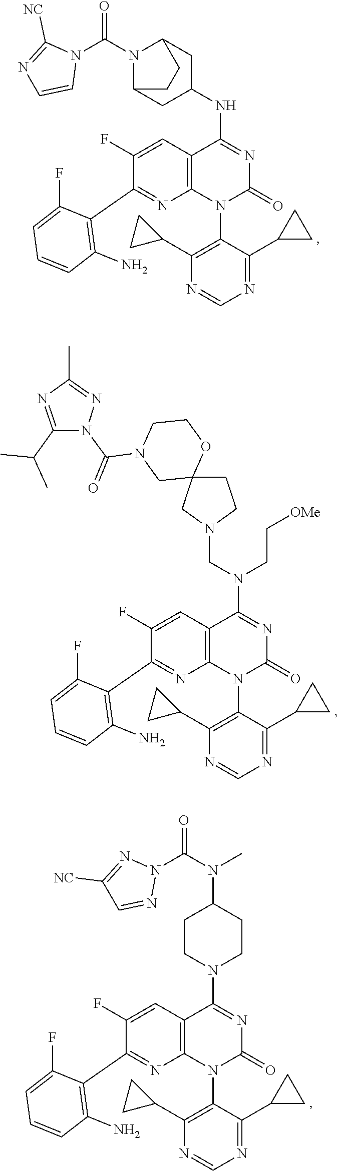 [SMILES: N#Cc1nccn1C(=O)N1C2CCC1CC(Nc1nc(=O)n(-c3c(C4CC4)ncnc3C3CC3)c3nc(-c4c(N)cccc4F)c(F)cc13)C2 ; COCCN(CN1CCC2(C1)CN(C(=O)n1nc(C)nc1C(C)C)CCO2)c1nc(=O)n(-c2c(C3CC3)ncnc2C2CC2)c2nc(-c3c(N)cccc3F)c(F)cc12 ; CN(C(=O)n1ncc(C#N)n1)C1CCN(c2nc(=O)n(-c3c(C4CC4)ncnc3C3CC3)c3nc(-c4c(N)cccc4F)c(F)cc23)CC1]

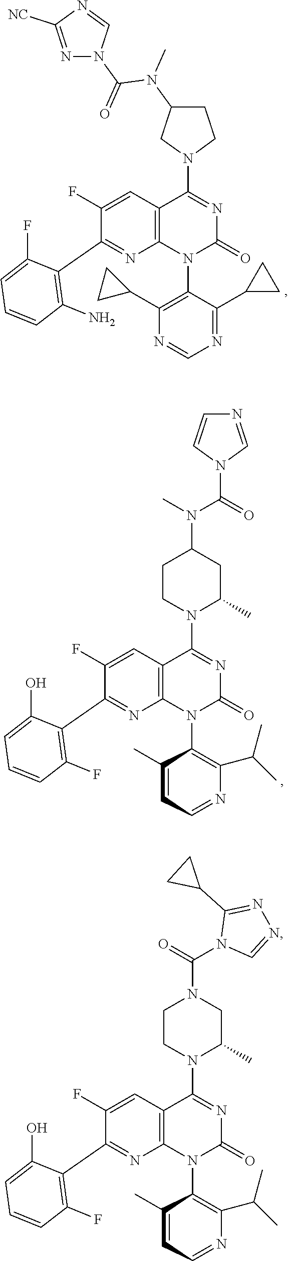

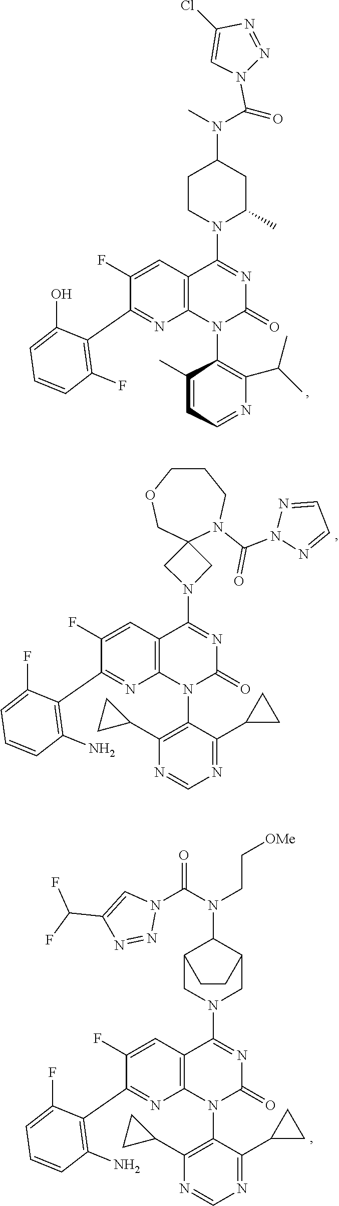

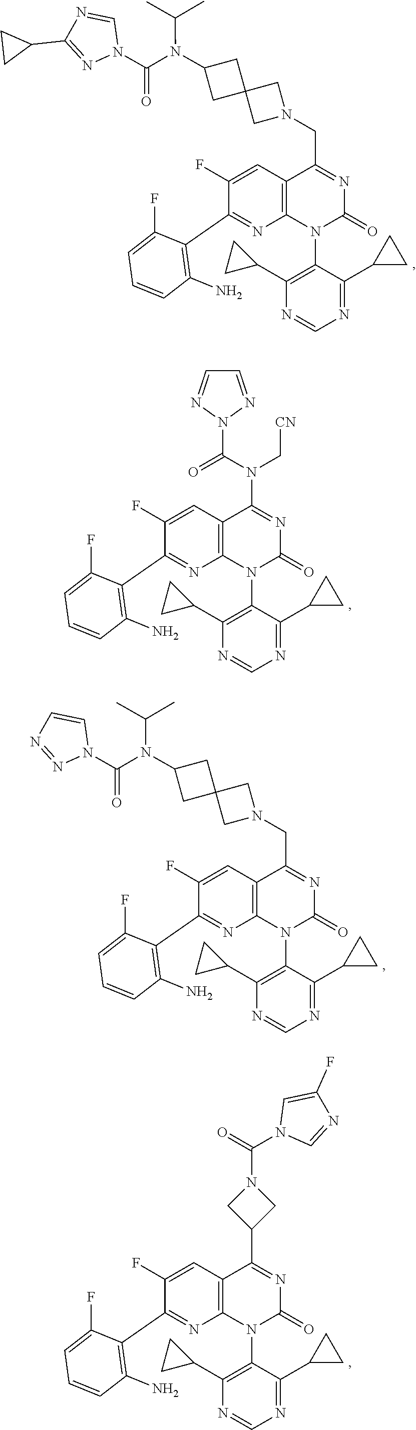 [SMILES: CC(C)N(C(=O)n1cnc(C2CC2)n1)C1CC2(C1)CN(Cc1nc(=O)n(-c3c(C4CC4)ncnc3C3CC3)c3nc(-c4c(N)cccc4F)c(F)cc13)C2 ; N#CCN(C(=O)n1nccn1)c1nc(=O)n(-c2c(C3CC3)ncnc2C2CC2)c2nc(-c3c(N)cccc3F)c(F)cc12 ; CC(C)N(C(=O)n1ccnn1)C1CC2(C1)CN(Cc1nc(=O)n(-c3c(C4CC4)ncnc3C3CC3)c3nc(-c4c(N)cccc4F)c(F)cc13)C2 ; Nc1cccc(F)c1-c1nc2c(cc1F)c(C1CN(C(=O)n3cnc(F)c3)C1)nc(=O)n2-c1c(C2CC2)ncnc1C1CC1]

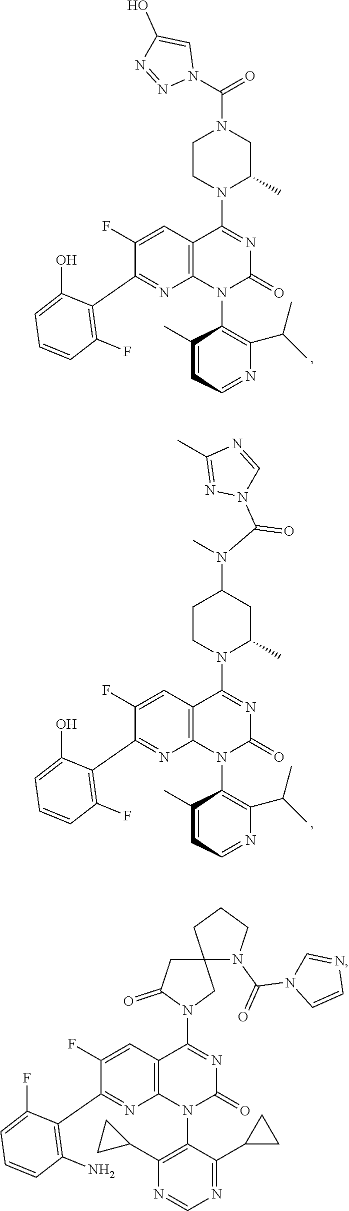

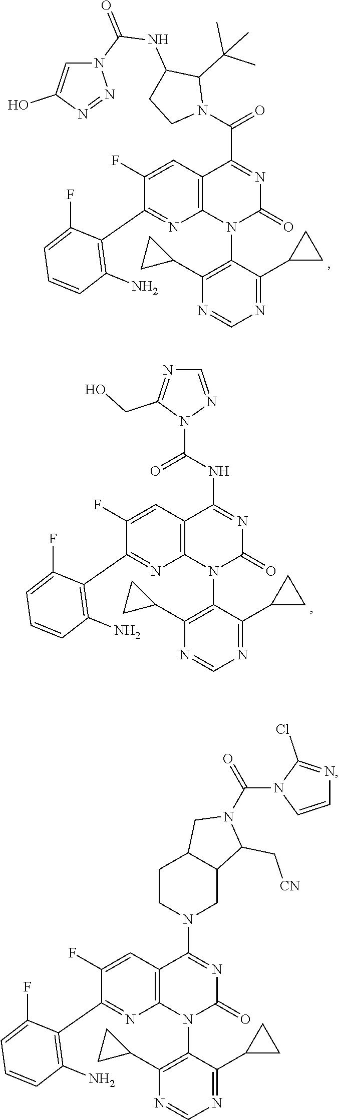 [SMILES: CC(C)(C)C1C(NC(=O)n2cc(O)nn2)CCN1C(=O)c1nc(=O)n(-c2c(C3CC3)ncnc2C2CC2)c2nc(-c3c(N)cccc3F)c(F)cc12 ; Nc1cccc(F)c1-c1nc2c(cc1F)c(NC(=O)n1ncnc1CO)nc(=O)n2-c1c(C2CC2)ncnc1C1CC1 ; N#CCC1C2CN(c3nc(=O)n(-c4c(C5CC5)ncnc4C4CC4)c4nc(-c5c(N)cccc5F)c(F)cc34)CCC2CN1C(=O)n1ccnc1Cl]

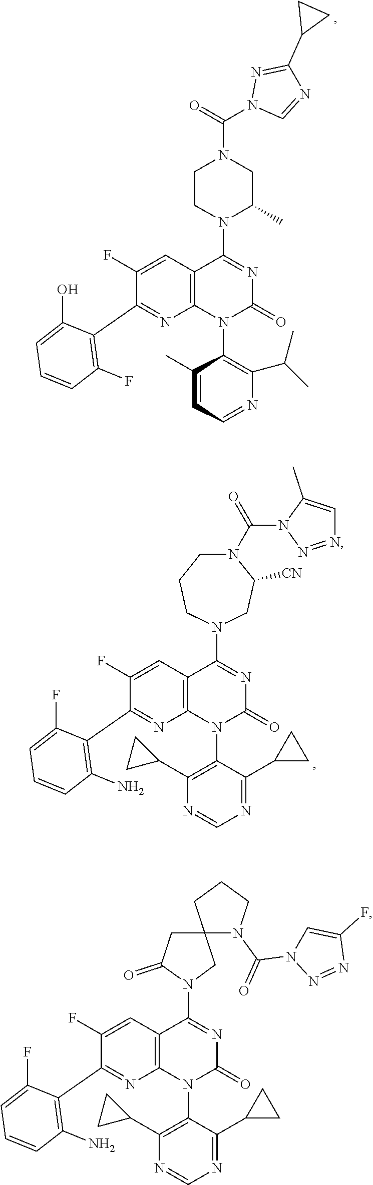

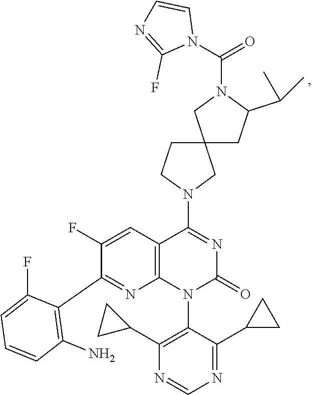 [SMILES: CC(C)C1CC2(CCN(c3nc(=O)n(-c4c(C5CC5)ncnc4C4CC4)c4nc(-c5c(N)cccc5F)c(F)cc34)C2)CN1C(=O)n1ccnc1F]

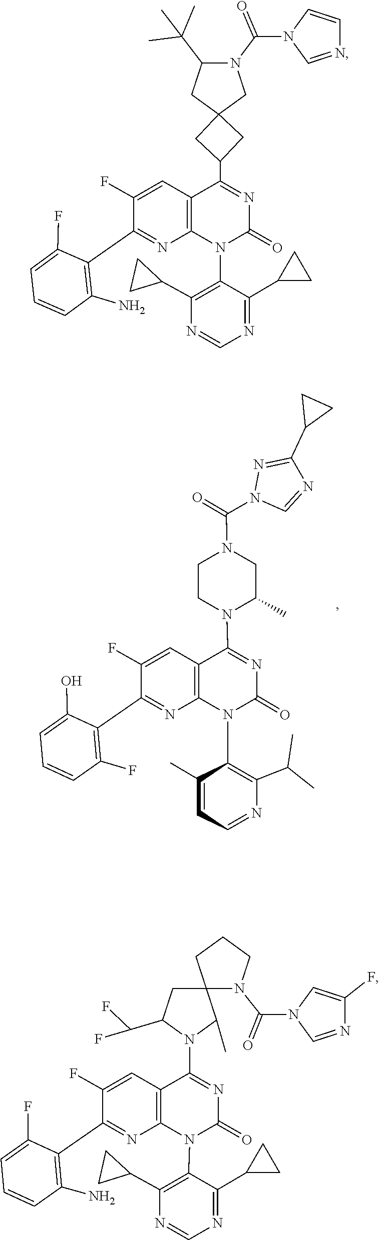

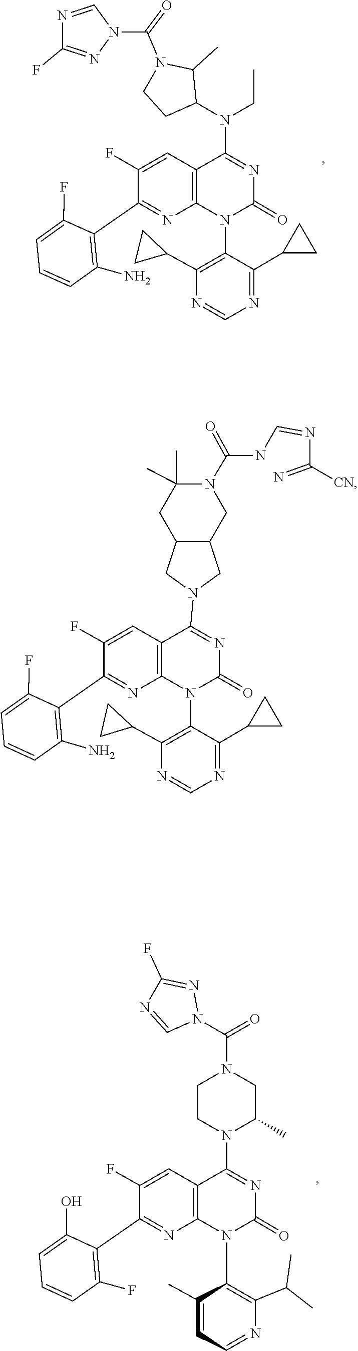

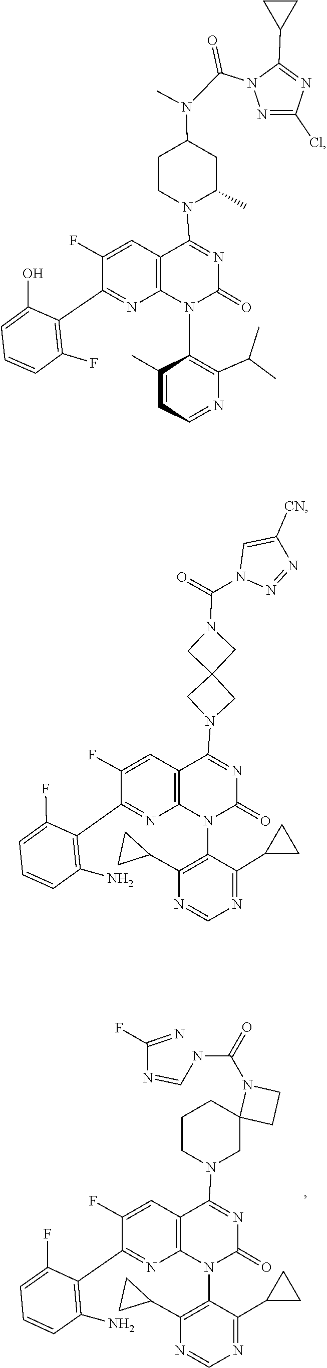

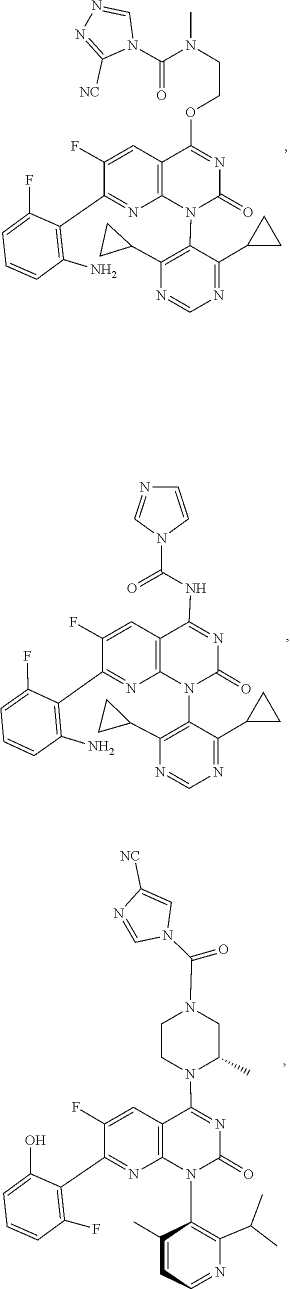

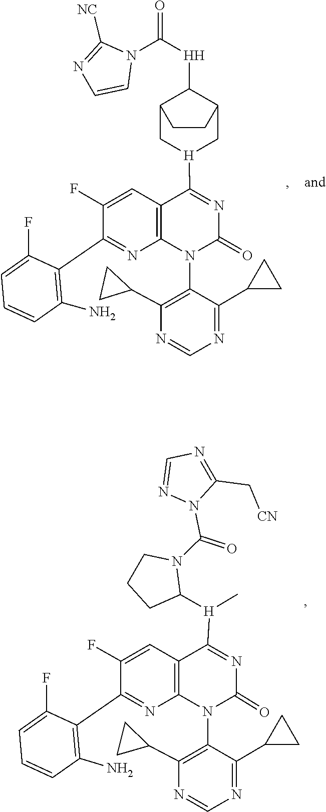

or a pharmaceutically acceptable salt, solvate, stereoisomer, or prodrug thereof.

In general, compounds of the disclosure may be prepared by the following reaction schemes:

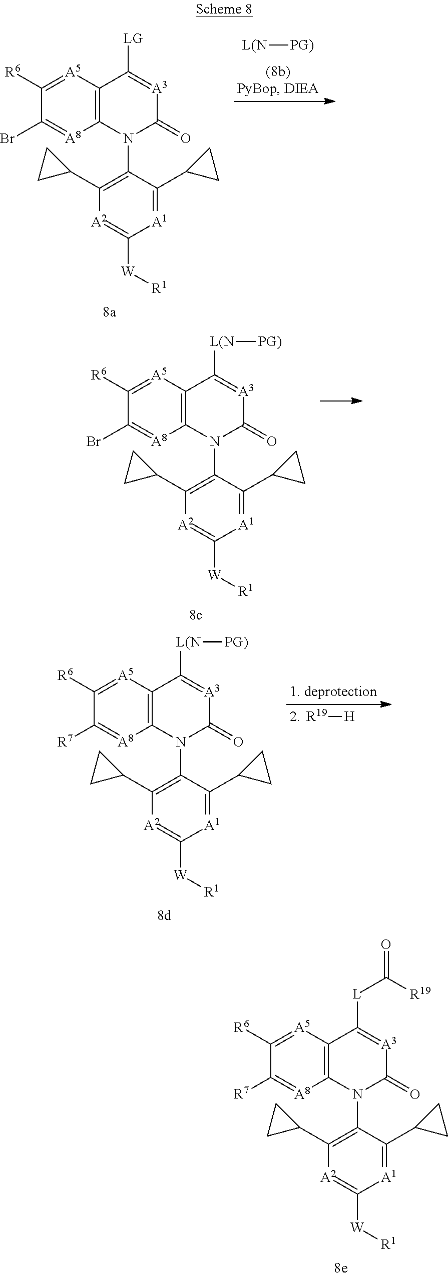

In some embodiments, a compound of Formula 8e may be prepared according to Scheme 8. For example, 8a, including a suitable leaving group (LG, e.g., halide or sulfonate leaving group), can be treated with 8b—wherein L comprises an unprotected primary or secondary amine, and further wherein L comprises a suitably protected nitrogen atom (e.g., wherein PG is Boc, Cbz, or Fmoc), in the presence of a suitable base to provide the aminated product (8c). Substitution of the aryl bromide with a suitable boronic ester can provide the corresponding R⁷-substituted compound (8d). Deprotection can be followed by reaction with R¹⁹—H in the presence of triphosgene bis(trichloromethyl) carbonate (BTC) and pyridine to provide a compound of Formula 8e.

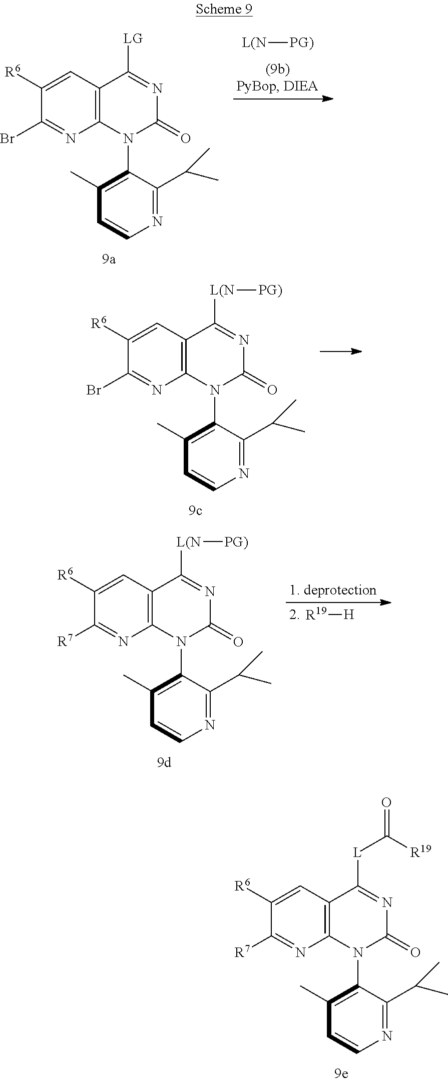

In some embodiments, similar reaction conditions and methods may be used following Scheme 9 above.

Synthetic procedures for certain intermediates may be found in WO 2020/233592 and WO 2018/217651, each of which is incorporated herein by reference in its entirety, including any synthetic methods, recitations of compound variables, combination therapies, and methods of use disclosed therein.

### Compounds of Formula (XVI), (XVII), and (XVIII)

In certain aspects, the present disclosure provides a compound of Formula (XVI):

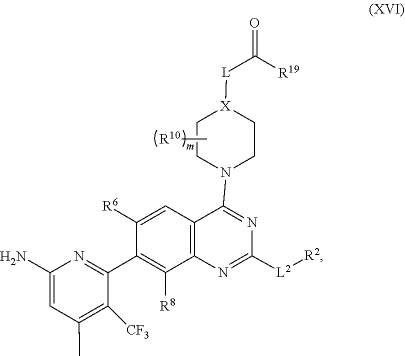

or a pharmaceutically acceptable salt or solvate thereof, wherein:
L is a bond and X is N; or L is —N(R¹²)— and X is CH;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3;
L² is selected from a bond, —O—, —N(R^{2a})—, and —S—;
R¹ is selected from hydrogen and C₁₋₃ alkyl, wherein C₁₋₃ alkyl is optionally substituted;
R² is selected from hydrogen, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), halogen, —CN, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁶ and R⁸ are each independently selected from hydrogen, —F, —Cl, C₁₋₄ alkyl, C₁₋₄ haloalkyl, and C₁₋₁₄ alkoxyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, the compound of Formula (XVI) is a compound of Formula (XVII):

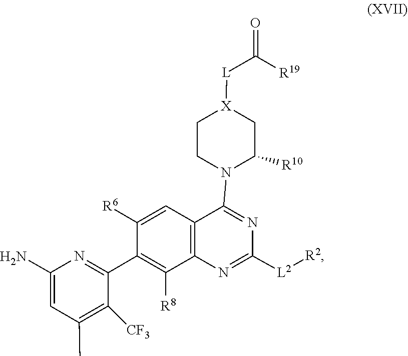

or a pharmaceutically acceptable salt or solvate thereof.

In some embodiments, for a compound of Formula (XVI) or (XVII), R⁶ is —Cl and R⁸ is —F. In some embodiments, L² is —O—. In some embodiments, R² is —C₀₋₆ alkyl-(3- to 12-membered heterocycle), wherein —C₀₋₆ alkyl-(3- to 12-membered heterocycle) is optionally substituted.

In some embodiments, for a compound of Formula (XVI) or (XVII), -L²-R² is selected from

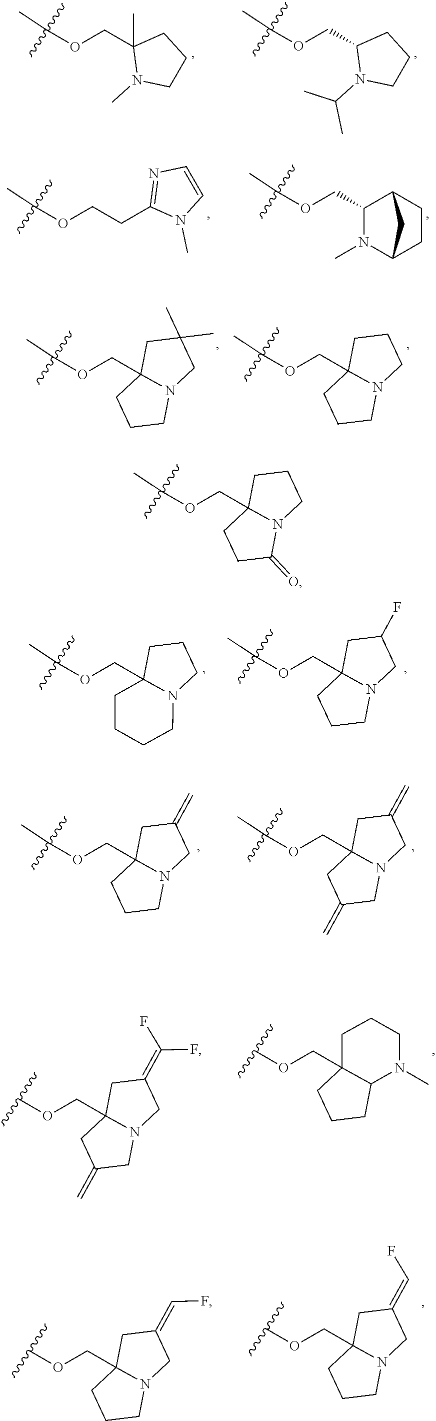

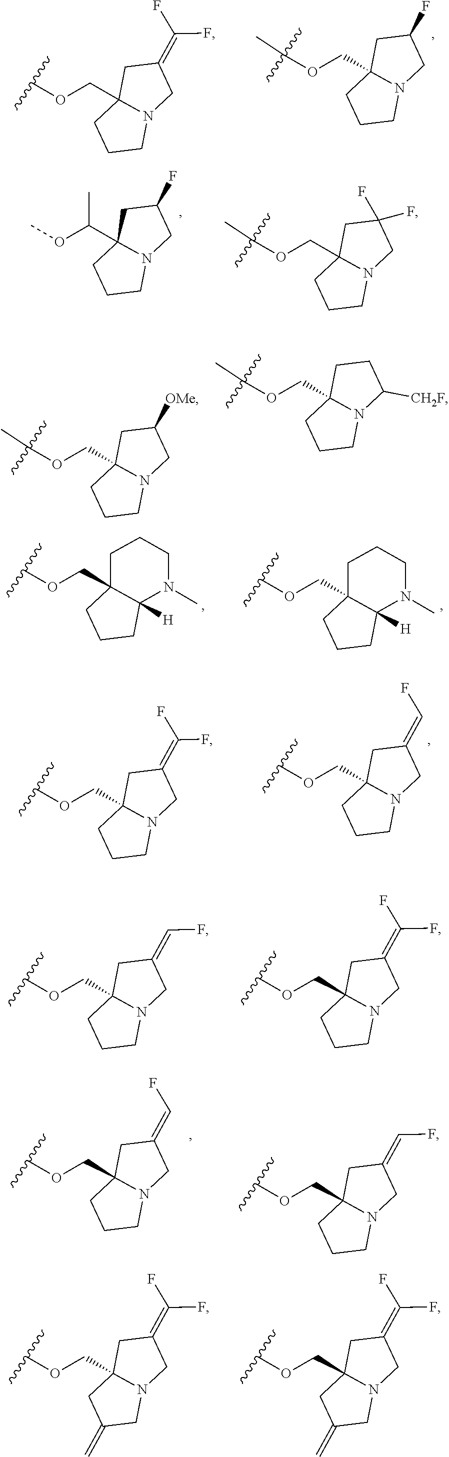

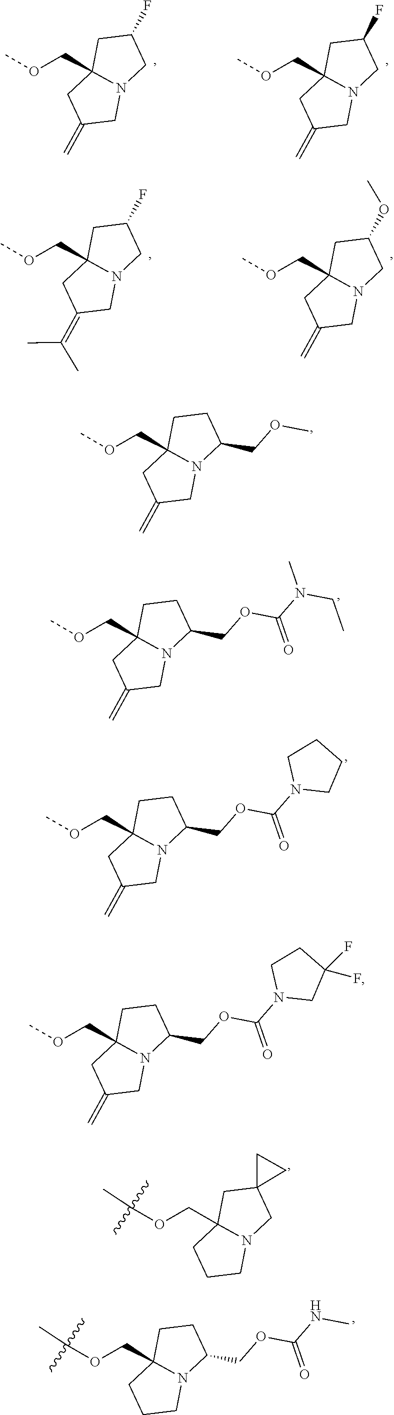

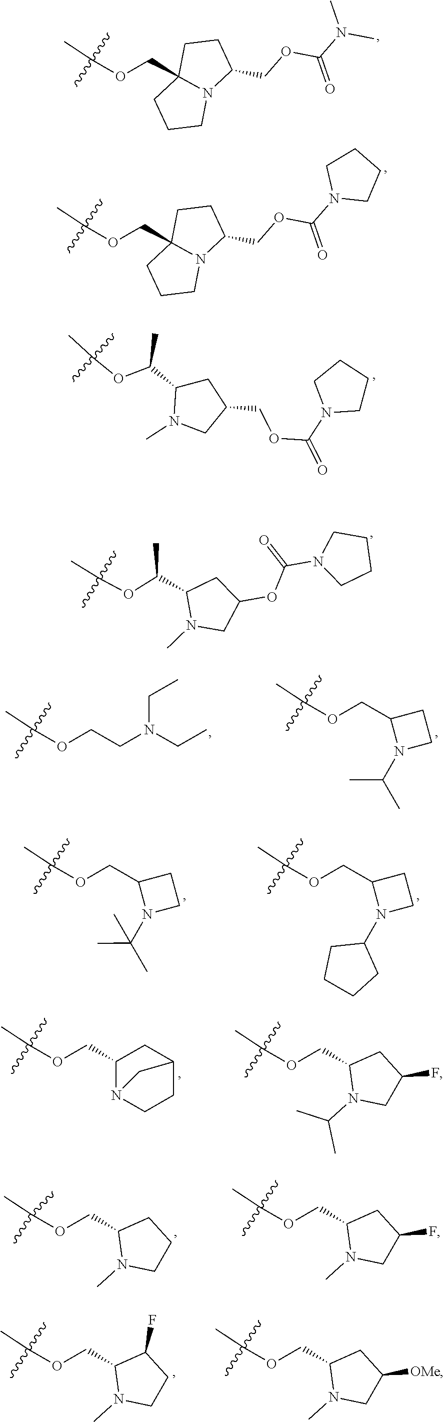

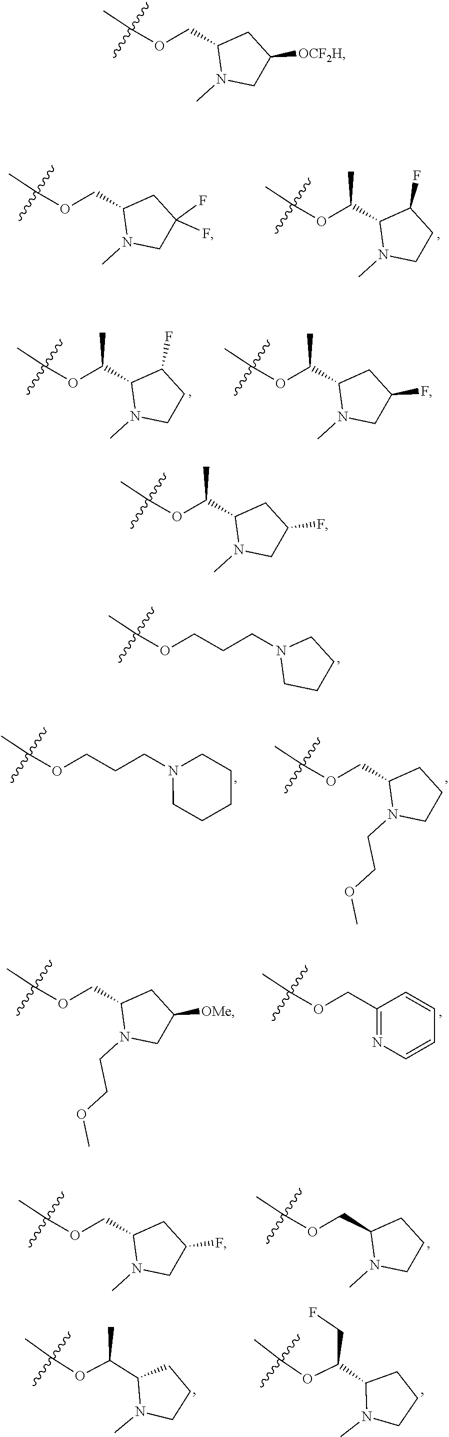

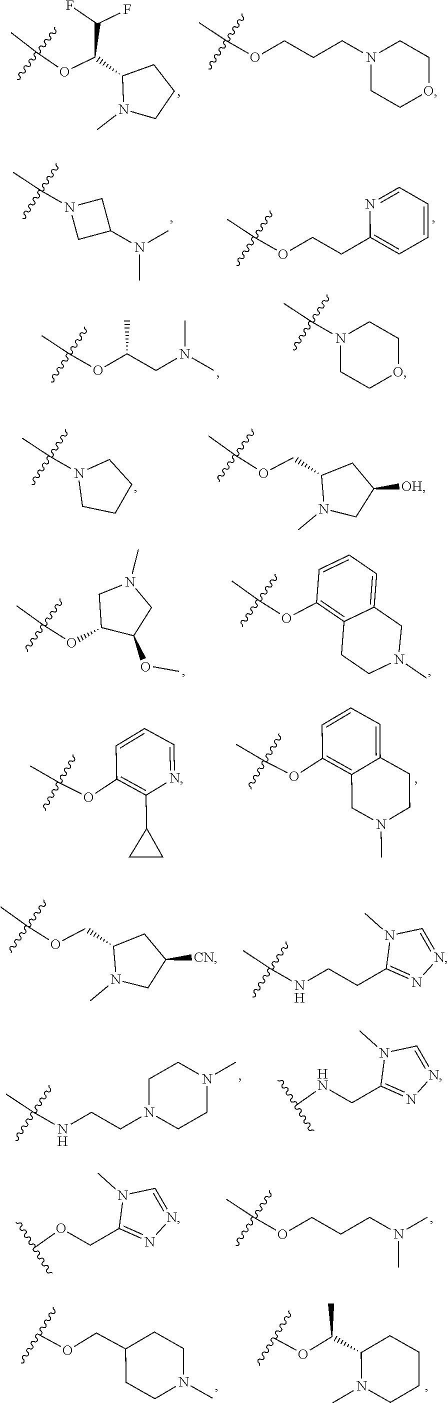

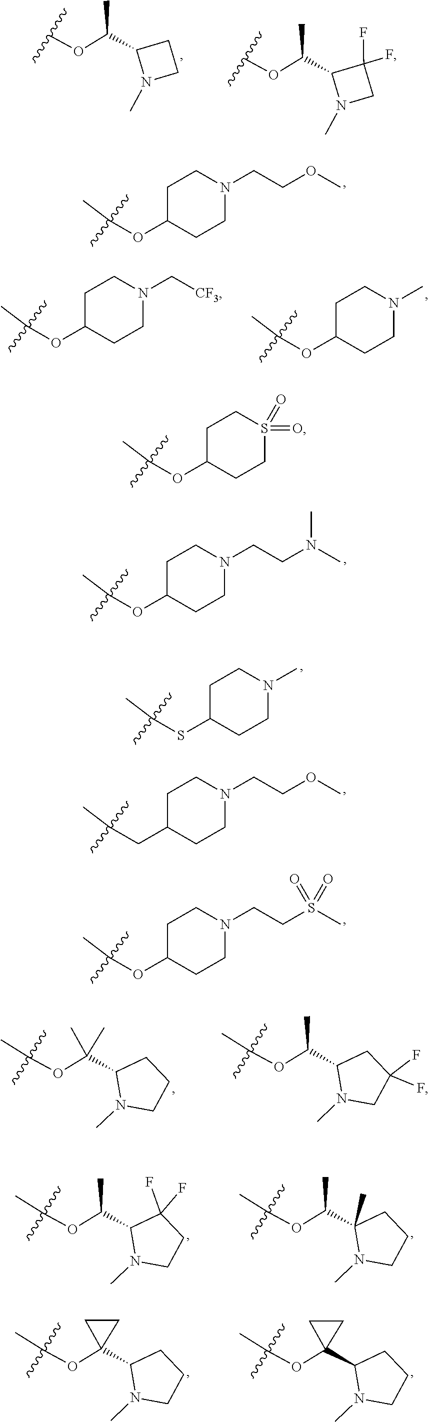

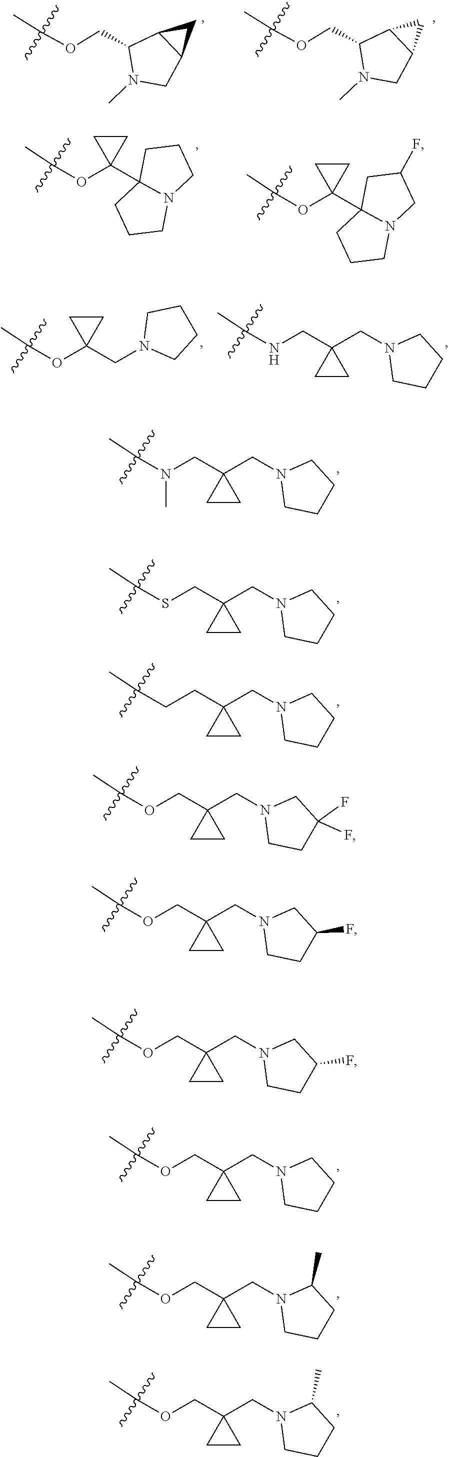

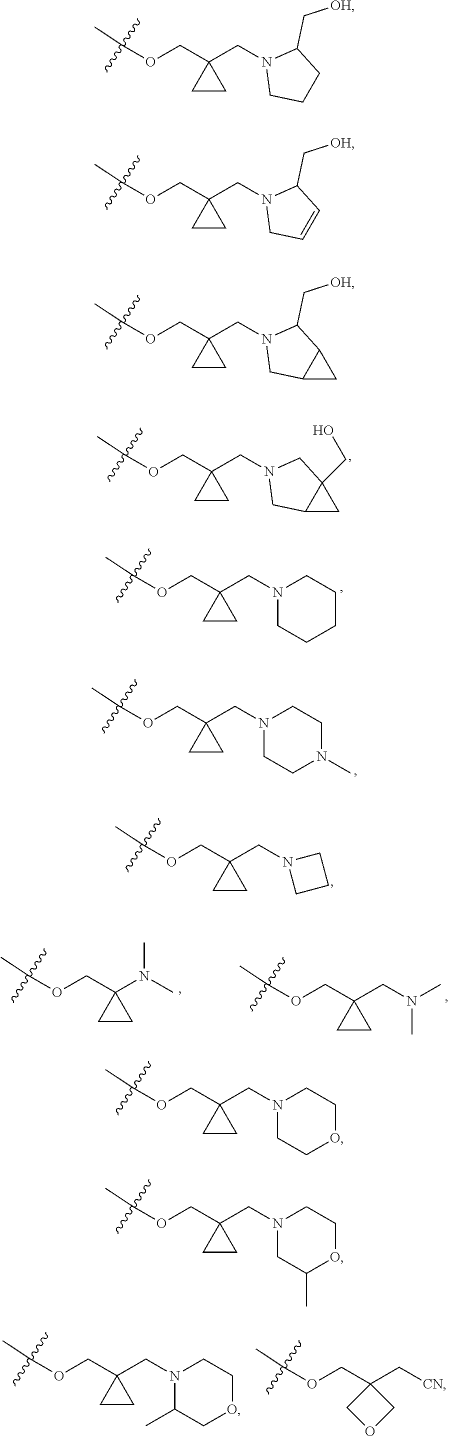

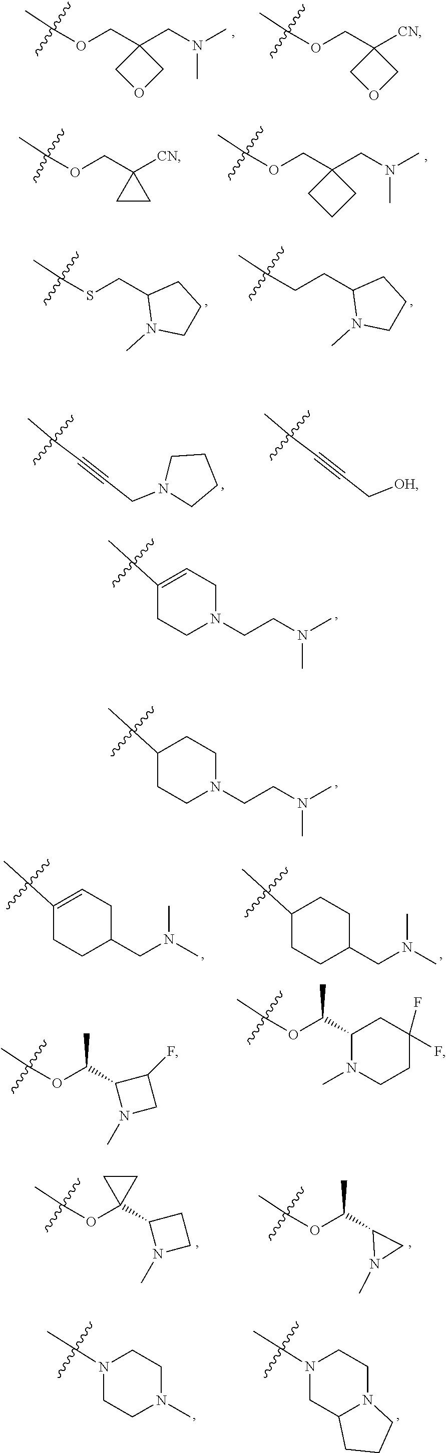

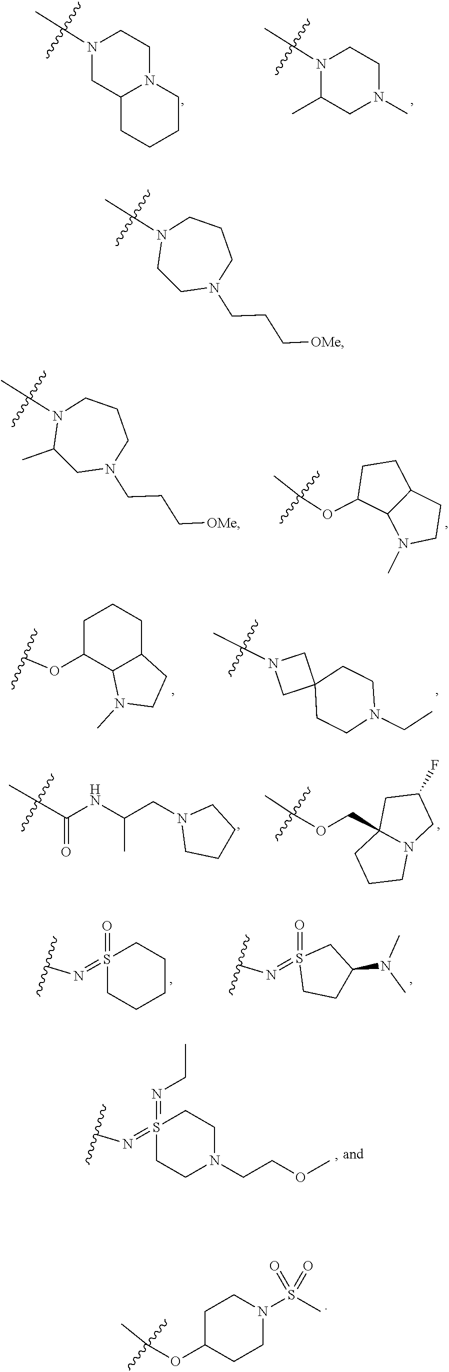

In some embodiments, -L²-R² is selected from

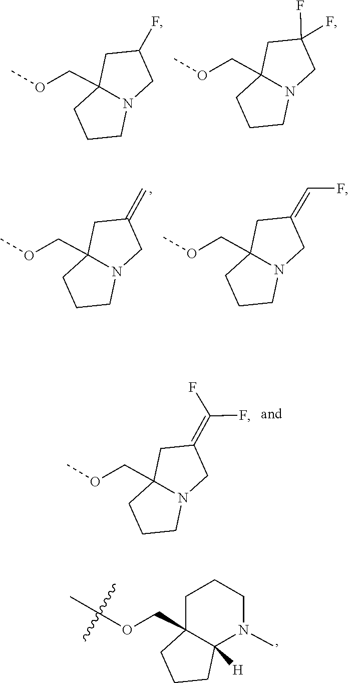

In some embodiments, -L²-R² is selected from

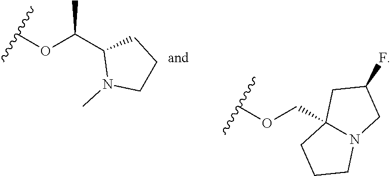

In some embodiments, -L²-R² is

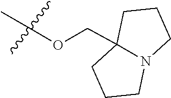 [SMILES: *OCC12CCCN1CCC2]

optionally substituted with one or more R²⁰. In some embodiments, -L²R² is

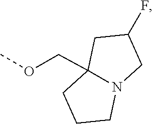

such as

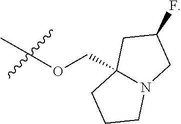 [SMILES: *OC[C@@]12CCCN1C[C@H](F)C2]

In some embodiments, -L²-R² is

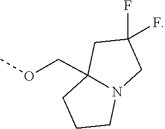

In some embodiments, -L²-R² is

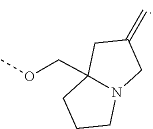

In some embodiments, -L²-R² is

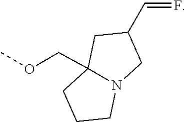

In some embodiments, -L²-R² is

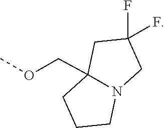

In some embodiments, -L²-R² is

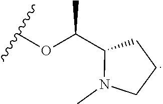 [SMILES: *O[C@@H](C)[C@@H]1CCCN1C]

In some embodiments, -L²-R² is

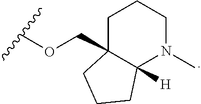 [SMILES: *OC[C@]12CCC[C@H]1N(C)CCC2]

In some embodiments, -L²-R² is

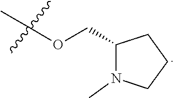 [SMILES: *OC[C@@H]1CCCN1C]

In some embodiments, for a compound of Formula (XVI) or (XVII), X is CHI and L is —N(R¹²)—. In some embodiments, X is CH and L is —N(CH₃)—. In some embodiments, X is N and L is a bond.

In some embodiments, for a compound of Formula (XVI) or (XVII), R¹⁰ is independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents independently selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2. In some embodiments, R¹⁰ is —CH₃.

In certain aspects, the present disclosure provides a compound of Formula (XVIII):

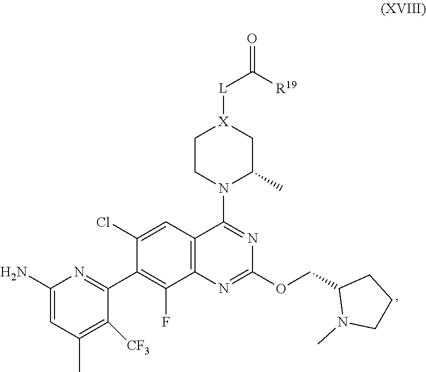

or a pharmaceutically acceptable salt or solvate thereof, wherein:
X is CH and L is selected from —N(CH₃)— and —NH—; or X is N and L is a bond; and
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted.

In certain aspects, the present disclosure provides a compound selected from:

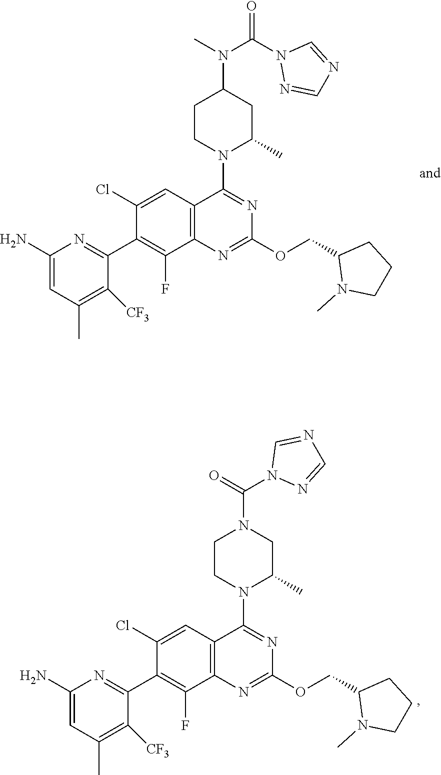

or a pharmaceutically acceptable salt, solvate, stereoisomer, or prodrug thereof.

In certain aspects, the present disclosure provides a compound selected from:

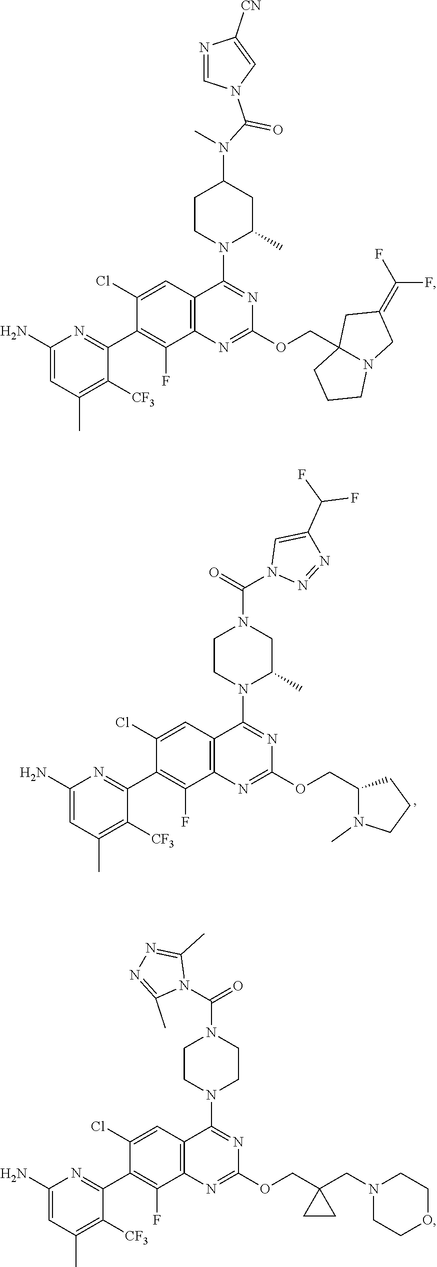 [SMILES: Cc1cc(N)nc(-c2c(Cl)cc3c(N4CCC(N(C)C(=O)n5cnc(C#N)c5)C[C@@H]4C)nc(OCC45CCCN4CC(=C(F)F)C5)nc3c2F)c1C(F)(F)F ; Cc1cc(N)nc(-c2c(Cl)cc3c(N4CCN(C(=O)n5cc(C(F)F)nn5)C[C@@H]4C)nc(OC[C@@H]4CCCN4C)nc3c2F)c1C(F)(F)F ; Cc1cc(N)nc(-c2c(Cl)cc3c(N4CCN(C(=O)n5c(C)nnc5C)CC4)nc(OCC4(CN5CCOCC5)CC4)nc3c2F)c1C(F)(F)F]

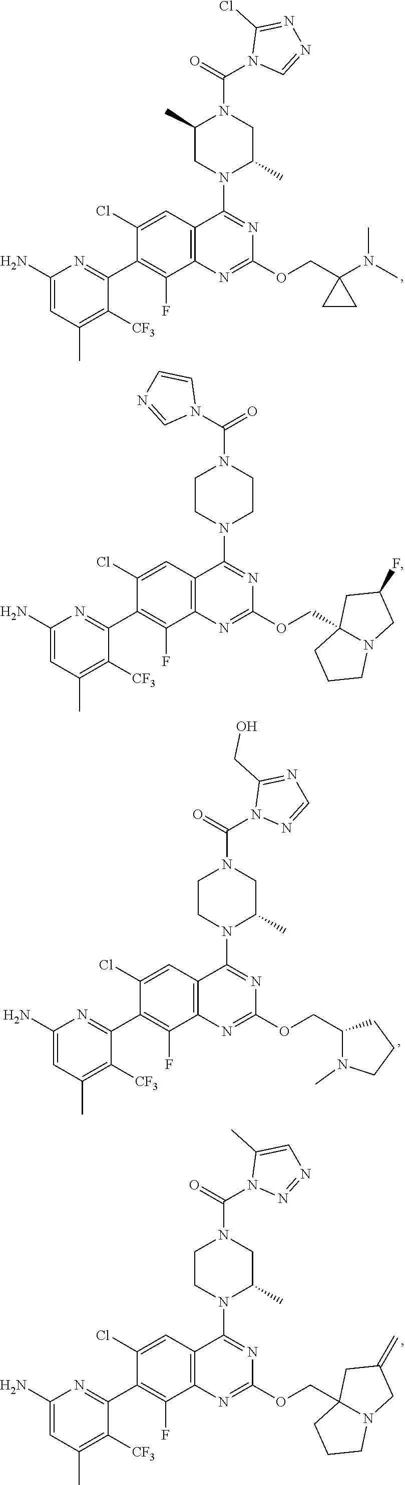

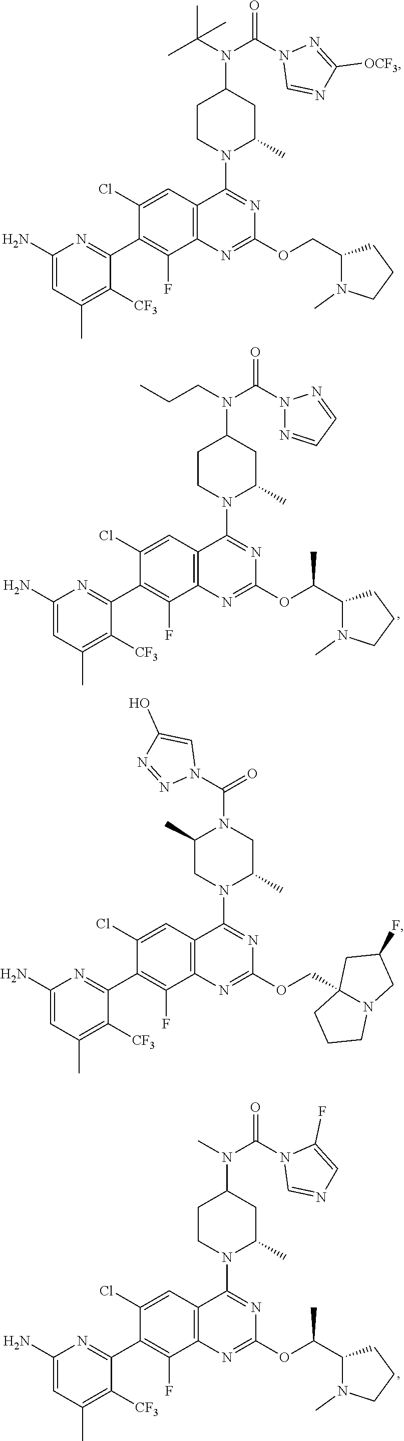 [SMILES: Cc1cc(N)nc(-c2c(Cl)cc3c(N4CCC(N(C(=O)n5cnc(OC(F)(F)F)n5)C(C)(C)C)C[C@@H]4C)nc(OC[C@@H]4CCCN4C)nc3c2F)c1C(F)(F)F ; CCCN(C(=O)n1nccn1)C1CCN(c2nc(O[C@@H](C)[C@@H]3CCCN3C)nc3c(F)c(-c4nc(N)cc(C)c4C(F)(F)F)c(Cl)cc23)[C@@H](C)C1 ; Cc1cc(N)nc(-c2c(Cl)cc3c(N4C[C@@H](C)N(C(=O)n5cc(O)nn5)C[C@@H]4C)nc(OC[C@@]45CCCN4C[C@H](F)C5)nc3c2F)c1C(F)(F)F ; Cc1cc(N)nc(-c2c(Cl)cc3c(N4CCC(N(C)C(=O)n5cncc5F)C[C@@H]4C)nc(O[C@@H](C)[C@@H]4CCCN4C)nc3c2F)c1C(F)(F)F]

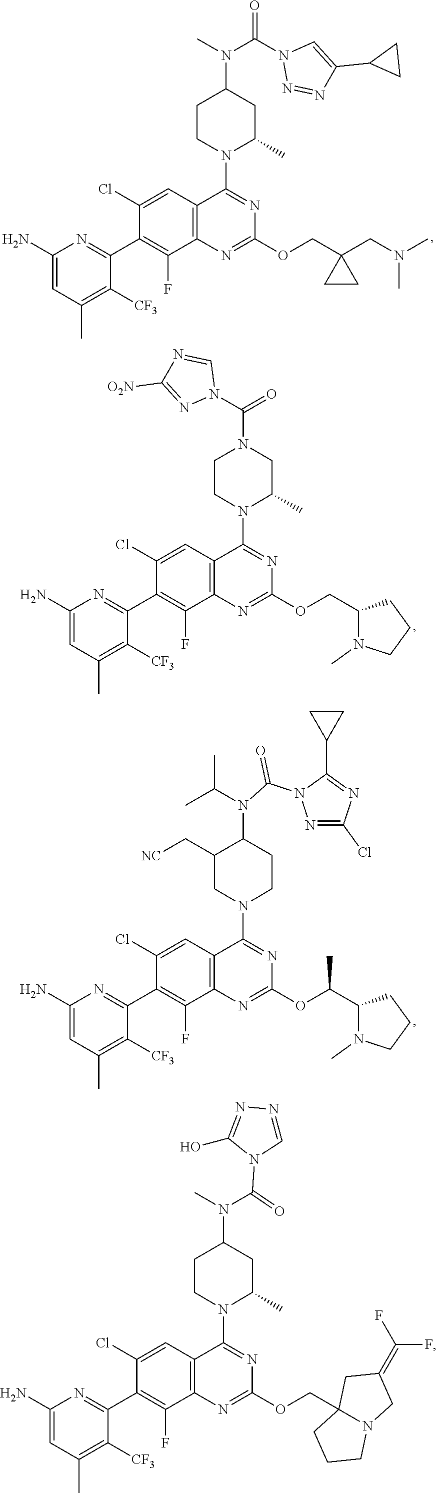

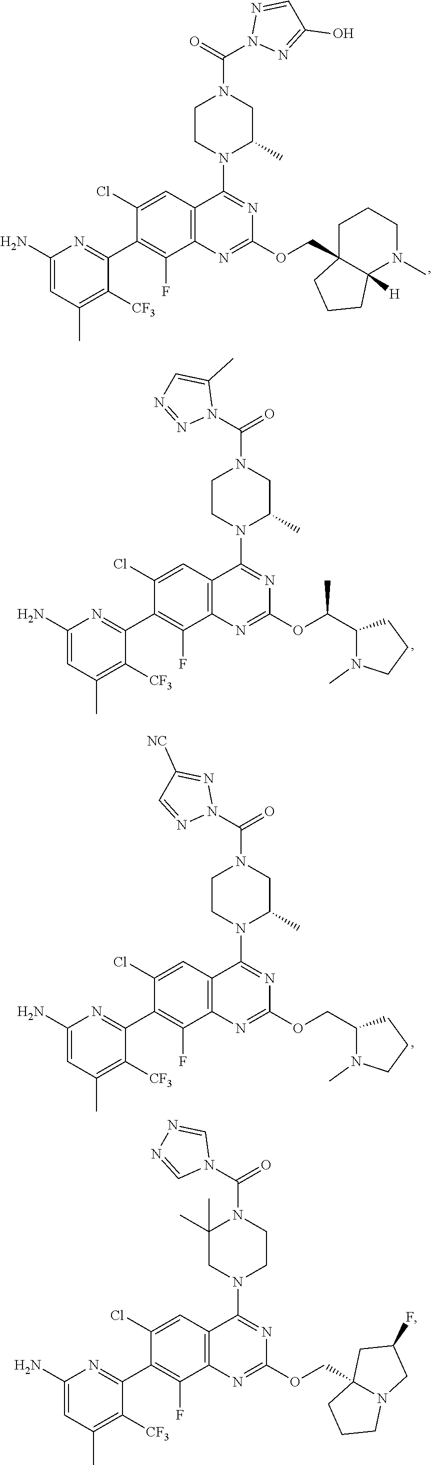 [SMILES: Cc1cc(N)nc(-c2c(Cl)cc3c(N4CCN(C(=O)n5ncc(O)n5)C[C@@H]4C)nc(OC[C@]45CCC[C@H]4N(C)CCC5)nc3c2F)c1C(F)(F)F ; Cc1cc(N)nc(-c2c(Cl)cc3c(N4CCN(C(=O)n5nncc5C)C[C@@H]4C)nc(O[C@@H](C)[C@@H]4CCCN4C)nc3c2F)c1C(F)(F)F ; Cc1cc(N)nc(-c2c(Cl)cc3c(N4CCN(C(=O)n5ncc(C#N)n5)C[C@@H]4C)nc(OC[C@@H]4CCCN4C)nc3c2F)c1C(F)(F)F ; Cc1cc(N)nc(-c2c(Cl)cc3c(N4CCN(C(=O)n5cnnc5)C(C)(C)C4)nc(OC[C@@]45CCCN4C[C@H](F)C5)nc3c2F)c1C(F)(F)F]

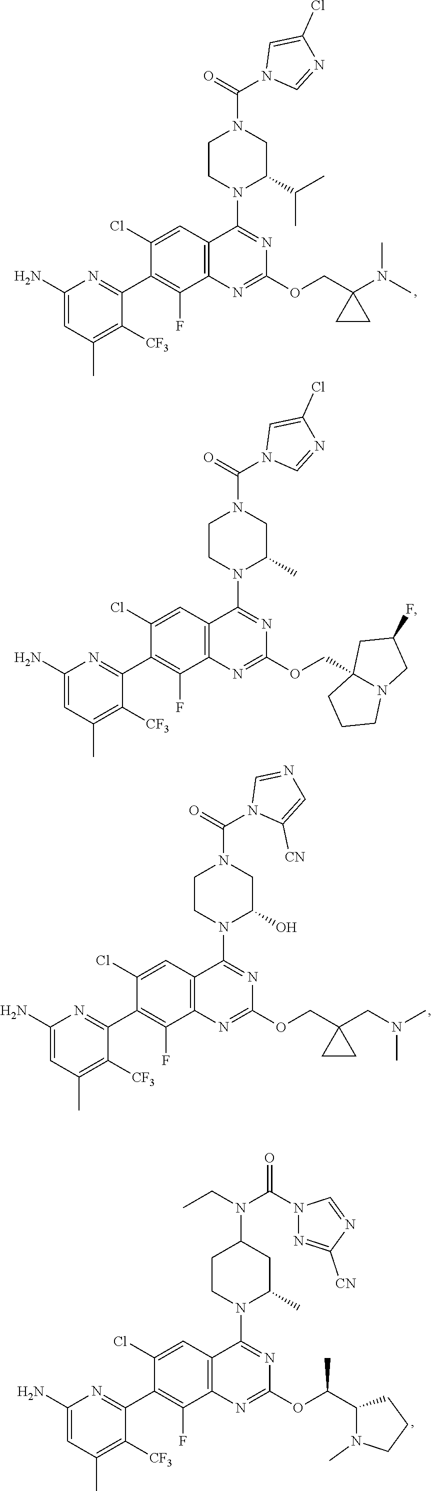 [SMILES: Cc1cc(N)nc(-c2c(Cl)cc3c(N4CCN(C(=O)n5cnc(Cl)c5)C[C@@H]4C(C)C)nc(OCC4(N(C)C)CC4)nc3c2F)c1C(F)(F)F ; Cc1cc(N)nc(-c2c(Cl)cc3c(N4CCN(C(=O)n5cnc(Cl)c5)C[C@@H]4C)nc(OC[C@@]45CCCN4C[C@H](F)C5)nc3c2F)c1C(F)(F)F ; Cc1cc(N)nc(-c2c(Cl)cc3c(N4CCN(C(=O)n5cncc5C#N)C[C@@H]4O)nc(OCC4(CN(C)C)CC4)nc3c2F)c1C(F)(F)F ; CCN(C(=O)n1cnc(C#N)n1)C1CCN(c2nc(O[C@@H](C)[C@@H]3CCCN3C)nc3c(F)c(-c4nc(N)cc(C)c4C(F)(F)F)c(Cl)cc23)[C@@H](C)C1]

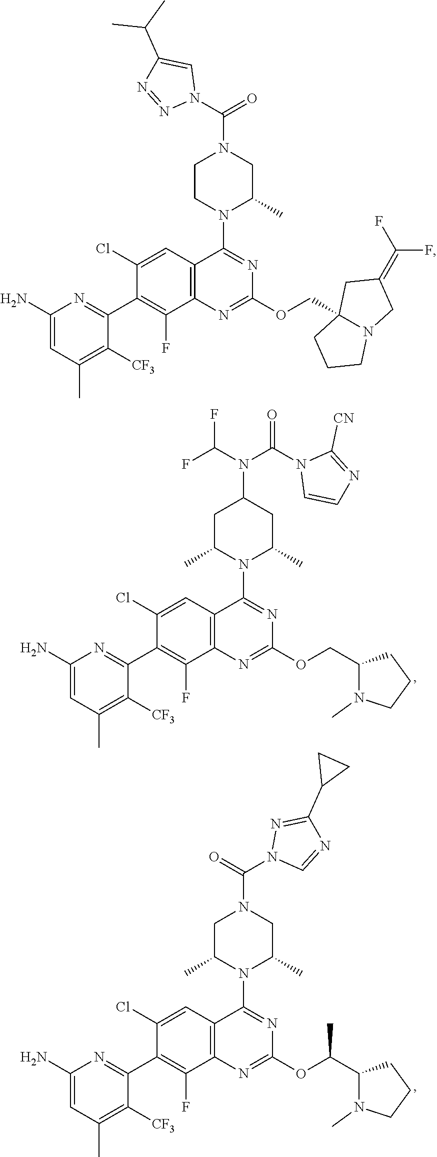

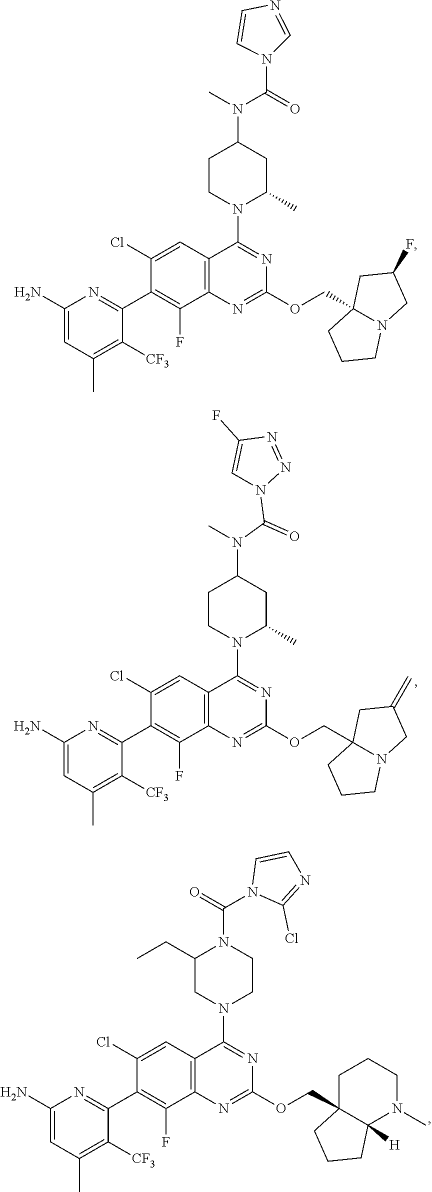 [SMILES: Cc1cc(N)nc(-c2c(Cl)cc3c(N4CCC(N(C)C(=O)n5ccnc5)C[C@@H]4C)nc(OC[C@@]45CCCN4C[C@H](F)C5)nc3c2F)c1C(F)(F)F ; C=C1CN2CCCC2(COc2nc(N3CCC(N(C)C(=O)n4cc(F)nn4)C[C@@H]3C)c3cc(Cl)c(-c4nc(N)cc(C)c4C(F)(F)F)c(F)c3n2)C1 ; CCC1CN(c2nc(OC[C@]34CCC[C@H]3N(C)CCC4)nc3c(F)c(-c4nc(N)cc(C)c4C(F)(F)F)c(Cl)cc23)CCN1C(=O)n1ccnc1Cl]

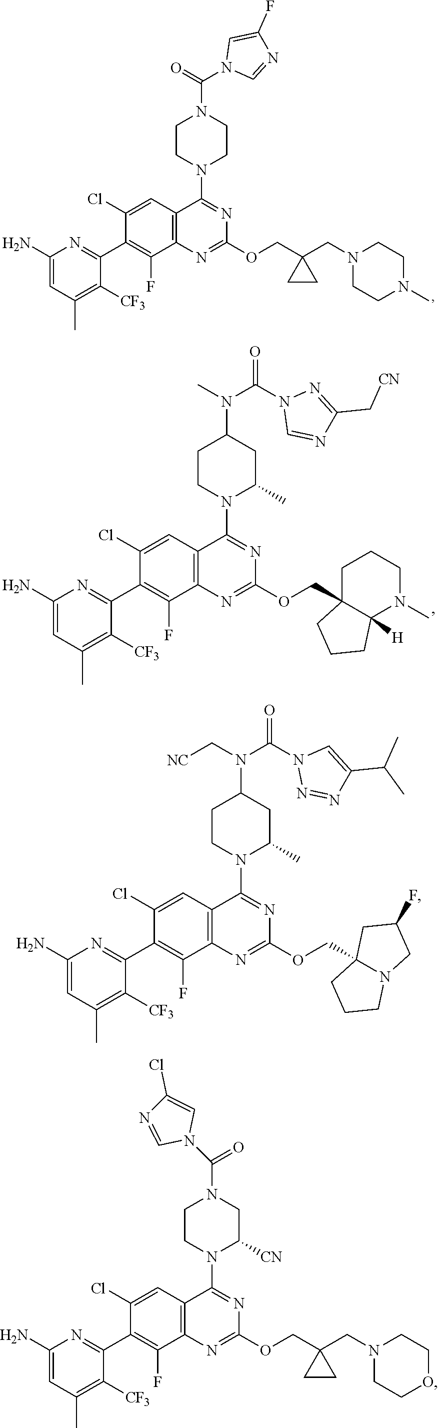 [SMILES: Cc1cc(N)nc(-c2c(Cl)cc3c(N4CCN(C(=O)n5cnc(F)c5)CC4)nc(OCC4(CN5CCN(C)CC5)CC4)nc3c2F)c1C(F)(F)F ; Cc1cc(N)nc(-c2c(Cl)cc3c(N4CCC(N(C)C(=O)n5cnc(CC#N)n5)C[C@@H]4C)nc(OC[C@]45CCC[C@H]4N(C)CCC5)nc3c2F)c1C(F)(F)F ; Cc1cc(N)nc(-c2c(Cl)cc3c(N4CCC(N(CC#N)C(=O)n5cc(C(C)C)nn5)C[C@@H]4C)nc(OC[C@@]45CCCN4C[C@H](F)C5)nc3c2F)c1C(F)(F)F ; Cc1cc(N)nc(-c2c(Cl)cc3c(N4CCN(C(=O)n5cnc(Cl)c5)C[C@@H]4C#N)nc(OCC4(CN5CCOCC5)CC4)nc3c2F)c1C(F)(F)F]

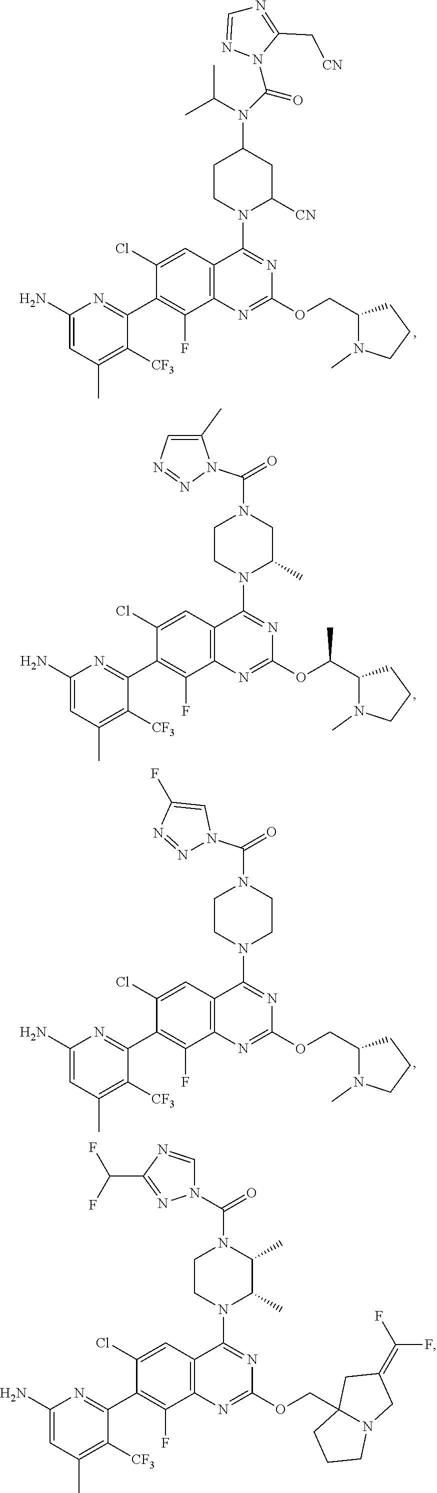

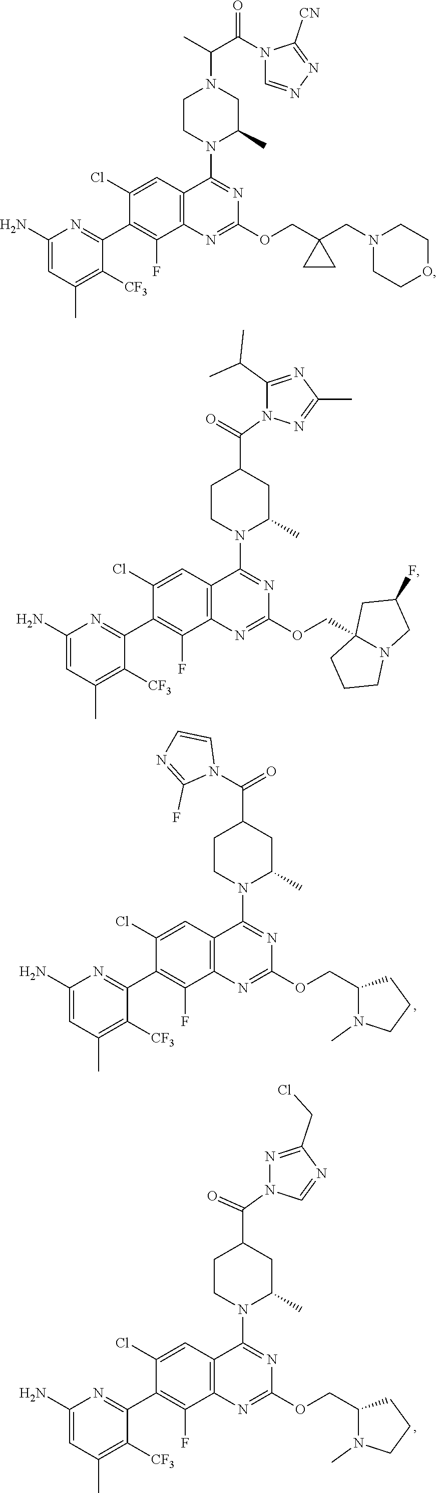 [SMILES: Cc1cc(N)nc(-c2c(Cl)cc3c(N4CCN(C(C)C(=O)n5cnnc5C#N)C[C@H]4C)nc(OCC4(CN5CCOCC5)CC4)nc3c2F)c1C(F)(F)F ; Cc1nc(C(C)C)n(C(=O)C2CCN(c3nc(OC[C@@]45CCCN4C[C@H](F)C5)nc4c(F)c(-c5nc(N)cc(C)c5C(F)(F)F)c(Cl)cc34)[C@@H](C)C2)n1 ; Cc1cc(N)nc(-c2c(Cl)cc3c(N4CCC(C(=O)n5ccnc5F)C[C@@H]4C)nc(OC[C@@H]4CCCN4C)nc3c2F)c1C(F)(F)F ; Cc1cc(N)nc(-c2c(Cl)cc3c(N4CCC(C(=O)n5cnc(CCl)n5)C[C@@H]4C)nc(OC[C@@H]4CCCN4C)nc3c2F)c1C(F)(F)F]

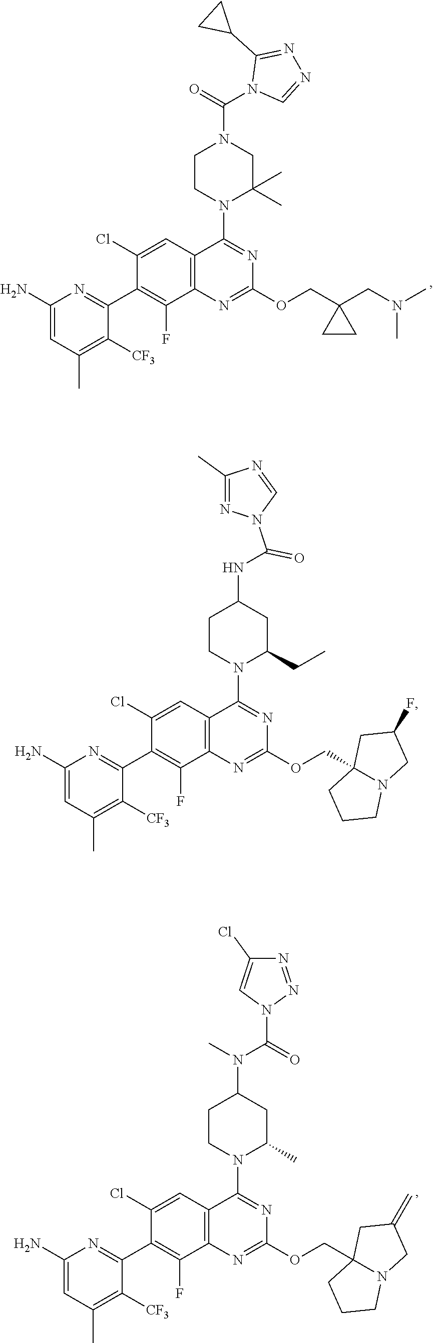 [SMILES: Cc1cc(N)nc(-c2c(Cl)cc3c(N4CCN(C(=O)n5cnnc5C5CC5)CC4(C)C)nc(OCC4(CN(C)C)CC4)nc3c2F)c1C(F)(F)F ; CC[C@@H]1CC(NC(=O)n2cnc(C)n2)CCN1c1nc(OC[C@@]23CCCN2C[C@H](F)C3)nc2c(F)c(-c3nc(N)cc(C)c3C(F)(F)F)c(Cl)cc12 ; C=C1CN2CCCC2(COc2nc(N3CCC(N(C)C(=O)n4cc(Cl)nn4)C[C@@H]3C)c3cc(Cl)c(-c4nc(N)cc(C)c4C(F)(F)F)c(F)c3n2)C1]

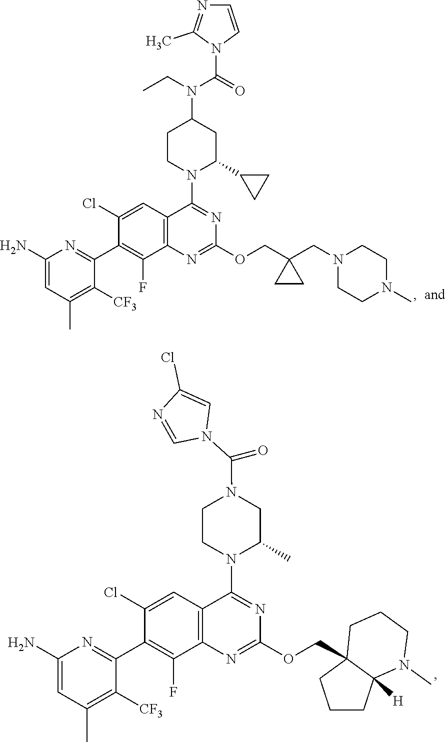

or a pharmaceutically acceptable salt, solvate, stereoisomer, or prodrug thereof.

In general, compounds of the disclosure may be prepared by the following reaction scheme:

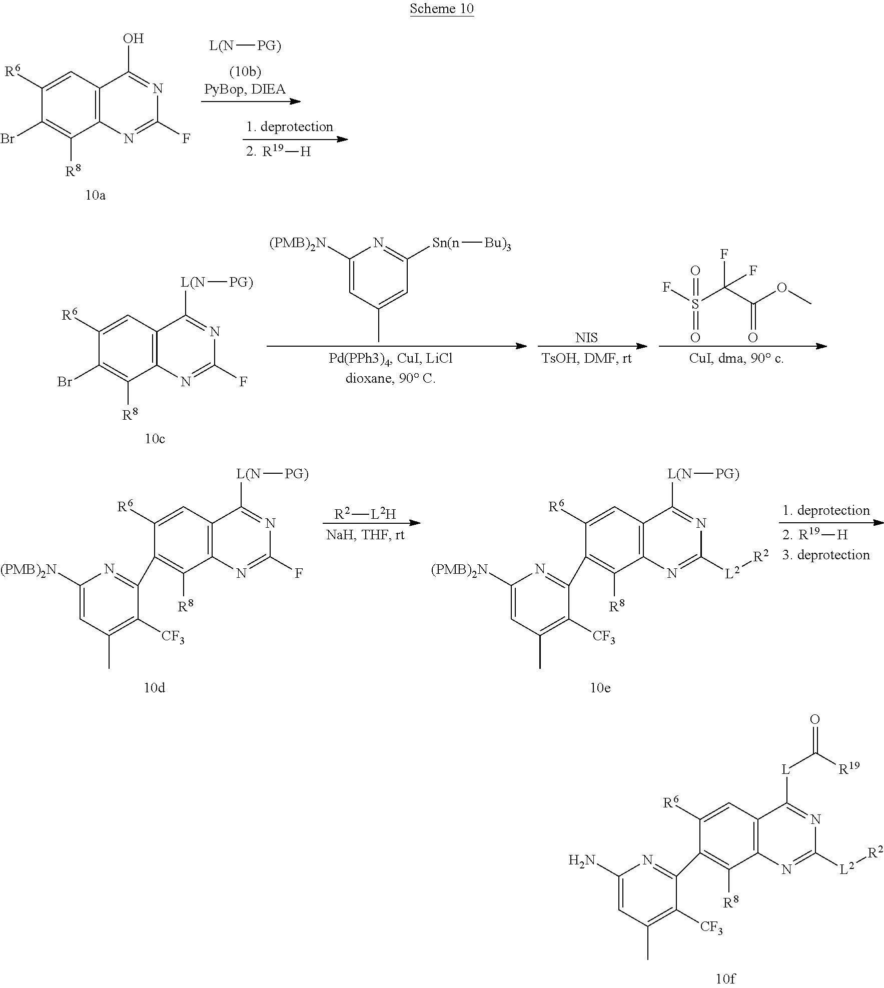

In some embodiments, a compound of Formula 10f may be prepared according to Scheme 10. For example, alcohol 10a can be treated with 10b—wherein L comprises an unprotected primary or secondary amine, and further wherein L comprises a suitably protected nitrogen atom (e.g., wherein PG is Boc, Cbz, or Fmoc)—in the presence of PyBop and a suitable base to provide the aminated product (10c). A cross-coupling reaction, such as a Stille reaction with a suitably-substituted pyridine, can provide the corresponding 7-substituted quinazoline. To the product is added N-iodosuccinimide and p-toluenesulfonic acid monohydrate in N,N-dimethylformamide, and the mixture is stirred. To the product is added methyl 2,2-difluoro-2-(fluorosulfonyl)acetate and cuprous iodide in N,N-dimethylacetamide with stirring followed by purification. To the product is added R²-L²-H, a solution of sodium hydride in THF, and the mixture is stirred and concentrated. Deprotection can be followed by reaction with R¹⁹—H in the presence of triphosgene bis(trichloromethyl) carbonate (BTC) and pyridine to provide a compound of Formula 10f.

Synthetic procedures for certain intermediates may be found in WO 2020/097537, which is incorporated herein by reference in its entirety, including any synthetic methods, recitations of compound variables, combination therapies, and methods of use disclosed therein.

### Compounds of Formula (XIX), (XX), (XXI), and (XXII)

In certain aspects, the present disclosure provides a compound of Formula (XIX):

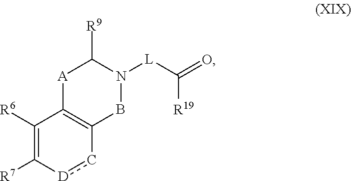

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from —OCH(R⁵)— and —N(R⁵)CH(R⁵)—;
B is selected from —CH₂— and —C(O)—;
C is selected from —C(R³)═, —C(R³)₂—, —N═, —N(R³)—, and C(O)—
D is selected from —C(R⁸)═, —C(R⁸)₂—, —N═, —N(R⁸)—, and C(O)—
wherein -C [symbol] D- is selected from —C(R³)═C(R⁸)—, —C(R³)═N—, —N═C(R⁸)—, —C(O)N(R⁸)—, —C(R³)₂—N(R⁸)—, —N(R³)—C(R⁸)₂—, —C(R³)₂—C(R⁸)₂—, or —N(R³)C(O)—;
R³ and R⁸ are each independently selected from hydrogen, halogen, CN, —OR¹², C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; or R³ and R⁸ are joined to form, together with the atoms to which they are attached, a C₅₋₆ carbocycle or 5- to 6-membered heterocycle, wherein the C₅₋₆ carbocycle and 5- to 6-membered heterocycle are optionally substituted;
R⁵ is independently selected from hydrogen and C₁₋₆ alkyl; wherein C₁₋₆ alkyl is optionally substituted;
R⁶ is selected from hydrogen, halogen, —CN, C₁₋₆ alkyl, C₂₋₆ alkenyl, C₂₋₆ alkynyl, and —O—C₁₋₆ alkyl, wherein—C₁₋₆ alkyl, C₂₋₆ alkenyl, C₂₋₆ alkynyl, and —O—C₁₋₆ alkyl are optionally substituted with one or more halogen;
R⁷ is independently selected from C₃₋₁₂ carbocycle, monocyclic 5- to 6-membered heterocycle, bicyclic 9-membered heterocycle comprising two or more ring nitrogen atoms, and bicyclic 10-membered heterocycle; wherein C₃₋₁₂ carbocycle, monocyclic 5- to 6-membered heterocycle, bicyclic 9-membered heterocycle comprising two or more ring nitrogen atoms, and bicyclic 10-membered heterocycle are each optionally substituted;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, for a compound of Formula (XIX), R³ is selected from hydrogen, halogen, —CN, —O(C₁₋₆ alkyl), —O(C₁₋₆ deuteroalkyl), —O(C₁₋₆ haloalkyl), —O(C₃₋₆ carbocycle), C₁₋₆ alkyl, C₂₋₆ alkynyl, C₁₋₆ haloalkyl, C₃₋₁₂ carbocycle, and 3- to 12-membered heterocycle; wherein C₁₋₆ alkyl is optionally substituted with —O(C₁₋₆ alkyl); C₂₋₆ alkynyl is optionally substituted with —N(C₁₋₆ alkyl)₂ or —O(C₁₋₆ alkyl); —O(C₁₋₆ alkyl) is optionally substituted with —N(C₁₋₆ alkyl)₂, —O(C₁₋₆ alkyl), or (3- to 12-membered heterocycle); and C₃₋₁₂ carbocycle and 3- to 12-membered heterocycle are each optionally substituted. In some embodiments, R³ is hydrogen.

In some embodiments, for a compound of Formula (XIX), R⁷ is optionally substituted phenyl. In some embodiments, R⁷ is optionally substituted naphthyl. In some embodiments, R⁷ is optionally substituted quinolinyl. In some embodiments, R⁷ is optionally substituted isoquinolinyl. In some embodiments, R⁷ is optionally substituted indazolyl. In some embodiments, R⁷ is optionally substituted benzoimidazolyl. In some embodiments, R⁷ is optionally substituted pyridyl. In some embodiments, R⁷ is optionally substituted C₃₋₁₂ carbocycle. In some embodiments, R⁷ is optionally substituted monocyclic 5- to 6-membered heterocycle. In some embodiments, R⁷ is optionally substituted bicyclic 9-membered heterocycle comprising two or more ring nitrogen atoms. In some embodiments, R⁷ is optionally substituted bicyclic 10-membered heterocycle.

In some embodiments, for a compound of Formula (XIX), R⁷ is selected from

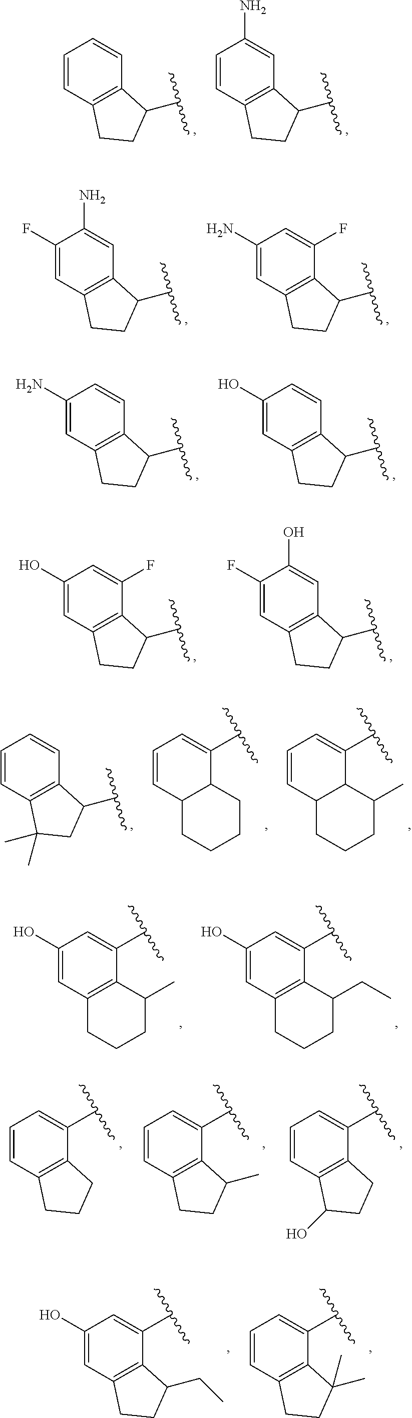

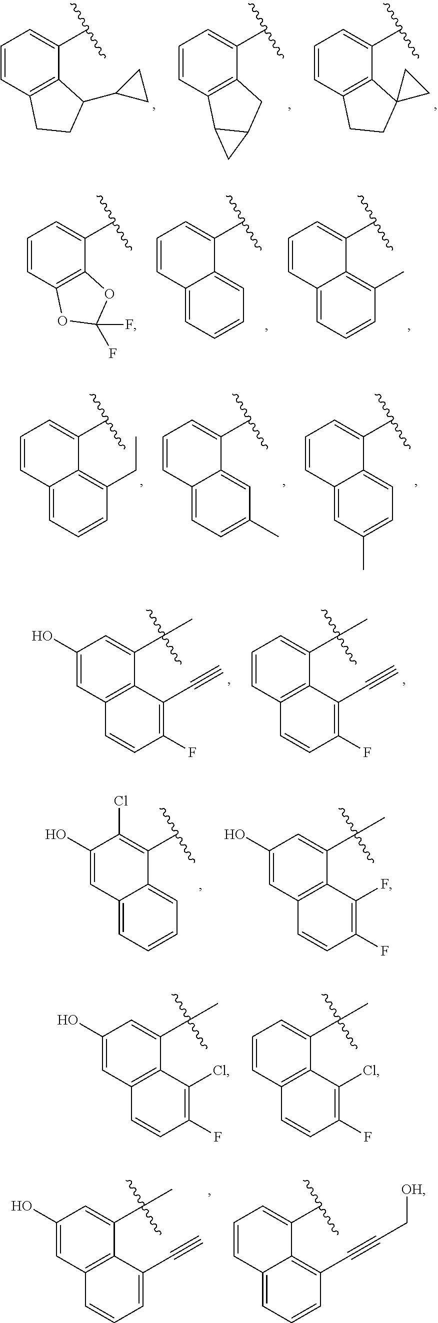

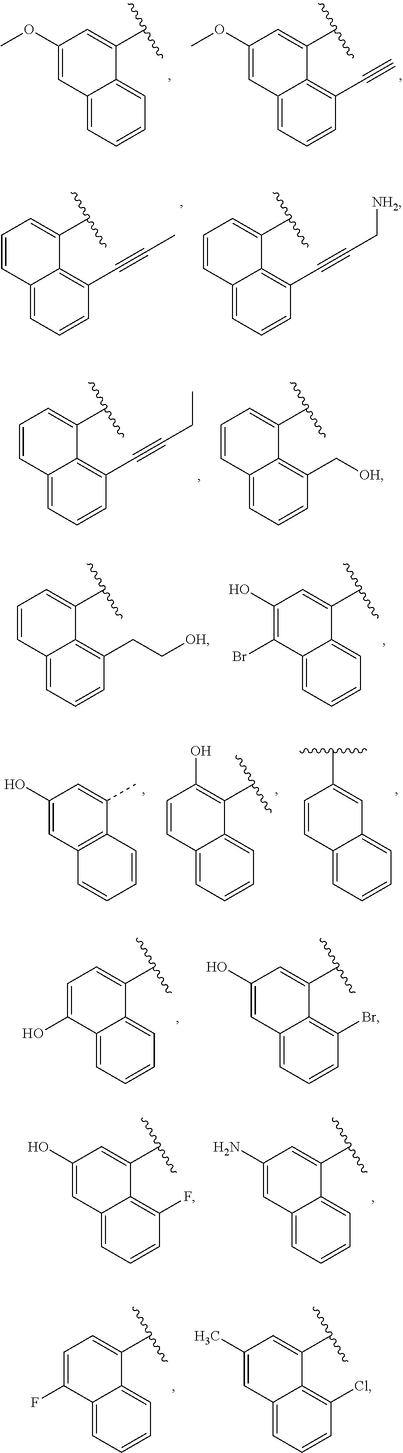

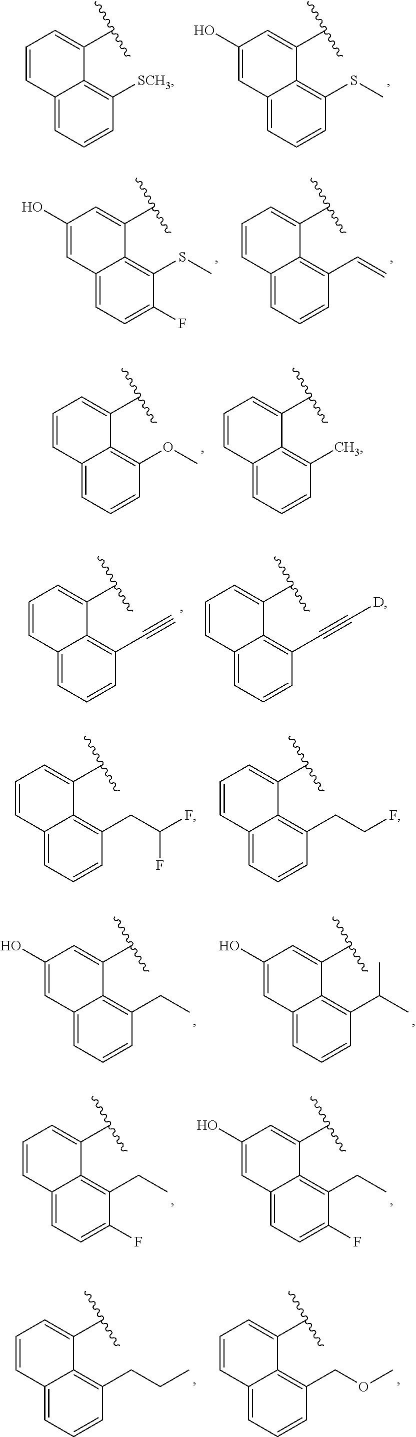

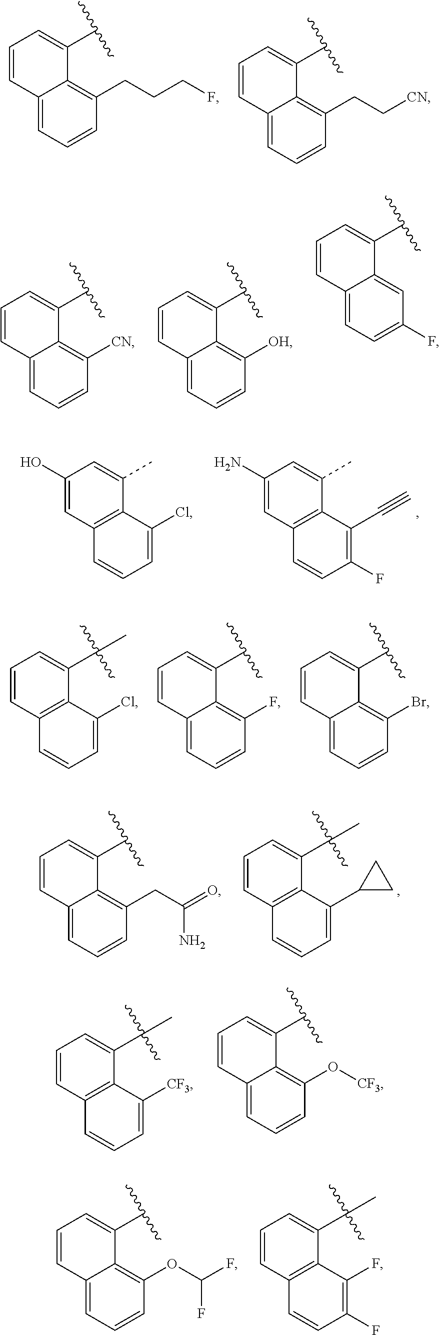

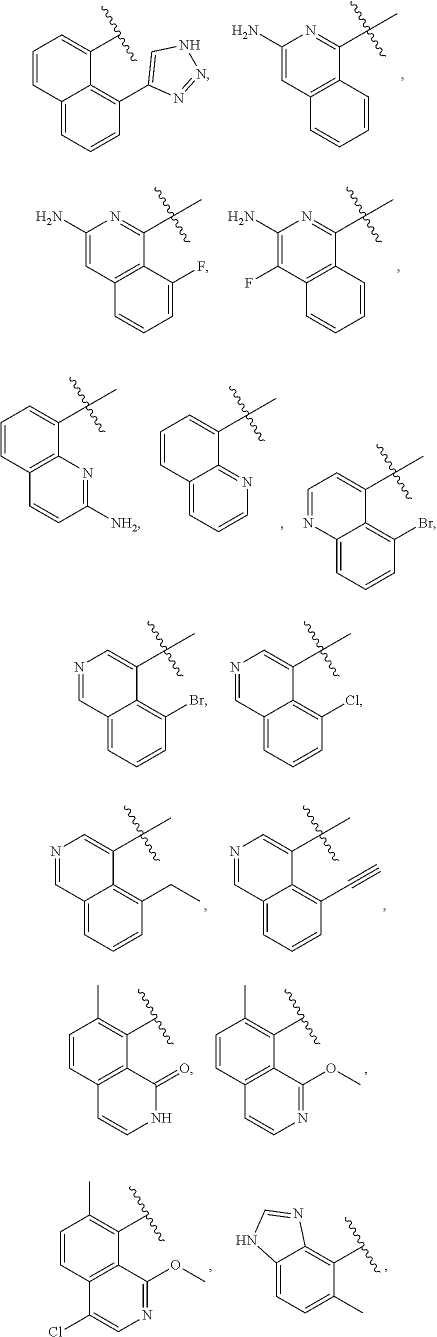

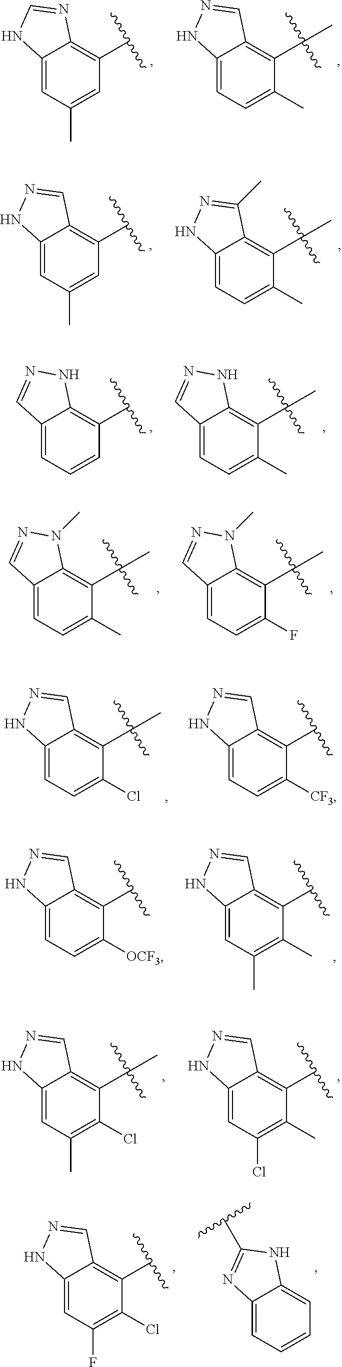

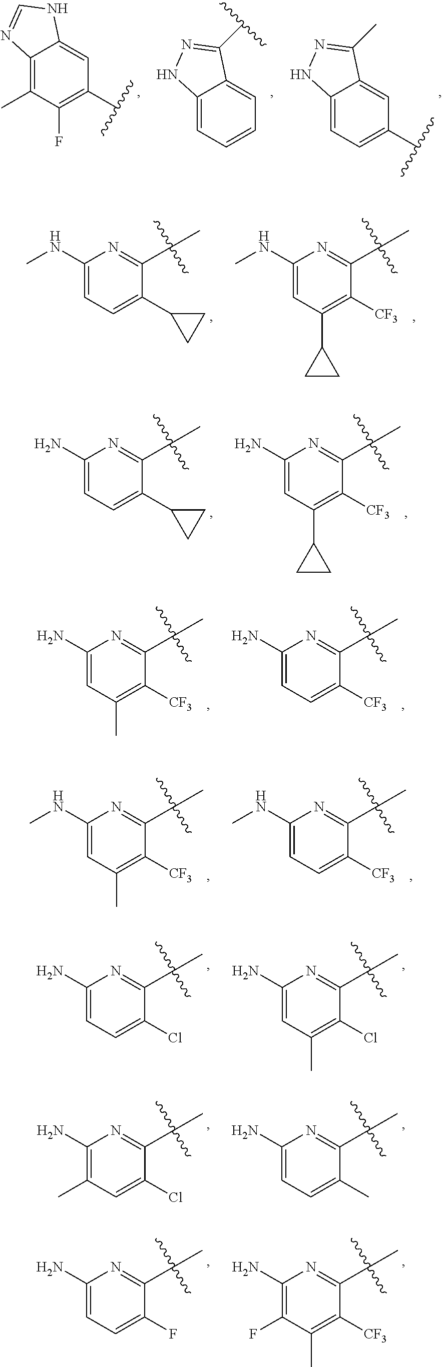

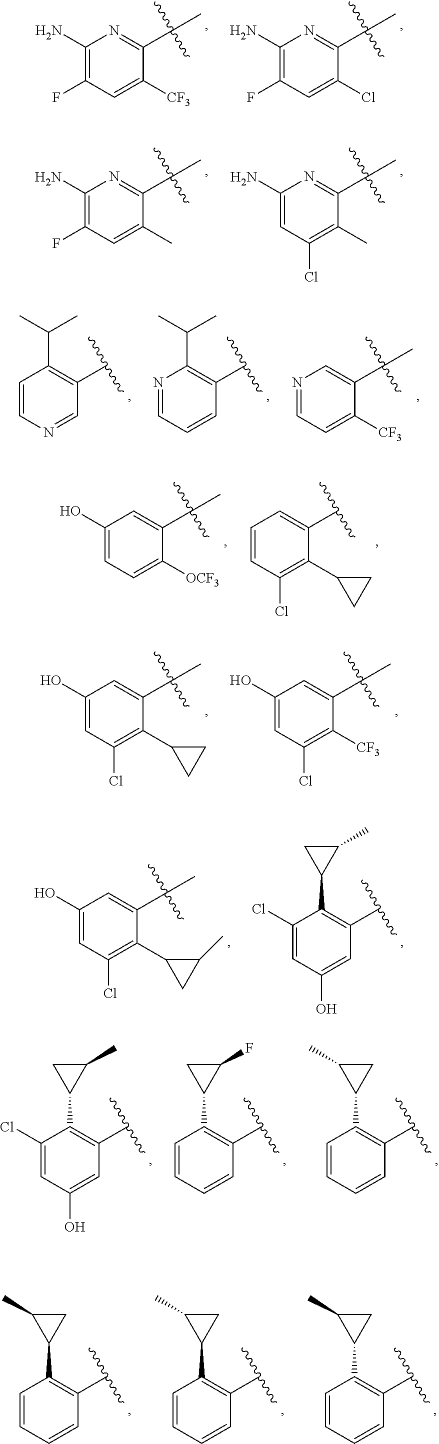

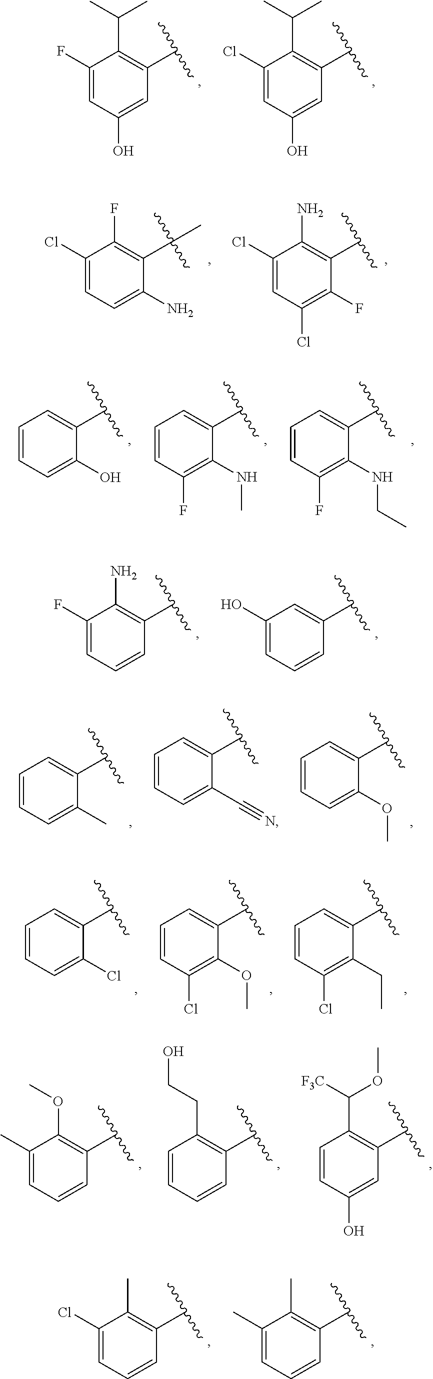

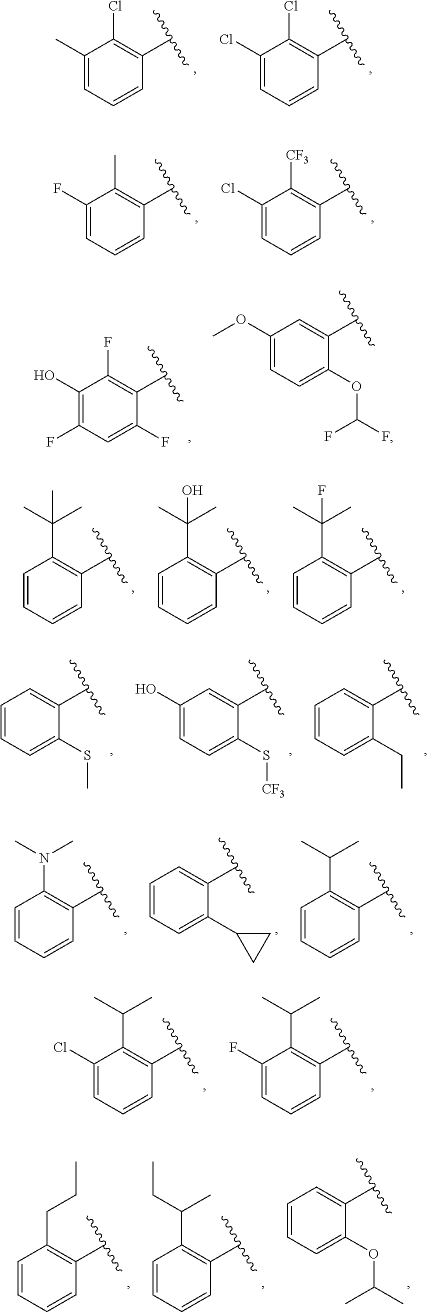

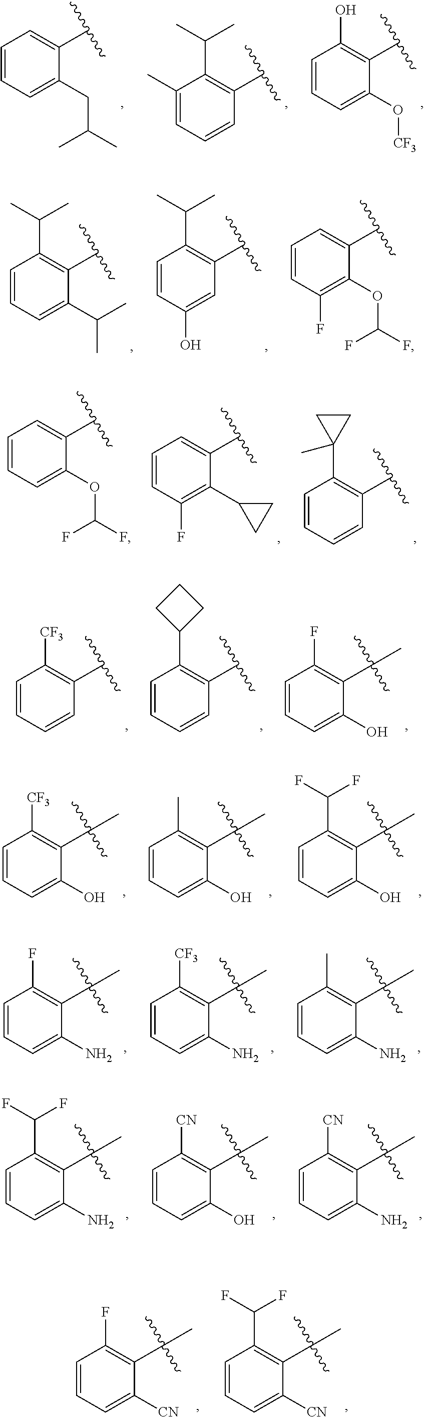

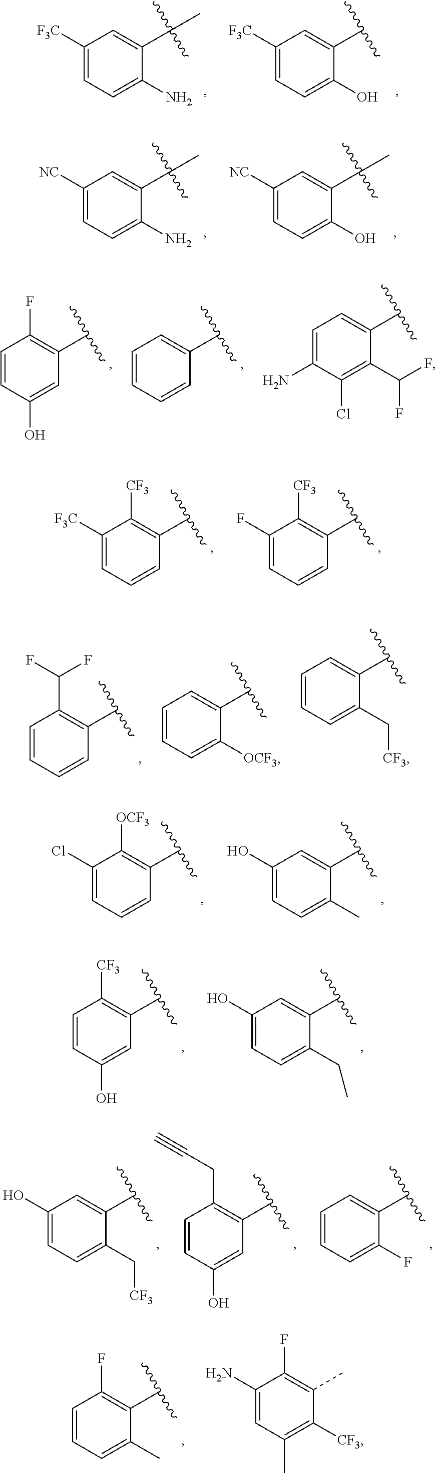

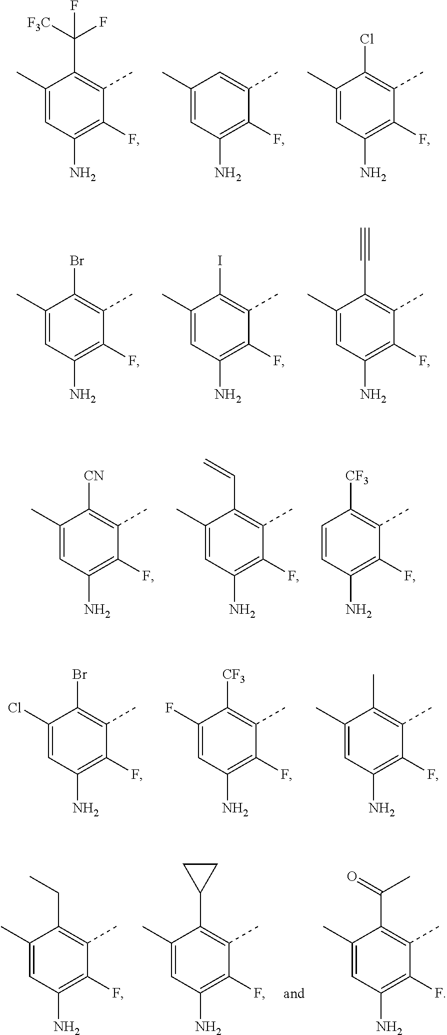

In certain aspects, the present disclosure provides a compound of Formula (XX):

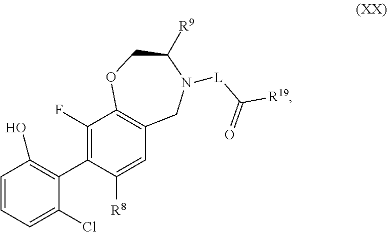

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R⁸ is selected from hydrogen, halogen, CN, —OR¹², C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, for a compound of Formula (XIX) or (XX), R⁸ is selected from hydrogen, halogen, —CN, —O(C₁₋₆ alkyl), —O(C₁₋₆ deuteroalkyl), —O(C₁₋₆ haloalkyl), —O(C₃₋₆ carbocycle), C₁₋₆ alkyl, C₂₋₆ alkynyl, C₁₋₆ haloalkyl, C₃₋₁₂ carbocycle, and 3- to 12-membered heterocycle; wherein C₁₋₆ alkyl is optionally substituted with —O(C₁₋₆ alkyl); C₂₋₆ alkynyl is optionally substituted with —N(C₁₋₆ alkyl)₂ or —O(C₁₋₆ alkyl); —O(C₁₋₆ alkyl) is optionally substituted with —N(C₁₋₆ alkyl)₂, —O(C₁₋₆ alkyl), or (3- to 12-membered heterocycle); and C₃₋₁₂ carbocycle and 3- to 12-membered heterocycle are each optionally substituted. In some embodiments, R⁸ is C₂₋₄ alkynyl. In some embodiments, R⁸ is —CCCH₃. In some embodiments, R⁸ is hydrogen.

In some embodiments, for a compound of Formula (XIX) or (XX), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea.

In some embodiments, for a compound of Formula (XIX) or (XX), L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH— or —CH₂N(CH₃)—. In some embodiments, L¹ is selected from a bond and 2- to 6-membered heteroalkyl. In some embodiments, L¹ is selected from a bond and optionally substituted 2- to 6-membered heteroalkyl.

In some embodiments, for a compound of Formula (XIX) or (XX), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (XIX) or (XX), L², R⁹, and the atoms to which they are attached form optionally substituted 3- to 12-membered heterocycle. In some embodiments, L², R⁹, and the atoms to which they are attached form optionally substituted 3- to 7-membered monocyclic heterocycle, such as optionally substituted 3- to 7-membered monocyclic heterocycloalkyl. In some embodiments, L², R⁹, and the atoms to which they are attached form optionally substituted 5- to 8-membered monocyclic heterocycle, such as optionally substituted 5- to 8-membered monocyclic heterocycloalkyl. In some embodiments, L², R⁹, and the atoms to which they are attached form optionally substituted 6- to 12-membered spirocyclic heterocycle, such as optionally substituted 6- to 12-membered spirocyclic heterocycloalkyl. In some embodiments, L², R⁹, and the atoms to which they are attached form optionally substituted 7- to 12-membered fused bicyclic heterocycle, such as optionally substituted 7- to 12-membered fused bicyclic heterocycle.

In some embodiments, for a compound of Formula (XIX) or (XX), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (XIX) or (XX), L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle. In some embodiments, L¹ is a bond; L², R⁹, and the atoms to which they are attached form optionally substituted 3- to 12-membered heterocycle; and L³ is selected from a bond and optionally substituted nitrene.

In some embodiments, the compound of Formula (XIX) is:

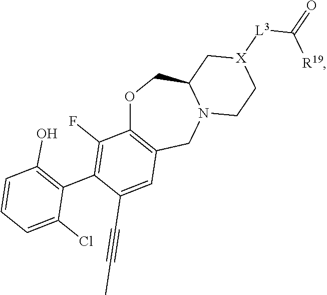

or a pharmaceutically acceptable salt or solvate thereof, wherein X is selected from CH and N. In some embodiments, X is N and L³ is a bond. In some embodiments, X is CH and L³ is optionally substituted nitrene.

In certain aspects, the present disclosure provides a compound selected from:

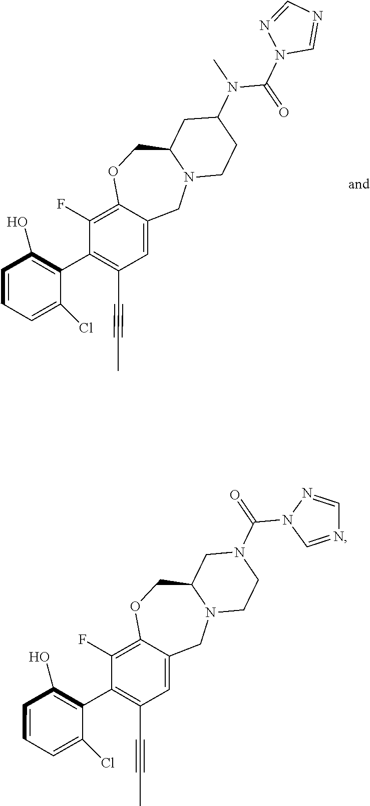

or a pharmaceutically acceptable salt, solvate, stereoisomer, or prodrug thereof.

In certain aspects, the present disclosure provides a compound selected from:

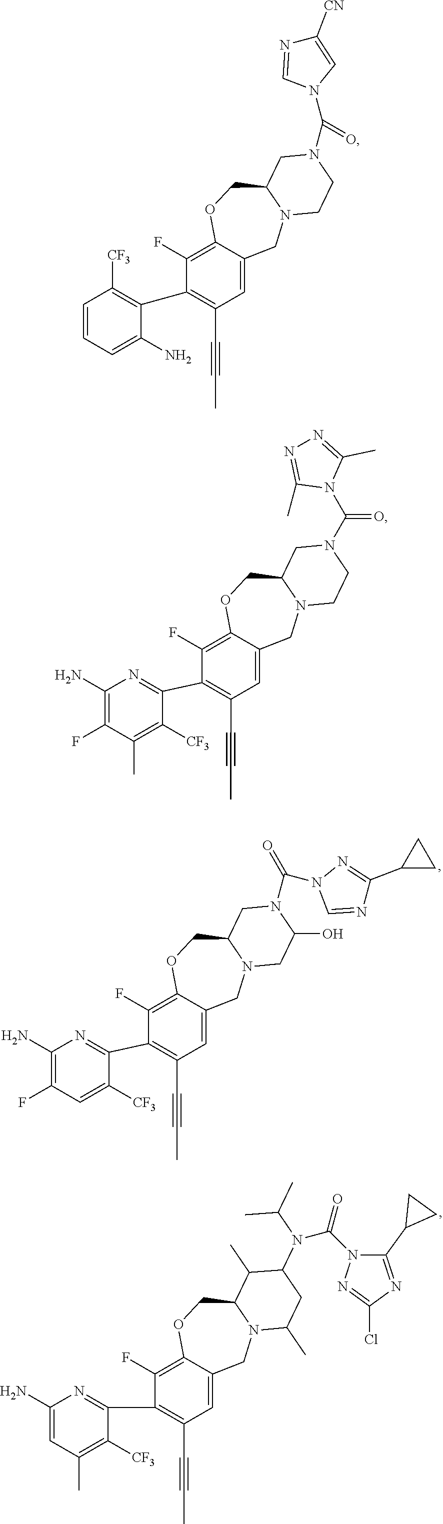 [SMILES: CC#Cc1cc2c(c(F)c1-c1c(N)cccc1C(F)(F)F)OC[C@H]1CN(C(=O)n3cnc(C#N)c3)CCN1C2 ; CC#Cc1cc2c(c(F)c1-c1nc(N)c(F)c(C)c1C(F)(F)F)OC[C@H]1CN(C(=O)n3c(C)nnc3C)CCN1C2 ; CC#Cc1cc2c(c(F)c1-c1nc(N)c(F)cc1C(F)(F)F)OC[C@H]1CN(C(=O)n3cnc(C4CC4)n3)C(O)CN1C2 ; CC#Cc1cc2c(c(F)c1-c1nc(N)cc(C)c1C(F)(F)F)OC[C@H]1C(C)C(N(C(=O)n3nc(Cl)nc3C3CC3)C(C)C)CC(C)N1C2]

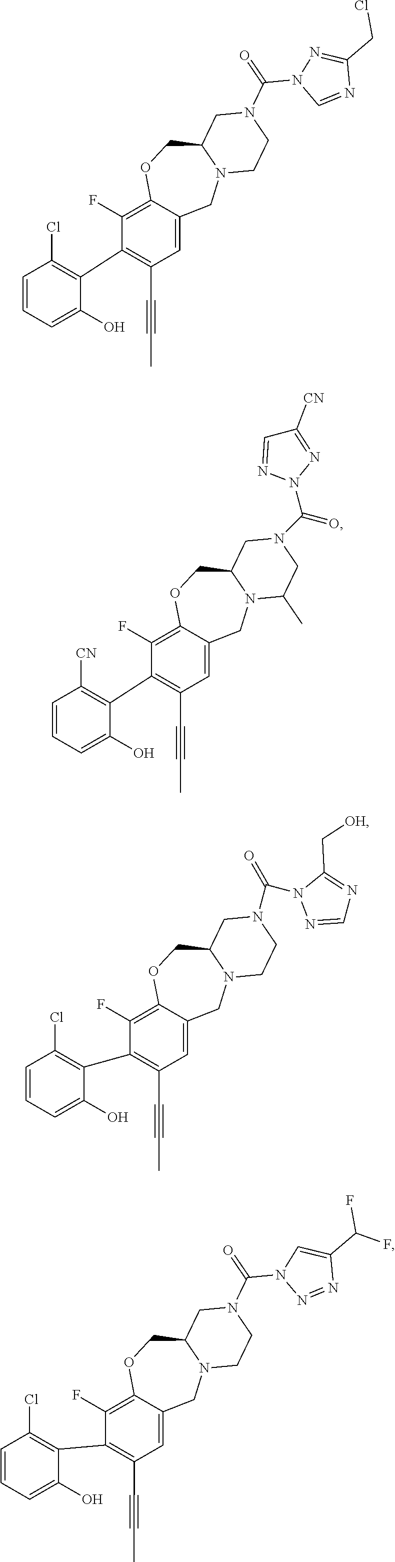 [SMILES: CC#Cc1cc2c(c(F)c1-c1c(O)cccc1Cl)OC[C@H]1CN(C(=O)n3cnc(CCl)n3)CCN1C2 ; CC#Cc1cc2c(c(F)c1-c1c(O)cccc1C#N)OC[C@H]1CN(C(=O)n3ncc(C#N)n3)CC(C)N1C2 ; CC#Cc1cc2c(c(F)c1-c1c(O)cccc1Cl)OC[C@H]1CN(C(=O)n3ncnc3CO)CCN1C2 ; CC#Cc1cc2c(c(F)c1-c1c(O)cccc1Cl)OC[C@H]1CN(C(=O)n3cc(C(F)F)nn3)CCN1C2]

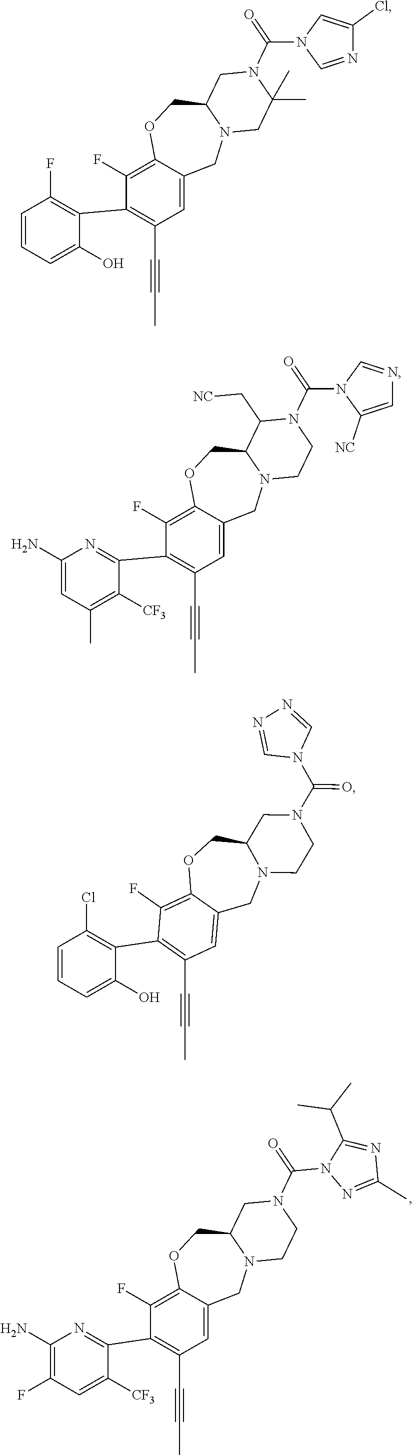 [SMILES: CC#Cc1cc2c(c(F)c1-c1c(O)cccc1F)OC[C@H]1CN(C(=O)n3cnc(Cl)c3)C(C)(C)CN1C2 ; CC#Cc1cc2c(c(F)c1-c1nc(N)cc(C)c1C(F)(F)F)OC[C@H]1C(CC#N)N(C(=O)n3cncc3C#N)CCN1C2 ; CC#Cc1cc2c(c(F)c1-c1c(O)cccc1Cl)OC[C@H]1CN(C(=O)n3cnnc3)CCN1C2 ; CC#Cc1cc2c(c(F)c1-c1nc(N)c(F)cc1C(F)(F)F)OC[C@H]1CN(C(=O)n3nc(C)nc3C(C)C)CCN1C2]

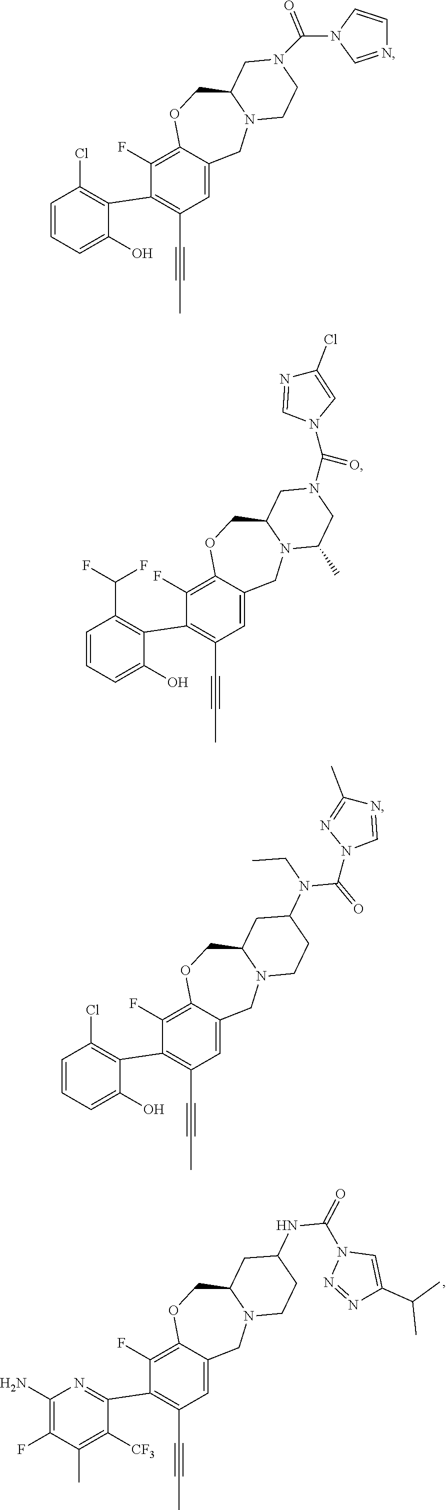 [SMILES: CC#Cc1cc2c(c(F)c1-c1c(O)cccc1Cl)OC[C@H]1CN(C(=O)n3ccnc3)CCN1C2 ; CC#Cc1cc2c(c(F)c1-c1c(O)cccc1C(F)F)OC[C@H]1CN(C(=O)n3cnc(Cl)c3)C[C@H](C)N1C2 ; CC#Cc1cc2c(c(F)c1-c1c(O)cccc1Cl)OC[C@H]1CC(N(CC)C(=O)n3cnc(C)n3)CCN1C2 ; CC#Cc1cc2c(c(F)c1-c1nc(N)c(F)c(C)c1C(F)(F)F)OC[C@H]1CC(NC(=O)n3cc(C(C)C)nn3)CCN1C2]

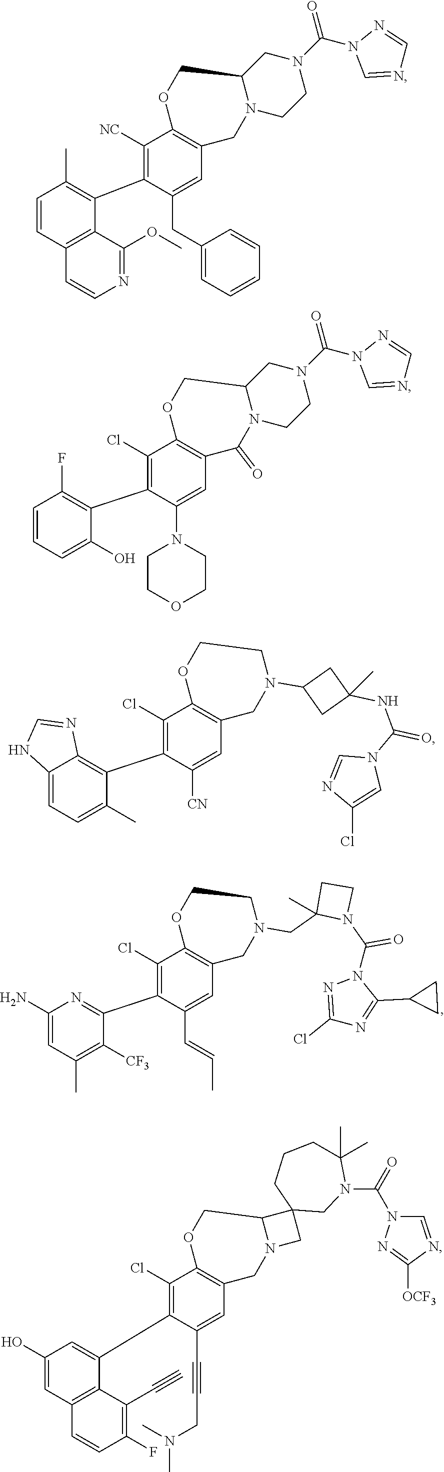

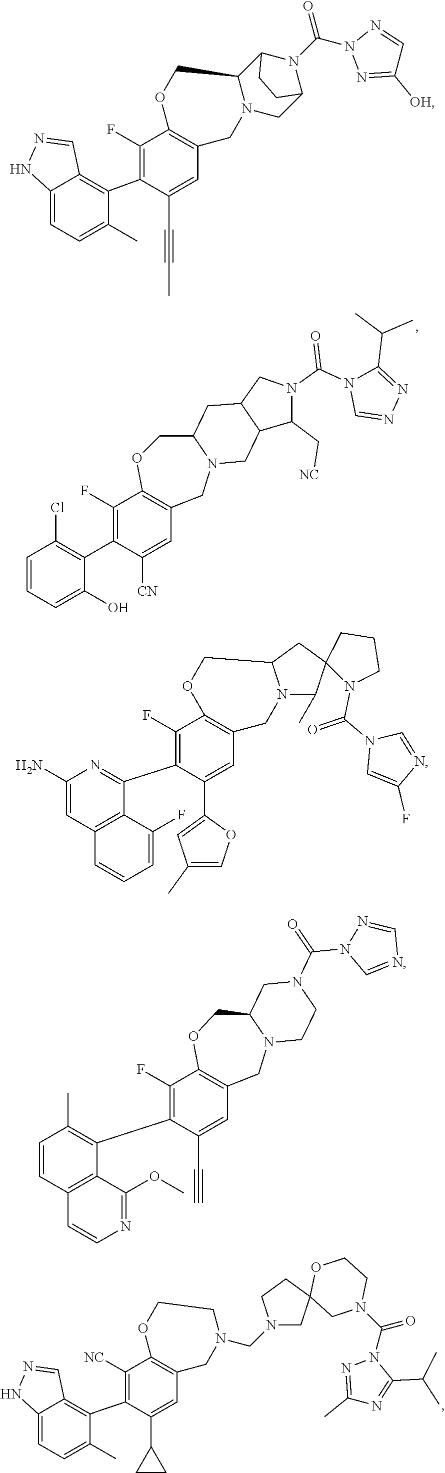 [SMILES: CC#Cc1cc2c(c(F)c1-c1c(C)ccc3[nH]ncc13)OC[C@H]1C3CCC(CN1C2)N3C(=O)n1ncc(O)n1 ; CC(C)c1nncn1C(=O)N1CC2CC3COc4c(cc(C#N)c(-c5c(O)cccc5Cl)c4F)CN3CC2C1CC#N ; Cc1coc(-c2cc3c(c(F)c2-c2nc(N)cc4cccc(F)c24)OCC2CC4(CCCN4C(=O)n4cnc(F)c4)C(C)N2C3)c1 ; C#Cc1cc2c(c(F)c1-c1c(C)ccc3ccnc(OC)c13)OC[C@H]1CN(C(=O)n3cncn3)CCN1C2 ; Cc1nc(C(C)C)n(C(=O)N2CCOC3(CCN(CN4CCOc5c(cc(C6CC6)c(-c6c(C)ccc7[nH]ncc67)c5C#N)C4)C3)C2)n1]

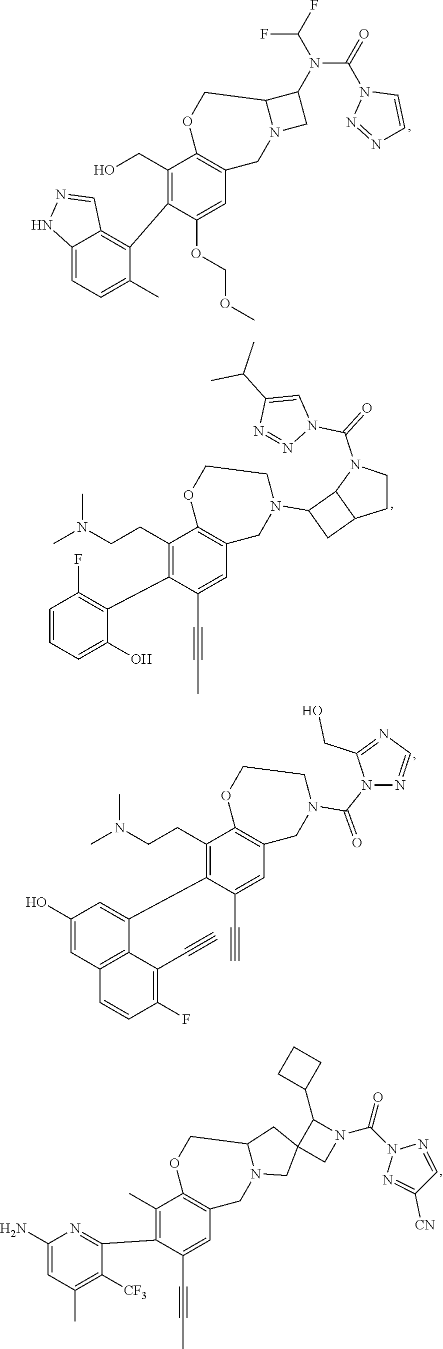

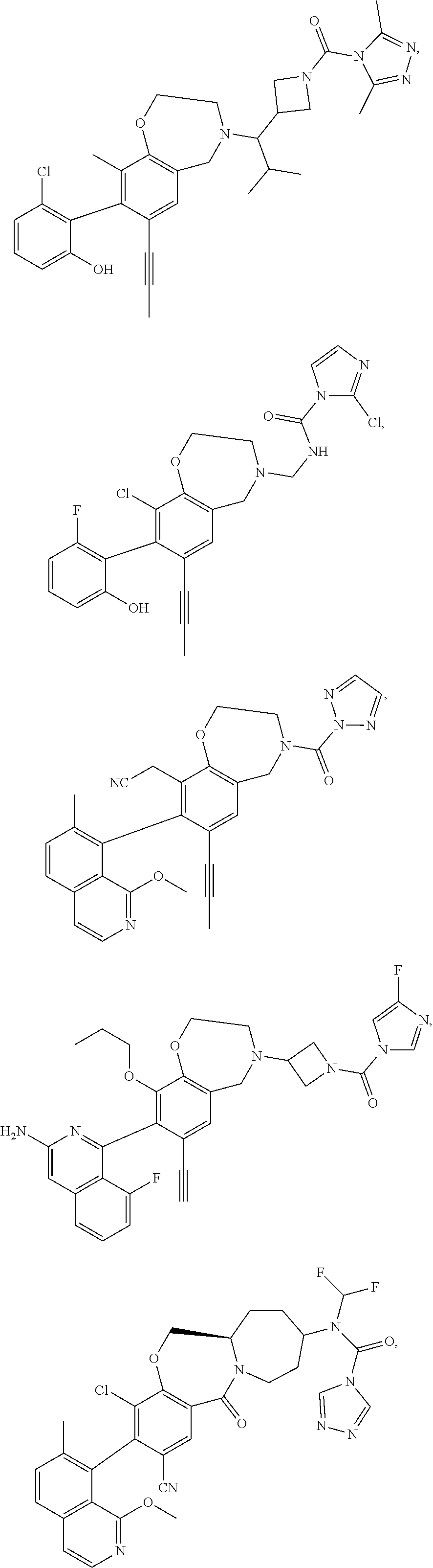 [SMILES: CC#Cc1cc2c(c(C)c1-c1c(O)cccc1Cl)OCCN(C(C(C)C)C1CN(C(=O)n3c(C)nnc3C)C1)C2 ; CC#Cc1cc2c(c(Cl)c1-c1c(O)cccc1F)OCCN(CNC(=O)n1ccnc1Cl)C2 ; CC#Cc1cc2c(c(CC#N)c1-c1c(C)ccc3ccnc(OC)c13)OCCN(C(=O)n1nccn1)C2 ; C#Cc1cc2c(c(OCCC)c1-c1nc(N)cc3cccc(F)c13)OCCN(C1CN(C(=O)n3cnc(F)c3)C1)C2 ; COc1nccc2ccc(C)c(-c3c(C#N)cc4c(c3Cl)OC[C@H]3CCC(N(C(=O)n5cnnc5)C(F)F)CCN3C4=O)c12]

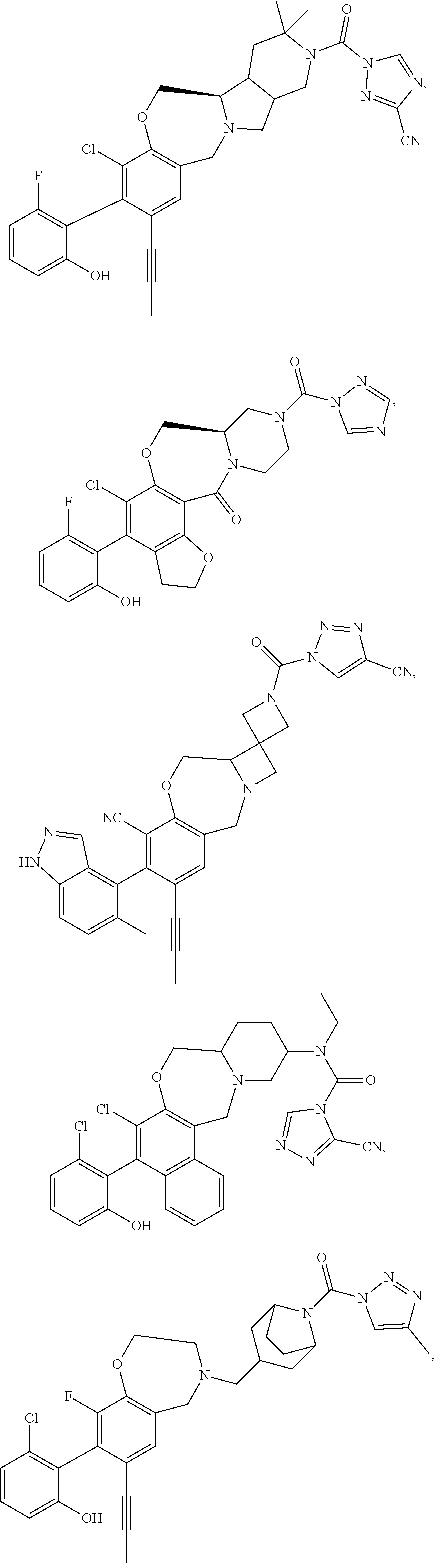 [SMILES: CC#Cc1cc2c(c(Cl)c1-c1c(O)cccc1F)OC[C@H]1C3CC(C)(C)N(C(=O)n4cnc(C#N)n4)CC3CN1C2 ; O=C1c2c(c(Cl)c(-c3c(O)cccc3F)c3c2OCC3)OC[C@H]2CN(C(=O)n3cncn3)CCN12 ; CC#Cc1cc2c(c(C#N)c1-c1c(C)ccc3[nH]ncc13)OCC1N(C2)CC12CN(C(=O)n1cc(C#N)nn1)C2 ; CCN(C(=O)n1cnnc1C#N)C1CCC2COc3c(Cl)c(-c4c(O)cccc4Cl)c4ccccc4c3CN2C1 ; CC#Cc1cc2c(c(F)c1-c1c(O)cccc1Cl)OCCN(CC1CC3CCC(C1)N3C(=O)n1cc(C)nn1)C2]

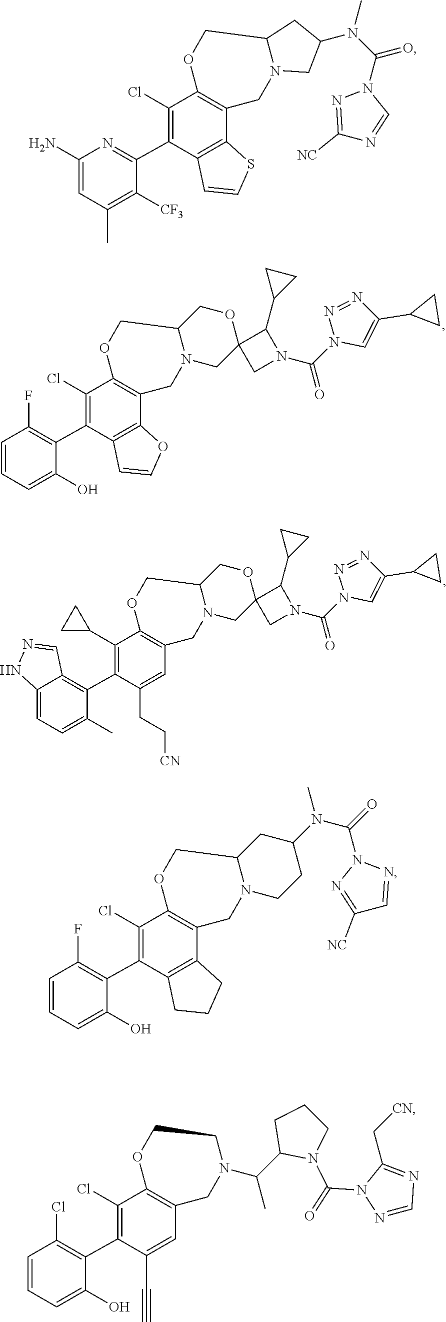

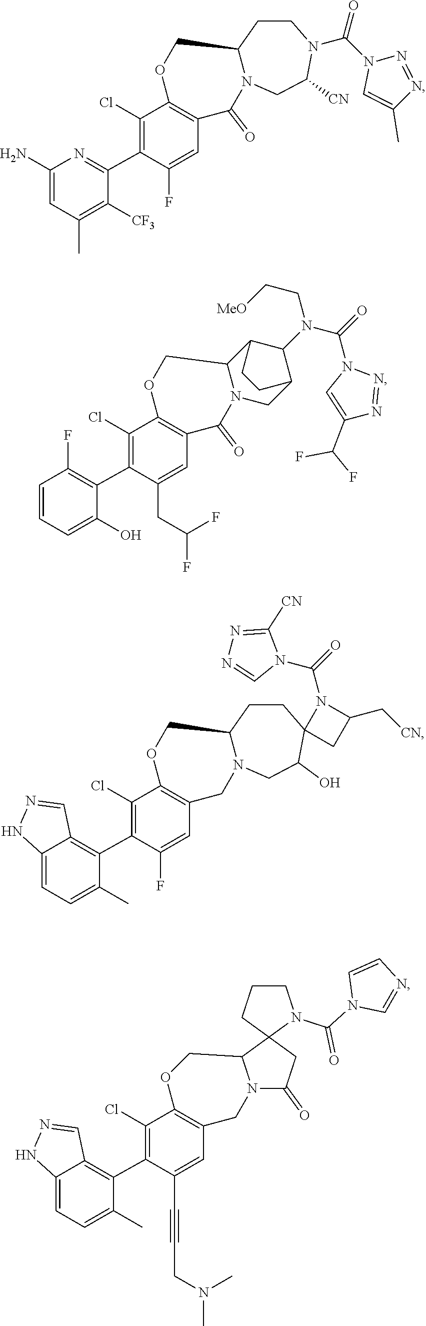 [SMILES: Cc1cn(C(=O)N2CC[C@@H]3COc4c(cc(F)c(-c5nc(N)cc(C)c5C(F)(F)F)c4Cl)C(=O)N3C[C@H]2C#N)nn1 ; COCCN(C(=O)n1cc(C(F)F)nn1)C1C2CCC1C1COc3c(cc(CC(F)F)c(-c4c(O)cccc4F)c3Cl)C(=O)N1C2 ; Cc1ccc2[nH]ncc2c1-c1c(F)cc2c(c1Cl)OC[C@H]1CCC3(CC(CC#N)N3C(=O)n3cnnc3C#N)C(O)CN1C2 ; Cc1ccc2[nH]ncc2c1-c1c(C#CCN(C)C)cc2c(c1Cl)OCC1N(C2)C(=O)CC12CCCN2C(=O)n1ccnc1]

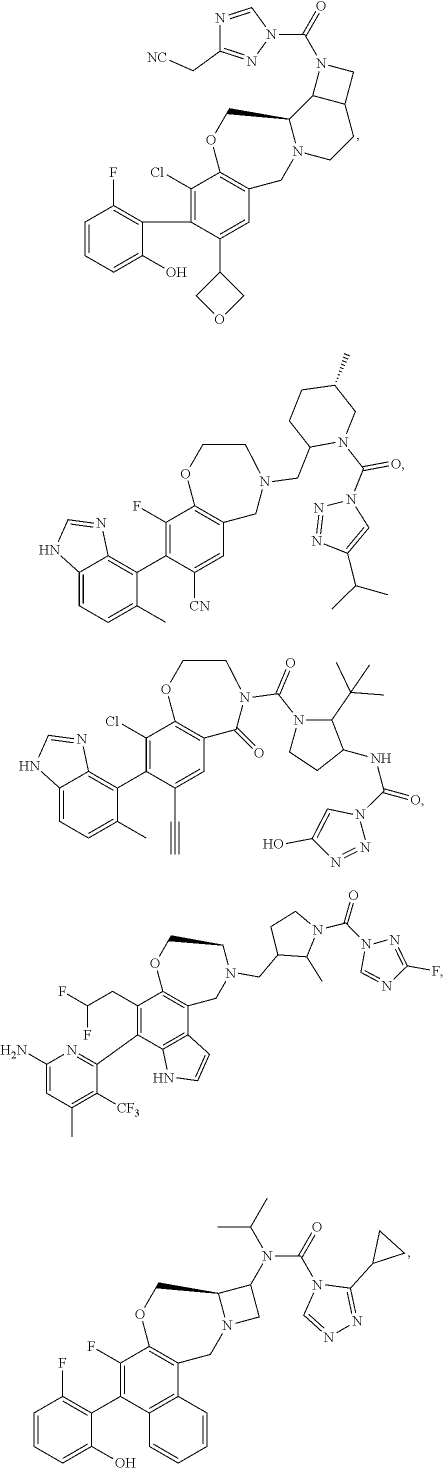

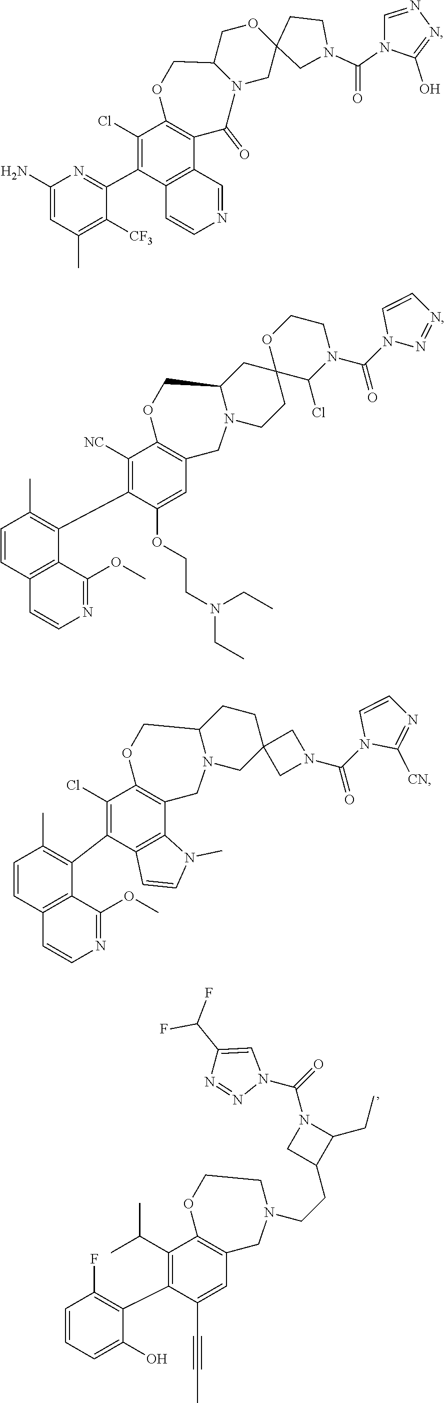 [SMILES: Cc1cc(N)nc(-c2c(Cl)c3c(c4cnccc24)C(=O)N2CC4(CCN(C(=O)n5cnnc5O)C4)OCC2CO3)c1C(F)(F)F ; CCN(CC)CCOc1cc2c(c(C#N)c1-c1c(C)ccc3ccnc(OC)c13)OC[C@H]1CC3(CCN1C2)OCCN(C(=O)n1ccnn1)C3Cl ; COc1nccc2ccc(C)c(-c3c(Cl)c4c(c5c3ccn5C)CN3CC5(CCC3CO4)CN(C(=O)n3ccnc3C#N)C5)c12 ; CC#Cc1cc2c(c(C(C)C)c1-c1c(O)cccc1F)OCCN(CCC1CN(C(=O)n3cc(C(F)F)nn3)C1CC)C2]

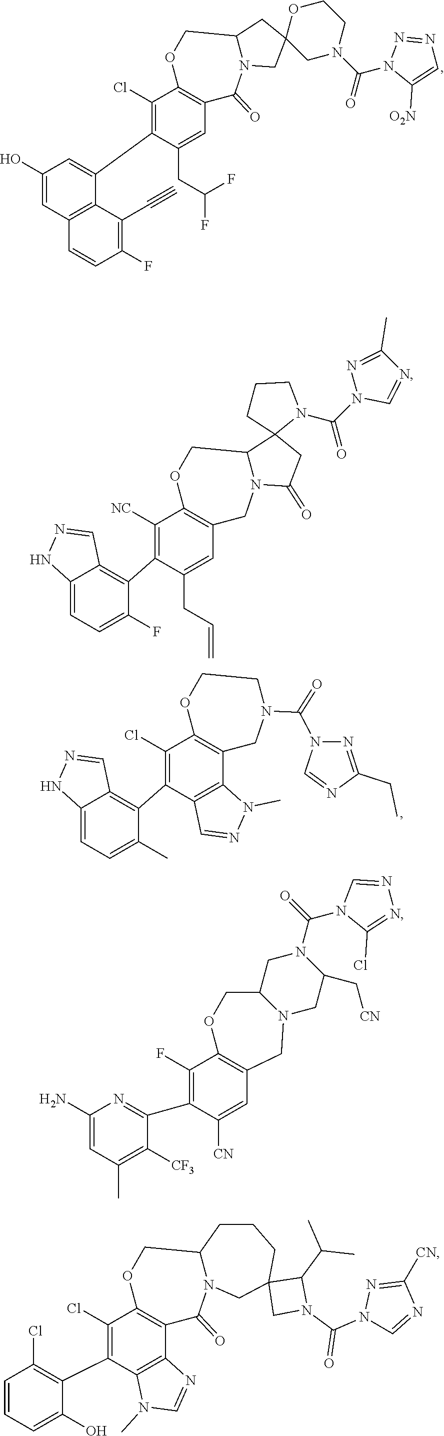

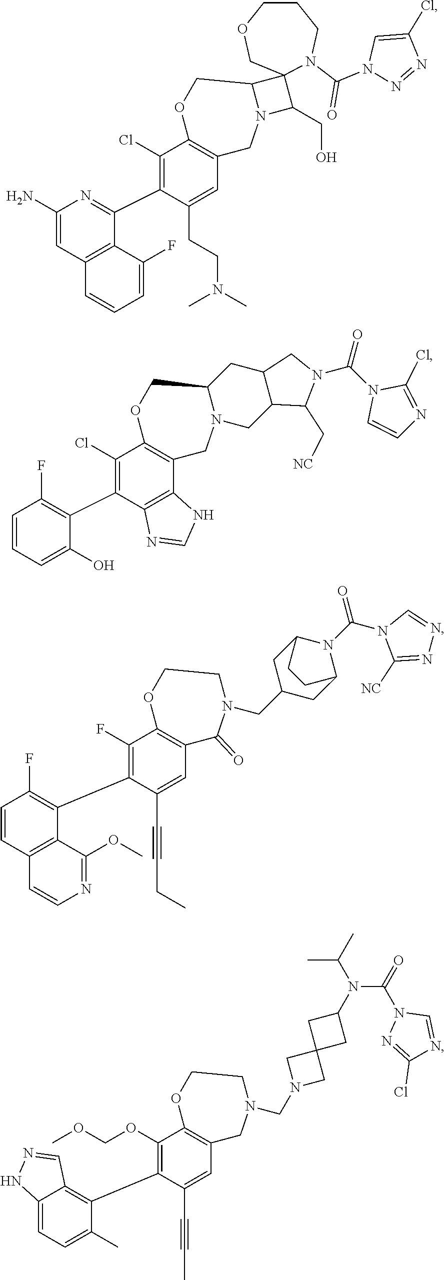 [SMILES: CN(C)CCc1cc2c(c(Cl)c1-c1nc(N)cc3cccc(F)c13)OCC1N(C2)C(CO)C12COCCCN2C(=O)n1cc(Cl)nn1 ; N#CCC1C2CN3Cc4c(c(Cl)c(-c5c(O)cccc5F)c5nc[nH]c45)OC[C@H]3CC2CN1C(=O)n1ccnc1Cl ; CCC#Cc1cc2c(c(F)c1-c1c(F)ccc3ccnc(OC)c13)OCCN(CC1CC3CCC(C1)N3C(=O)n1cnnc1C#N)C2=O ; CC#Cc1cc2c(c(OCOC)c1-c1c(C)ccc3[nH]ncc13)OCCN(CN1CC3(CC(N(C(=O)n4cnc(Cl)n4)C(C)C)C3)C1)C2]

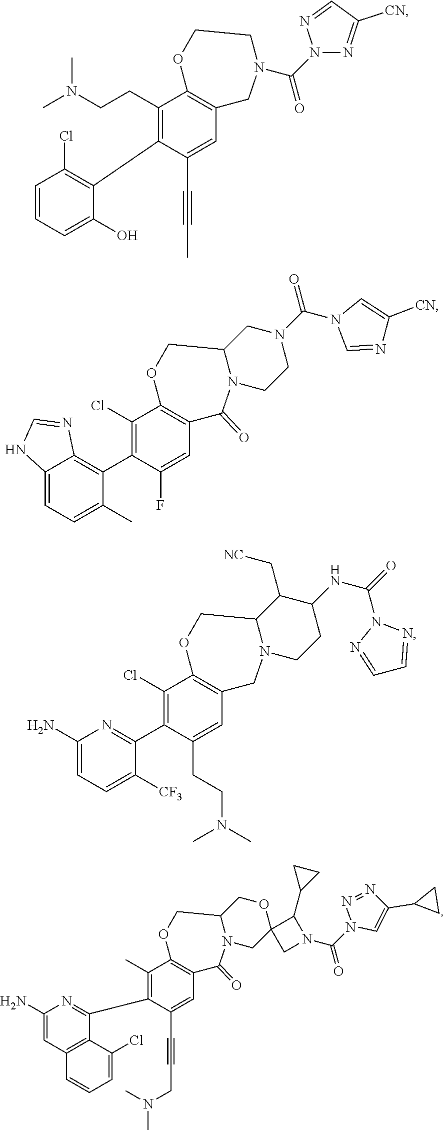 [SMILES: CC#Cc1cc2c(c(CCN(C)C)c1-c1c(O)cccc1Cl)OCCN(C(=O)n1ncc(C#N)n1)C2 ; Cc1ccc2[nH]cnc2c1-c1c(F)cc2c(c1Cl)OCC1CN(C(=O)n3cnc(C#N)c3)CCN1C2=O ; CN(C)CCc1cc2c(c(Cl)c1-c1nc(N)ccc1C(F)(F)F)OCC1C(CC#N)C(NC(=O)n3nccn3)CCN1C2 ; Cc1c2c(cc(C#CCN(C)C)c1-c1nc(N)cc3cccc(Cl)c13)C(=O)N1CC3(CN(C(=O)n4cc(C5CC5)nn4)C3C3CC3)OCC1CO2]

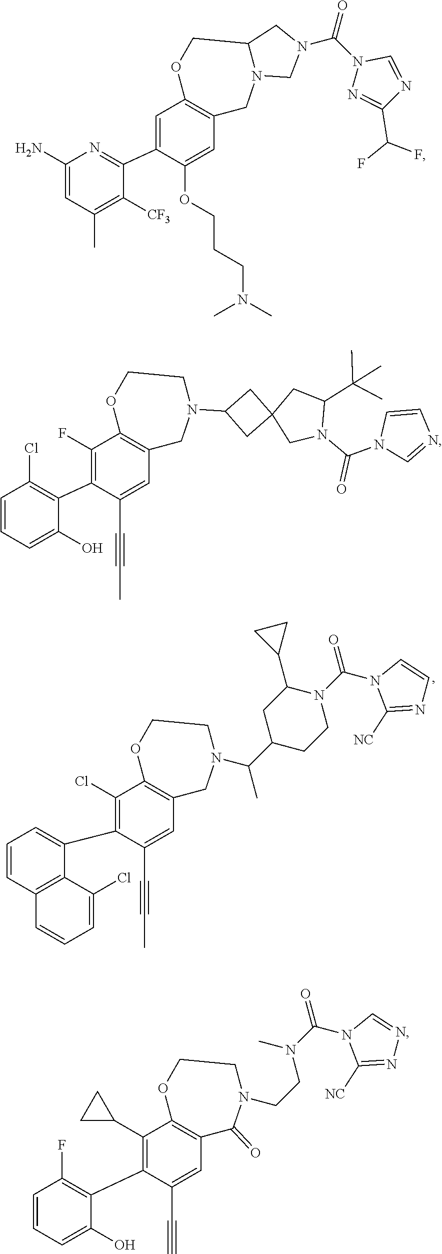 [SMILES: Cc1cc(N)nc(-c2cc3c(cc2OCCCN(C)C)CN2CN(C(=O)n4cnc(C(F)F)n4)CC2CO3)c1C(F)(F)F ; CC#Cc1cc2c(c(F)c1-c1c(O)cccc1Cl)OCCN(C1CC3(C1)CC(C(C)(C)C)N(C(=O)n1ccnc1)C3)C2 ; CC#Cc1cc2c(c(Cl)c1-c1cccc3cccc(Cl)c13)OCCN(C(C)C1CCN(C(=O)n3ccnc3C#N)C(C3CC3)C1)C2 ; C#Cc1cc2c(c(C3CC3)c1-c1c(O)cccc1F)OCCN(CCN(C)C(=O)n1cnnc1C#N)C2=O]

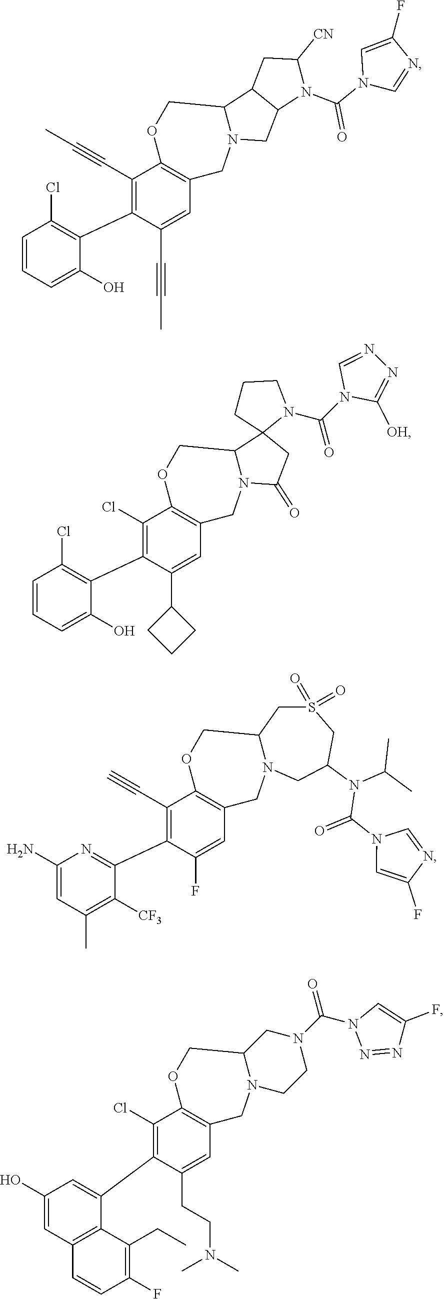 [SMILES: CC#Cc1cc2c(c(C#CC)c1-c1c(O)cccc1Cl)OCC1C3CC(C#N)N(C(=O)n4cnc(F)c4)C3CN1C2 ; O=C1CC2(CCCN2C(=O)n2cnnc2O)C2COc3c(cc(C4CCC4)c(-c4c(O)cccc4Cl)c3Cl)CN12 ; C#Cc1c2c(cc(F)c1-c1nc(N)cc(C)c1C(F)(F)F)CN1CC(N(C(=O)n3cnc(F)c3)C(C)C)CS(=O)(=O)CC1CO2 ; CCc1c(F)ccc2cc(O)cc(-c3c(CCN(C)C)cc4c(c3Cl)OCC3CN(C(=O)n5cc(F)nn5)CCN3C4)c12]

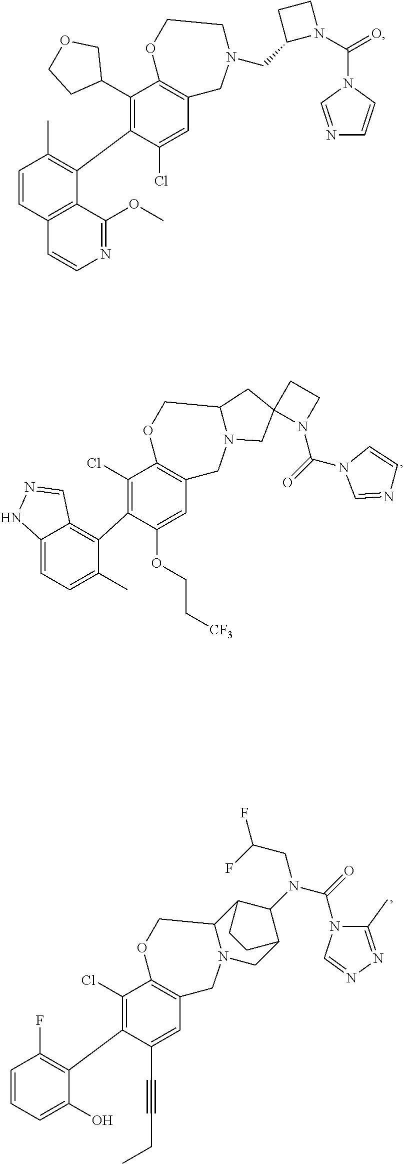 [SMILES: COc1nccc2ccc(C)c(-c3c(Cl)cc4c(c3C3CCOC3)OCCN(C[C@@H]3CCN3C(=O)n3ccnc3)C4)c12 ; Cc1ccc2[nH]ncc2c1-c1c(OCCC(F)(F)F)cc2c(c1Cl)OCC1CC3(CCN3C(=O)n3ccnc3)CN1C2 ; CCC#Cc1cc2c(c(Cl)c1-c1c(O)cccc1F)OCC1C3CCC(CN1C2)C3N(CC(F)F)C(=O)n1cnnc1C]

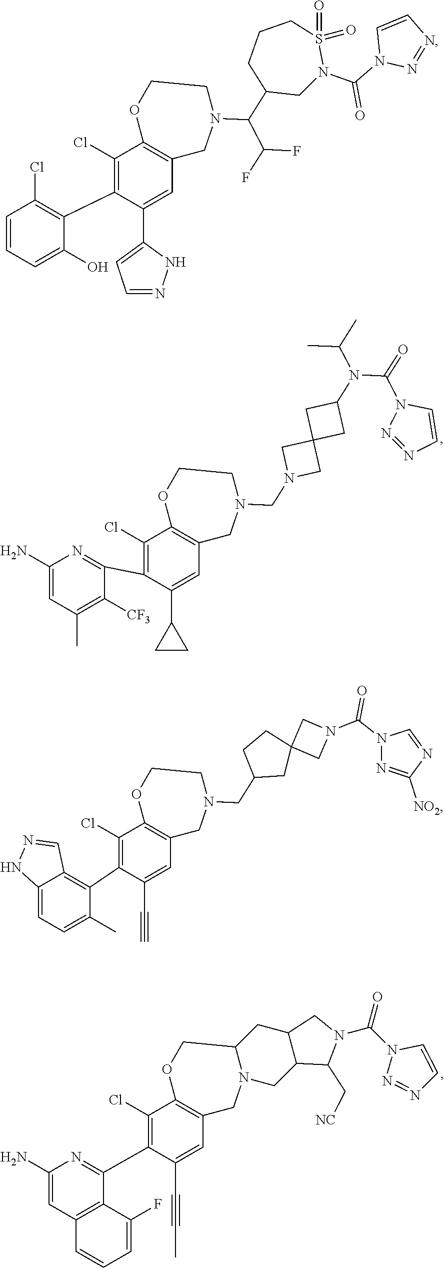 [SMILES: O=C(N1CC(C(C(F)F)N2CCOc3c(cc(-c4ccn[nH]4)c(-c4c(O)cccc4Cl)c3Cl)C2)CCCS1(=O)=O)n1ccnn1 ; Cc1cc(N)nc(-c2c(C3CC3)cc3c(c2Cl)OCCN(CN2CC4(CC(N(C(=O)n5ccnn5)C(C)C)C4)C2)C3)c1C(F)(F)F ; C#Cc1cc2c(c(Cl)c1-c1c(C)ccc3[nH]ncc13)OCCN(CC1CCC3(C1)CN(C(=O)n1cnc([N+](=O)[O-])n1)C3)C2 ; CC#Cc1cc2c(c(Cl)c1-c1nc(N)cc3cccc(F)c13)OCC1CC3CN(C(=O)n4ccnn4)C(CC#N)C3CN1C2]

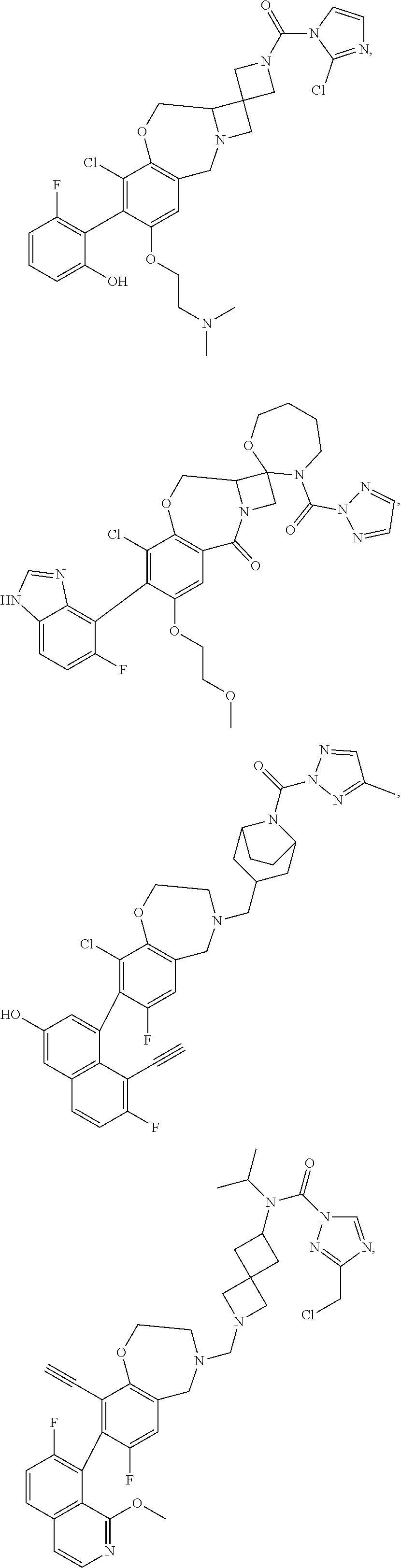 [SMILES: CN(C)CCOc1cc2c(c(Cl)c1-c1c(O)cccc1F)OCC1N(C2)CC12CN(C(=O)n1ccnc1Cl)C2 ; COCCOc1cc2c(c(Cl)c1-c1c(F)ccc3[nH]cnc13)OCC1N(CC13OCCCCN3C(=O)n1nccn1)C2=O ; C#Cc1c(F)ccc2cc(O)cc(-c3c(F)cc4c(c3Cl)OCCN(CC3CC5CCC(C3)N5C(=O)n3ncc(C)n3)C4)c12 ; C#Cc1c2c(cc(F)c1-c1c(F)ccc3ccnc(OC)c13)CN(CN1CC3(CC(N(C(=O)n4cnc(CCl)n4)C(C)C)C3)C1)CCO2]

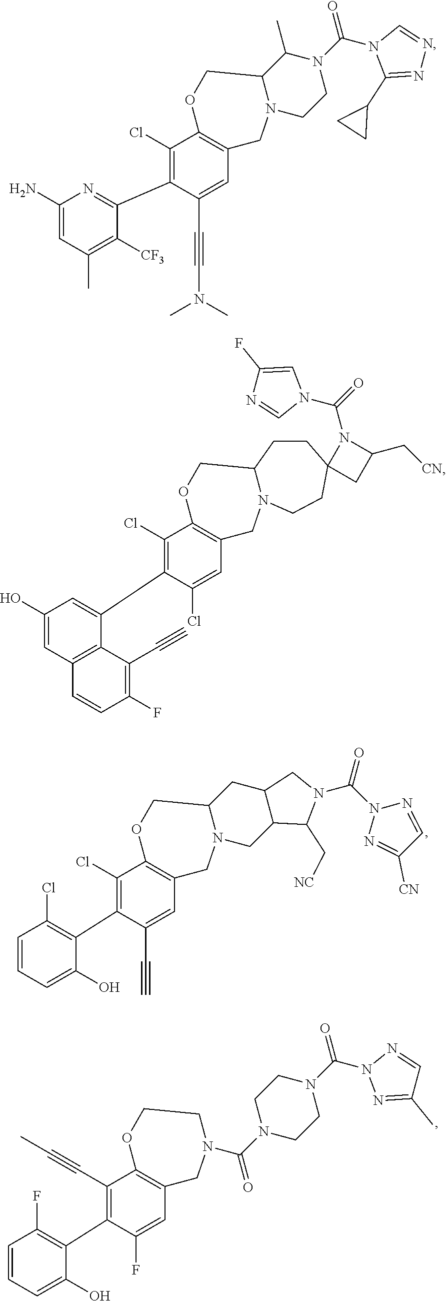

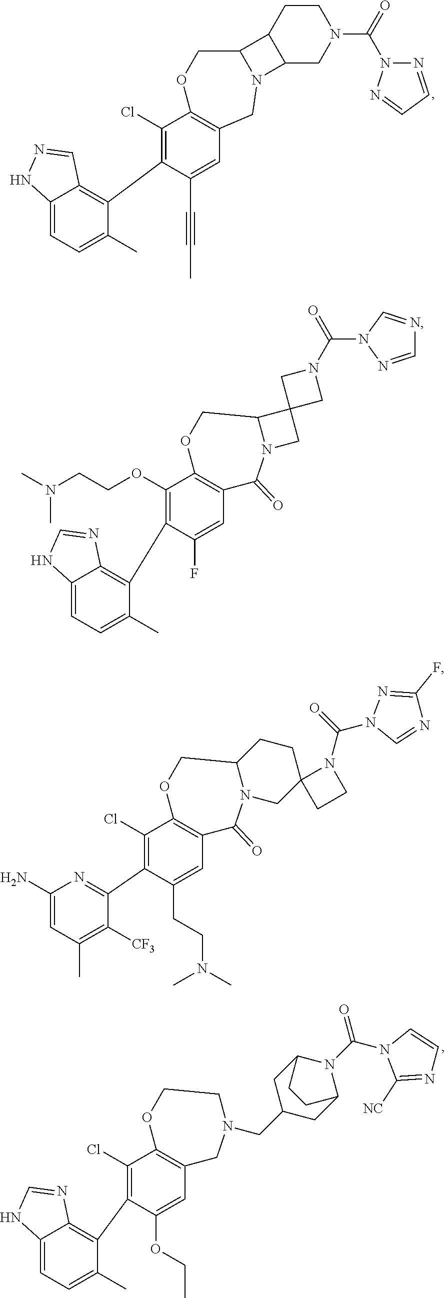 [SMILES: CC#Cc1cc2c(c(Cl)c1-c1c(C)ccc3[nH]ncc13)OCC1C3CCN(C(=O)n4nccn4)CC3N1C2 ; Cc1ccc2[nH]cnc2c1-c1c(F)cc2c(c1OCCN(C)C)OCC1N(CC13CN(C(=O)n1cncn1)C3)C2=O ; Cc1cc(N)nc(-c2c(CCN(C)C)cc3c(c2Cl)OCC2CCC4(CCN4C(=O)n4cnc(F)n4)CN2C3=O)c1C(F)(F)F ; CCOc1cc2c(c(Cl)c1-c1c(C)ccc3[nH]cnc13)OCCN(CC1CC3CCC(C1)N3C(=O)n1ccnc1C#N)C2]

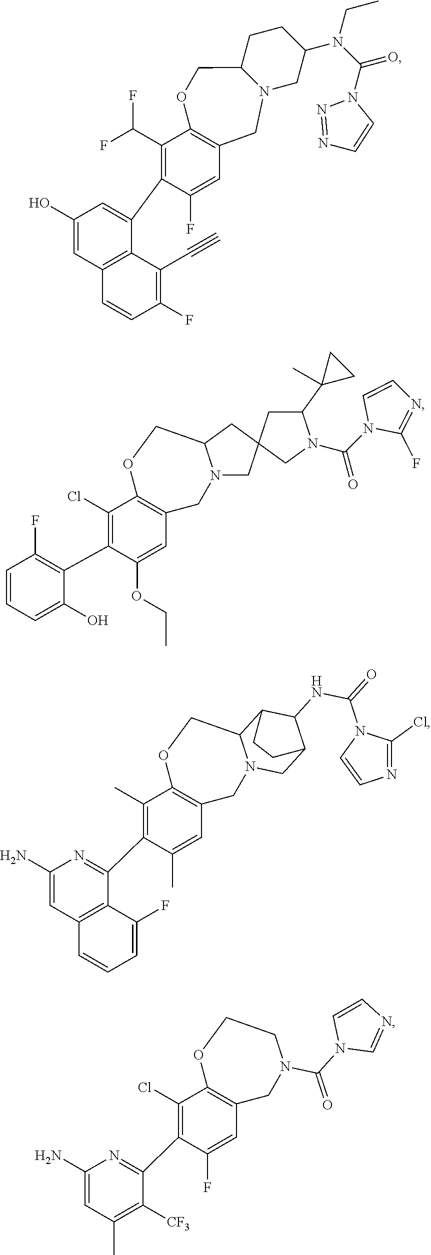 [SMILES: C#Cc1c(F)ccc2cc(O)cc(-c3c(F)cc4c(c3C(F)F)OCC3CCC(N(CC)C(=O)n5ccnn5)CN3C4)c12 ; CCOc1cc2c(c(Cl)c1-c1c(O)cccc1F)OCC1CC3(CC(C4(C)CC4)N(C(=O)n4ccnc4F)C3)CN1C2 ; Cc1cc2c(c(C)c1-c1nc(N)cc3cccc(F)c13)OCC1C3CCC(CN1C2)C3NC(=O)n1ccnc1Cl ; Cc1cc(N)nc(-c2c(F)cc3c(c2Cl)OCCN(C(=O)n2ccnc2)C3)c1C(F)(F)F]

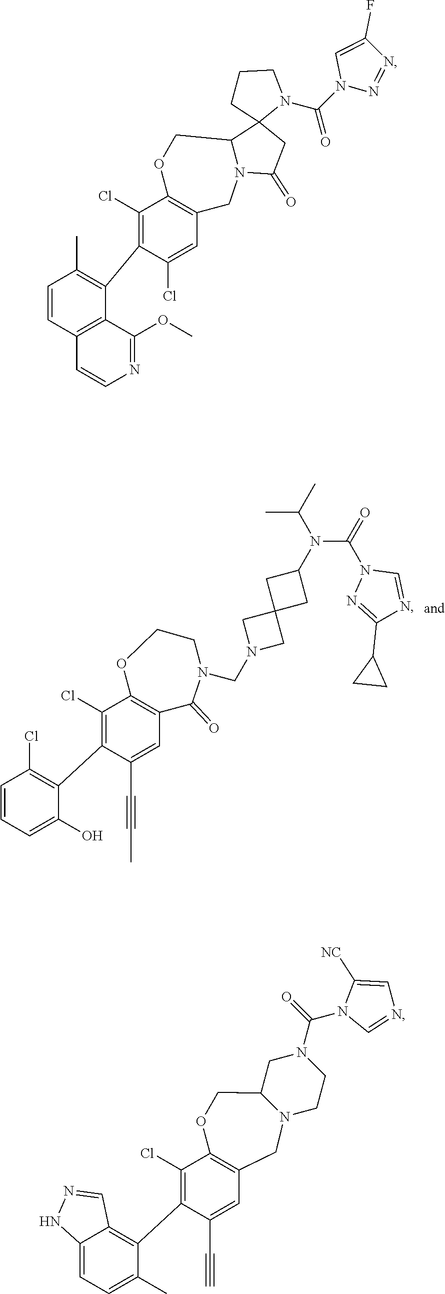 [SMILES: COc1nccc2ccc(C)c(-c3c(Cl)cc4c(c3Cl)OCC3N(C4)C(=O)CC34CCCN4C(=O)n3cc(F)nn3)c12 ; CC#Cc1cc2c(c(Cl)c1-c1c(O)cccc1Cl)OCCN(CN1CC3(CC(N(C(=O)n4cnc(C5CC5)n4)C(C)C)C3)C1)C2=O ; C#Cc1cc2c(c(Cl)c1-c1c(C)ccc3[nH]ncc13)OCC1CN(C(=O)n3cncc3C#N)CCN1C2]

or a pharmaceutically acceptable salt, solvate, stereoisomer, or prodrug thereof.

In an aspect is provided a compound of Formula (XXI):

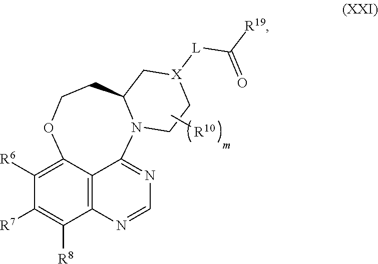

or a pharmaceutically acceptable salt or solvate thereof, wherein:
L is a bond and X is N; or L is —N(R¹²)— and X is CH;
X-L is bound to —C(O)R¹⁹ via a nitrogen atom, thereby forming a urea;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R⁷ is selected from a phenyl and pyridyl; wherein the phenyl and pyridyl are optionally substituted;
R¹⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3;
R⁶ is hydrogen, —CN, halogen, C₁₋₄ alkyl, C₁₋₄ haloalkyl, and C₁₋₄ alkoxyl;
R⁸ is hydrogen, halogen, C₁₋₄ alkyl, C₂₋₄ alkynyl, C₁₋₄ haloalkyl, and C₁₋₄ alkoxyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, the compound of Formula (XXI) is a compound of Formula (XXII):

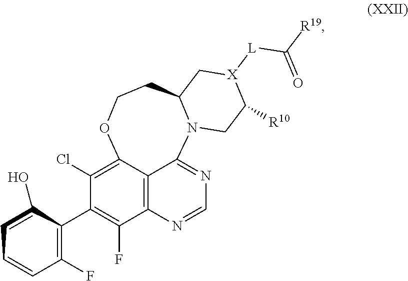

or a pharmaceutically acceptable salt or solvate thereof.

In some embodiments, for a compound of Formula (XXI) or (XXII), X is CH and L is —N(R¹²)—. In some embodiments, X is N and L is a bond.

In some embodiments, for a compound of Formula (XXI) or (XXII), R¹⁰ is independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R¹⁰ is —CH₃.

In some embodiments, for a compound of Formula (XXI) or (XXII), X is selected from CH and N; L is selected from a bond, —N(CH₃)—, and —NH—; and R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted.

In some embodiments, for a compound of Formula (XIX) or (XXI), R⁷ is selected from a phenyl and pyridyl; wherein the phenyl and pyridyl are optionally substituted. In some embodiments, R⁷ is an optionally substituted phenyl. In some embodiments, R⁷ is an optionally substituted pyridyl. In some embodiments, R⁷ is selected from

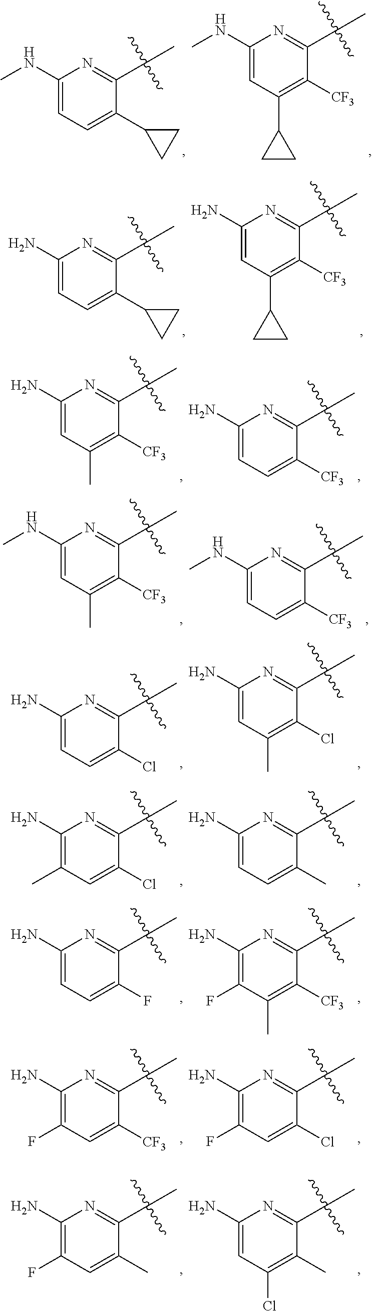

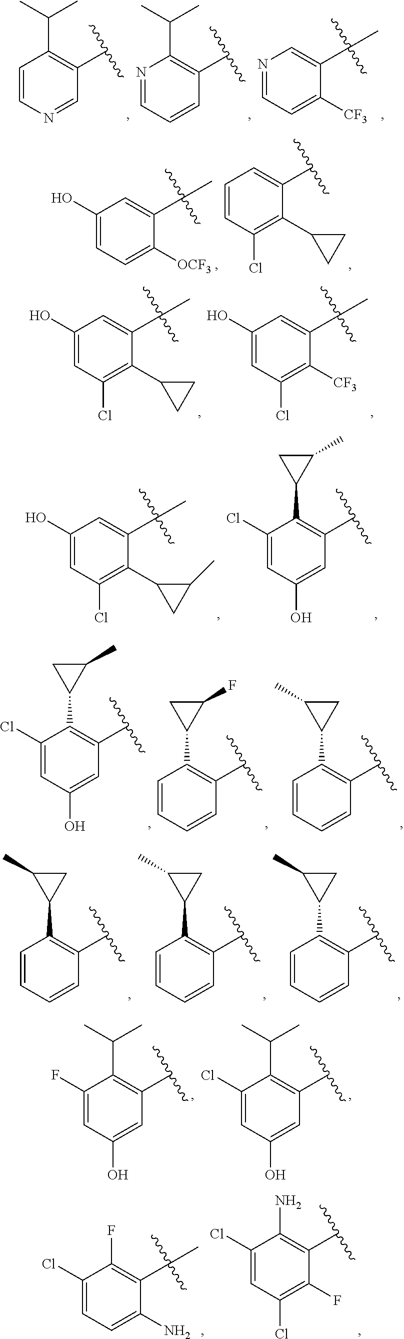

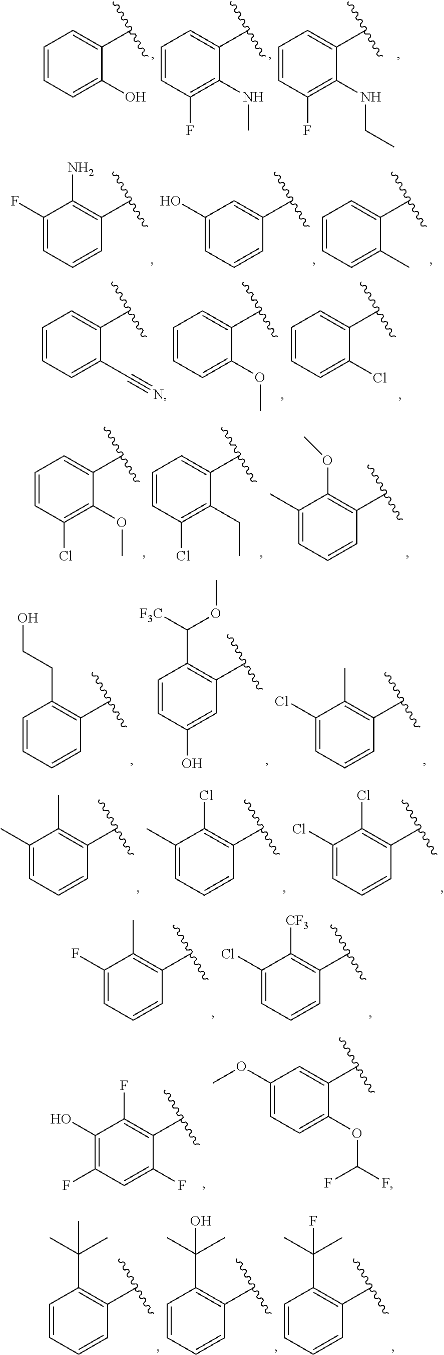

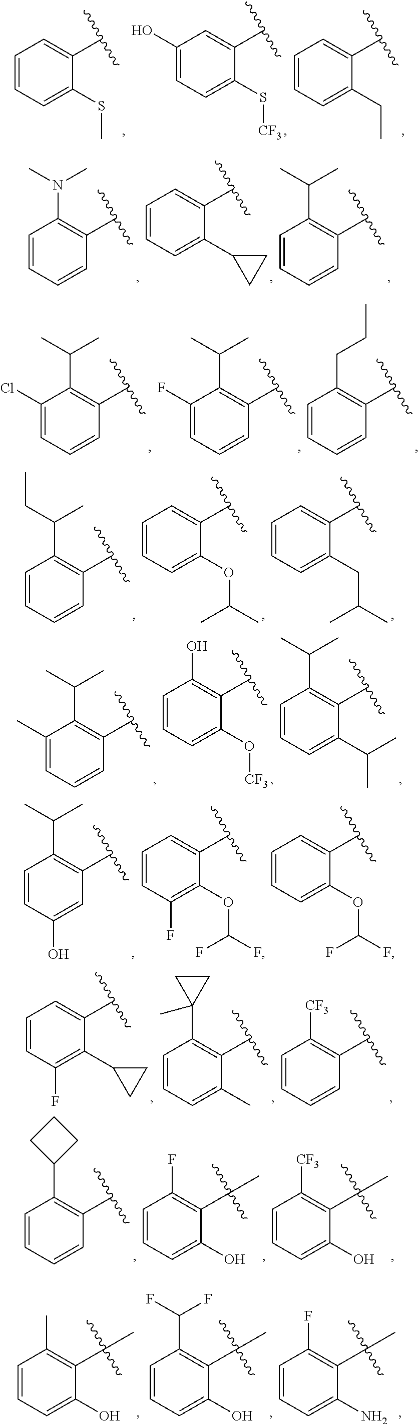

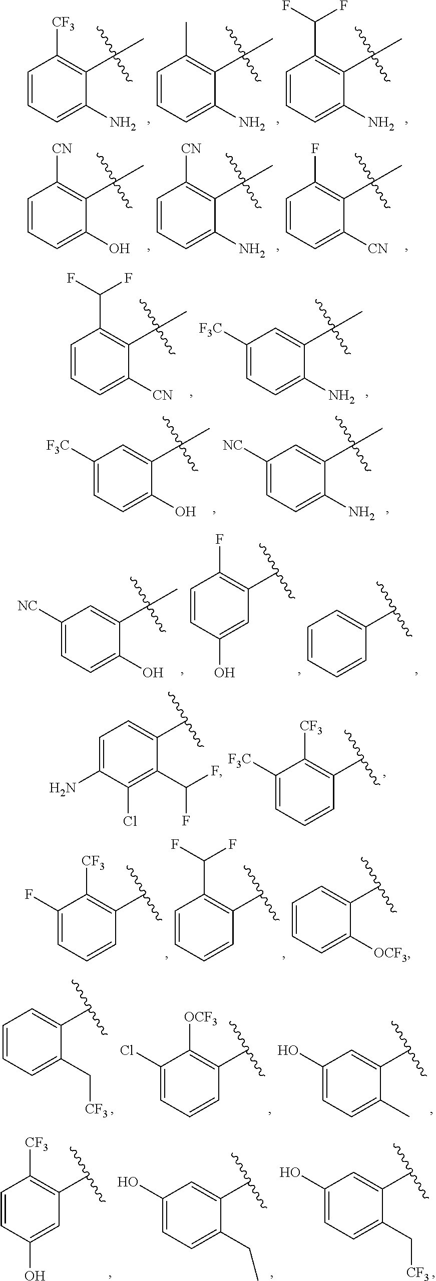

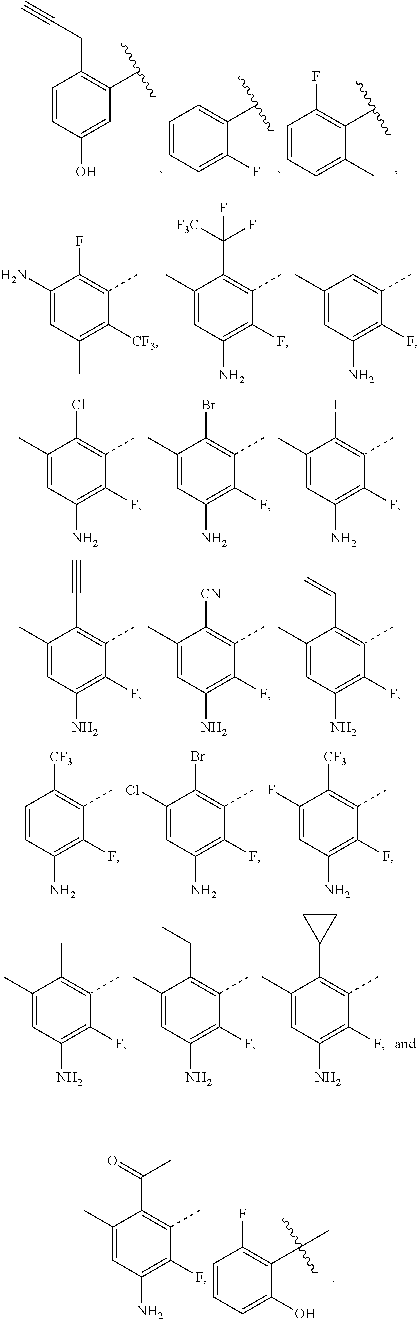

In some embodiments, R⁷ is

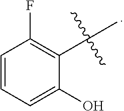 [SMILES: *c1c(O)cccc1F]

In some embodiments, R⁷ is

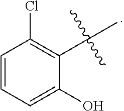 [SMILES: *c1c(O)cccc1Cl]

In certain aspects, the present disclosure provides a compound selected from:

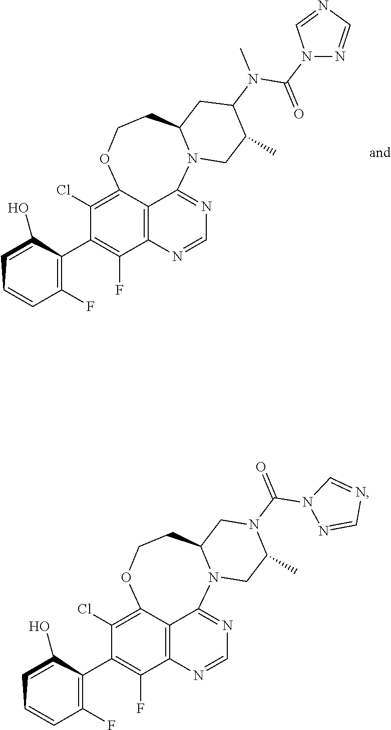

or a pharmaceutically acceptable salt, solvate, stereoisomer, or prodrug thereof.

In certain aspects, the present disclosure provides a compound selected from:

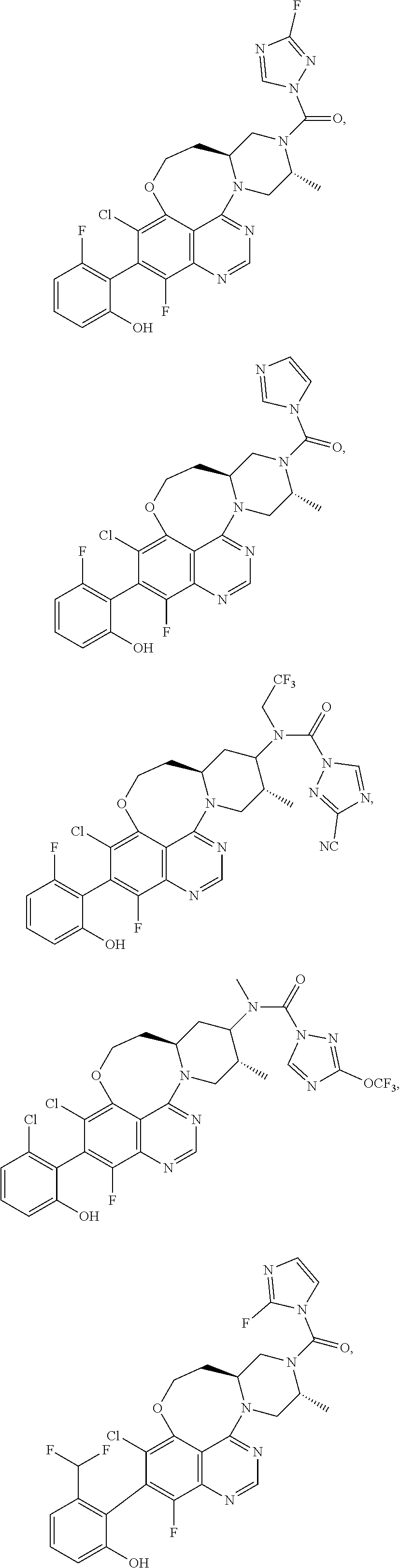 [SMILES: C[C@@H]1CN2c3ncnc4c(F)c(-c5c(O)cccc5F)c(Cl)c(c34)OCC[C@H]2CN1C(=O)n1cnc(F)n1 ; C[C@@H]1CN2c3ncnc4c(F)c(-c5c(O)cccc5F)c(Cl)c(c34)OCC[C@H]2CN1C(=O)n1ccnc1 ; C[C@@H]1CN2c3ncnc4c(F)c(-c5c(O)cccc5F)c(Cl)c(c34)OCC[C@H]2CC1N(CC(F)(F)F)C(=O)n1cnc(C#N)n1 ; C[C@@H]1CN2c3ncnc4c(F)c(-c5c(O)cccc5Cl)c(Cl)c(c34)OCC[C@H]2CC1N(C)C(=O)n1cnc(OC(F)(F)F)n1 ; C[C@@H]1CN2c3ncnc4c(F)c(-c5c(O)cccc5C(F)F)c(Cl)c(c34)OCC[C@H]2CN1C(=O)n1ccnc1F]

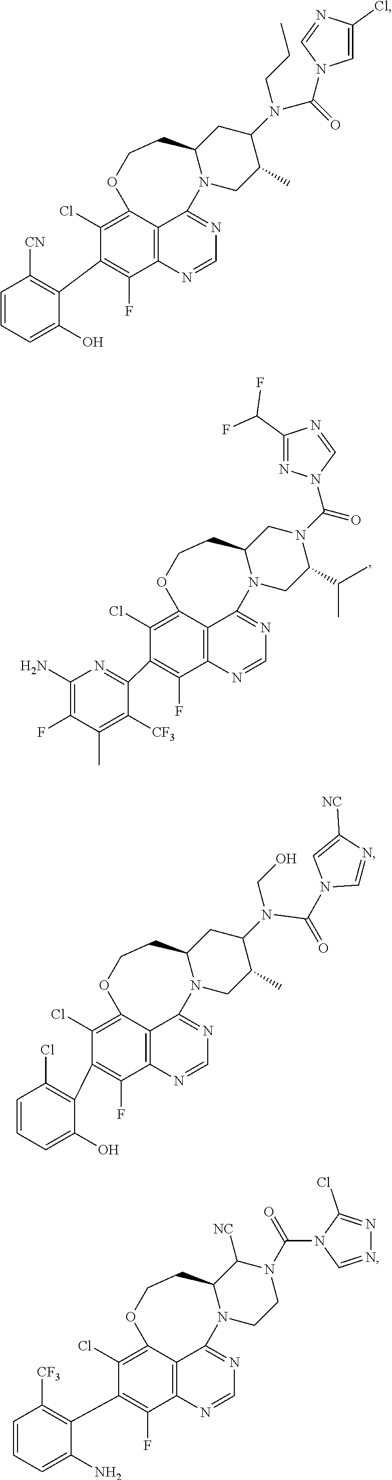 [SMILES: CCCN(C(=O)n1cnc(Cl)c1)C1C[C@@H]2CCOc3c(Cl)c(-c4c(O)cccc4C#N)c(F)c4ncnc(c34)N2C[C@H]1C ; Cc1c(F)c(N)nc(-c2c(Cl)c3c4c(ncnc4c2F)N2C[C@@H](C(C)C)N(C(=O)n4cnc(C(F)F)n4)C[C@@H]2CCO3)c1C(F)(F)F ; C[C@@H]1CN2c3ncnc4c(F)c(-c5c(O)cccc5Cl)c(Cl)c(c34)OCC[C@H]2CC1N(CO)C(=O)n1cnc(C#N)c1 ; N#CC1[C@@H]2CCOc3c(Cl)c(-c4c(N)cccc4C(F)(F)F)c(F)c4ncnc(c34)N2CCN1C(=O)n1cnnc1Cl]

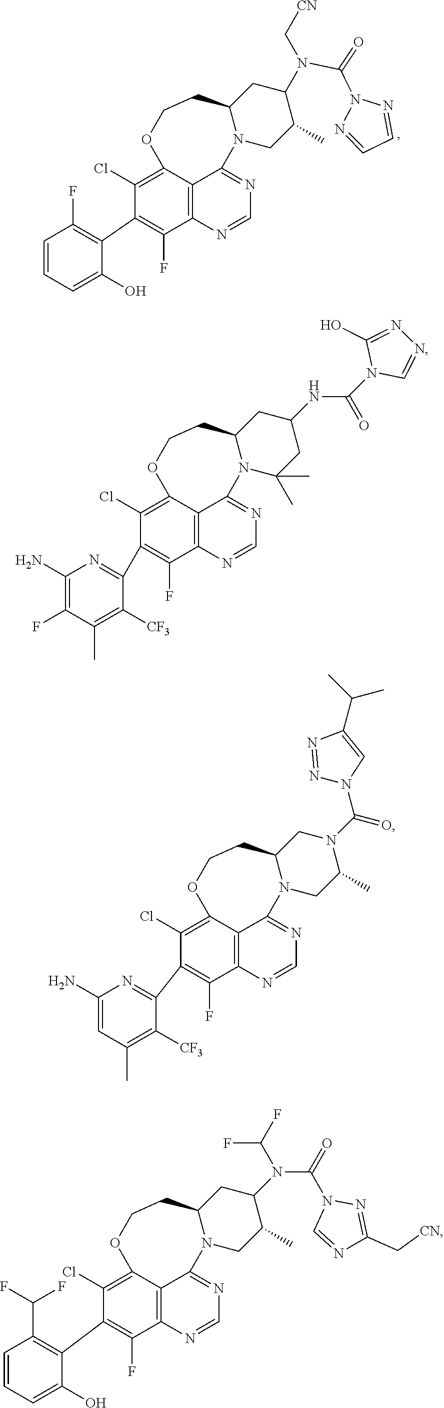 [SMILES: C[C@@H]1CN2c3ncnc4c(F)c(-c5c(O)cccc5F)c(Cl)c(c34)OCC[C@H]2CC1N(CC#N)C(=O)n1nccn1 ; Cc1c(F)c(N)nc(-c2c(Cl)c3c4c(ncnc4c2F)N2[C@@H](CCO3)CC(NC(=O)n3cnnc3O)CC2(C)C)c1C(F)(F)F ; Cc1cc(N)nc(-c2c(Cl)c3c4c(ncnc4c2F)N2C[C@@H](C)N(C(=O)n4cc(C(C)C)nn4)C[C@@H]2CCO3)c1C(F)(F)F ; C[C@@H]1CN2c3ncnc4c(F)c(-c5c(O)cccc5C(F)F)c(Cl)c(c34)OCC[C@H]2CC1N(C(=O)n1cnc(CC#N)n1)C(F)F]

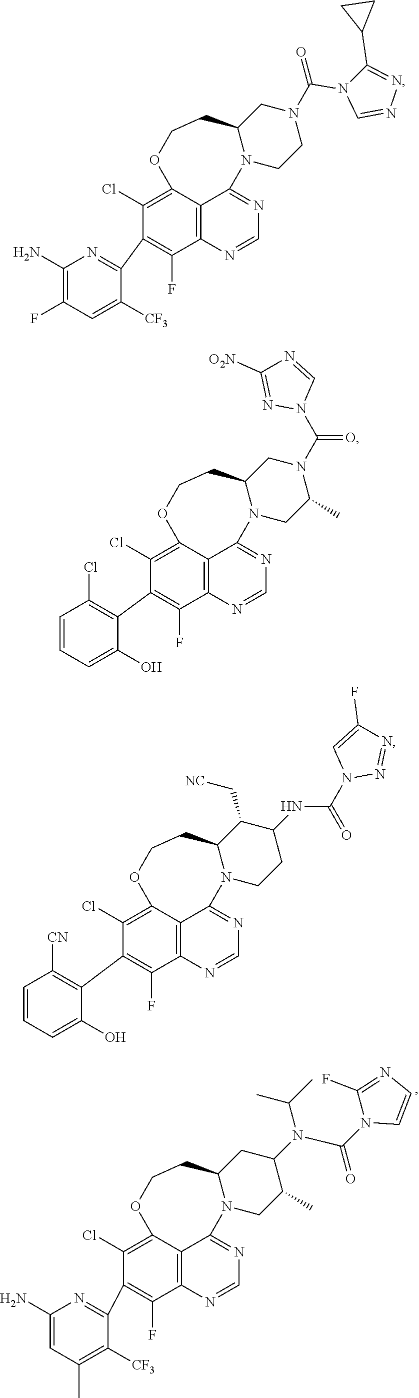 [SMILES: Nc1nc(-c2c(Cl)c3c4c(ncnc4c2F)N2CCN(C(=O)n4cnnc4C4CC4)C[C@@H]2CCO3)c(C(F)(F)F)cc1F ; C[C@@H]1CN2c3ncnc4c(F)c(-c5c(O)cccc5Cl)c(Cl)c(c34)OCC[C@H]2CN1C(=O)n1cnc([N+](=O)[O-])n1 ; N#CC[C@@H]1C(NC(=O)n2cc(F)nn2)CCN2c3ncnc4c(F)c(-c5c(O)cccc5C#N)c(Cl)c(c34)OCC[C@@H]12 ; Cc1cc(N)nc(-c2c(Cl)c3c4c(ncnc4c2F)N2C[C@@H](C)C(N(C(=O)n4ccnc4F)C(C)C)C[C@@H]2CCO3)c1C(F)(F)F]

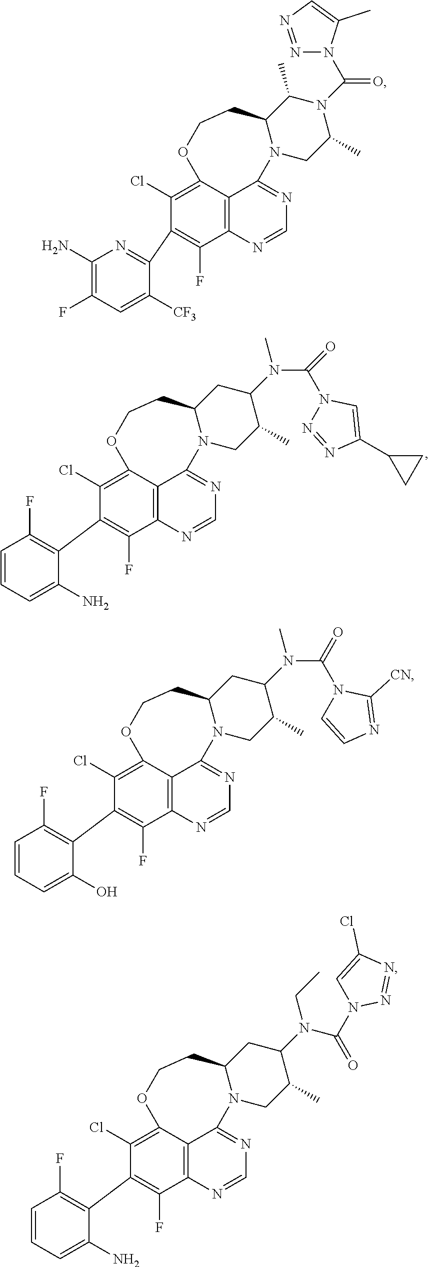 [SMILES: Cc1cnnn1C(=O)N1[C@H](C)CN2c3ncnc4c(F)c(-c5nc(N)c(F)cc5C(F)(F)F)c(Cl)c(c34)OCC[C@H]2[C@@H]1C ; C[C@@H]1CN2c3ncnc4c(F)c(-c5c(N)cccc5F)c(Cl)c(c34)OCC[C@H]2CC1N(C)C(=O)n1cc(C2CC2)nn1 ; C[C@@H]1CN2c3ncnc4c(F)c(-c5c(O)cccc5F)c(Cl)c(c34)OCC[C@H]2CC1N(C)C(=O)n1ccnc1C#N ; CCN(C(=O)n1cc(Cl)nn1)C1C[C@@H]2CCOc3c(Cl)c(-c4c(N)cccc4F)c(F)c4ncnc(c34)N2C[C@H]1C]

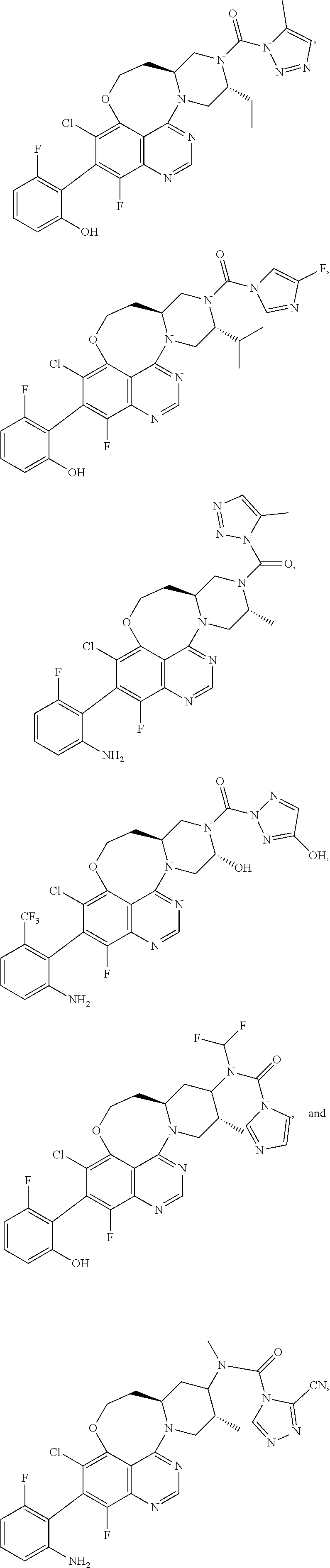 [SMILES: CC[C@@H]1CN2c3ncnc4c(F)c(-c5c(O)cccc5F)c(Cl)c(c34)OCC[C@H]2CN1C(=O)n1nncc1C ; CC(C)[C@@H]1CN2c3ncnc4c(F)c(-c5c(O)cccc5F)c(Cl)c(c34)OCC[C@H]2CN1C(=O)n1cnc(F)c1 ; Cc1cnnn1C(=O)N1C[C@@H]2CCOc3c(Cl)c(-c4c(N)cccc4F)c(F)c4ncnc(c34)N2C[C@H]1C ; Nc1cccc(C(F)(F)F)c1-c1c(Cl)c2c3c(ncnc3c1F)N1C[C@@H](O)N(C(=O)n3ncc(O)n3)C[C@@H]1CCO2 ; C[C@@H]1CN2c3ncnc4c(F)c(-c5c(O)cccc5F)c(Cl)c(c34)OCC[C@H]2CC1N(C(=O)n1ccnc1)C(F)F ; C[C@@H]1CN2c3ncnc4c(F)c(-c5c(N)cccc5F)c(Cl)c(c34)OCC[C@H]2CC1N(C)C(=O)n1cnnc1C#N]

or a pharmaceutically acceptable salt, solvate, stereoisomer, or prodrug thereof.

In general, compounds of the disclosure may be prepared by the following reaction schemes:

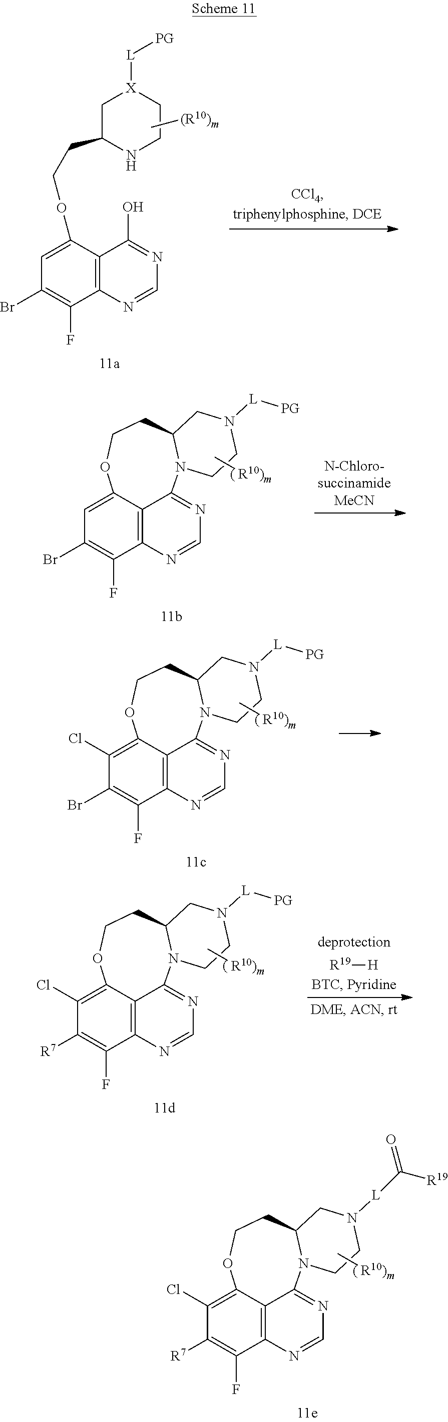

In some embodiments, a compound of Formula 11e may be prepared according to Scheme 11. For example, to a solution of compound 11a is added tetrachloromethane and triphenylphosphine in DCE and the resulting 11b is purified. To a solution of 11b is added N-Chlorosuccinimide in MeCN and the resulting 11c is purified. To a solution of 11c is added RuPhos-Pd-G3, R⁷-boronic acid, potassium carbonate, dicyclohexyl(2′,6′-diisopropoxy-[1,1′-biphenyl]-2-yl)phosphane in 1,4-dioxane/water under nitrogen and the resulting 11d is purified. Following deprotection of 11d, pyridine and a solution of R¹⁹—H in pyridine with BTC is added to the resulting solution under nitrogen. After completion, the mixture is poured into water and is purified to provide a compound of Formula 11e.

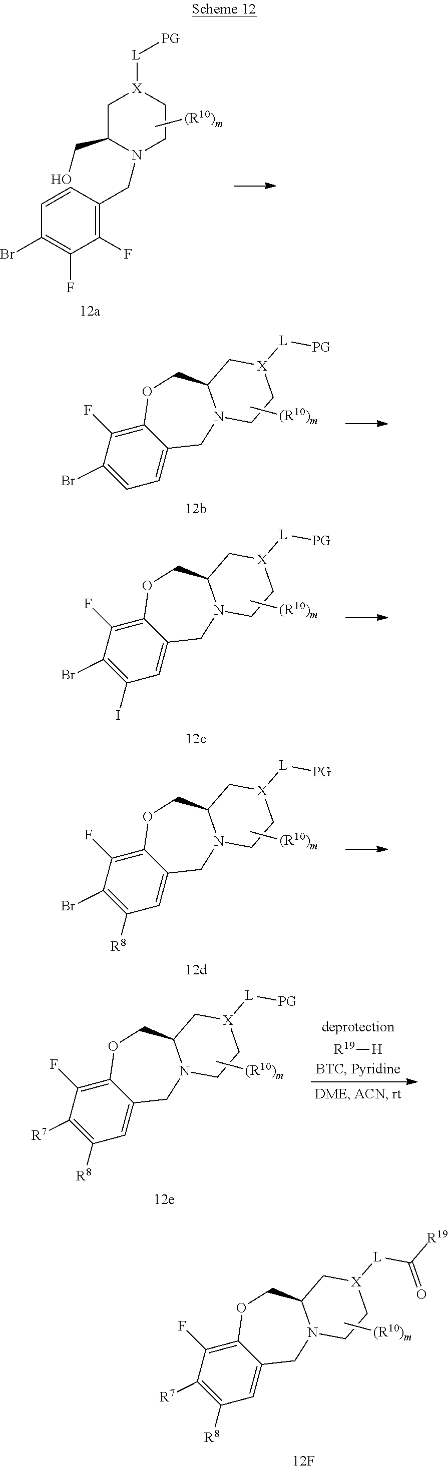

In some embodiments, a compound of Formula 12f may be prepared according to Scheme 12. For example, to a solution of compound 12a is added NaH and DMF and the resulting compound 12b is purified. To a solution of 12b is added N-iodosuccinimide and H₂SO₄, and the resulting compound 12c is purified. To a solution of 12c is added trimethyl(R⁸)silane, Pd(PPh₃)₄, CuI, NEt₃, TBAF in THF, and toluene; and the resulting compound 12d is purified. To a solution of 12d is added R⁷-boronic acid, RuPhos-G3, RuPhos, NaCO₃, dioxane, and the resulting compound 12e is purified. Following deprotection of 12e, pyridine and a solution of R¹⁹—H in pyridine with BTC is added to the resulting solution under nitrogen. After completion, the mixture is poured into water and purified to provide a compound of Formula 12f.

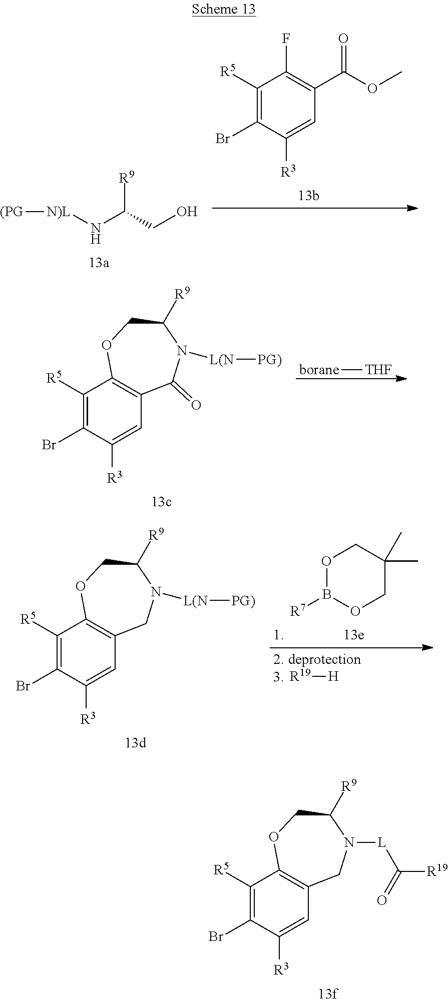

In some embodiments, a compound of Formula 13f may be prepared according to Scheme 13. For example, coupling of amine 13a (wherein L of compound 13a comprises a suitably protected nitrogen atom (e.g., wherein PG is Boc, Cbz, or Fmoc)) with methyl ester 13b and subsequent cyclization can give tricycle 13c using a suitable base, such as cesium carbonate, in solvent (e.g., DMF) with heating. Addition of borane-THF complex in THF to 13c with stirring, followed by quenching in MeOH, can provide compound 13d. Substitution of the aryl bromide can proceed via a Suzuki reaction with 13e. Global deprotection of the resulting compound can be followed by reaction with R¹⁹—H in the presence of triphosgene bis(trichloromethyl) carbonate (BTC) and pyridine to provide a compound of Formula 13f.

Synthetic procedures for certain intermediates may be found in WO 2019/215203, WO 2020/178282, or J. Med. Chem. 2023, 66, 13, 9147-9160, each of which is incorporated herein by reference in its entirety, including any synthetic methods, recitations of compound variables, combination therapies, and methods of use disclosed therein.

### Compounds of Formula (XXIII) and (XXIV)

In certain aspects, the present disclosure provides a compound of Formula (XXIII):

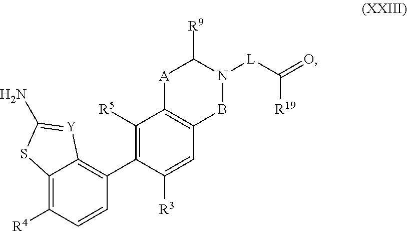

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from —OCH₂—, —N(R⁶)CH₂—, —OCH₂CH₂—, —N(R⁶)CH₂CH₂—, —CH₂OCH₂—, and —CH₂N(R⁶)CH₂—;
B is selected from —CH₂— and —C(O)—;
Y is selected from —C(CN)— and —N—;
R³ and R⁵ are each independently selected from hydrogen, halogen, —C₀₋₃ alkyl-cyclopropyl, —C₁₋₆ alkyl, and —O—C₁₋₆ alkyl, wherein —C₁₋₆ alkyl and —O—C₁₋₆ alkyl are optionally substituted with one, two, or three R¹⁰;
R⁴ is selected from hydrogen, halogen, and —C₁₋₆ alkyl optionally substituted with one, two, or three R¹⁰;
R⁶ is selected from hydrogen and C₁₋₆ alkyl optionally substituted with one, two, or three R¹⁰;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹⁰ is independently selected at each occurrence from halogen, ═O, hydroxy, —C₁₋₄ alkyl, and —O—C₁₋₄ alkyl.
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In certain aspects, the present disclosure provides a compound of Formula (XXIV):

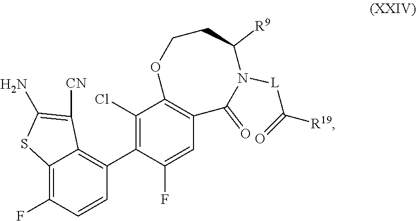

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, the compound of Formula (XXIII) is selected from:

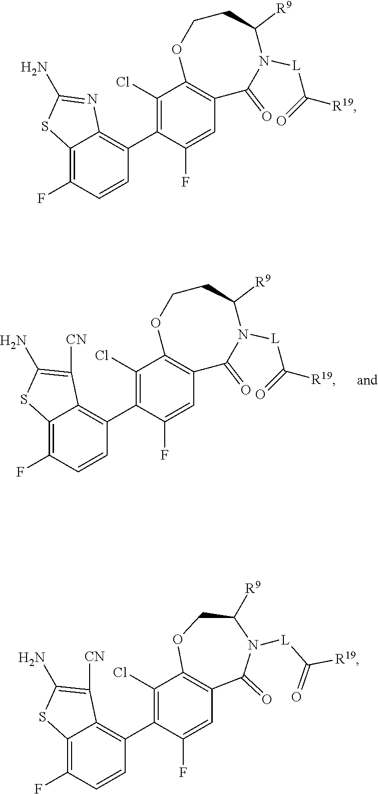

or a pharmaceutically acceptable salt or solvate thereof.

In some embodiments, for a compound of Formula (XXIII) or (XXIV), L is -L¹-L²-L³-, wherein L³ is bound to —C(O)R¹⁹ via a nitrogen atom, thereby forming a urea. In some embodiments, L is -L¹-L²-, wherein L² is bound to —C(O)R¹⁹ via a nitrogen atom, thereby forming a urea. In some embodiments, L is -L²-L³-, wherein L³ is bound to —C(O)R¹⁹ via a nitrogen atom, thereby forming a urea. In some embodiments, L is -L¹-, wherein L¹ is bound to —C(O)R¹⁹ via a nitrogen atom, thereby forming a urea. In some embodiments, L is -L²-, wherein L² is bound to —C(O)R¹⁹ via a nitrogen atom, thereby forming a urea. In some embodiments, L is -L³-, wherein L³ is bound to —C(O)R¹⁹ via a nitrogen atom, thereby forming a urea.

In some embodiments, for a compound of Formula (XXIII) or (XXIV), L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—, wherein C₁₋₆ alkyl and 2- to 6-membered heteroalkyl are optionally substituted. In some embodiments, L¹ is selected from a bond, C₁₋₆ alkyl, 2- to 6-membered heteroalkyl, —C(O)—, —C(O)N(R¹²)—, —S(O)N(R¹²)—, and —S(O)₂N(R¹²)—. In some embodiments, L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted. In some embodiments, L¹ is a bond. In some embodiments, L¹ is 2- to 6-membered heteroalkyl, such as —CH₂NH— or —CH₂N(CH₃)—. In some embodiments, L¹ is selected from a bond and 2- to 6-membered heteroalkyl. In some embodiments, L¹ is selected from a bond and optionally substituted 2- to 6-membered heteroalkyl.

In some embodiments, for a compound of Formula (XXIII) or (XXIV), L² is selected from C₃₋₁₂ carbocycle and 3- to 12-membered heterocycle, each of which is optionally substituted. In some embodiments, L² is a bond. In some embodiments, L² is optionally substituted 3- to 7-membered monocyclic heterocycle, such as optionally substituted 3- to 7-membered monocyclic heterocycloalkyl. In some embodiments, L² is optionally substituted 6- to 12-membered spirocyclic heterocycle, such as optionally substituted 6- to 12-membered spirocyclic heterocycloalkyl. In some embodiments, L² is optionally substituted 7- to 12-membered fused bicyclic heterocycle, such as optionally substituted 7- to 12-membered fused bicyclic heterocycloalkyl.

In some embodiments, for a compound of Formula (XXIII) or (XXIV), L², R⁹, and the atoms to which they are attached form optionally substituted 3- to 12-membered heterocycle. In some embodiments, L², R⁹, and the atoms to which they are attached form optionally substituted 3- to 7-membered monocyclic heterocycle, such as optionally substituted 3- to 7-membered monocyclic heterocycloalkyl. In some embodiments, L², R⁹, and the atoms to which they are attached form optionally substituted 5- to 8-membered monocyclic heterocycle, such as optionally substituted 5- to 8-membered monocyclic heterocycloalkyl. In some embodiments, L², R⁹, and the atoms to which they are attached form optionally substituted 6- to 12-membered spirocyclic heterocycle, such as optionally substituted 6- to 12-membered spirocyclic heterocycloalkyl. In some embodiments, L², R⁹, and the atoms to which they are attached form optionally substituted 7- to 12-membered fused bicyclic heterocycle, such as optionally substituted 7- to 12-membered fused bicyclic heterocycle.

In some embodiments, for a compound of Formula (XXIII) or (XXIV), L³ is selected from a bond, 2- to 6-membered heteroalkyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, nitrene, —C₀₋₆ alkyl-(3- to 12-membered heterocycle), and -(2- to 6-membered heteroalkyl)-(3- to 12-membered heterocycle) are optionally substituted. In some embodiments, L³ is selected from a bond, 2- to 6-membered heteroalkyl, and nitrene, wherein 2- to 6-membered heteroalkyl and nitrene are optionally substituted. In some embodiments, L³ is optionally substituted nitrene, such as —NH—, —N(CH₃)—, —N(CH₂CH₃)—, or —N(CH(CH₃)₂)—. In some embodiments, L³ is 2- to 6-membered heteroalkyl, such as —CH₂NH—, —CH₂N(CH₃)—, —NHCH₂—, or —N(CH₃)CH₂—. In some embodiments, L³ is a bond.

In some embodiments, for a compound of Formula (XXIII) or (XXIV), L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond, C₃₋₁₂ carbocycle and 3- to 12-membered heterocycle, wherein C₃₋₁₂ carbocycle and 3- to 12-membered heterocycle are optionally substituted; and L³ is selected from a bond, 2- to 6-membered heteroalkyl, and nitrene, wherein 2- to 6-membered heteroalkyl and nitrene are optionally substituted. In some embodiments, L¹ is a bond; L² is selected from a bond and optionally substituted 3- to 12-membered heterocycle; and L³ is selected from a bond, 2- to 6-membered heteroalkyl, and nitrene, wherein 2- to 6-membered heteroalkyl and nitrene are optionally substituted. In some embodiments, L¹ is a bond; L² is optionally substituted 3- to 12-membered heterocycle; and L³ is a bond. In some embodiments, L² is optionally substituted 3- to 12-membered heterocycle; and L³ is a bond. In some embodiments, L¹ is a bond; L² is selected from C₃₋₁₂ carbocycle and 3- to 12-membered heterocycle, each of which is optionally substituted; and L³ is optionally substituted nitrene. In some embodiments, L² is selected from C₃₋₁₂ carbocycle and 3- to 12-membered heterocycle, each of which is optionally substituted; and L³ is optionally substituted nitrene. In some embodiments, L² is optionally substituted 6- to 12-membered spirocyclic heterocycle. In some embodiments, L¹ is a bond; L², R⁹, and the atoms to which they are attached form optionally substituted 3- to 12-membered heterocycle; and L³ is selected from a bond and optionally substituted nitrene.

In some embodiments, the compound of Formula (XXIII) is:

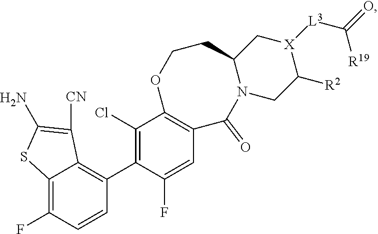

or a pharmaceutically acceptable salt or solvate thereof, wherein:
X is selected from CH and N; and
R² is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted.

In some embodiments, for a compound of Formula (XXIII), R² is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R² is selected from hydrogen, —CH₃, and —CH₂CN. In some embodiments, R² is hydrogen. In some embodiments, X is N and L³ is a bond. In some embodiments, X is CH and L³ is optionally substituted nitrene.

In certain aspects, the present disclosure provides a compound selected from:

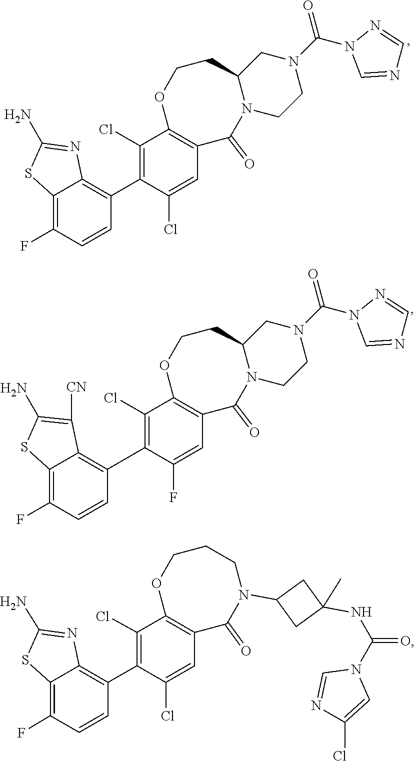 [SMILES: Nc1nc2c(-c3c(Cl)cc4c(c3Cl)OCC[C@H]3CN(C(=O)n5cncn5)CCN3C4=O)ccc(F)c2s1 ; N#Cc1c(N)sc2c(F)ccc(-c3c(F)cc4c(c3Cl)OCC[C@H]3CN(C(=O)n5cncn5)CCN3C4=O)c12 ; CC1(NC(=O)n2cnc(Cl)c2)CC(N2CCCOc3c(cc(Cl)c(-c4ccc(F)c5sc(N)nc45)c3Cl)C2=O)C1]

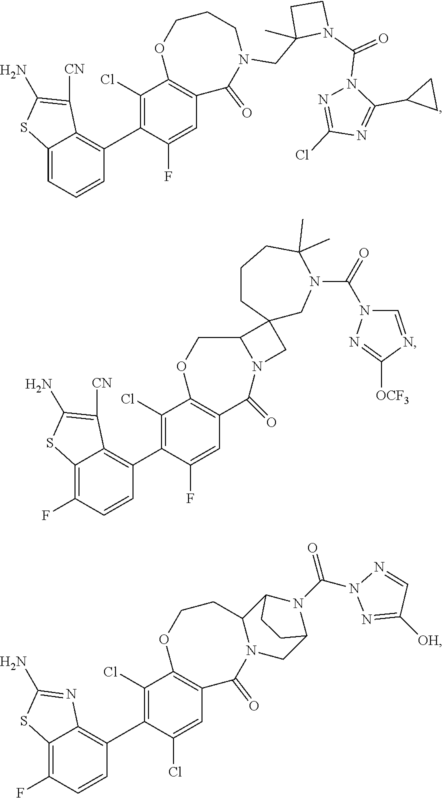 [SMILES: CC1(CN2CCCOc3c(cc(F)c(-c4cccc5sc(N)c(C#N)c45)c3Cl)C2=O)CCN1C(=O)n1nc(Cl)nc1C1CC1 ; CC1(C)CCCC2(CN3C(=O)c4cc(F)c(-c5ccc(F)c6sc(N)c(C#N)c56)c(Cl)c4OCC32)CN1C(=O)n1cnc(OC(F)(F)F)n1 ; Nc1nc2c(-c3c(Cl)cc4c(c3Cl)OCCC3C5CCC(CN3C4=O)N5C(=O)n3ncc(O)n3)ccc(F)c2s1]

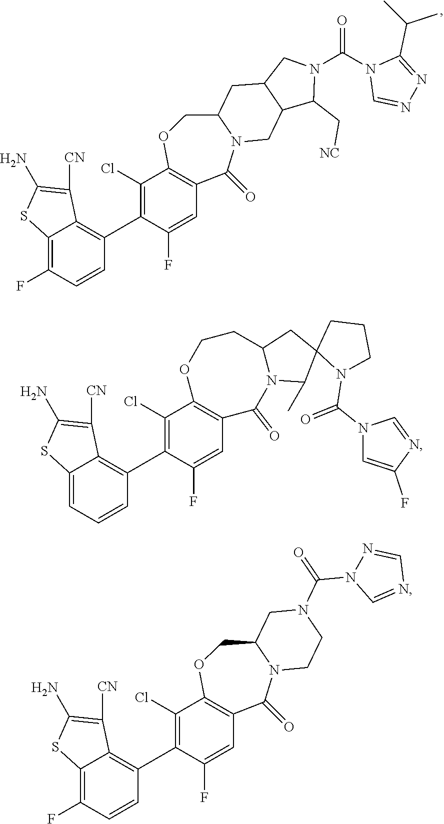 [SMILES: CC(C)c1nncn1C(=O)N1CC2CC3COc4c(cc(F)c(-c5ccc(F)c6sc(N)c(C#N)c56)c4Cl)C(=O)N3CC2C1CC#N ; CC1N2C(=O)c3cc(F)c(-c4cccc5sc(N)c(C#N)c45)c(Cl)c3OCCC2CC12CCCN2C(=O)n1cnc(F)c1 ; N#Cc1c(N)sc2c(F)ccc(-c3c(F)cc4c(c3Cl)OC[C@H]3CN(C(=O)n5cncn5)CCN3C4=O)c12]

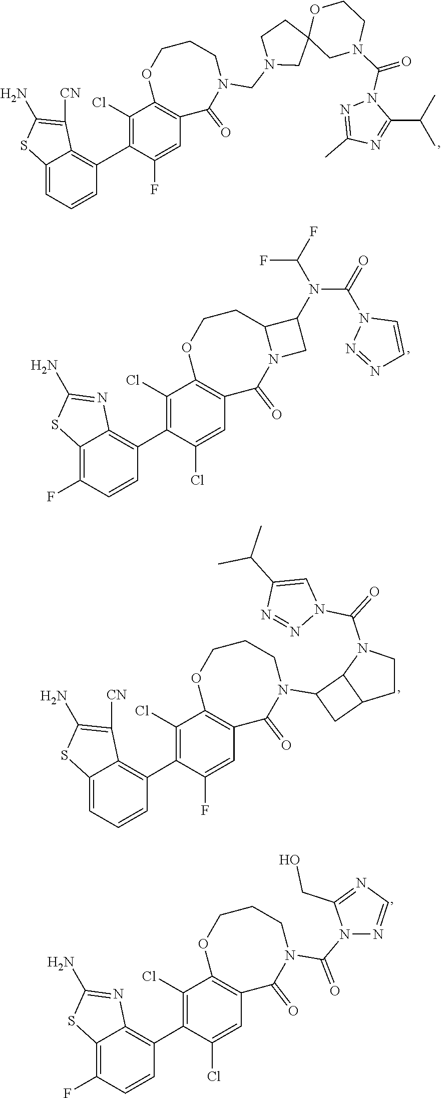 [SMILES: Cc1nc(C(C)C)n(C(=O)N2CCOC3(CCN(CN4CCCOc5c(cc(F)c(-c6cccc7sc(N)c(C#N)c67)c5Cl)C4=O)C3)C2)n1 ; Nc1nc2c(-c3c(Cl)cc4c(c3Cl)OCCC3C(N(C(=O)n5ccnn5)C(F)F)CN3C4=O)ccc(F)c2s1 ; CC(C)c1cn(C(=O)N2CCC3CC(N4CCCOc5c(cc(F)c(-c6cccc7sc(N)c(C#N)c67)c5Cl)C4=O)C32)nn1 ; Nc1nc2c(-c3c(Cl)cc4c(c3Cl)OCCCN(C(=O)n3ncnc3CO)C4=O)ccc(F)c2s1]

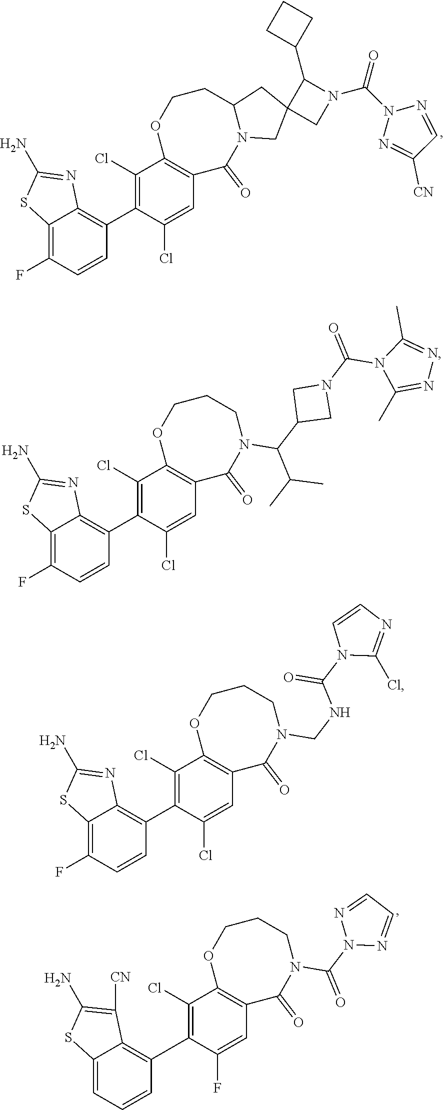 [SMILES: N#Cc1cnn(C(=O)N2CC3(CC4CCOc5c(cc(Cl)c(-c6ccc(F)c7sc(N)nc67)c5Cl)C(=O)N4C3)C2C2CCC2)n1 ; Cc1nnc(C)n1C(=O)N1CC(C(C(C)C)N2CCCOc3c(cc(Cl)c(-c4ccc(F)c5sc(N)nc45)c3Cl)C2=O)C1 ; Nc1nc2c(-c3c(Cl)cc4c(c3Cl)OCCCN(CNC(=O)n3ccnc3Cl)C4=O)ccc(F)c2s1 ; N#Cc1c(N)sc2cccc(-c3c(F)cc4c(c3Cl)OCCCN(C(=O)n3nccn3)C4=O)c12]

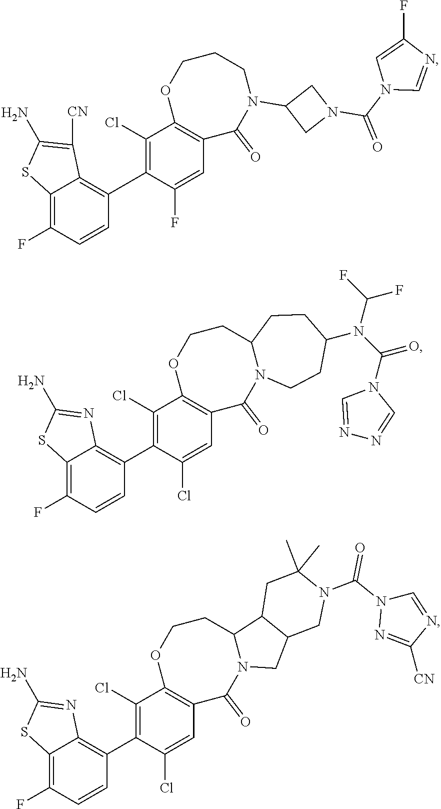 [SMILES: N#Cc1c(N)sc2c(F)ccc(-c3c(F)cc4c(c3Cl)OCCCN(C3CN(C(=O)n5cnc(F)c5)C3)C4=O)c12 ; Nc1nc2c(-c3c(Cl)cc4c(c3Cl)OCCC3CCC(N(C(=O)n5cnnc5)C(F)F)CCN3C4=O)ccc(F)c2s1 ; CC1(C)CC2C(CN3C(=O)c4cc(Cl)c(-c5ccc(F)c6sc(N)nc56)c(Cl)c4OCCC23)CN1C(=O)n1cnc(C#N)n1]

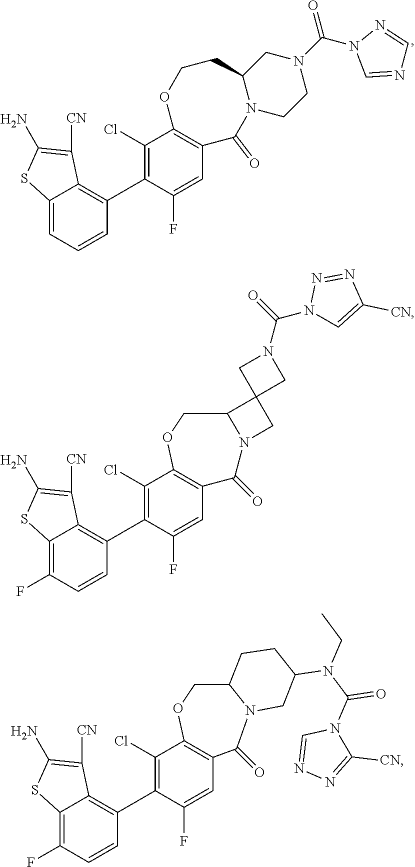 [SMILES: N#Cc1c(N)sc2cccc(-c3c(F)cc4c(c3Cl)OCC[C@H]3CN(C(=O)n5cncn5)CCN3C4=O)c12 ; N#Cc1cn(C(=O)N2CC3(C2)CN2C(=O)c4cc(F)c(-c5ccc(F)c6sc(N)c(C#N)c56)c(Cl)c4OCC23)nn1 ; CCN(C(=O)n1cnnc1C#N)C1CCC2COc3c(cc(F)c(-c4ccc(F)c5sc(N)c(C#N)c45)c3Cl)C(=O)N2C1]

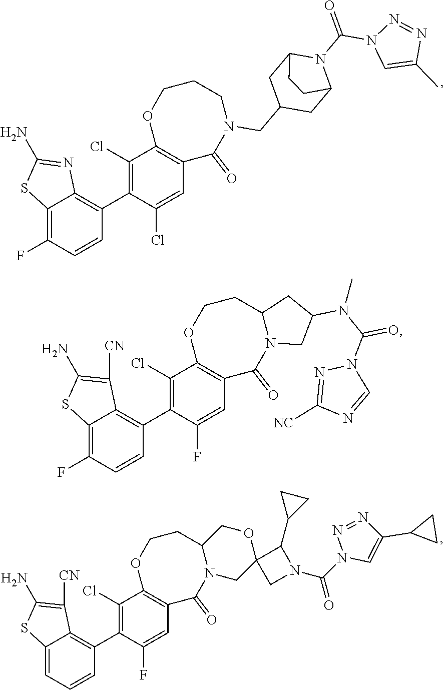 [SMILES: Cc1cn(C(=O)N2C3CCC2CC(CN2CCCOc4c(cc(Cl)c(-c5ccc(F)c6sc(N)nc56)c4Cl)C2=O)C3)nn1 ; CN(C(=O)n1cnc(C#N)n1)C1CC2CCOc3c(cc(F)c(-c4ccc(F)c5sc(N)c(C#N)c45)c3Cl)C(=O)N2C1 ; N#Cc1c(N)sc2cccc(-c3c(F)cc4c(c3Cl)OCCC3COC5(CN3C4=O)CN(C(=O)n3cc(C4CC4)nn3)C5C3CC3)c12]

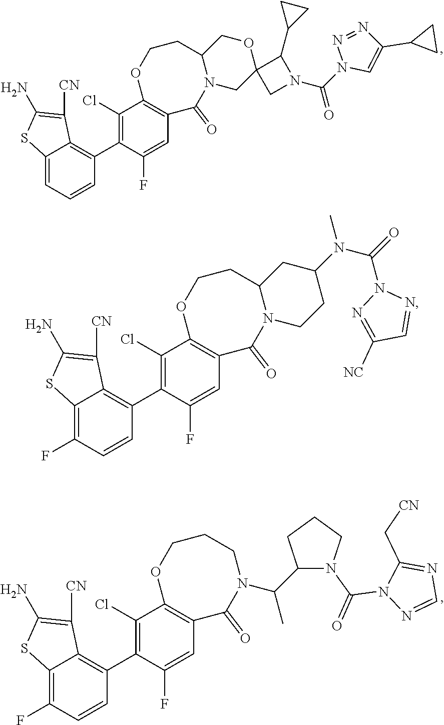 [SMILES: N#Cc1c(N)sc2cccc(-c3c(F)cc4c(c3Cl)OCCC3COC5(CN3C4=O)CN(C(=O)n3cc(C4CC4)nn3)C5C3CC3)c12 ; CN(C(=O)n1ncc(C#N)n1)C1CCN2C(=O)c3cc(F)c(-c4ccc(F)c5sc(N)c(C#N)c45)c(Cl)c3OCCC2C1 ; CC(C1CCCN1C(=O)n1ncnc1CC#N)N1CCCOc2c(cc(F)c(-c3ccc(F)c4sc(N)c(C#N)c34)c2Cl)C1=O]

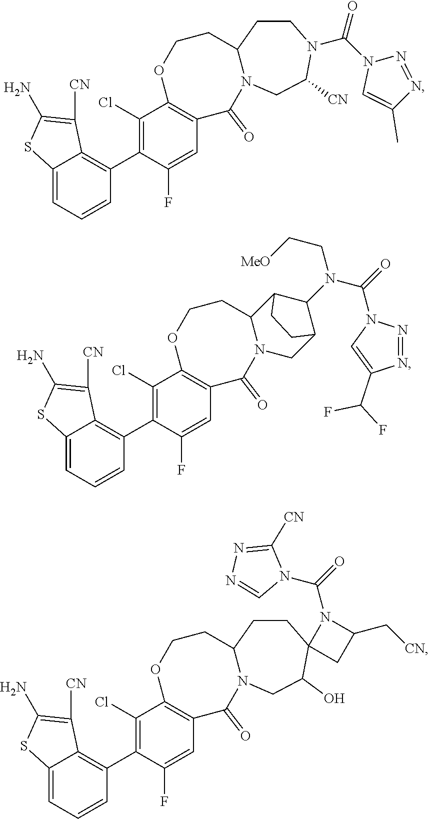 [SMILES: Cc1cn(C(=O)N2CCC3CCOc4c(cc(F)c(-c5cccc6sc(N)c(C#N)c56)c4Cl)C(=O)N3C[C@H]2C#N)nn1 ; COCCN(C(=O)n1cc(C(F)F)nn1)C1C2CCC1C1CCOc3c(cc(F)c(-c4cccc5sc(N)c(C#N)c45)c3Cl)C(=O)N1C2 ; N#CCC1CC2(CCC3CCOc4c(cc(F)c(-c5cccc6sc(N)c(C#N)c56)c4Cl)C(=O)N3CC2O)N1C(=O)n1cnnc1C#N]

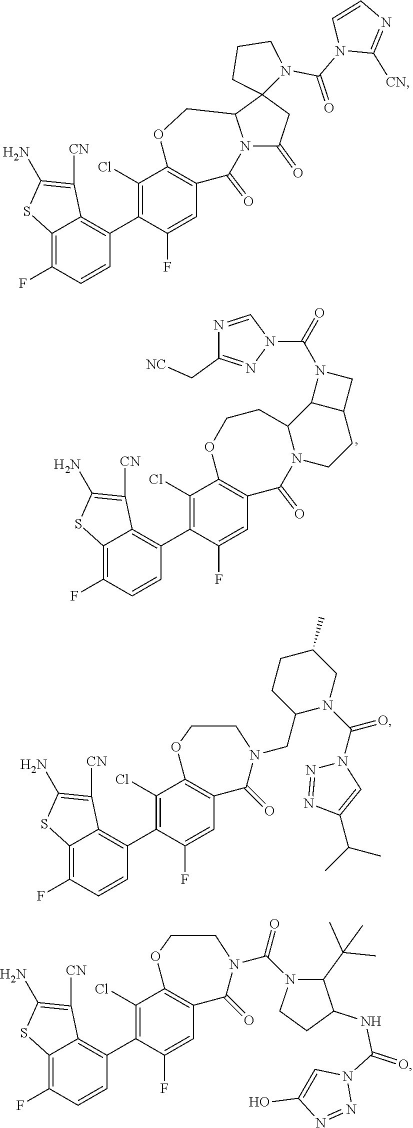 [SMILES: N#Cc1c(N)sc2c(F)ccc(-c3c(F)cc4c(c3Cl)OCC3N(C(=O)CC35CCCN5C(=O)n3ccnc3C#N)C4=O)c12 ; N#CCc1ncn(C(=O)N2CC3CCN4C(=O)c5cc(F)c(-c6ccc(F)c7sc(N)c(C#N)c67)c(Cl)c5OCCC4C32)n1 ; CC(C)c1cn(C(=O)N2C[C@@H](C)CCC2CN2CCOc3c(cc(F)c(-c4ccc(F)c5sc(N)c(C#N)c45)c3Cl)C2=O)nn1 ; CC(C)(C)C1C(NC(=O)n2cc(O)nn2)CCN1C(=O)N1CCOc2c(cc(F)c(-c3ccc(F)c4sc(N)c(C#N)c34)c2Cl)C1=O]

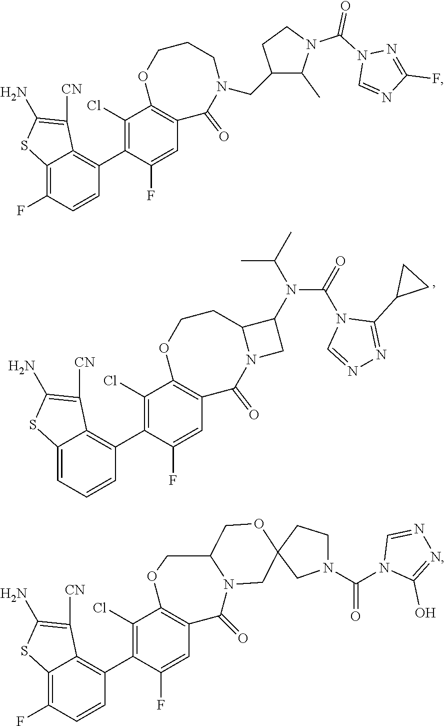 [SMILES: CC1C(CN2CCCOc3c(cc(F)c(-c4ccc(F)c5sc(N)c(C#N)c45)c3Cl)C2=O)CCN1C(=O)n1cnc(F)n1 ; CC(C)N(C(=O)n1cnnc1C1CC1)C1CN2C(=O)c3cc(F)c(-c4cccc5sc(N)c(C#N)c45)c(Cl)c3OCCC12 ; N#Cc1c(N)sc2c(F)ccc(-c3c(F)cc4c(c3Cl)OCC3COC5(CCN(C(=O)n6cnnc6O)C5)CN3C4=O)c12]

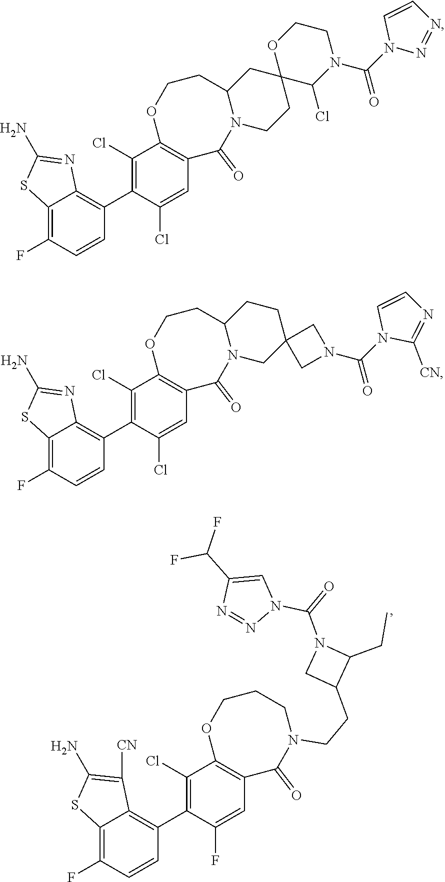 [SMILES: Nc1nc2c(-c3c(Cl)cc4c(c3Cl)OCCC3CC5(CCN3C4=O)OCCN(C(=O)n3ccnn3)C5Cl)ccc(F)c2s1 ; N#Cc1nccn1C(=O)N1CC2(CCC3CCOc4c(cc(Cl)c(-c5ccc(F)c6sc(N)nc56)c4Cl)C(=O)N3C2)C1 ; CCC1C(CCN2CCCOc3c(cc(F)c(-c4ccc(F)c5sc(N)c(C#N)c45)c3Cl)C2=O)CN1C(=O)n1cc(C(F)F)nn1]

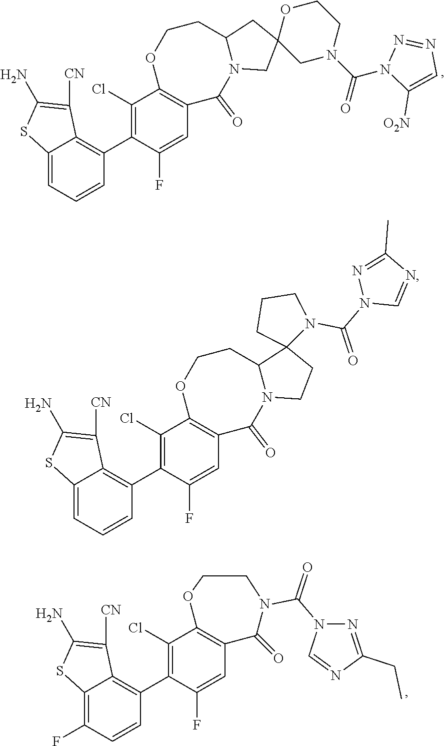 [SMILES: N#Cc1c(N)sc2cccc(-c3c(F)cc4c(c3Cl)OCCC3CC5(CN(C(=O)n6nncc6[N+](=O)[O-])CCO5)CN3C4=O)c12 ; Cc1ncn(C(=O)N2CCCC23CCN2C(=O)c4cc(F)c(-c5cccc6sc(N)c(C#N)c56)c(Cl)c4OCCC23)n1 ; CCc1ncn(C(=O)N2CCOc3c(cc(F)c(-c4ccc(F)c5sc(N)c(C#N)c45)c3Cl)C2=O)n1]

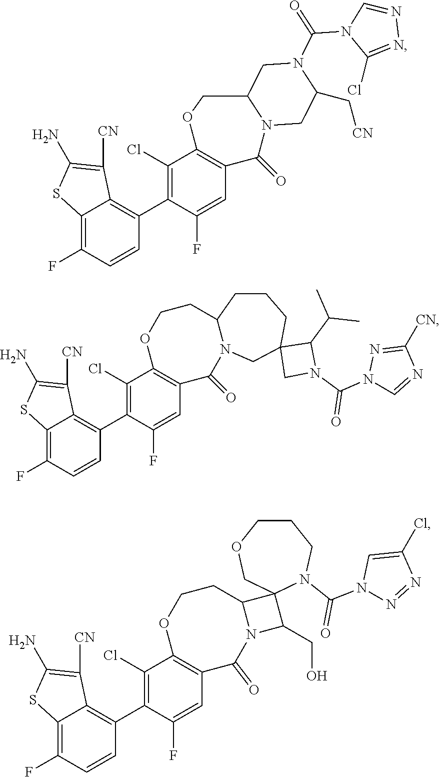 [SMILES: N#CCC1CN2C(=O)c3cc(F)c(-c4ccc(F)c5sc(N)c(C#N)c45)c(Cl)c3OCC2CN1C(=O)n1cnnc1Cl ; CC(C)C1N(C(=O)n2cnc(C#N)n2)CC12CCCC1CCOc3c(cc(F)c(-c4ccc(F)c5sc(N)c(C#N)c45)c3Cl)C(=O)N1C2 ; N#Cc1c(N)sc2c(F)ccc(-c3c(F)cc4c(c3Cl)OCCC3N(C4=O)C(CO)C34COCCCN4C(=O)n3cc(Cl)nn3)c12]

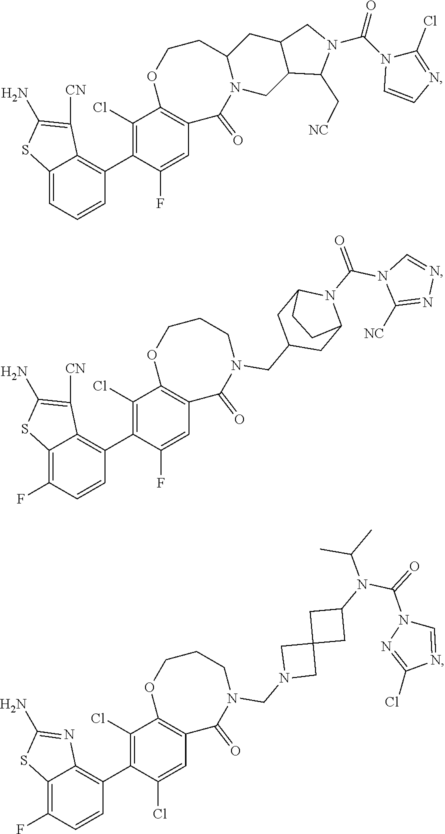 [SMILES: N#CCC1C2CN3C(=O)c4cc(F)c(-c5cccc6sc(N)c(C#N)c56)c(Cl)c4OCCC3CC2CN1C(=O)n1ccnc1Cl ; N#Cc1c(N)sc2c(F)ccc(-c3c(F)cc4c(c3Cl)OCCCN(CC3CC5CCC(C3)N5C(=O)n3cnnc3C#N)C4=O)c12 ; CC(C)N(C(=O)n1cnc(Cl)n1)C1CC2(C1)CN(CN1CCCOc3c(cc(Cl)c(-c4ccc(F)c5sc(N)nc45)c3Cl)C1=O)C2]

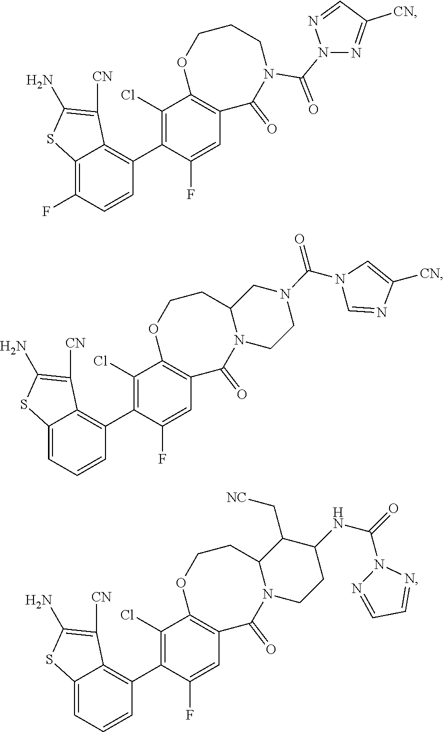 [SMILES: N#Cc1cnn(C(=O)N2CCCOc3c(cc(F)c(-c4ccc(F)c5sc(N)c(C#N)c45)c3Cl)C2=O)n1 ; N#Cc1cn(C(=O)N2CCN3C(=O)c4cc(F)c(-c5cccc6sc(N)c(C#N)c56)c(Cl)c4OCCC3C2)cn1 ; N#CCC1C(NC(=O)n2nccn2)CCN2C(=O)c3cc(F)c(-c4cccc5sc(N)c(C#N)c45)c(Cl)c3OCCC12]

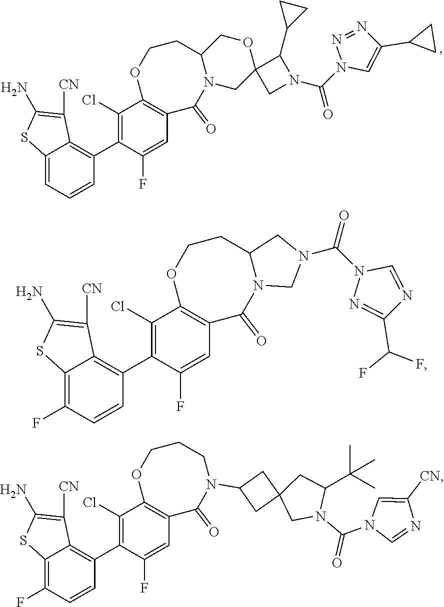

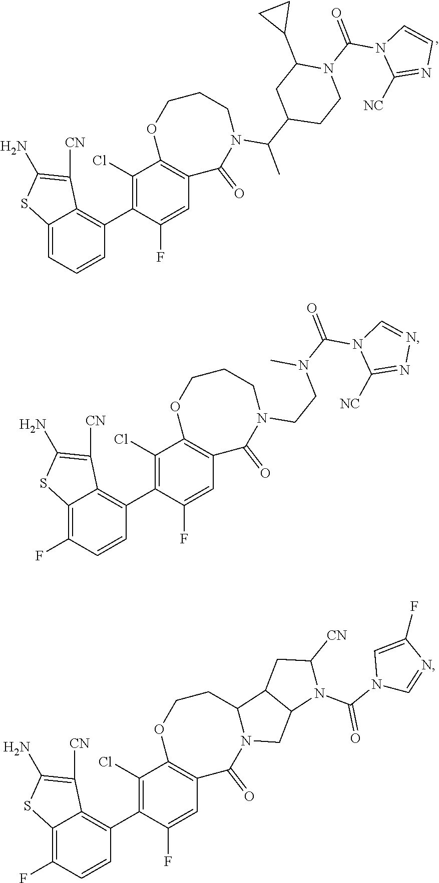 [SMILES: CC(C1CCN(C(=O)n2ccnc2C#N)C(C2CC2)C1)N1CCCOc2c(cc(F)c(-c3cccc4sc(N)c(C#N)c34)c2Cl)C1=O ; CN(CCN1CCCOc2c(cc(F)c(-c3ccc(F)c4sc(N)c(C#N)c34)c2Cl)C1=O)C(=O)n1cnnc1C#N ; N#Cc1c(N)sc2c(F)ccc(-c3c(F)cc4c(c3Cl)OCCC3C5CC(C#N)N(C(=O)n6cnc(F)c6)C5CN3C4=O)c12]

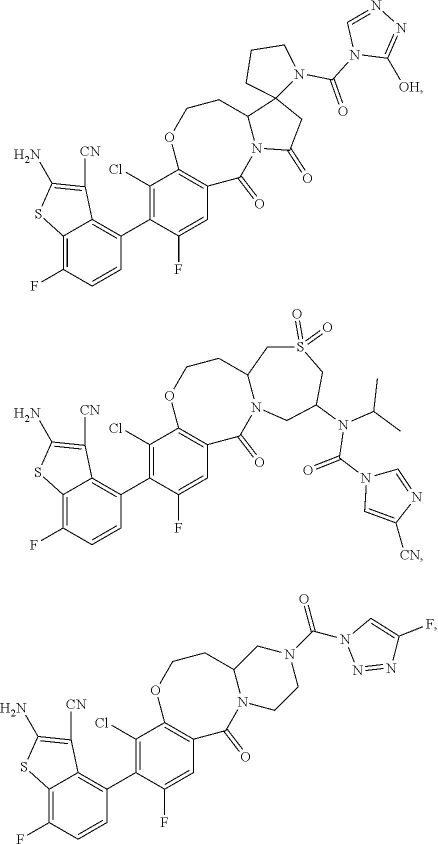 [SMILES: N#Cc1c(N)sc2c(F)ccc(-c3c(F)cc4c(c3Cl)OCCC3N(C(=O)CC35CCCN5C(=O)n3cnnc3O)C4=O)c12 ; CC(C)N(C(=O)n1cnc(C#N)c1)C1CN2C(=O)c3cc(F)c(-c4ccc(F)c5sc(N)c(C#N)c45)c(Cl)c3OCCC2CS(=O)(=O)C1 ; N#Cc1c(N)sc2c(F)ccc(-c3c(F)cc4c(c3Cl)OCCC3CN(C(=O)n5cc(F)nn5)CCN3C4=O)c12]

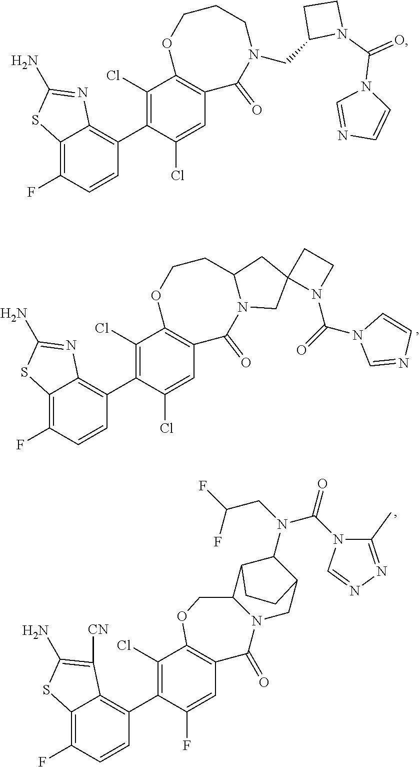 [SMILES: Nc1nc2c(-c3c(Cl)cc4c(c3Cl)OCCCN(C[C@@H]3CCN3C(=O)n3ccnc3)C4=O)ccc(F)c2s1 ; Nc1nc2c(-c3c(Cl)cc4c(c3Cl)OCCC3CC5(CCN5C(=O)n5ccnc5)CN3C4=O)ccc(F)c2s1 ; Cc1nncn1C(=O)N(CC(F)F)C1C2CCC1C1COc3c(cc(F)c(-c4ccc(F)c5sc(N)c(C#N)c45)c3Cl)C(=O)N1C2]

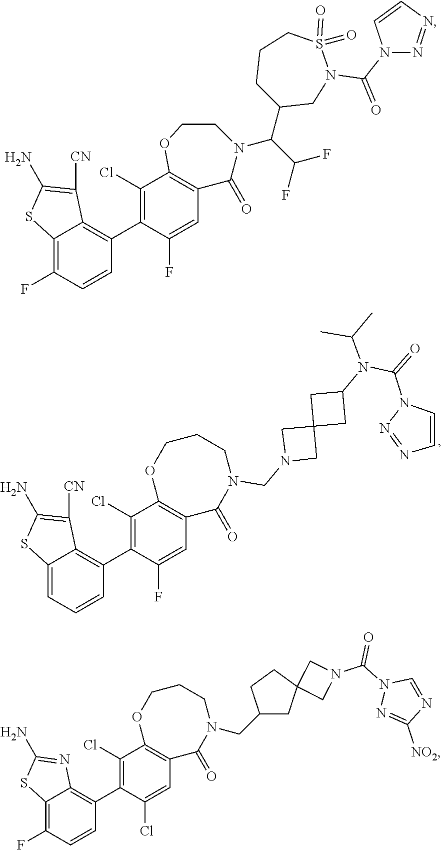 [SMILES: N#Cc1c(N)sc2c(F)ccc(-c3c(F)cc4c(c3Cl)OCCN(C(C(F)F)C3CCCS(=O)(=O)N(C(=O)n5ccnn5)C3)C4=O)c12 ; CC(C)N(C(=O)n1ccnn1)C1CC2(C1)CN(CN1CCCOc3c(cc(F)c(-c4cccc5sc(N)c(C#N)c45)c3Cl)C1=O)C2 ; Nc1nc2c(-c3c(Cl)cc4c(c3Cl)OCCCN(CC3CCC5(C3)CN(C(=O)n3cnc([N+](=O)[O-])n3)C5)C4=O)ccc(F)c2s1]

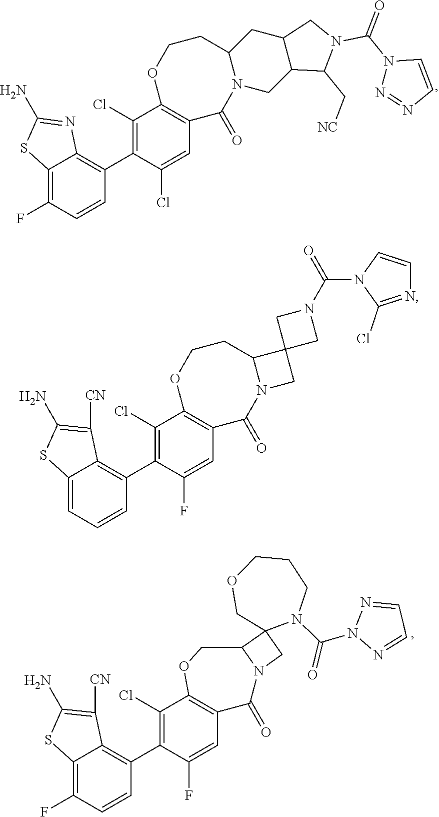 [SMILES: N#CCC1C2CN3C(=O)c4cc(Cl)c(-c5ccc(F)c6sc(N)nc56)c(Cl)c4OCCC3CC2CN1C(=O)n1ccnn1 ; N#Cc1c(N)sc2cccc(-c3c(F)cc4c(c3Cl)OCCC3N(CC35CN(C(=O)n3ccnc3Cl)C5)C4=O)c12 ; N#Cc1c(N)sc2c(F)ccc(-c3c(F)cc4c(c3Cl)OCC3N(CC35COCCCN5C(=O)n3nccn3)C4=O)c12]

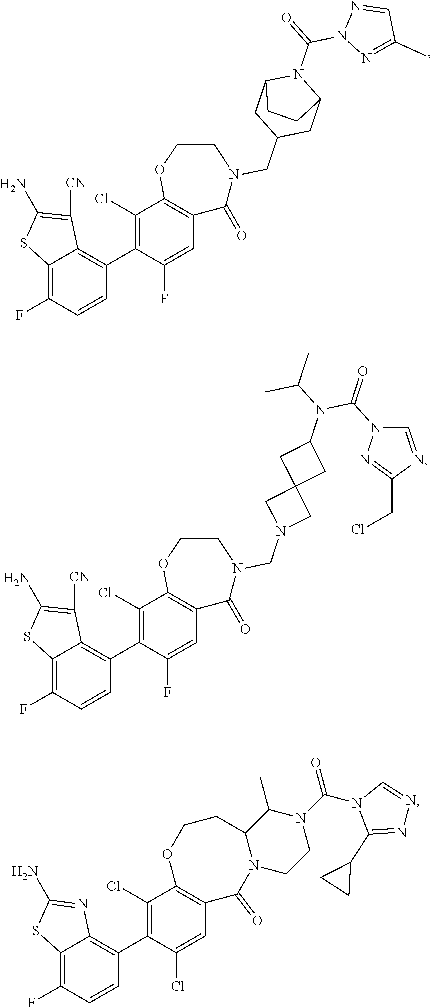 [SMILES: Cc1cnn(C(=O)N2C3CCC2CC(CN2CCOc4c(cc(F)c(-c5ccc(F)c6sc(N)c(C#N)c56)c4Cl)C2=O)C3)n1 ; CC(C)N(C(=O)n1cnc(CCl)n1)C1CC2(C1)CN(CN1CCOc3c(cc(F)c(-c4ccc(F)c5sc(N)c(C#N)c45)c3Cl)C1=O)C2 ; CC1C2CCOc3c(cc(Cl)c(-c4ccc(F)c5sc(N)nc45)c3Cl)C(=O)N2CCN1C(=O)n1cnnc1C1CC1]

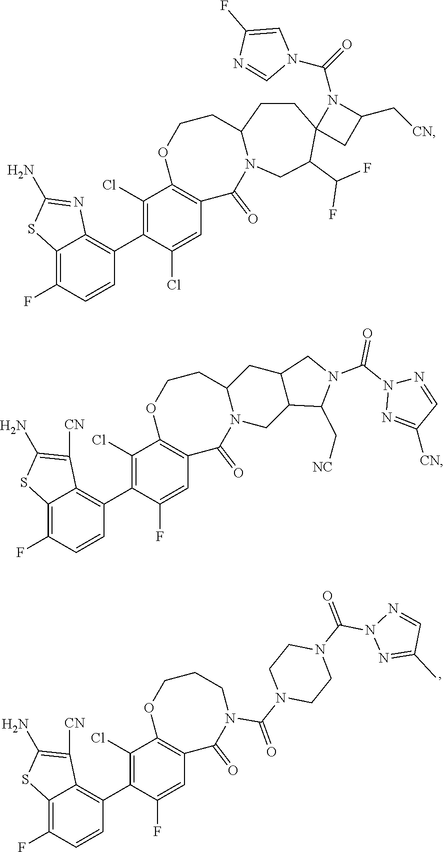 [SMILES: N#CCC1CC2(CCC3CCOc4c(cc(Cl)c(-c5ccc(F)c6sc(N)nc56)c4Cl)C(=O)N3CC2C(F)F)N1C(=O)n1cnc(F)c1 ; N#CCC1C2CN3C(=O)c4cc(F)c(-c5ccc(F)c6sc(N)c(C#N)c56)c(Cl)c4OCCC3CC2CN1C(=O)n1ncc(C#N)n1 ; Cc1cnn(C(=O)N2CCN(C(=O)N3CCCOc4c(cc(F)c(-c5ccc(F)c6sc(N)c(C#N)c56)c4Cl)C3=O)CC2)n1]

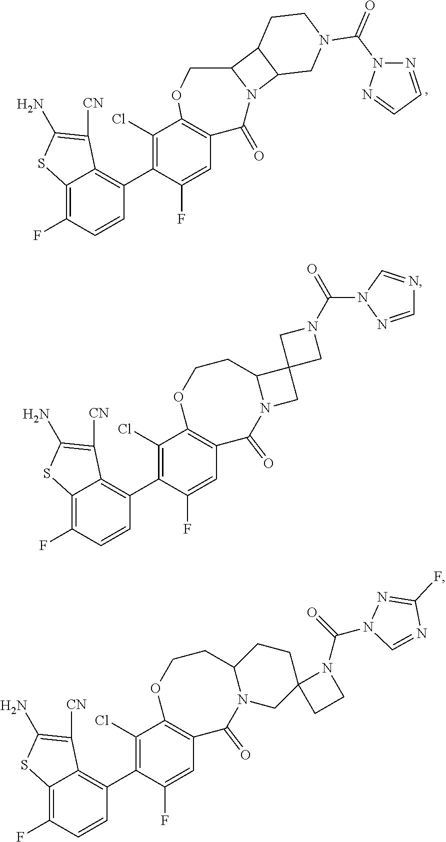 [SMILES: N#Cc1c(N)sc2c(F)ccc(-c3c(F)cc4c(c3Cl)OCC3C5CCN(C(=O)n6nccn6)CC5N3C4=O)c12 ; N#Cc1c(N)sc2c(F)ccc(-c3c(F)cc4c(c3Cl)OCCC3N(CC35CN(C(=O)n3cncn3)C5)C4=O)c12 ; N#Cc1c(N)sc2c(F)ccc(-c3c(F)cc4c(c3Cl)OCCC3CCC5(CCN5C(=O)n5cnc(F)n5)CN3C4=O)c12]

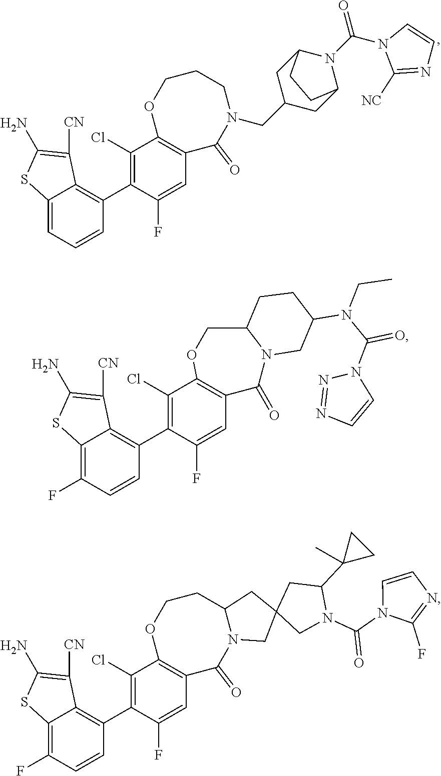 [SMILES: N#Cc1c(N)sc2cccc(-c3c(F)cc4c(c3Cl)OCCCN(CC3CC5CCC(C3)N5C(=O)n3ccnc3C#N)C4=O)c12 ; CCN(C(=O)n1ccnn1)C1CCC2COc3c(cc(F)c(-c4ccc(F)c5sc(N)c(C#N)c45)c3Cl)C(=O)N2C1 ; CC1(C2CC3(CC4CCOc5c(cc(F)c(-c6ccc(F)c7sc(N)c(C#N)c67)c5Cl)C(=O)N4C3)CN2C(=O)n2ccnc2F)CC1]

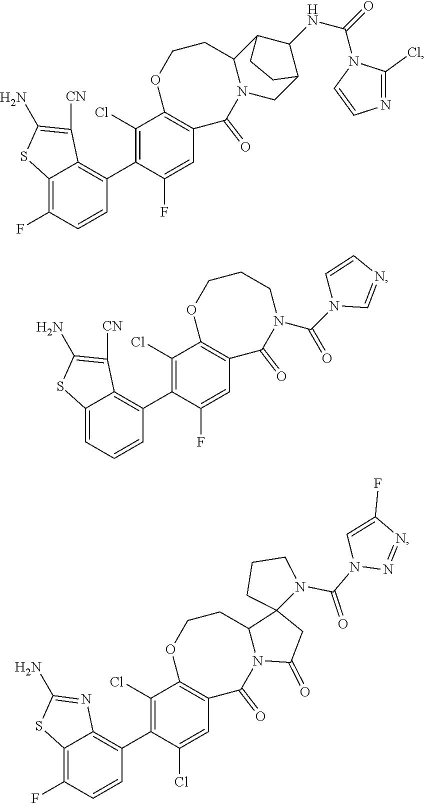 [SMILES: N#Cc1c(N)sc2c(F)ccc(-c3c(F)cc4c(c3Cl)OCCC3C5CCC(CN3C4=O)C5NC(=O)n3ccnc3Cl)c12 ; N#Cc1c(N)sc2cccc(-c3c(F)cc4c(c3Cl)OCCCN(C(=O)n3ccnc3)C4=O)c12 ; Nc1nc2c(-c3c(Cl)cc4c(c3Cl)OCCC3N(C(=O)CC35CCCN5C(=O)n3cc(F)nn3)C4=O)ccc(F)c2s1]

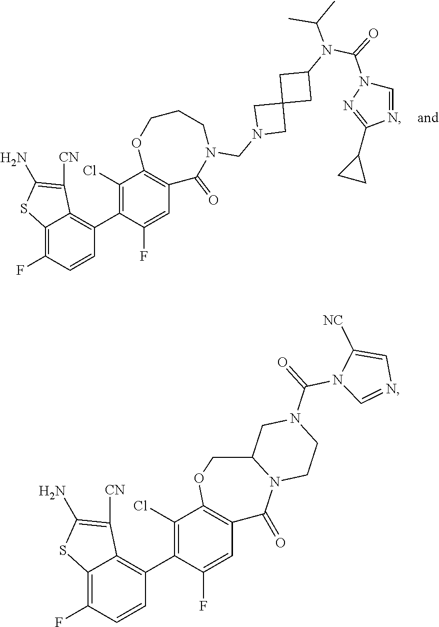 [SMILES: CC(C)N(C(=O)n1cnc(C2CC2)n1)C1CC2(C1)CN(CN1CCCOc3c(cc(F)c(-c4ccc(F)c5sc(N)c(C#N)c45)c3Cl)C1=O)C2 ; N#Cc1c(N)sc2c(F)ccc(-c3c(F)cc4c(c3Cl)OCC3CN(C(=O)n5cncc5C#N)CCN3C4=O)c12]

or a pharmaceutically acceptable salt, solvate, stereoisomer, or prodrug thereof.

In general, compounds of the disclosure may be prepared by the following reaction schemes:

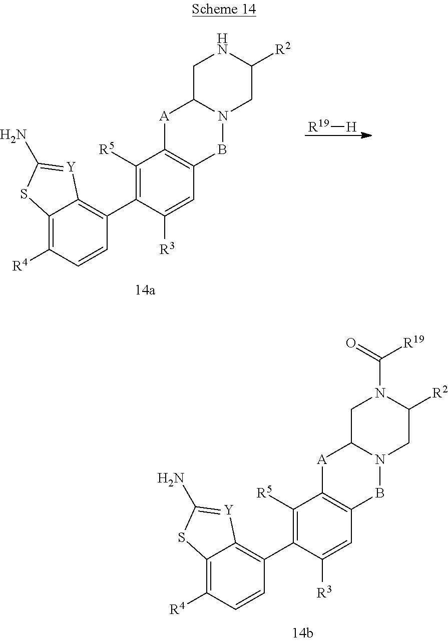

In some embodiments, a compound of Formula 14b may be prepared according to Scheme 14. For example, reaction of amine 14a with R¹⁹—H in the presence of triphosgene bis(trichloromethyl) carbonate (BTC) and pyridine can provide a compound of Formula 14b.

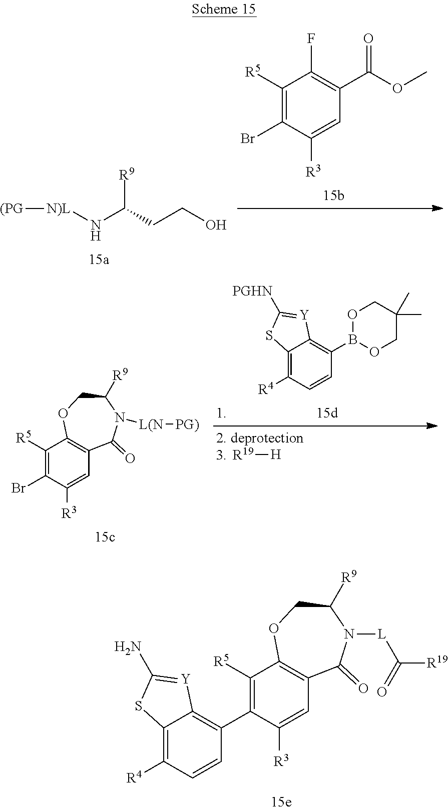

In some embodiments, a compound of Formula 15e may be prepared according to Scheme 15. For example, coupling of amine 15a (wherein L of compound 15a comprises a suitably protected nitrogen atom (e.g., wherein PG is Boc, Cbz, or Fmoc)) with methyl ester 15b and subsequent cyclization can give tricycle 15c using a suitable base, such as cesium carbonate, in solvent (e.g., DMF) with heating. Substitution of the aryl bromide can proceed via a Suzuki reaction with 15d. Global deprotection of the resulting compound can be followed by reaction with R¹⁹—H in the presence of triphosgene bis(trichloromethyl) carbonate (BTC) and pyridine to provide a compound of Formula 15e.

Synthetic procedures for certain intermediates, such as a compound of Formula 14a, may be found in WO 2021/118877, which is incorporated herein by reference in its entirety, including any synthetic methods, recitations of compound variables, combination therapies, and methods of use disclosed therein.

### Additional Compound Embodiments

In some embodiments, for a compound of Formula (I), (II), (III), (IV), (V), (VI), (VII), (VIII), (IX), (X), (XI), (XII), (XIII), (XIV), (XIX), (XX), (XXIII), or (XXIV), -L²-L³-C(O)R¹⁹ is selected from:

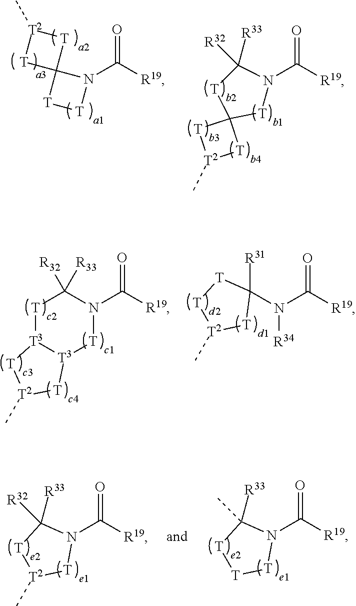

wherein:
a1, b1, b3, and b4 are independently 1, 2, 3, 4, or 5;
a2, a3, and b2 are independently 0, 1, 2, 3, 4, or 5;
c1, c2, c3, c4, d1, d2, e1, and e2 are independently 0, 1, 2, 3, or 4;
wherein the sum of a1, a2, and a3 is less than 9; the sum of b1, b2, b3, and b4 is less than 9; the sum of c1, c2, c3, and c4 is less than 8; the sum of d1 and d2 is less than 6; and the sum of e1 and e2 is less than 6;
T is independently selected at each occurrence from N(R³⁵), C(R³⁶)₂, C(O), O, S(O), and S(O)₂;
T² and T³ are independently selected at each occurrence from N and C(R³⁶);
R³¹, R³², R³³, and R³⁶ are independently selected at each occurrence from hydrogen and R⁴⁰;
R³⁴ and R³⁵ are independently selected at each occurrence from hydrogen and R⁴¹;
R⁴⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R⁴⁰ attached to the same carbon atom optionally join to form ═NR¹², ═C(R¹⁴)₂, or ═O; wherein two R⁴⁰ and the atom(s) to which they are attached optionally form C₃₋₁₂ carbocycle or 3- to 12-membered heterocycle; wherein R⁴⁰ and R⁴¹ and the atoms to which they are attached optionally form 3- to 12-membered heterocycle; and wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁴¹ is independently selected at each occurrence from —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

In some embodiments, for a compound of Formula (IX), (X), (XIX), (XX), (XXIII), or (XXIV),

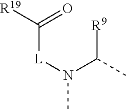

is selected from:

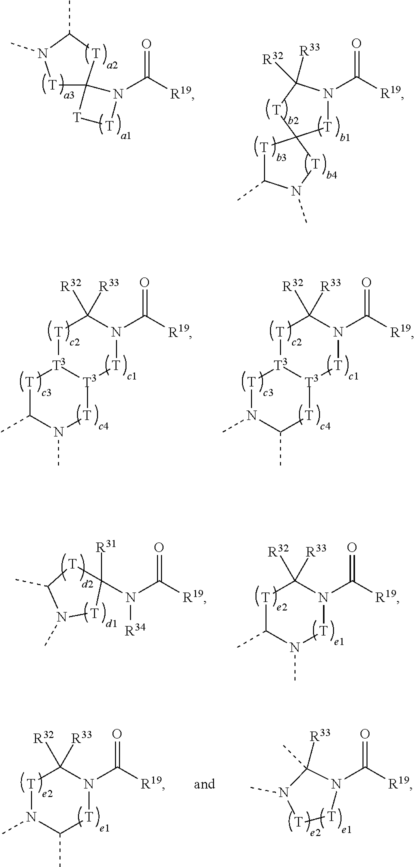

wherein:
a1, b1, b3, and b4 are independently 1, 2, 3, 4, or 5;
a2, a3, and b2 are independently 0, 1, 2, 3, 4, or 5;
c1, c2, c3, c4, d1, d2, e1, and e2 are independently 0, 1, 2, 3, or 4;
wherein the sum of a1, a2, and a3 is less than 8; the sum of b1, b2, b3, and b4 is less than 8; the sum of c1, c2, c3, and c4 is less than 7; the sum of d1 and d2 is less than 6; and the sum of e1 and e2 is less than 6;
T is independently selected at each occurrence from N(R³⁵), C(R³⁶)₂, C(O), O, S(O), and S(O)₂;
T³ is independently selected at each occurrence from N and C(R³⁶);
R³¹, R³², R³³, and R³⁶ are independently selected at each occurrence from hydrogen and R⁴⁰;
R³⁴ and R³⁵ are independently selected at each occurrence from hydrogen and R⁴¹;
R⁴⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R⁴⁰ attached to the same carbon atom optionally join to form ═NR¹², ═C(R¹⁴)₂, or ═O; wherein two R⁴⁰ and the atom(s) to which they are attached optionally form C₃₋₁₂ carbocycle or 3- to 12-membered heterocycle; wherein R⁴⁰ and R⁴¹ and the atoms to which they are attached optionally form 3- to 12-membered heterocycle; and wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
R⁴¹ is independently selected at each occurrence from —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted.

In some embodiments, R⁴⁰ is independently selected at each occurrence from halogen, —CN, C₁₋₆ alkyl, and C₃₋₆ cycloalkyl, or two R⁴⁰ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R⁴¹ is independently selected at each occurrence from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, R³¹ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R³¹ is selected from hydrogen and C₁₋₆ alkyl. In some embodiments, R³¹ is hydrogen.

In some embodiments, R³² is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R³² is selected from hydrogen and C₁₋₆ alkyl. In some embodiments, R³² is hydrogen.

In some embodiments, R³³ is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R³³ is selected from hydrogen and C₁₋₆ alkyl. In some embodiments, R³³ is hydrogen.

In some embodiments, R³⁶ is independently selected at each occurrence from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R³⁶ is independently selected at each occurrence from hydrogen and C₁₋₆ alkyl. In some embodiments, R³⁶ is hydrogen.

In some embodiments, R³⁴ is selected from hydrogen, C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl). In some embodiments, R³⁴ is selected from hydrogen and C₁₋₆ alkyl. In some embodiments, R³⁴ is hydrogen. In some embodiments, R³⁴ is C₁₋₆ alkyl.

In some embodiments, R³⁵ is independently selected at each occurrence from hydrogen, C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl). In some embodiments, R³⁴ is independently selected at each occurrence from hydrogen and C₁₋₆ alkyl. In some embodiments, R³⁴ is hydrogen.

In some embodiments, for a compound described herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), R¹⁹ is selected from 1,2,3-triazol-1-yl, 1,2,3-triazol-2-yl, 1,2,4-triazol-1-yl, and 1,2,4-triazol-4-yl, each of which is optionally substituted. In some embodiments, R¹⁹ is 1,2,3-triazol-1-yl. In some embodiments, R¹⁹ is 1,2,3-triazol-2-yl. In some embodiments, R¹⁹ is 1,2,4-triazol-1-yl. In some embodiments, R¹⁹ is 1,2,4-triazol-4-yl. In some embodiments, R¹⁹ is optionally substituted imidazol-1-yl.

In some embodiments, for a compound described herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), R¹⁹ is optionally substituted with one or two substituents independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents independently selected from halogen, —CN, C₁₋₆ alkyl, —O(C₁₋₆ alkyl), and —O(C₁₋₆ haloalkyl). In some embodiments, R¹⁹ is optionally substituted with one or two substituents independently selected from halogen, —CN, C₁₋₆ alkyl, C₁₋₆ haloalkyl, and C₃₋₆ cycloalkyl. In some embodiments, R¹⁹ is substituted with one or two substituents independently selected from halogen, —CN, C₁₋₆ alkyl, and C₁₋₆ haloalkyl. In some embodiments, R¹⁹ is unsubstituted.

In some embodiments, for a compound described herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), R¹⁹ is selected from

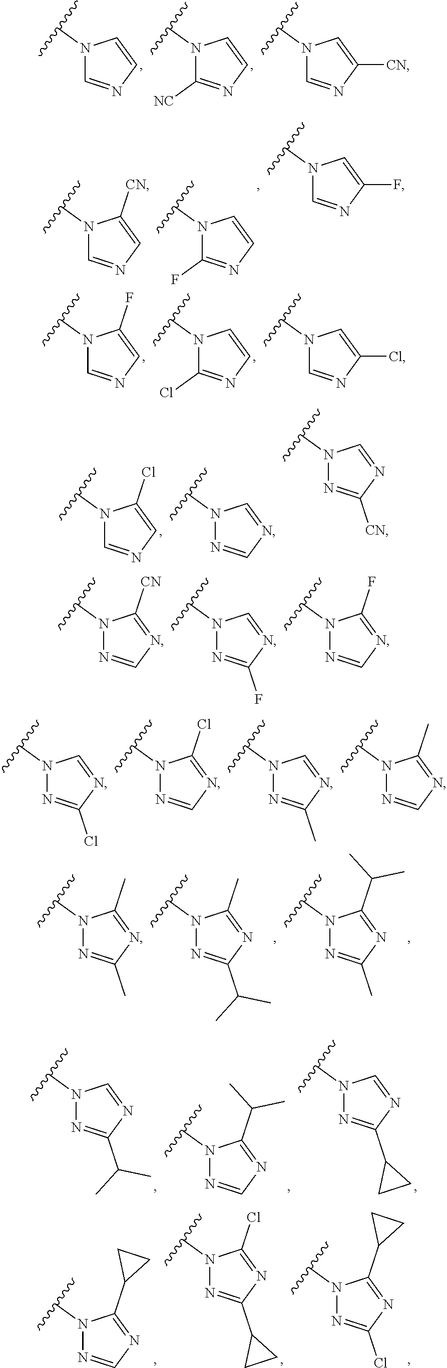

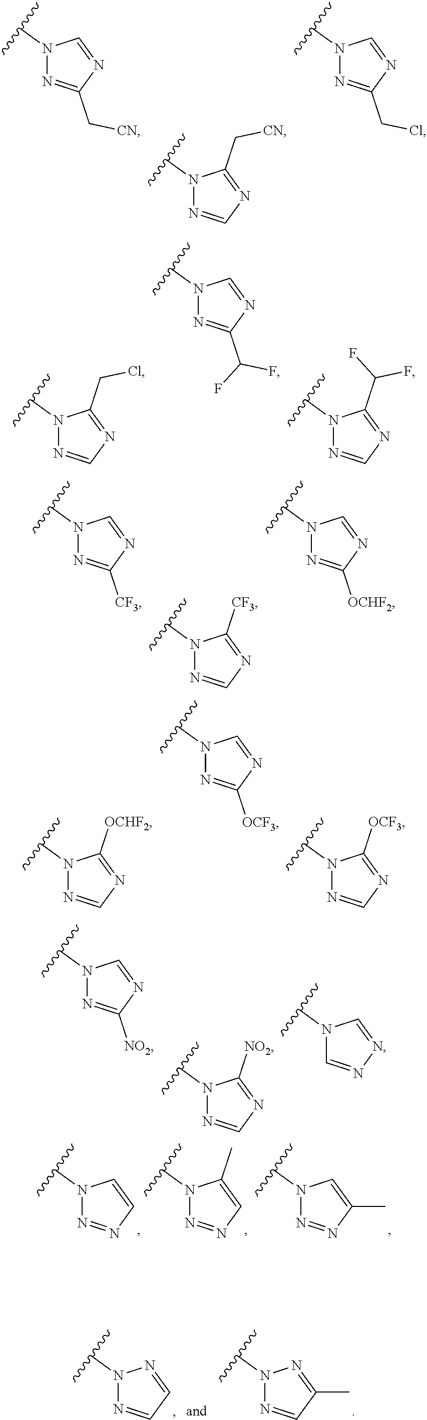

Unless stated otherwise, an optionally substituted group of the present disclosure may be unsubstituted or substituted with one or more, such as one, two, three, four, or five, substituents independently selected from R²⁰. In some embodiments, an optionally substituted group of the present disclosure is unsubstituted or substituted with one, two, or three substituents independently selected from R²⁰. In some embodiments, R²⁰ is independently selected at each occurrence from:
halogen, oxo, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³)—, —S(═O)(═NR²²)N(R²²)(R²³), and —OCH₂C(O)OR²²; wherein two R²⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), and —S(═O)(═NR²²)N(R²²)(R²³);
R²¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R²¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH; R²² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle); and
R²³ is independently selected at each occurrence from hydrogen and C₁₋₆ alkyl; or R²² and R²³ attached to the same nitrogen atom form 3- to 10 membered heterocycle.

In some embodiments, a compound disclosed herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), exhibits selective and potent inhibition of K-Ras G12S and/or K-Ras G12C relative to wildtype K-Ras or other K-Ras mutants (e.g., K-Ras G12V or K-Ras G12D). In some embodiments, a subject warhead exhibits selective engagement of K-Ras G12S and/or K-Ras G12C relative to K-Ras G12D or wildtype K-Ras by at least 1-fold, and in some instances greater than 2-, 3-, 4-, 5-, 10-, 15-, or 20-fold, or even higher. In some embodiments, a subject warhead exhibits a selective and rapid engagement of K-Ras G12S and/or K-Ras G12C yielding at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, or even higher engagement of G12S and/or K-Ras G12C within, 10 mins, 20 mins, 30 mins, 1 hr, 2 hrs, 3 hrs, 4 hrs, 5 hrs, 6 hrs, 7 hrs, 8 hrs, 9 hrs, 10 hrs, 20 hrs, or 24 hours. In some embodiments, a selective and rapid engagement of K-Ras G12S and/or K-Ras G12C is evidenced by at least 50% engagement within 24 hours. In some embodiments, subject compounds specifically engage K-Ras G12S and/or K-Ras G12C covalently with essentially no detectable labeling of K-Ras G12D when assayed under comparable conditions.

The inclusion of a warhead of the present disclosure may enhance the efficacy or potency of K-Ras G12S and/or K-Ras G12C inhibition. In some embodiments, a subject compound comprising a subject warhead inhibits K-Ras G12S and/or K-Ras G12C with higher potency as evidenced by an IC50 value that is at least 10%, 20%, 50%, 100%, 200%, 300%, 400%, or at least 500% lower than the IC50 value of a corresponding control compound that does not comprise the warhead. In some embodiments, a subject compound comprising a subject warhead inhibits K-Ras G12S and/or K-Ras G12C with higher potency as evidenced by an IC50 value that is at least 1.1-times, 1.2-times, 1.5-times, 2-times, 3-times, 4-times, 5-times, 6-times, 7-times, 8-times, 9-times, 10-times, 15-times, or at least 20-times lower than the IC50 value of a corresponding control compound that does not comprise the warhead, as ascertained in a biochemical assay exemplified in Example 4.

The inclusion of a warhead of the present disclosure may enhance the efficacy or potency with which a subject compound inhibits the proliferation of cells that express a K-Ras G12S mutation and/or a K-Ras G12C mutation. In some embodiments, a subject compound comprising a subject warhead inhibits the proliferation of cells that express a K-Ras G12S mutation and/or a K-Ras G12C mutation with higher potency as evidenced by an IC50 value that is at least 10%, 20%, 50%, 100%, 200%, 300%, 400%, or at least 500% lower than the IC50 value of a corresponding control compound that does not comprise the warhead. In some embodiments, a subject compound comprising a subject warhead inhibits the proliferation of cells that express a K-Ras G12S mutation and/or a K-Ras G12C mutation with higher potency as evidenced by an IC50 value that is at least 1.1-times, 1.2-times, 1.5-times, 2-times, 3-times, 4-times, 5-times, 6-times, 7-times, 8-times, 9-times, 10-times, 15-times, or at least 20-times lower than the IC50 value of a corresponding control compound that does not comprise the warhead, as ascertained in a cellular inhibition assay exemplified in Example 8.

In some embodiments, a compound described herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, the present disclosure provides an atropisomer of a compound described herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV). In some embodiments, the atropisomer is provided in enantiomeric excess. In some embodiments, the atropisomer is provided in at least 80% enantiomeric excess, such as at least 85%, at least 90%, at least 95%, at least 96%, at least 97%, at least 98%, at least 99%, or at least 99.9% enantiomeric excess. In some embodiments, the compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV) is preferably used as a non-racemic mixture, wherein one atropisomer is present in excess of its corresponding enantiomer or epimer. Typically, such mixture contains a mixture of the two isomers in a ratio of at least 9:1, preferably at least 19:1. In some embodiments, the atropisomer is provided in at least 96% enantiomeric excess, meaning the compound has less than 2% of the corresponding enantiomer. In some embodiments, the atropisomer is provided in at least 96% diastereomeric excess, meaning the compound has less than 2% of the corresponding diastereomer.

The term “atropisomers” refers to conformational stereoisomers which occur when rotation about a single bond in the molecule is prevented, restricted, or greatly slowed as a result of steric interactions with other parts of the molecule and wherein the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom. Atropisomers are typically considered stable if the barrier to interconversion is high enough to permit the atropisomers to undergo little or no interconversion at room temperature for a least a week, preferably at least a year. In some embodiments, an atropisomeric compound of the disclosure does not undergo more than about 5% interconversion to its opposite atropisomer at room temperature during one week when the atropisomeric compound is in substantially pure form, which is generally a solid state. In some embodiments, an atropisomeric compound of the disclosure does not undergo more than about 5% interconversion to its opposite atropisomer at room temperature (approximately 25° C.) during one year. The present chemical entities, pharmaceutical compositions, and methods are meant to include all such possible atropisomers, including racemic mixtures, diastereomeric mixtures, epimeric mixtures, optically pure forms of single atropisomers, and intermediate mixtures.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases or inorganic or organic acids to form a pharmaceutically acceptable salt. In some embodiments, such salts are prepared in situ during the final isolation and purification of the compounds described herein, or by separately reacting a purified compound in its free form with a suitable acid or base, and isolating the salt thus formed.

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

The chemical entities described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1-15, the steps in some cases may be performed in a different order than the order shown in Schemes 1-15. Various modifications to these synthetic reaction schemes may be made and will be suggested to one skilled in the art having referred to the present disclosure. Numberings or R groups in each scheme typically have the same meanings as those defined elsewhere herein unless otherwise indicated.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

In some embodiments, the compounds of the present disclosure exhibit one or more functional characteristics disclosed herein. For example, a subject compound binds to a Ras protein, KRAS protein or a mutant form thereof. In some embodiments, a subject compound binds specifically and also inhibits a Ras protein, KRAS protein or a mutant form thereof. In some embodiments, a subject compound selectively inhibits a KRAS mutant relative to a wildtype KRAS. In some embodiments, the IC50 of a subject compound for a KRAS mutant (e.g., G12S and/or G12C) is less than about 5 μM, less than about 1 μM, less than about 500 nM, less than 250 nM, less than 100 nM, less than 50 nM, or even less, as measured in an in vitro assay known in the art or exemplified herein. In some embodiments, a subject compound covalently binds to a KRAS mutant (e.g., KRAS G12S and/or KRAS G12C).

In some embodiments, a compound of the present disclosure is capable of reducing Ras signaling output. Such reduction may be evidenced by one or more of the following: (i) an increase in steady state level of GDP-bound Ras protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, and (vi) reduction (e.g., inhibition) of cell growth of Ras-driven tumor cells (e.g., those derived from a tumor cell line disclosed herein). In some cases, the reduction in Ras signaling output can be evidenced by two, three, four, five, or all of (i)-(vi) above.

It shall be understood that different aspects of the disclosure can be appreciated individually, collectively, or in combination with each other. Various aspects described herein may be applied to any of the particular applications disclosed herein. The compositions of matter, including compounds of any formulae disclosed in the compound section, of the present disclosure may be utilized in the method section, including methods of use and production disclosed herein, or vice versa.

### Methods

The compounds described herein, or a pharmaceutically acceptable salt or solvate thereof, are Ras inhibitors capable of inhibiting a Ras protein, such as wild-type Ras or a Ras mutant protein (e.g., G12S, G12C, G12D, G12V, G13C, and/or G13D) from K-Ras, H-Ras or N-Ras. Compounds, including pharmaceutically acceptable salts or solvates thereof, disclosed herein have a wide range of applications in therapeutics, diagnostics, and other biomedical research.

In certain aspects, the present disclosure provides a method of treating cancer in a subject in need thereof, comprising administering to the subject a therapeutically effective amount of a compound described herein, or a pharmaceutically acceptable salt or solvate thereof.

In certain aspects, the present disclosure provides a method of treating a cancer comprising amplified wildtype Ras or a Ras mutant (e.g., G12S, G12C, G12D, G12V, G13C, and/or G13D) protein in a subject, comprising inhibiting amplified wildtype Ras or the Ras mutant protein of said subject by administering to said subject a compound, wherein the compound is characterized in that upon contacting the Ras protein, the Ras protein activity or function is inhibited (e.g., partially inhibited or completely inhibited), such that said inhibited Ras protein exhibits reduced Ras signaling output (e.g., compared to a corresponding Ras protein not contacted by the compound).

In certain aspects, the present disclosure provides a method of modulating activity of a Ras protein (e.g., K-Ras, mutant K-Ras, K-Ras G12S, K-Ras G12C, K-Ras G12D, K-Ras G12V, K-Ras G13C, and/or K-Ras G13D), comprising contacting a Ras protein with an effective amount of a compound described herein, or a pharmaceutically acceptable salt or solvate thereof, thereby modulating the activity of the Ras protein.

In certain aspects, the present disclosure provides a method of inhibiting cell growth, comprising administering an effective amount of a compound described herein, or a pharmaceutically acceptable salt or solvate thereof, to a cell expressing a Ras (e.g., K-Ras) protein, thereby inhibiting growth of said cells. In some embodiments, the subject method comprises administering an additional agent to said cell.

In certain aspects, the present disclosure provides a method of treating a disease mediated at least in part by a Ras protein, such as K-Ras or a mutant thereof, in a subject in need thereof, comprising administering to the subject an effective amount of a compound disclosed herein, or a pharmaceutically acceptable salt or solvate thereof. In some embodiments, the disease is cancer, such as a solid tumor or a hematological cancer. In some embodiments, the method further comprises administering an additional agent to the subject, such as a SHP2 inhibitor, a SOS inhibitor, an EGFR inhibitor, a MEK inhibitor, an ERK inhibitor, a CDK4/6 inhibitor, a BRAF inhibitor, or a combination thereof.

In certain aspects, the present disclosure provides a method of inhibiting activity of a Ras protein, such as K-Ras or a mutant thereof, comprising contacting the Ras protein with a compound disclosed herein, or a pharmaceutically acceptable salt or solvate thereof. In some embodiments, the compound exhibits an IC50 against the Ras protein of less than 10 μM, such as less than 5 μM, 1 μM, 500 nM, 100 nM, 50 nM, 10 nM, 5 nM, 1 nM, 500 pM, 50 pM, 10 pM or less.

In certain aspects, the present disclosure provides a method of treating a Ras-mediated cancer in a subject in need thereof, comprising administering to the subject a SHP2 inhibitor, a SOS inhibitor, an EGFR inhibitor, a MEK inhibitor, an ERK inhibitor, a CDK4/6 inhibitor, or a BRAF inhibitor and an effective amount of a compound disclosed herein, such as a compound of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), or a pharmaceutically acceptable salt or solvate thereof. In some embodiments, the cancer is a solid tumor. In some embodiments, the cancer is a hematological cancer.

In practicing any of the methods disclosed herein, the Ras target to which a subject compound binds, either covalently or reversibly, can be a Ras mutant (e.g., G12S, G12C, G12D, G12V, G13C, and/or G13D), including a mutant of K-Ras, H-Ras, or N-Ras. In some embodiments, the methods of treating cancer can be applied to treat a solid tumor or a hematological cancer. In some embodiments, the cancer being treated can be, without limitation, prostate cancer, brain cancer, colon cancer, rectal cancer, renal-cell carcinoma, liver cancer, various lung cancers including non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein, or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

Any of the treatment methods disclosed herein can be administered alone or in combination or in conjunction with another therapy or another agent. By “combination” it is meant to include (a) formulating a subject composition containing a subject compound together with another agent, or (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiments, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof. In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present disclosure include: anthracyclines; alkylating agents; antimetabolites; drugs that inhibit either the calcium dependent phosphatase calcineurin or the p70S6 kinase FK506 or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors: methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes: uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, RevimmuneT), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In certain aspects, compositions provided herein can be administered in combination with radiotherapy, such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-11BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody. In some embodiments, the checkpoint immune blockade agents are selected from the group consisting of anti-PD-L1 antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab, cemiplimab), anti-LAG3 antibodies (e.g., C9B7W, 410C9), anti-B7-H3 antibodies (e.g., DS-5573a), anti-TIM3 antibodies (e.g., F38-2E2), and combinations thereof. In one embodiment, the checkpoint immune blockade agent is an anti-PD-L1 antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldesleukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In some embodiments, a compound described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is administered in combination or in conjunction with one or more pharmacologically active agents selected from (1) an inhibitor of MEK (e.g., MEK1, MEK2) or of mutants thereof (e.g., trametinib, cobimetinib, binimetinib, selumetinib, refametinib, AZD6244); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or FGFR4 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120, sorafenib, vemurafenib, dabrafenib, encorafenib, regorafenib, GDC-879); (19) an inhibitor of ERK and/or of mutants thereof (e.g., ulixertinib, MK-8353, LTT462, AZD0364, SCH772984, BIX02189, LY3214996, ravoxertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g. tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of Src, FLT3, HDAC, VEGFR, PDGFR, LCK, Bcr-Abl or AKT, (33) an inhibitor of KRAS G12C mutant (e.g., including but not limited to AMG510, MRTX849, and any covalent inhibitors binding to the cysteine residue 12 of KRAS, the structures of which are publicly known) (e.g., an inhibitor of Ras G12C as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety), (34) an SHC inhibitor (e.g., PP2, AID371185), (35) a GAB inhibitor (e.g., GAB-0001), (36) a GRB inhibitor, (37) a PI-3 kinase inhibitor (e.g., idelalisib, copanlisib, duvelisib, alpelisib, taselisib, perifosine, buparlisib, umbralisib, NVP-BEZ235-AN), (38) a MARPK inhibitor, (39) a CDK4/6 inhibitor (e.g., palbociclib, ribociclib, abemaciclib), (40) a MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197), or (41) a SHP pathway inhibitor, such as a SHP2 inhibitor (e.g., RMC-4630, ERAS-601,

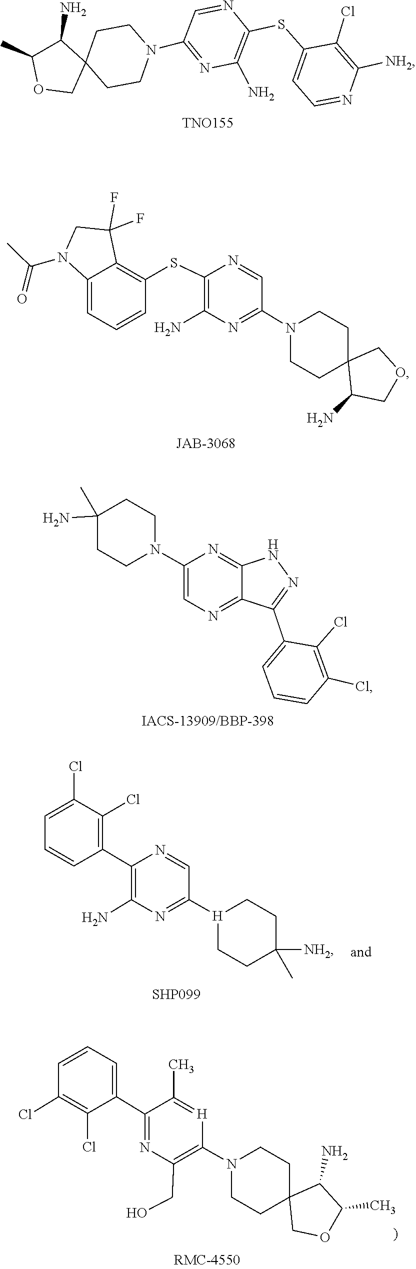

or a SHP1 inhibitor. In some embodiments, a Ras inhibitor described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is administered in combination or in conjunction with one or more checkpoint immune blockade agents (e.g., anti-PD-1 and/or anti-PD-L1 antibody, anti-CLTA-4 antibody). In some embodiments, a Ras inhibitor described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is administered in combination or in conjunction with one or more pharmacologically active agents comprising an inhibitor against one or more targets selected from: MEK, epidermal growth factor receptor (EGFR), FGFR1, FGFR2, FGFR3, mitotic kinase, topoisomerase, ALK, ALK5, c-MET, ErbB2, AXL, NTRK1, RET, A-Raf, B-Raf, C-Raf, ERK, MDM2, mTOR, BET, IGF1/2, IGF1-R, CDK9, SHIP1, SHIP2, SHP2, SRC, JAK, PARP, BTK, FLT3, HDAC, VEGFR, PDGFR, LCK, Bcr-Abl, AKT, KRAS G12C mutant, and ROS1. In some embodiments, a Ras inhibitor described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is administered in combination or in conjunction with one or more additional pharmacologically active agents comprising an inhibitor of SOS (e.g., SOS1, SOS2) or of mutants thereof, such as

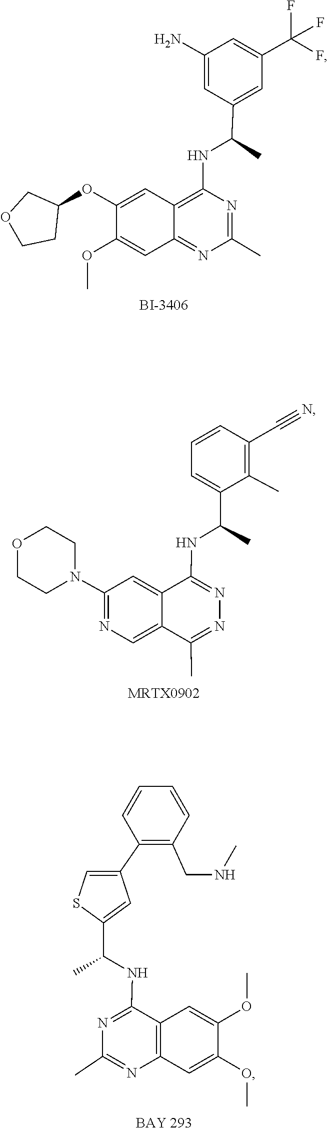

RMC-5845, or BI-1701963. In some embodiments, a Ras inhibitor described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is administered in combination or in conjunction with an inhibitor of SOS described in WO2021092115, WO2018172250, WO2019201848, WO2019122129, WO2018115380, WO2021127429, WO2020180768, or WO2020180770, each of which is herein incorporated by reference in its entirety for all purposes.

In some embodiments, a Ras inhibitor described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), is administered in combination or in conjunction with one or more checkpoint immune blockade agents (e.g., anti-PD-1 and/or anti-PD-L1 antibody, anti-CLTA-4 antibody).

In some embodiments, a compound described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), and one or more pharmacologically active agents are administered either simultaneously, concurrently or sequentially with no specific time limits, wherein such administration provides therapeutically effective levels of the two or more compounds in the body of the patient.

In some embodiments, a compound described herein, such as a compound, salt, or solvate of Formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), or (XXIV), and one or more pharmacologically active agents are administered sequentially in any order by a suitable route, such as infusion or orally. The dosing regimen may vary depending upon the stage of the disease, physical fitness of the patient, safety profiles of the individual drugs, and tolerance of the individual drugs, as well as other criteria known to the attending physician and medical practitioner(s) administering the combination. The compound of the present disclosure and other pharmacologically active agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In some cases, a treatment regime may be dosed according to a body weight of a subject. In subjects who are determined obese (BMI>35) a practical weight may need to be utilized. BMI is calculated by: BMI=weight (kg)/[height (m)]². Body weight may be calculated for men as 50 kg+2.3*(number of inches over 60 inches) or for women 45.5 kg+2.3*(number of inches over 60 inches). An adjusted body weight may be calculated for subjects who are more than 20% of their ideal body weight. An adjusted body weight may be the sum of an ideal body weight+(0.4*(Actual body weight−ideal body weight)). In some cases, a body surface area may be utilized to calculate a dosage. A body surface area (BSA) may be calculated by: BSA (m²)=√Height (cm)*Weight (kg)/3600.

In certain aspects, the present disclosure provides a method of modulating activity of a Ras (e.g., K-Ras) protein, comprising contacting a Ras protein with an effective amount of a compound described herein, or a pharmaceutically acceptable salt or solvate thereof, thereby modulating the activity of the Ras (e.g., K-Ras) protein. In some embodiments, the subject method comprises administering an additional agent or therapy.

In certain aspects, the present disclosure provides a method of modulating activity of a Ras protein, comprising contacting a Ras protein with an effective amount of a compound described, or a pharmaceutically acceptable salt or solvate thereof, wherein said modulating comprises inhibiting the Ras (e.g., K-Ras) protein activity. In certain aspects, the present disclosure provides a method of modulating activity of a Ras protein, such as Ras mutant (e.g., G12S, G12C, G12D, G12V, G13C, and/or G13D) proteins of K-Ras, H-Ras, and N-Ras, comprising contacting the Ras protein with an effective amount of a compound described herein, or a pharmaceutically acceptable salt or solvate thereof.

In certain aspects, the present disclosure provides a method of reducing Ras signaling output in a cell by contacting the cell with a compound described herein. A reduction in Ras signaling can be evidenced by one or more of the following: (i) an increase in steady state level of GDP-bound modified protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, (vi) a reduction of cell growth of a tumor cell expressing a Ras mutant (e.g., G12S, G12C, G12D, G12V, G13C, and/or G13D) protein, and (vii) a reduction in Ras interaction with a Ras-pathway signaling protein. Non-limiting examples of Ras-pathway signaling proteins include SOS (including SOS1 and SOS2), RAF, SHC, SHP (including SHP1 and SHP2), MEK, MAPK, ERK, GRB, RASA1, and GNAQ. In some embodiments, the reduction in Ras signaling output can be evidenced by two, three, four, five, six, or all of (i)-(vii) above. In some embodiments, the reduction of any one or more of (i)-(vii) can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control not treated with a subject compound. A reduction in cell growth can be demonstrated with the use of tumor cells or cell lines. A tumor cell line can be derived from a tumor in one or more tissues, e.g., pancreas, lung, ovary, biliary tract, intestine (e.g., small intestine, large intestine, colon), endometrium, stomach, hematopoietic tissue (e.g., lymphoid tissue), etc. Examples of tumor cell lines comprising a K-Ras mutation include, but are not limited to, A549 (e.g., K-Ras G12S), AGS (e.g., K-Ras G12D), ASPC1 (e.g., K-Ras G12D), Calu-6 (e.g., K-Ras Q61K), CFPAC-1 (e.g., K-Ras G12V), CL40 (e.g., K-Ras G12D), COLO678 (e.g., K-Ras G12D), COR-L23 (e.g., K-Ras G12V), DAN-G (e.g., K-Ras G12V), GP2D (e.g., K-Ras G12D), GSU (e.g., K-Ras G12F), HCT116 (e.g., K-Ras G13D), HEC1A (e.g., K-Ras G12D), HEC1B (e.g., K-Ras G12F), HEC50B (e.g., K-Ras G12F), HEYA8 (e.g., K-Ras G12D or G13D), HPAC (e.g., K-Ras G12D), HPAFII (e.g., K-Ras G12D), HUCCT1 (e.g., K-Ras G12D), KARPAS620 (e.g., K-Ras G13D), KOPN8 (e.g., K-Ras G13D), KP-3 (e.g., K-Ras G12V), KP-4 (e.g., K-Ras G12D), L3.3 (e.g., K-Ras G12D), LoVo (e.g., K-Ras G13D), LS180 (e.g., K-Ras G12D), LS513 (e.g., K-Ras G12D), MCAS (e.g., K-Ras G12D), NB4 (e.g., K-Ras A18D), NCI-H1355 (e.g., K-Ras G13C), NCI-H1573 (e.g., K-Ras G12A), NCI-H1944 (e.g., K-Ras G13D), NCI-H2009 (e.g., K-Ras G12A), NCI-H441 (e.g., K-Ras G12V), NCI-H747 (e.g., K-Ras G13D), NOMO-1 (e.g., K-Ras G12D), OV7 (e.g., K-Ras G12D), PANC0203 (e.g., K-Ras G12D), PANC0403 (e.g., K-Ras G12D), PANC0504 (e.g., K-Ras G12D), PANC0813 (e.g., K-Ras G12D), PANC1 (e.g., K-Ras G12D), Panc-10.05 (e.g., K-Ras G12D), PaTu-8902 (e.g., K-Ras G12V), PK1 (e.g., K-Ras G12D), PK45H (e.g., K-Ras G12D), PK59 (e.g., K-Ras G12D), SK-CO-1 (e.g., K-Ras G12V), SKLU1 (e.g., K-Ras G12D), SKM-1 (e.g., K-Ras K117N), SNU1 (e.g., K-Ras G12D), SNU1033 (e.g., K-Ras G12D), SNU1197 (e.g., K-Ras G12D), SNU407 (e.g., K-Ras G12D), SNU410 (e.g., K-Ras G12D), SNU601 (e.g., K-Ras G12D), SNU61 (e.g., K-Ras G12D), SNU8 (e.g., K-Ras G12D), SNU869 (e.g., K-Ras G12D), SNU-C2A (e.g., K-Ras G12D), SU.86.86 (e.g., K-Ras G12D), SUIT2 (e.g., K-Ras G12D), SW1990 (e.g., K-Ras G12D), SW403 (e.g., K-Ras G12V), SW480 (e.g., K-Ras G12V), SW620 (e.g., K-Ras G12V), SW948 (e.g., K-Ras Q61L), T3M10 (e.g., K-Ras G12D), TCC-PAN2 (e.g., K-Ras G12R), TGBC11TKB (e.g., K-Ras G12D), and MIA Pa-Ca (e.g., MIA Pa-Ca 2 (e.g., K-Ras G12C)).

In an aspect is provided a modified Ras mutant protein comprising a compound described herein (or a remnant of a compound described herein wherein the remnant of said compound is modified from a stand-alone compound described herein upon covalently bonding to an amino acid) covalently bonded to the amino acid corresponding to position 12 or 13 of SEQ ID No: 1. In some embodiments, such covalently bonded modified Ras mutant protein exhibits a reduced Ras signaling output (e.g., compared to a corresponding unmodified Ras mutant absent of the covalently bonded compound). In some embodiments, the modified Ras mutant protein comprises a compound described herein covalently bonded to the amino acid residue corresponding to position 12 or 13 of SEQ ID No: 1. In some embodiments, the modified Ras mutant protein comprises a compound described herein covalently bonded to the amino acid residue corresponding to position 12 or 13 of SEQ ID No: 1, wherein the Ras mutant protein is a human protein selected from KRAS G12S, KRAS G12C, KRAS G12D, KRAS G12V, KRAS G13C, and KRAS G13D. In some embodiments, the modified Ras mutant protein comprises a compound described herein covalently bonded to the amino acid residue corresponding to position 12 or 13 of SEQ ID No: 1, wherein the Ras mutant protein is a human KRAS mutant protein (e.g., G12S, G12C, G12D, G12V, G13C, and/or G13D). In some embodiments, the modified Ras mutant protein comprises a compound described herein covalently bonded to the amino acid residue corresponding to position 12 or 13 of SEQ ID No: 1, wherein the Ras mutant protein is a human KRAS G12S protein. In some embodiments, the modified Ras mutant protein comprises a compound described herein covalently bonded to the amino acid residue corresponding to position 12 or 13 of SEQ ID No: 1, wherein the Ras mutant protein is a human KRAS G12C protein. In some embodiments, the modified Ras mutant protein comprises a compound described herein covalently bonded to a protein of SEQ ID No. 4. In some embodiments, the modified Ras mutant protein comprises a compound described herein covalently bonded to a protein of SEQ ID No. 9. In some embodiments, the modified Ras mutant protein comprises a compound described herein covalently bonded to the serine residue at position 12 of SEQ ID No. 4. In some embodiments, the modified Ras mutant protein comprises a compound described herein covalently bonded to the cysteine residue at position 12 of SEQ ID No. 9. In some embodiments, the modified Ras mutant protein comprises a compound described herein covalently bonded to the amino acid residue corresponding to position 12 or 13 of SEQ ID No: 5, wherein the Ras mutant protein is a mammalian Ras protein (including human protein) selected from NRAS G12C, NRAS G12S, NRAS G13C, and NRAS G13S. In some embodiments, the modified Ras mutant protein comprises a compound described herein covalently bonded to the amino acid residue corresponding to position 12 or 13 of SEQ ID No: 7, wherein the Ras mutant protein is a mammalian protein (including human protein) selected from HRAS G12C, HRAS G12S, HRAS G13C, and HRAS G13S. It will be understood that a compound described herein may be modified upon covalently binding an amino acid (e.g., mutant amino acid other than G) corresponding to position 12 or 13 of human KRAS (e.g., SEQ ID. No: 1). A subject compound of the present disclosure encompasses a compound described herein immediately prior to covalently bonding the Ras mutant protein as well as the resulting compound covalently bonded to the modified Ras mutant protein.

In some embodiments, the modified Ras mutant protein described herein is formed by contacting a compound described herein with the serine residue of an unmodified Ras G12S mutant protein, wherein the compound comprises a moiety susceptible to reacting with a nucleophilic serine residue corresponding to position 12 of SEQ ID No: 4. In some embodiments, the compound comprises a staying group and a leaving group, wherein said contacting results in release of the leaving group and formation of said modified protein. In some embodiments, the compound selectively labels the serine residue corresponding to position 12 of SEQ ID No. 4 (a G12S mutant) relative to a valine (G12V) residue or glycine residue (wildtype KRAS) at the same position. In some embodiments, the compound selectively labels the serine residue as compared to (i) an aspartate residue of a K-Ras G12D mutant protein, said aspartate corresponding to residue 12 of SEQ ID NO: 2, and/or (ii) a valine residue of a K-Ras G12V mutant protein, said valine corresponding to residue 12 of SEQ ID NO: 3, by at least 1, 2, 3, 4, 5, or 10 fold or more, when assayed under comparable conditions.

In some embodiments, the modified Ras mutant protein described herein is formed by contacting a compound described herein with the cysteine residue of an unmodified Ras G12C mutant protein, wherein the compound comprises a moiety susceptible to reacting with a nucleophilic cysteine residue corresponding to position 12 of SEQ ID No: 9. In some embodiments, the compound comprises a staying group and a leaving group, wherein said contacting results in release of the leaving group and formation of said modified protein. In some embodiments, the compound selectively labels the cysteine residue corresponding to position 12 of SEQ ID No. 9 (a G12C mutant) relative to a valine (G12V) residue or glycine residue (wildtype KRAS) at the same position. In some embodiments, the compound selectively labels the cysteine residue as compared to (i) an aspartate residue of a K-Ras G12D mutant protein, said aspartate corresponding to residue 12 of SEQ ID NO: 2, and/or (ii) a valine residue of a K-Ras G12V mutant protein, said valine corresponding to residue 12 of SEQ ID NO: 3, by at least 1, 2, 3, 4, 5, or 10 fold or more, when assayed under comparable conditions.

In some embodiments of the modified Ras mutant protein described herein, the compound covalently binds to the serine residue of an unmodified Ras G12S protein corresponding to position 12 of SEQ ID No: 4 in vitro. In some embodiments of the modified Ras mutant protein described herein, the compound covalently binds to the serine residue of an unmodified K-Ras G12S protein corresponding to position 12 of SEQ ID No: 4 in vivo. In some embodiments of the modified Ras mutant protein described herein, the compound covalently binds to the cysteine residue of an unmodified Ras G12C protein corresponding to position 12 of SEQ ID No: 9 in vitro. In some embodiments of the modified Ras mutant protein described herein, the compound covalently binds to the cysteine residue of an unmodified K-Ras G12C protein corresponding to position 12 of SEQ ID No: 9 in vivo. In some embodiments of the modified Ras mutant protein described herein, the compound covalently binds to both the serine residue and the cysteine residue of an unmodified K-Ras G12S and K-Ras G12C protein, respectively, at position 12 of the respective protein in vitro or in vivo.

In an aspect is provided a method of treating cancer in a subject comprising a Ras mutant protein (e.g., KRAS G12D, KRAS G12C, KRAS G12S, KRAS G12V, KRAS G13D, KRAS G13C, NRAS G12D, NRAS G12C, NRAS G12S, NRAS G13D, NRAS G13C, HRAS G12D, HRAS G12C, HRAS G12S, HRAS G13D, or HRAS G13C), the method comprising modifying the Ras mutant protein of said subject by administering to said subject a compound described herein, wherein the compound is characterized in that upon contacting a Ras mutant protein, said Ras mutant protein is modified covalently at a residue corresponding to residue 12 or 13 of SEQ ID No: 1, such that said modified Ras mutant protein exhibits reduced Ras signaling output (e.g., compared to a control, such as an unmodified Ras mutant protein not covalently bonded with any compound such as a compound disclosed herein).

In some aspects, a subject compound exhibits one or more of the following characteristics: it is capable of reacting with a mutant residue (e.g., KRAS G12D, KRAS G12C, KRAS G12S, KRAS G12V, KRAS G13D, KRAS G13C, NRAS G12D, NRAS G12C, NRAS G12S, NRAS G13D, NRAS G13C, HRAS G12D, HRAS G12C, HRAS G12S, HRAS G13D, or HRAS G13C) of a Ras mutant protein and covalently modifying such Ras mutant and/or it comprises a moiety susceptible to reacting with a nucleophilic amino acid residue corresponding to position 12 or 13 of SEQ ID No: 1. In some embodiments, a subject compound, when used to modify a Ras mutant protein, reduces the signaling output of the Ras protein. In some embodiments, a subject compound exhibits an IC50 against a mutant Ras as ascertained by reduction of Ras::SOS1 interaction of less than 10 μM, such as less than 5 μM, 1 μM, 500 nM, 100 nM, 50 nM, 10 nM, 5 nM, 1 nM, 500 μM, 50 μM, 10 μM or less.

In some embodiments, a modified Ras mutant protein disclosed herein exhibits a reduced Ras signaling output. A reduction of signaling output can be ascertained by a wide variety of methods known in the art. For example, phosphorylation of a substrate or a specific amino acid residue thereof can be detected and/or quantified using one or more techniques, such as kinase activity assays, phospho-specific antibodies, Western blot, enzyme-linked immunosorbent assays (ELISA), cell-based ELISA, intracellular flow cytometry, mass spectrometry, and multi-analyte profiling. A host of readout can evidence a reduction of Ras signaling output, including without limitation: (i) an increase in steady state level of GDP-bound modified protein; (ii) a reduction in steady state level of GTP-bound Ras protein; (iii) a reduction of phosphorylated AKTs473, (iv) a reduction of phosphorylated ERK T202/Y204, (v) a reduction of phosphorylated S6 S235/236, (vi) a reduction of cell growth of a tumor cell expressing a Ras mutant protein (e.g., KRAS G12D, KRAS G12C, KRAS G12S, KRAS G12V, KRAS G13D, KRAS G13C, NRAS G12D, NRAS G12C, NRAS G12S, NRAS G13D, NRAS G13C, HRAS G12D, HRAS G12C, HRAS G12S, HRAS G13D, or HRAS G13C), and (vii) a reduction in Ras interaction with a Ras-pathway signaling protein. In some embodiments, a reduction is evidenced by 2, 3, 4 or more of items (i)-(vii). In some embodiments, the reduction in Ras signaling output can be evidenced by any one of (i)-(vii) as compared to control unmodified corresponding Ras protein that is not covalently bonded to any compound disclosed herein. For example, a control Ras protein, as described herein, can be a Ras protein (e.g., wildtype or mutated) that is not complexed with any subject compound of the present disclosure. The increase in item (i) or reduction in items (ii) through (vi) can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the control Ras protein. In some embodiments, a reduction in Ras interaction with a Ras-pathway signaling protein is established by a reduced interaction with SOS (including SOS1 and SOS2), RAF, SHC, SHP (including SHP1 and SHP2), MEK, MAPK, ERK, GRB, RASA1, or GNAQ.

Signaling output measured in terms of IC50 values can be obtained and a ratio of IC50 against one mutant relative to another mutant can be calculated. For instance, a selective reduction of K-Ras G12S or K-Ras G12C signaling output can be evidenced by a ratio greater than one. In particular, a selective reduction of K-Ras G12S or K-Ras G12C signaling relative to K-Ras G12D signaling or wildtype K-Ras signaling is evidenced if the ratio of IC50 (against K-Ras G12D or wildtype) to IC50 (against K-Ras G12S or K-Ras G12C) is greater than 1.

It will be understood that when a compound described herein selectively labels the serine and/or cysteine residue of a K-Ras G12S or K-Ras G12C protein compared to another K-Ras protein(s) (e.g., WT, G12D, or G12V), the compound labels the K-Ras G12S or K-Ras G12C protein with greater speed or to a greater degree or by any other quantifiable measurement compared to the other K-Ras protein (e.g., WT, G12D, G12V), under similar or identical reaction conditions for the proteins being compared. In some embodiments, the greater labeling of K-Ras G12S and/or K-Ras G12C can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to another K-Ras protein (e.g., WT, G12D, or G12V).

In some embodiments, the compounds described herein, or a pharmaceutically acceptable salt or solvate thereof, are Ras modulators (including Ras inhibitors) capable of covalently modifying a Ras protein. Ras proteins being modified can be Ras G12S mutants or G12C mutants from K-Ras, H-Ras or N-Ras. The compounds disclosed herein, or pharmaceutically acceptable salts or solvates thereof, have a wide range of applications in therapeutics, diagnostics, and other biomedical research.

In an aspect is provided a method of treating cancer in a subject comprising a Ras G12S mutant protein, comprising modifying the Ras G12S mutant protein of said subject by administering to said subject a compound described herein, wherein said compound is characterized in that upon contacting the Ras G12S mutant protein, the Ras G12S mutant protein is modified covalently at a serine residue corresponding to residue 12 of SEQ ID No: 4, such that said modified K-Ras G12S protein exhibits reduced Ras signaling output (e.g., compared to a corresponding unmodified Ras protein unbound to the covalent compound).

In an aspect is provided a method of treating cancer in a subject comprising a Ras G12C mutant protein, comprising modifying the Ras G12C mutant protein of said subject by administering to said subject a compound described herein, wherein said compound is characterized in that upon contacting the Ras G12C mutant protein, the Ras G12C mutant protein is modified covalently at a cysteine residue corresponding to residue 12 of SEQ ID No: 9, such that said modified K-Ras G12C protein exhibits reduced Ras signaling output (e.g., compared to a corresponding unmodified Ras protein unbound to the covalent compound).

In an aspect is provided a method of modulating activity of a Ras protein (e.g., K-Ras, mutant K-Ras, K-Ras G12S, K-Ras G12C), comprising contacting a Ras protein with an effective amount of a compound described herein, or a pharmaceutically acceptable salt or solvate thereof, thereby modulating the activity of the Ras protein.

In practicing any of the methods disclosed herein, the Ras target to which a subject compound binds covalently can be a Ras mutant (e.g., KRAS G12D, KRAS G12C, KRAS G12S, KRAS G12V, KRAS G13D, KRAS G13C, NRAS G12D, NRAS G12C, NRAS G12S, NRAS G13D, NRAS G13C, HRAS G12D, HRAS G12C, HRAS G12S, HRAS G13D, or HRAS G13C).

### Pharmaceutical Compositions and Methods of Administration

In an aspect is provided a pharmaceutical composition comprising a compound described herein, or a pharmaceutically acceptable salt or solvate thereof, and a pharmaceutically acceptable excipient.

In some embodiments, a compound described herein, or a pharmaceutically acceptable salt or solvate thereof, is administered to a subject in a biologically compatible form suitable for administration to treat or prevent diseases, disorders, or conditions. Administration of a compound described herein can be in any pharmacological form including a therapeutically effective amount of a compound described herein, or a pharmaceutically acceptable salt or solvate thereof, alone or in combination with a pharmaceutically acceptable carrier.

In some embodiments, a compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments of the methods described herein, a compound described herein is administered either alone or in combination with pharmaceutically acceptable carriers, excipients or diluents, in a pharmaceutical composition. Administration of a compound or composition described herein can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, a compound described herein can be administered locally to the area in need of treatment, by, for example, local infusion during surgery, topical application such as creams or ointments, injection, catheter, or implant. The administration can also be by direct injection at the site of a diseased tissue or organ. In some embodiments, a compound described herein, or a pharmaceutically acceptable salt or solvate thereof, is administered orally.

In some embodiments of the methods described herein, a pharmaceutical composition suitable for oral administration is presented as a discrete unit such as a capsule, cachet or tablet, each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary, or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compound which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### Embodiments of the Disclosure

Embodiment 1. A compound of Formula (III):

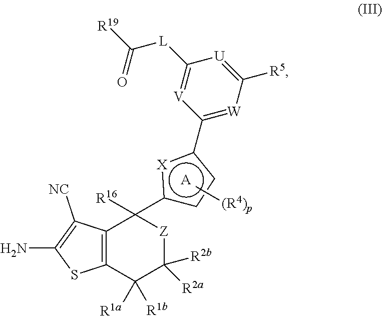

or a pharmaceutically acceptable salt or solvate thereof, wherein:
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
Z is —(CR^{6a}R^{6b})ₙ—;
X is ═C(R¹⁵)— or —N(R¹⁵)—;
R¹⁵ and R¹⁶, together with the atoms to which they are attached, form C₅₋₇ cycloalkyl or 5- to 7-membered heterocycloalkyl, each of which is optionally substituted with one or more substituents independently selected from C₁₋₄ alkyl, C₁₋₄ haloalkyl, and halogen;
W is —N═ or —CH═;
V is —N═ or —CH═;
U is —N═ or —C(R¹¹)═;
ring A is selected from pyrrole, furan, thiophene, imidazole, pyrazole, oxazole, isoxazole, thiazole, isothiazole, and triazole;
n is 0, 1, or 2;
p is 0, 1, 2, or 3;
R^{1a}, R^{1b}, R^{2a}, R^{2b}, R^{6a}, and R^{6b} are independently selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; optionally wherein one of R^{1a} or R^{1b} and one of R^{2a} or R^{2b}, together with the carbon atoms to which they are attached form a cyclopropane ring; and optionally wherein R^{6a} and R^{6b}, together with the carbon atom to which they are attached, form a cyclopropane ring;
R⁴ is selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁵ is selected from halogen, —OC₁₋₆ alkyl substituted with optionally substituted 3- to 12-membered heterocycle, and optionally substituted 3- to 12-membered heterocycle;
R¹¹ is selected from hydrogen, halogen, and C₁₋₄ alkoxy;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

Embodiment 2. The compound of embodiment 1, having the structure of Formula (IV):

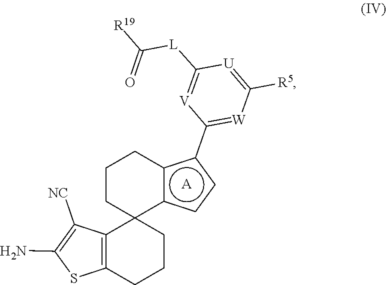

or a pharmaceutically acceptable salt or solvate thereof.

Embodiment 3. The compound, salt, or solvate of embodiment 1 or 2, wherein ring A is selected from pyrazole, isoxazole, and 1,2,3-triazole.

Embodiment 4. The compound of embodiment 1, having the structure:

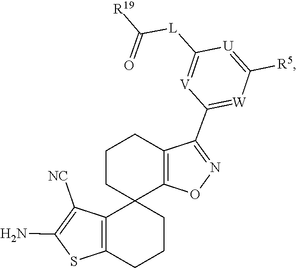

or a pharmaceutically acceptable salt or solvate thereof.

Embodiment 5. The compound, salt, or solvate of any one of embodiments 1 to 4, wherein W is —N═, V is —CH═, and U is —N═.

Embodiment 6. The compound, salt, or solvate of any one of embodiments 1 to 4, wherein W is —N═, V is —N═, and U is —CH═.

Embodiment 7. The compound, salt, or solvate of any one of embodiments 1 to 6, wherein R⁵ is selected from —OC₁₋₃ alkyl substituted with optionally substituted 5- to 10-membered heterocycle, and optionally substituted 5- to 10-membered heterocycle.

Embodiment 8. The compound, salt, or solvate of any one of embodiments 1 to 7, wherein R⁵ is selected from

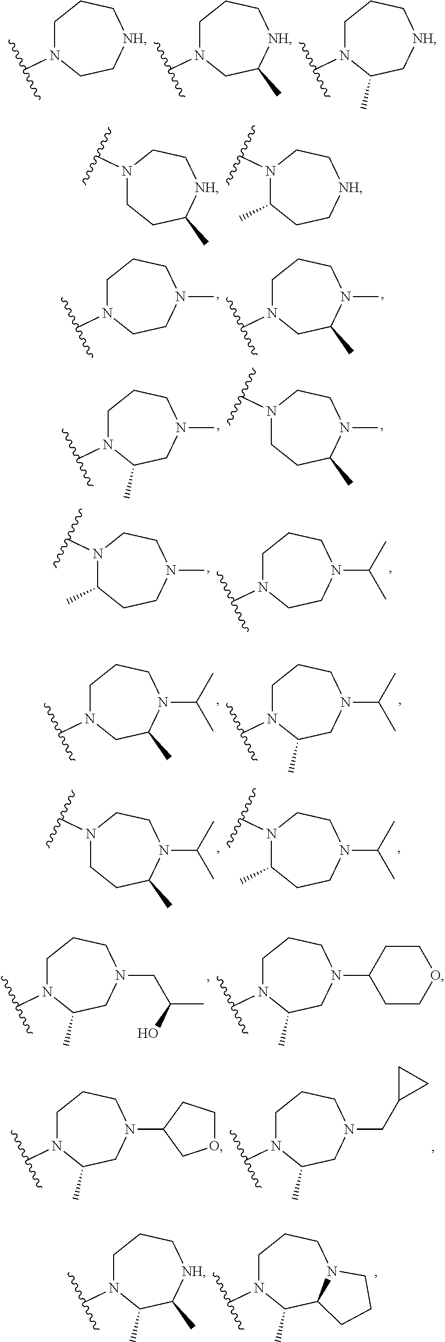

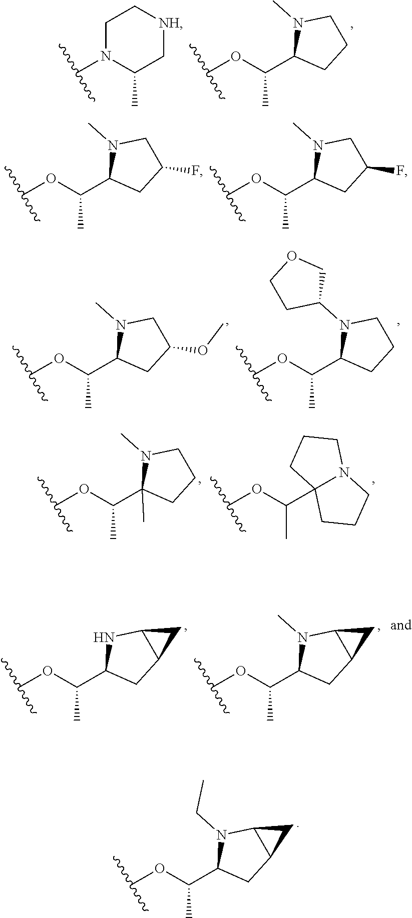

Embodiment 9. The compound of embodiment 1, having the structure:

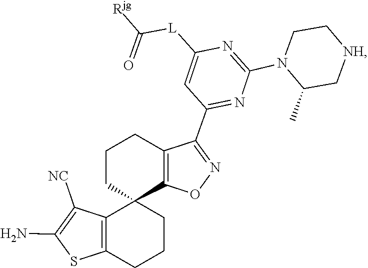

or a pharmaceutically acceptable salt or solvate thereof.

Embodiment 10. A compound of Formula (V):

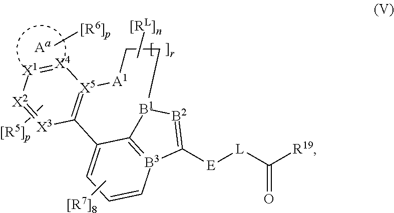

or a pharmaceutically acceptable salt or solvate thereof, wherein:
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
E is selected from a bond and optionally substituted —C(O)-phen-1,4-diyl-;
one of B¹ and B³ is C and the other is N;
B² is N or C(R^{B}), wherein R^{B} is selected from hydrogen, C₁₋₆ alkyl, C₂₋₆ alkenyl, C₂₋₆ alkynyl, and C₃₋₆ cycloalkyl;
ring A^{a} is absent or 5- to 6-membered heteroaryl containing 1 to 3 heteroatoms selected from N, O, and S;
X¹, X², X³, X⁴, and X⁵ are independently selected from N and C, with the proviso that no more than two of X¹, X², X³, X⁴, and X⁵ are N;
R⁵, R⁶, R⁷, and R^{L} are independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and optionally wherein two geminally substituted R^{L} substituents together with the carbon atom to which they are attached form an optionally substituted 3- to 6-membered spirocyclic ring containing 0 to 1 heteroatoms selected from N, O, and S;
A¹ is selected from —CH₂—, —CH₂N(R^{L})—, —C(O)N(R^{L})—, —CH₂O—, —CH(R^{L})—, —NH—, —N(R^{L})—, —S—, —S(O)—, and —O—;
n, q, r, and s are independently 0, 1, 2, or 3;
p is 0, 1, 2, 3, or 4;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 11. The compound of embodiment 10, having the structure of Formula (VI):

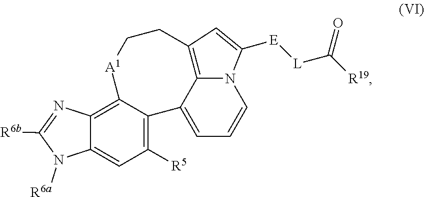

or a pharmaceutically acceptable salt or solvate thereof, wherein:
R^{6a} and R^{6b} are independently selected from hydrogen and R⁶.

Embodiment 12. The compound of embodiment 10, having the structure:

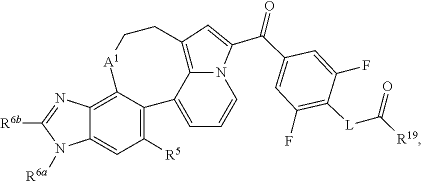

or a pharmaceutically acceptable salt or solvate thereof, wherein:
R^{6a} and R^{6b} are independently selected from hydrogen and R⁶.

Embodiment 13. The compound, salt, or solvate of any one of embodiments 10 to 12, wherein A¹ is selected from —N(R^{L})—, —S—, —S(O)—, and —O—.

Embodiment 14. The compound, salt, or solvate of any one of embodiments 10 to 13, wherein A¹ is selected from —N(CH₃)—, —S—, and —O—.

Embodiment 15. The compound, salt, or solvate of any one of embodiments 10 to 14, wherein R⁵ is selected from —CH₃, —CF₃, —OCH₃, and —OCHF₂.

Embodiment 16. The compound, salt, or solvate of any one of embodiments 10 to 15, wherein R⁵ is selected from —CF₃ and —OCH₃.

Embodiment 17. The compound, salt, or solvate of any one of embodiments 10 to 16, wherein R⁶ is selected from —CH₃, —CH₂OCH₃, and —CH₂CH₂OCH₃.

Embodiment 18. The compound, salt, or solvate of any one of embodiments 11 to 17, wherein Ra is selected from hydrogen, —CH₃, and —CH₂CH₂OCH₃; and R^{6b} is selected from hydrogen, —CH₃, and —CH₂OCH₃.

Embodiment 19. The compound, salt, or solvate of any one of embodiments 11 to 17, wherein R^{6a} is —CH₃ and R^{6b} is hydrogen.

Embodiment 20. The compound of embodiment 10, having the structure:

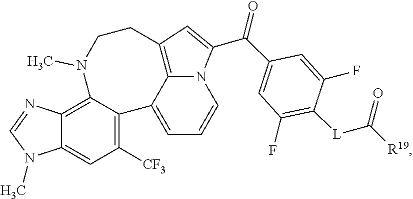

or a pharmaceutically acceptable salt or solvate thereof.

Embodiment 21. A compound of Formula (VII):

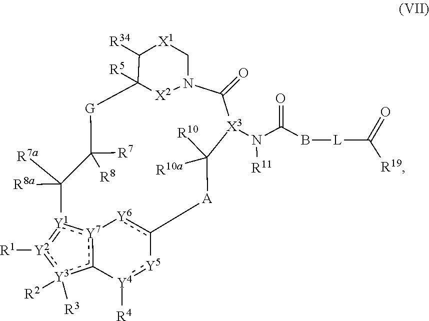

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from 2- to 6-membered heteroalkyl, C₃₋₁₂ carbocycle, and 3- to 12-membered heterocycle, each of which is optionally substituted;
B is selected from a bond, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted;
G is selected from C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)O—CH(R⁶)—, —C(O)NHCH(R⁶)—, C₃₋₁₂ carbocycle, and 3- to 12-membered heterocycle, wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, C₃-12 carbocycle, and 3- to 12-membered heterocycle are optionally substituted;
X¹ is selected from NR⁹, O, S(O), S(O)₂, and optionally substituted C₁₋₂ alkylene;
X² is selected from NH and O;
X³ is selected from N and CH;
Y¹ is selected from C, CH, and N;
Y², Y³, Y⁴, and Y⁷ are independently selected from C and N;
Y⁵ is selected from CH, CH₂, and N;
Y⁶ is selected from C(O), CH, CH₂, and N;
R¹ is selected from —CN, C₁₋₆ alkyl, 2- to 6-membered heteroalkyl, C₃₋₁₂ carbocycle, and 3- to 12-membered heterocycle, wherein C₁₋₆ alkyl, 2- to 6-membered heteroalkyl, C₃₋₁₂ carbocycle, and 3- to 12-membered heterocycle are optionally substituted; or R¹ and R², together with the atoms to which they are attached, form optionally substituted 3- to 12-membered heterocycle;
R² is absent or is selected from hydrogen, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; R³ is absent; or
R² and R³, together with the atom to which they are attached, form C₃₋₁₂ carbocycle or 3- to 12-membered heterocycle, each of which is optionally substituted;
R⁴ is absent or is selected from hydrogen, halogen, —CN, C₁₋₃ alkyl, and C₁₋₃ haloalkyl;
R⁵ is selected from hydrogen, C₁₋₆ alkyl, and C₃₋₆ carbocycle, wherein C₁₋₆ alkyl and C₃₋₆ carbocycle are optionally substituted;
R⁶ is selected from hydrogen and methyl; R⁷ is selected from hydrogen, halogen, and optionally substituted C₁₋₃ alkyl; or
R⁶ and R⁷, together with the carbon atoms to which they are attached, form C₃₋₆ carbocycle or 3- to 7-membered heterocycle, each of which is optionally substituted;
R⁸ is selected from hydrogen, halogen, —OR¹², —CN, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; or
R⁷ and R⁸, together with the carbon atom to which they are attached, form C═CR^{7′}R^{8′}, C═N(OH), C═N(O—C₁₋₃ alkyl), C═O, C═S, C═NH, C₃₋₆ carbocycle, or 3- to 7-membered heterocycle, wherein C₃₋₆ carbocycle and 3- to 7-membered heterocycle are optionally substituted;
R^{7a} and R^{8a} are independently selected from hydrogen, halogen, and optionally substituted C₁₋₆ alkyl; or R^{7a} and R^{8a}, together with the carbon atom to which they are attached, form C═O;
R^{7′} is selected from hydrogen, halogen, and optionally substituted C₁₋₆ alkyl;
R^{8′} is selected from hydrogen, halogen, —OR¹², —CN, C₁₋₆ alkyl, C₂₋₆ alkenyl, C₂₋₆ alkynyl, C₃₋₁₂ carbocycle, and 3- to 12-membered heterocycle, wherein C₁₋₆ alkyl, C₂₋₆ alkenyl, C₂₋₆ alkynyl, C₃₋₁₂ carbocycle, and 3- to 12-membered heterocycle are optionally substituted; or
R^{7′} and R^{8′}, together with the carbon atom to which they are attached, form C₃₋₆ carbocycle or 3- to 7-membered heterocycle, each of which is optionally substituted;
R⁹ is selected from hydrogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹⁰ is selected from hydrogen, halogen, —OR¹², and optionally substituted C₁₋₃ alkyl;
R^{10a} is selected from hydrogen and halogen;
R¹¹ is selected from hydrogen and optionally substituted C₁₋₃ alkyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle;
R³⁴ is selected from hydrogen and optionally substituted C₁₋₃ alkyl; and
[symbol] indicates a single or double bond such that all valences are satisfied.

Embodiment 22. The compound of embodiment 21, having the structure of Formula (VIII):

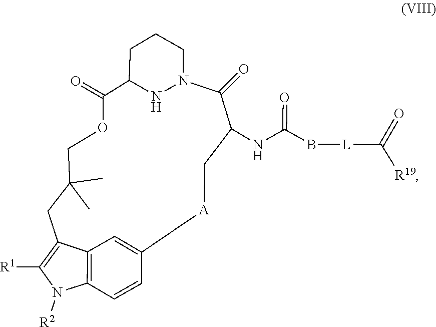

or a pharmaceutically acceptable salt or solvate thereof.

Embodiment 23. The compound, salt, or solvate of embodiment 21 or 22, wherein R¹ is selected from C₆₋₁₀ carbocycle and 6- to 10-membered heterocycle, each of which is optionally substituted.

Embodiment 24. The compound, salt, or solvate of any one of embodiments 21 to 23, wherein R¹ is optionally substituted pyrid-3-yl.

Embodiment 25. The compound, salt, or solvate of any one of embodiments 21 to 24, wherein R¹ is substituted with one or more substituents independently selected from C₁₋₆ alkyl, 2- to 6-membered heteroalkyl, and optionally substituted 3- to 8-membered heterocycle.

Embodiment 26. The compound, salt, or solvate of any one of embodiments 21 to 25, wherein R¹ is substituted with one or more substituents independently selected from —CH(CH₃)OCH₃, —CH₂OCH₃, 4-methylpiperazin-1-yl, and 4-cyclopropylpiperazin-1-yl.

Embodiment 27. The compound, salt, or solvate of any one of embodiments 21 to 26, wherein R¹ is selected from

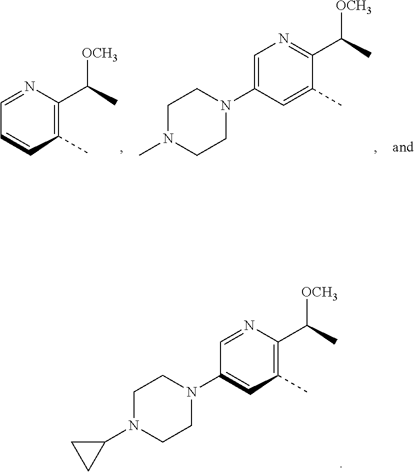

Embodiment 28. The compound, salt, or solvate of any one of embodiments 21 to 27, wherein R² is selected from C₁₋₆ alkyl, C₁₋₆ fluoroalkyl, and C₃₋₆ cycloalkyl.

Embodiment 29. The compound, salt, or solvate of any one of embodiments 21 to 28, wherein R² is selected from —CH₂CH₃ and —CH₂CF₃.

Embodiment 30. The compound, salt, or solvate of any one of embodiments 21 to 29, wherein A is selected from 1,2,3,6-tetrahydropyrid-1,5-diyl, thiazol-2,4-diyl, morpholin-2,4-diyl, 5-hydroxyphen-1,3-diyl, and oxazol-2,5-diyl.

Embodiment 31. The compound, salt, or solvate of any one of embodiments 21 to 30, wherein B is —CHR^{B}—, wherein R^{B} is selected from halogen, C₁₋₆ alkyl, C₁₋₆ haloalkyl, 2- to 6-membered heteroalkyl, C₃₋₆ cycloalkyl, and 3- to 7-membered heterocycloalkyl.

Embodiment 32. The compound, salt, or solvate of any one of embodiments 21 to 31, wherein B is selected from —CH(CH(CH₃)₂)— and —CH(cyclopentyl)-.

Embodiment 33. The compound of embodiment 22, having a structure selected from:

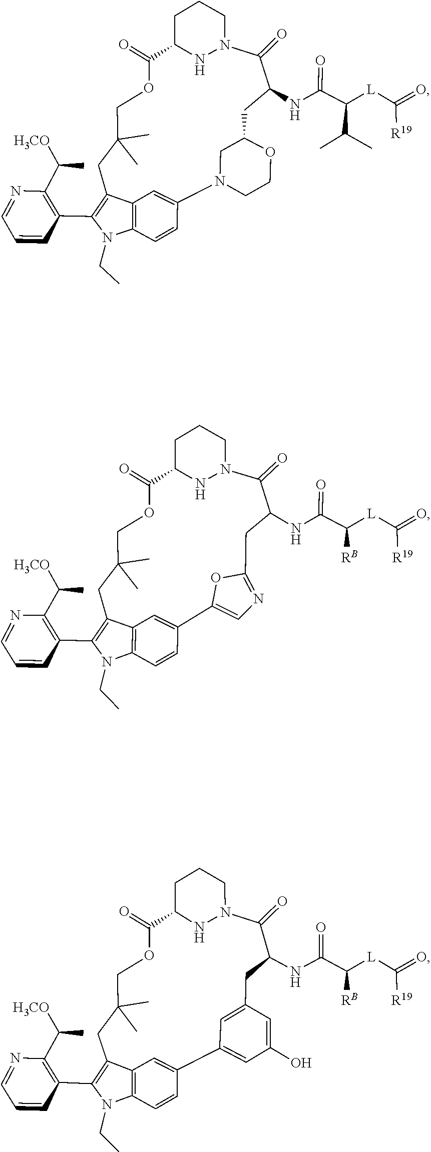

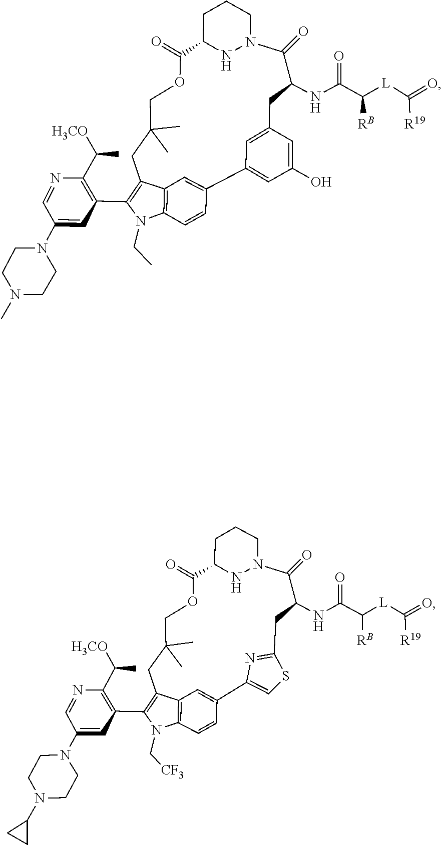

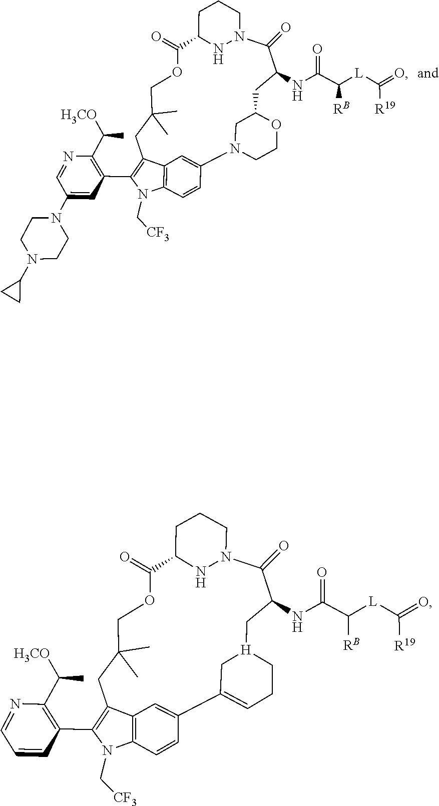

or a pharmaceutically acceptable salt or solvate thereof, wherein:
R^{B} is selected from —CH(CH₃)₂ and cyclopentyl.

Embodiment 34. A compound of Formula (XI):

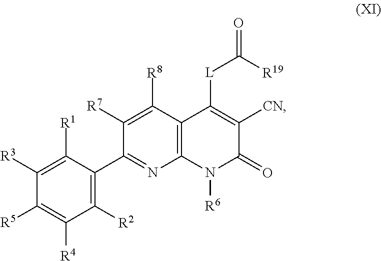

or a pharmaceutically acceptable salt or solvate thereof, wherein:
L is -L¹-L²-L³-, wherein L² or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R¹, R², R³, R, R⁵, R⁷, and R⁸ are independently selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁶ is selected from C₁₋₁₄ alkyl, C₂₋₁₄ alkenyl, C₂₋₁₄ alkynyl, C₃₋₁₂ carbocycle, and 3- to 12-membered heterocycle, each of which is optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

Embodiment 35. The compound, salt, or solvate of embodiment 34, wherein R¹, R², R³, R⁴, and R⁵ are independently selected from halogen, —NH₂, —OH, and —CF₃.

Embodiment 36. The compound, salt, or solvate of embodiment 34 or 35, wherein R¹ is —NH₂ and R², R³, R⁴, and R⁵ are each —F.

Embodiment 37. The compound, salt, or solvate of any one of embodiments 34 to 36, wherein R⁷ is —Cl and R⁸ is hydrogen.

Embodiment 38. The compound of embodiment 34, having the structure of Formula (XII):

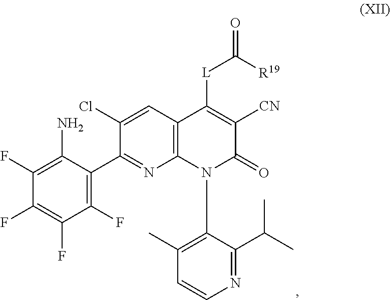

or a pharmaceutically acceptable salt or solvate thereof.

Embodiment 39. A compound, salt, or solvate of Formula (XIII):

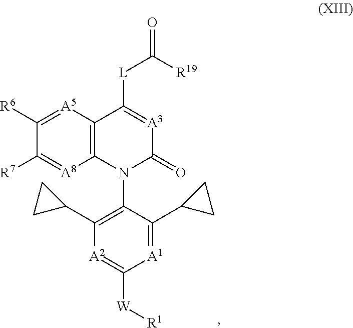

or a pharmaceutically acceptable salt or solvate thereof, wherein:
W is O, NR^{1a}, S, SO₂, or an optionally substituted 4- to 7-membered heterocycle;
R¹ is hydrogen, optionally substituted C₁₋₄ alkyl, or -L^{1b}-R^{1b},
L^{1b} is absent or an optionally substituted C₁₋₄ alkylene, optionally substituted 2- to 4-membered heteroalkylene, optionally substituted C₃₋₆ carbocycle, or optionally substituted 4- to 7-membered heterocycle,
R^{1b} is hydrogen, optionally substituted C₁₋₄ alkyl, optionally substituted C₂₋₄ alkenyl, optionally substituted C₂₋₄ alkynyl, —NR²¹R²², —OR²³, an optionally substituted 4 to 7 membered heterocycle, or
W—R¹ is hydrogen, —COOH, —COOR^{1e}, —CONR^{1e}R^{1d}, —CN, C₁₋₆ alkyl, C₂₋₆ alkenyl, C₂₋₆ alkynyl, or C₃₋₆ carbocycle, wherein the C₁-6 alkyl, C₂₋₆ alkenyl, C₂₋₆ alkynyl, or C₃₋₆ carbocycle is optionally substituted,
each of R^{1a}, R^{1c} and R^{1d} at each occurrence is independently hydrogen, an optionally substituted C₁₋₄ alkyl, optionally substituted C₂₋₄ alkenyl, optionally substituted C₂₋₄ alkynyl, optionally substituted 2- to 4-membered heteroalkyl, optionally substituted C₃₋₆ carbocycle, optionally substituted 4- to 7-membered heterocycle, or a nitrogen protecting group;
R^{1e} at each occurrence is independently hydrogen, an optionally substituted C₁₋₄ alkyl, optionally substituted C₂₋₄ alkenyl, optionally substituted C₂₋₄ alkynyl, optionally substituted 2- to 4-membered heteroalkyl, optionally substituted C₃₋₆ carbocycle, optionally substituted 4- to 7-membered heterocycle, or an oxygen protecting group;
each of A¹, A², A³, A⁵, and A⁸ is independently CR³⁰ or N,
R³⁰ at each occurrence is independently hydrogen, —F, —Cl, C₁₋₄ alkyl, or C₁₋₄ alkoxyl; or
R¹, W, and A¹ together form an optionally substituted heterocycle;
L is -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —O—, —N(R¹²)—, —C(O)—, —S—, —S(O)—, —S(O)₂—, —P(O)R¹²—, —P(O)R¹²O—, —N(R¹²)C(O)—, —N(R¹²)S(O)—, —N(R¹²)S(O)₂—, —N(R¹²)P(O)R¹²—, —OP(O)R¹²—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₅ carbocycle and 3- to 15-membered heterocycle, wherein C₃₋₁₅ carbocycle and 3- to 15-membered heterocycle are optionally substituted;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R⁶ is hydrogen, halogen, CN, a C₃₋₄ carbocycle, optionally substituted C₁₋₄ alkyl, optionally substituted C₂₋₄ alkenyl, optionally substituted C₂₋₄ alkynyl, or optionally substituted C₁₋₄ alkoxyl;
R⁷ is selected from optionally substituted C₃₋₁₂ carbocycle and optionally substituted 3 to 12-membered heterocycle;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle.

Embodiment 40. The compound, salt, or solvate of embodiment 39, wherein A¹, A², A³, and A⁸ are each N.

Embodiment 41. The compound, salt, or solvate of embodiment 39 or 40, wherein A⁵ is CH.

Embodiment 42. The compound, salt, or solvate of any one of embodiments 39 to 41, wherein R⁶ is —F and R⁷ is optionally substituted phenyl.

Embodiment 43. The compound of embodiment 39, having the structure of Formula (XIV):

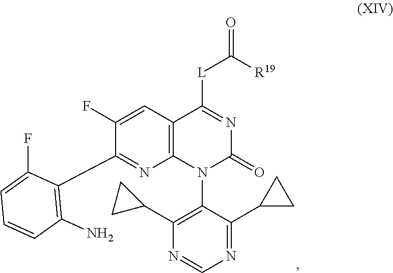

or a pharmaceutically acceptable salt or solvate thereof.

Embodiment 44. The compound, salt, or solvate of any one of embodiments 1 to 43, wherein:
L¹ is selected from a bond, C₁₋₆ alkyl, 2- to 6-membered heteroalkyl, —N(R¹²)—, —C(O)—, —N(R¹²)C(O)—, and —C(O)N(R¹²)—, wherein C₁₋₆ alkyl and 2- to 6-membered heteroalkyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; and
L³ is selected from a bond, 2- to 6-membered heteroalkyl, and nitrene, wherein 2- to 6-membered heteroalkyl and nitrene are optionally substituted.

Embodiment 45. The compound, salt, or solvate of any one of embodiments 1 to 44, wherein:
L² is optionally substituted 3- to 12-membered heterocycle; and
L³ is a bond.

Embodiment 46. The compound, salt, or solvate of any one of embodiments 1 to 44, wherein:
L² is selected from C₃₋₁₂ carbocycle and 3- to 12-membered heterocycle, each of which is optionally substituted; and
L³ is optionally substituted nitrene.

Embodiment 47. The compound, salt, or solvate of any one of embodiments 1 to 46, wherein L² is optionally substituted 6- to 12-membered spirocyclic heterocycle.

Embodiment 48. The compound, salt, or solvate of any one of embodiments 1 to 44, wherein -L²-L³-C(O)R¹⁹ is selected from:

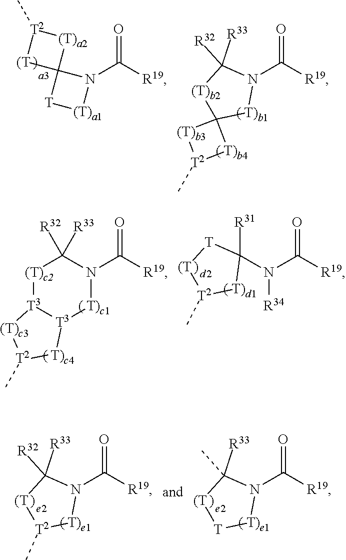

wherein:
a1, b1, b3, and b4 are independently 1, 2, 3, 4, or 5;
a2, a3, and b2 are independently 0, 1, 2, 3, 4, or 5;
c1, c2, c3, c4, d1, d2, e1, and e2 are independently 0, 1, 2, 3, or 4;
wherein the sum of a1, a2, and a3 is less than 9; the sum of b1, b2, b3, and b4 is less than 9; the sum of c1, c2, c3, and c4 is less than 8; the sum of d1 and d2 is less than 6; and the sum of e1 and e2 is less than 6;
T is independently selected at each occurrence from N(R³⁵), C(R³⁶)₂, C(O), O, S(O), and S(O)₂;
T² and T³ are independently selected at each occurrence from N and C(R³⁶);
R³¹, R³², R³³, and R³⁶ are independently selected at each occurrence from hydrogen and R⁴⁰;
R³⁴ and R³⁵ are independently selected at each occurrence from hydrogen and R⁴¹;
R⁴⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R⁴⁰ attached to the same carbon atom optionally join to form ═NR¹², ═C(R¹⁴)₂, or ═O; wherein two R⁴⁰ and the atom(s) to which they are attached optionally form C₃₋₁₂ carbocycle or 3- to 12-membered heterocycle; wherein R⁴⁰ and R⁴¹ and the atoms to which they are attached optionally form 3- to 12-membered heterocycle; and wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁴¹ is independently selected at each occurrence from —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 49. The compound, salt, or solvate of embodiment 48, wherein R⁴⁰ is independently selected at each occurrence from halogen, —CN, C₁₋₆ alkyl, and C₃₋₆ cycloalkyl, or two R⁴⁰ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl).

Embodiment 50. The compound, salt, or solvate of embodiment 48 or 49, wherein R⁴¹ is independently selected at each occurrence from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

Embodiment 51. The compound of embodiment 34, having the structure:

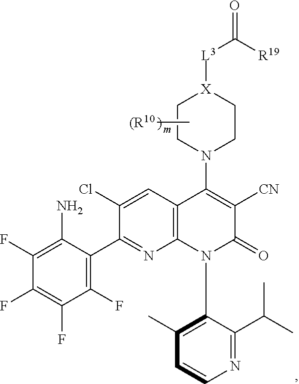

or a pharmaceutically acceptable salt or solvate thereof, wherein:
X is selected from CH and N;
R¹⁰ is selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
m is 0, 1, 2, or 3.

Embodiment 52. The compound, salt, or solvate of embodiment 51, wherein R¹⁰ is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2.

Embodiment 53. The compound, salt, or solvate of embodiment 51, wherein R¹⁰ is selected from —CH₃, and —CH₂CN; and m is 1 or 2.

Embodiment 54. The compound, salt, or solvate of embodiment 51, wherein m is 0.

Embodiment 55. The compound, salt, or solvate of any one of embodiments 51 to 54, wherein X is N and L³ is a bond.

Embodiment 56. The compound, salt, or solvate of any one of embodiments 51 to 54, wherein X is CH and L³ is optionally substituted nitrene.

Embodiment 57. The compound of embodiment 39, having the structure:

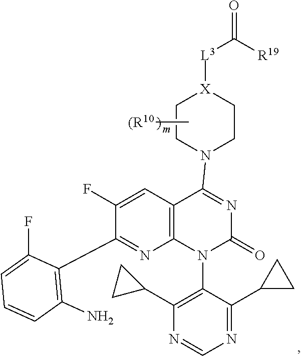

or a pharmaceutically acceptable salt or solvate thereof, wherein:
X is selected from CH and N;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 58. The compound, salt, or solvate of embodiment 57, wherein R¹⁰ is independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2.

Embodiment 59. The compound, salt, or solvate of embodiment 57, wherein R¹⁰ is —CH₃.

Embodiment 60. The compound, salt, or solvate of any one of embodiments 57 to 59, wherein m is 2.

Embodiment 61. The compound, salt, or solvate of any one of embodiments 57 to 60, wherein X is N and L³ is a bond.

Embodiment 62. The compound, salt, or solvate of any one of embodiments 57 to 60, wherein X is CH and L³ is optionally substituted nitrene.

Embodiment 63. A compound of Formula (XV):

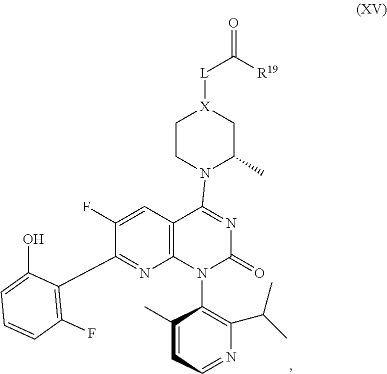

or a pharmaceutically acceptable salt or solvate thereof, wherein:
X is —CH— and L is —N(R¹²)— or X is N and L is a bond;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted; and
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted.

Embodiment 64. The compound, salt, or solvate of embodiment 63, wherein X is —CH— and L is —N(R¹²)—.

Embodiment 65. The compound, salt, or solvate of embodiment 63, X is N and L is a bond.

Embodiment 66. A compound of Formula (XVI):

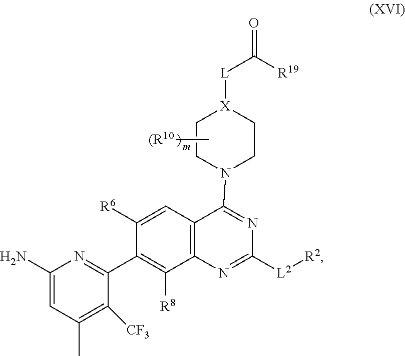

or a pharmaceutically acceptable salt or solvate thereof, wherein:
L is a bond and X is N; or L is —N(R¹²)— and X is CH;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3;
L² is selected from a bond, —O—, —N(R^{2a})—, and —S—;
R^{2a} is selected from hydrogen and C₁₋₃ alkyl, wherein C₁₋₃ alkyl is optionally substituted;
R² is selected from hydrogen, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), halogen, —CN, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁶ and R⁸ are each independently selected from hydrogen, —F, —Cl, C₁₋₄ alkyl, C₁₋₄ haloalkyl, and C₁₋₄ alkoxyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 67. The compound of embodiment 66, having the structure of Formula (XVII):

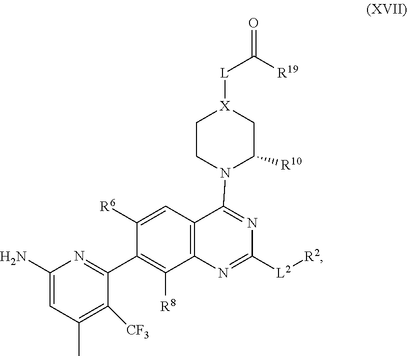

or a pharmaceutically acceptable salt or solvate thereof.

Embodiment 68. The compound, salt, or solvate of embodiment 66 or 67, wherein R⁶ is —Cl and R⁸ is —F.

Embodiment 69. The compound, salt, or solvate of one of embodiments 66 to 68, wherein L² is —O—.

Embodiment 70. The compound, salt, or solvate of any one of embodiments 66 to 69, wherein R² is —C₀₋₆ alkyl-(3- to 12-membered heterocycle), wherein —C₀₋₆ alkyl-(3- to 12-membered heterocycle) is optionally substituted.

Embodiment 71. The compound, salt, or solvate of any one of embodiments 66 to 70, wherein -L²-R² is

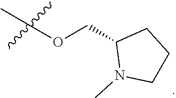 [SMILES: *OC[C@@H]1CCCN1C]

Embodiment 72. The compound, salt, or solvate of any one of embodiments 66 to 71, wherein X is CH and L is —N(R¹²)—.

Embodiment 73. The compound, salt, or solvate of any one of embodiments 66 to 71, wherein X is N and L is a bond.

Embodiment 74. The compound, salt, or solvate of any one of embodiments 66 to 73, wherein R¹⁰ is independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2.

Embodiment 75. The compound, salt, or solvate of any one of embodiments 66 to 74, wherein R¹⁰ is —CH₃.

Embodiment 76. A compound of Formula (IX):

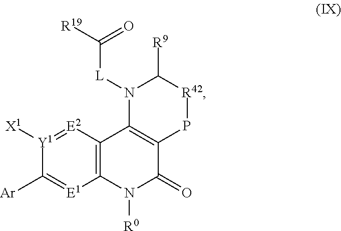

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
P is selected from O, NH, and NR^{m};
R⁴² is selected from —C(O)— and optionally substituted —C₁₋₃ alkyl-;
Y¹ is C and X¹ is selected from hydrogen and R¹⁰; or Y¹ is N and X¹ is absent;
E¹ is selected from N and CR⁵;
E² is selected from N and CR⁶;
provided that Y¹, E¹, and E² are not simultaneously N;
Ar is selected from C₃₋₁₂ carbocycle and 3- to 12-membered heterocycle, each of which is optionally substituted;
R⁰ is selected from C₃₋₁₂ carbocycle and 3- to 12-membered heterocycle, each of which is optionally substituted;
R^{m} is selected from C₁₋₆ alkyl and C₁₋₆ deuteroalkyl, each of which is optionally substituted;
R⁵ and R⁶ are independently selected from hydrogen and R¹⁰;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹⁰ is selected from halogen, —CN, —NO₂, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 77. The compound, salt, or solvate of embodiment 76, wherein:
P is NR^{m};
R⁴² is —C(O)—;
Y¹ is C and X¹ is halogen;
E¹ is N;
E² is CH;
Ar is 3-fluoro-1-hydroxy-phen-2-yl;
R⁰ is selected from optionally substituted 5- to 6-membered heteroaryl; and
R^{m} is selected from CH₃ and CD₃.

Embodiment 78. The compound of embodiment 76 or 77, having the structure of Formula (X):

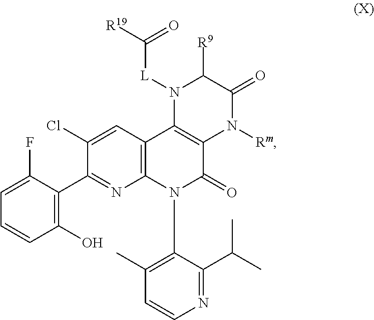

or a pharmaceutically acceptable salt or solvate thereof.

Embodiment 79. The compound of any one of embodiments 76 to 78, having a structure selected from:

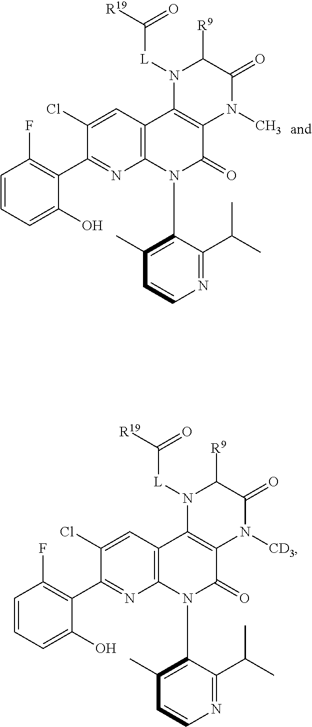

or a pharmaceutically acceptable salt or solvate thereof.

Embodiment 80. A compound of Formula (XIX):

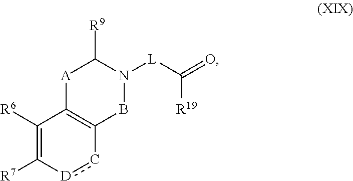

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from —OCH(R⁵)— and —N(R⁵)CH(R⁵)—;
B is selected from —CH₂— and —C(O)—;
C is selected from —C(R³)═, —C(R³)₂—, —N═, —N(R³)—, and C(O)—
D is selected from —C(R⁸)═, —C(R⁸)₂—, —N═, —N(R⁸)—, and C(O)—
wherein -C [symbol] D- is selected from —C(R³)═C(R⁸)—, —C(R³)═N—, —N═C(R⁸)—, —C(O)N(R⁸)—, —C(R³)₂—N(R⁸)—, —N(R³)—C(R⁸)₂—, —C(R³)₂—C(R⁸)₂—, or —N(R³)C(O)—;
R³ and R⁸ are each independently selected from hydrogen, halogen, CN, —OR¹², C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; or R³ and R⁸ are joined to form, together with the atoms to which they are attached, a C₅₋₆ carbocycle or 5- to 6-membered heterocycle, wherein the C₅₋₆ carbocycle and 5- to 6-membered heterocycle are optionally substituted;
R⁵ is independently selected from hydrogen and C₁₋₆ alkyl; wherein C₁₋₆ alkyl is optionally substituted;
R⁶ are each independently selected from hydrogen, halogen, —CN, C₁₋₆ alkyl, C₂₋₆ alkenyl, C₂₋₆ alkynyl, and —O—C₁₋₆ alkyl, wherein C₁₋₆ alkyl, C₂₋₆ alkenyl, C₂₋₆ alkynyl, and —O—C₁₋₆ alkyl are optionally substituted with one or more halogen;
R⁷ is independently selected from C₃₋₁₂ carbocycle, monocyclic 5- to 6-membered heterocycle, bicyclic 9-membered heterocycle comprising two or more ring nitrogen atoms, and 10-membered bicyclic heterocycle; wherein C₃₋₁₂ carbocycle, monocyclic 5- to 6-membered heterocycle, bicyclic 9-membered heterocycle comprising two or more ring nitrogen atoms, and 10-membered bicyclic heterocycle are each optionally substituted;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 81. The compound of embodiment 80, having the structure of Formula (XX):

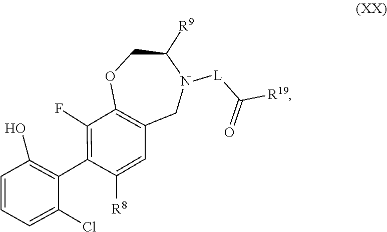

or a pharmaceutically acceptable salt or solvate thereof.

Embodiment 82. A compound of Formula (XXIII):

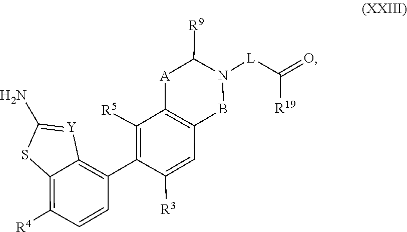

or a pharmaceutically acceptable salt or solvate thereof, wherein:
L is selected from a bond and -L¹-L²-L³-, wherein L¹, L², or L³ is bound to —C(O)R¹⁹ via a nitrogen atom, thereby forming a urea;
L¹ is selected from a bond, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C(O)—, —S(O)—, —S(O)₂—, —P(O)R¹²—, —P(O)R¹²O—, —C(O)N(R¹²)—, —S(O)N(R¹²)—, —S(O)₂N(R¹²)—, and —P(O)R¹²N(R¹²)—, wherein C₁₋₆ alkyl, C₂₋₆ alkenyl, C₂₋₆ alkynyl, 2- to 6-membered heteroalkyl, 3- to 6-membered heteroalkenyl, and 3- to 6-membered heteroalkynyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; or L², R⁹, and the atoms to which they are attached form optionally substituted 3- to 12-membered heterocycle;
L³ is selected from a bond, 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle), wherein 2- to 6-membered heteroalkyl, 3- to 6-membered heteroalkenyl, 3- to 6-membered heteroalkynyl, nitrene, —C₀₋₆ alkyl-(3- to 12-membered heterocycle), and -(2- to 6-membered heteroalkyl)-(3- to 12-membered heterocycle) are optionally substituted;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
A is selected from —OCH₂—, —N(R⁶)CH₂—, —OCH₂CH₂—, —N(R⁶)CH₂CH₂—, —CH₂OCH₂—, and —CH₂N(R⁶)CH₂—;
B is selected from —CH₂— and —C(O)—;
Y is selected from —C(CN)— and —N—;
R³ and R⁵ are each independently selected from hydrogen, halogen, —C₀₋₃ alkyl-cyclopropyl, —C₁₋₆ alkyl, and —O—C₁₋₆ alkyl, wherein —C₁₋₆ alkyl and —O—C₁₋₆ alkyl are optionally substituted with one, two, or three R¹⁰;
R⁴ is selected from hydrogen, halogen, and —C₁₋₆ alkyl optionally substituted with one, two, or three R¹⁰;
R⁶ is selected from hydrogen and C₁₋₆ alkyl optionally substituted with one, two, or three R¹⁰;
R⁹ is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R¹⁰ is independently selected at each occurrence from halogen, ═O, hydroxy, —C₁₋₄ alkyl, and —O—C₁₋₄ alkyl.
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 83. The compound of embodiment 82, having the structure of Formula (XXIV):

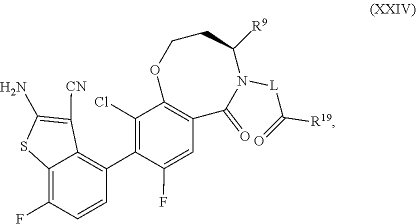

or a pharmaceutically acceptable salt or solvate thereof.

Embodiment 84. The compound of embodiment 82, having a structure selected from:

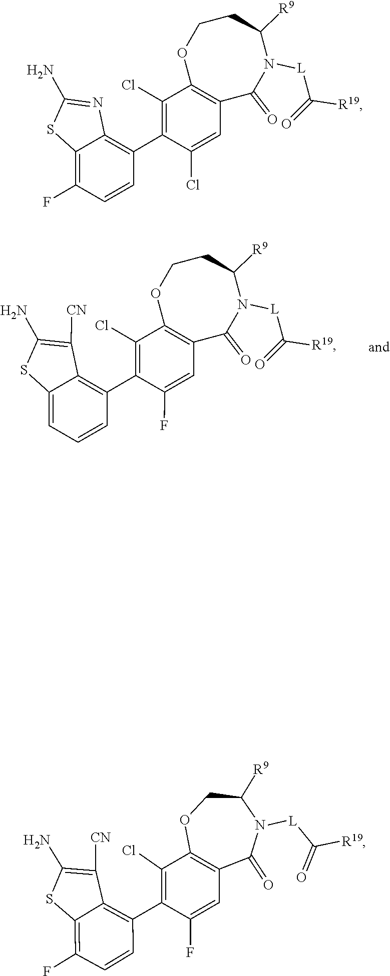

or a pharmaceutically acceptable salt or solvate thereof.

Embodiment 85. The compound, salt, or solvate of any one of embodiments 76 to 84, wherein:
L¹ is selected from a bond, C₁₋₆ alkyl, 2- to 6-membered heteroalkyl, —C(O)—, and —C(O)N(R¹²)—, wherein C₁₋₆ alkyl and 2- to 6-membered heteroalkyl are optionally substituted;
L² is selected from a bond, C₃₋₁₂ carbocycle and 3- to 12-membered heterocycle, wherein C₃₋₁₂ carbocycle and 3- to 12-membered heterocycle are optionally substituted; and
L³ is selected from a bond, 2- to 6-membered heteroalkyl, and nitrene, wherein 2- to 6-membered heteroalkyl and nitrene are optionally substituted.

Embodiment 86. The compound, salt, or solvate of any one of embodiments 76 to 85, wherein:
L² is optionally substituted 3- to 12-membered heterocycle; and
L³ is a bond.

Embodiment 87. The compound, salt, or solvate of any one of embodiments 76 to 85, wherein:
L² is selected from C₃₋₁₂ carbocycle and 3- to 12-membered heterocycle, each of which is optionally substituted; and
L³ is optionally substituted nitrene.

Embodiment 88. The compound, salt, or solvate of any one of embodiments 76 to 87, wherein L¹ is selected from a bond and optionally substituted 2- to 6-membered heteroalkyl.

Embodiment 89. The compound, salt, or solvate of any one of embodiments 76 to 88, wherein L² is optionally substituted 6- to 12-membered spirocyclic heterocycle.

Embodiment 90. The compound, salt, or solvate of any one of embodiments 76 to 85, wherein -L²-L³-C(O)R¹⁹ is selected from:

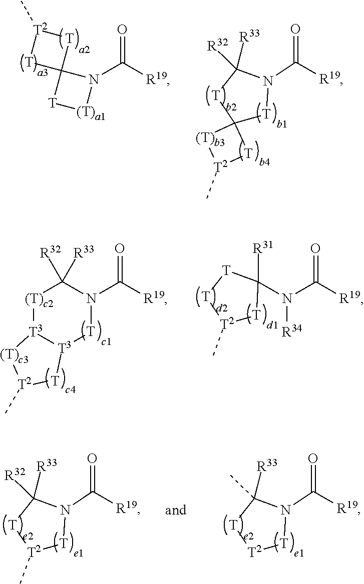

wherein:
a1, b1, b3, and b4 are independently 1, 2, 3, 4, or 5;
a2, a3, and b2 are independently 0, 1, 2, 3, 4, or 5;
c1, c2, c3, c4, d1, d2, e1, and e2 are independently 0, 1, 2, 3, or 4;
wherein the sum of a1, a2, and a3 is less than 9; the sum of b1, b2, b3, and b4 is less than 9; the sum of c1, c2, c3, and c4 is less than 8; the sum of d1 and d2 is less than 6; and the sum of e1 and e2 is less than 6;
T is independently selected at each occurrence from N(R³¹), C(R³⁶)₂, C(O), O, S(O), and S(O)₂;
T² and T³ are independently selected at each occurrence from N and C(R³⁶);
R³¹, R³², R³³, and R³⁶ are independently selected at each occurrence from hydrogen and R⁴⁰;
R³⁴ and R³⁵ are independently selected at each occurrence from hydrogen and R⁴¹;
R⁴⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R⁴⁰ attached to the same carbon atom optionally join to form ═NR¹², ═C(R¹⁴)₂, or ═O; wherein two R⁴⁰ and the atom(s) to which they are attached optionally form C₃₋₁₂ carbocycle or 3- to 12-membered heterocycle; wherein R⁴⁰ and R⁴¹ and the atoms to which they are attached optionally form 3- to 12-membered heterocycle; and wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
R⁴¹ is independently selected at each occurrence from —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted.

Embodiment 91. The compound, salt, or solvate of any one of embodiments 76 to 84, wherein:
L¹ is a bond;
L², R⁹, and the atoms to which they are attached form optionally substituted 3- to 12-membered heterocycle; and
L³ is selected from a bond and optionally substituted nitrene.

Embodiment 92. The compound, salt, or solvate of any one of embodiments 76 to 84 or 91, wherein L², R⁹, and the atoms to which they are attached form optionally substituted 5- to 8-membered monocyclic heterocycle.

Embodiment 93. The compound, salt, or solvate of any one of embodiments 76 to 84 or 91, wherein L², R⁹, and the atoms to which they are attached form optionally substituted 6- to 12-membered spirocyclic heterocycle.

Embodiment 94. The compound, salt, or solvate of any one of embodiments 76 to 84 or 91, wherein L², R⁹, and the atoms to which they are attached form optionally substituted 7- to 12-membered fused bicyclic heterocycle.

Embodiment 95. The compound, salt, or solvate of any one of embodiments 76 to 84 or 91, wherein

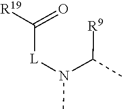

is selected from:

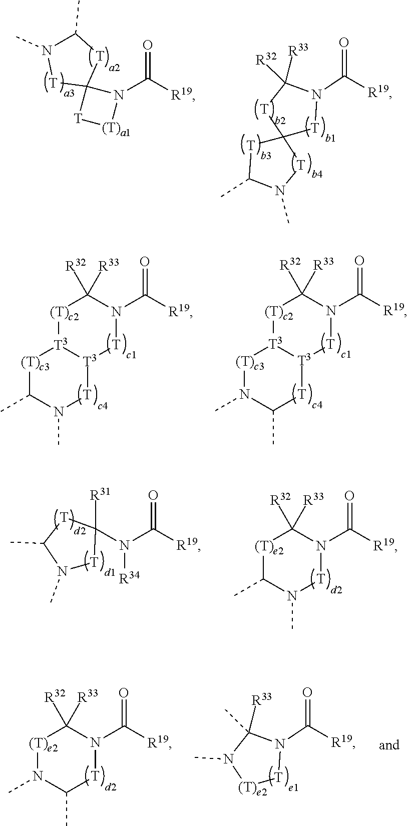

wherein:
a1, b1, b3, and b4 are independently 1, 2, 3, 4, or 5;
a2, a3, and b2 are independently 0, 1, 2, 3, 4, or 5;
c1, c2, c3, c4, d1, d2, e1, and e2 are independently 0, 1, 2, 3, or 4;
wherein the sum of a1, a2, and a3 is less than 8; the sum of b1, b2, b3, and b4 is less than 8; the sum of c1, c2, c3, and c4 is less than 7; the sum of d1 and d2 is less than 6; and the sum of e1 and e2 is less than 6;
T is independently selected at each occurrence from N(R³⁵), C(R³⁶)₂, C(O), O, S(O), and S(O)₂;
T³ is independently selected at each occurrence from N and C(R³⁶);
R³¹, R³², R³³, and R³⁶ are independently selected at each occurrence from hydrogen and R⁴⁰;
R³⁴ and R³⁵ are independently selected at each occurrence from hydrogen and R⁴¹;
R⁴⁰ is independently selected at each occurrence from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R⁴⁰ attached to the same carbon atom optionally join to form ═NR¹², ═C(R¹⁴)₂, or ═O; wherein two R⁴⁰ and the atom(s) to which they are attached optionally form C₃₋₁₂ carbocycle or 3- to 12-membered heterocycle; wherein R⁴⁰ and R⁴¹ and the atoms to which they are attached optionally form 3- to 12-membered heterocycle; and wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
R⁴¹ is independently selected at each occurrence from —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —C(O)OR¹², —C(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted.

Embodiment 96. The compound, salt, or solvate of embodiment 90 or 95, wherein R⁴⁰ is independently selected at each occurrence from halogen, —CN, C₁₋₆ alkyl, and C₃₋₆ cycloalkyl, or two R⁴⁰ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl).

Embodiment 97. The compound, salt, or solvate of embodiment 90, 95, or 96, wherein R⁴¹ is independently selected at each occurrence from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

Embodiment 98. The compound of embodiment 76, having the structure:

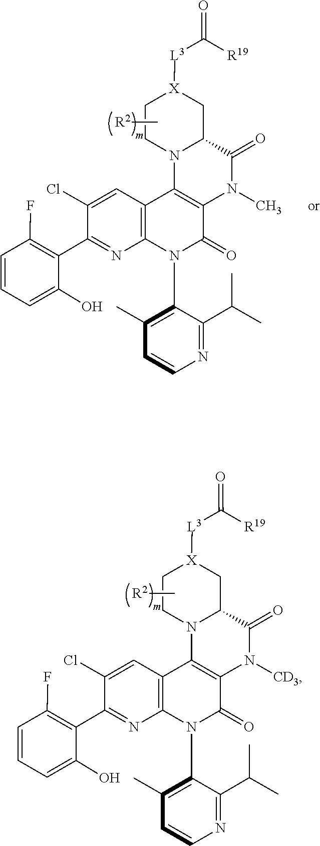

or a pharmaceutically acceptable salt or solvate thereof, wherein:
X is selected from CH and N;
R² is selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R² and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted; and
m is 0, 1, 2, or 3.

Embodiment 99. The compound, salt, or solvate of embodiment 98, wherein R² is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2.

Embodiment 100. The compound, salt, or solvate of embodiment 98, wherein R² is selected from —CH₃, and —CH₂CN; and m is 1 or 2.

Embodiment 101. The compound, salt, or solvate of embodiment 98, wherein m is 0.

Embodiment 102. The compound, salt, or solvate of any one of embodiments 98 to 101, wherein X is N and L³ is a bond.

Embodiment 103. The compound, salt, or solvate of any one of embodiments 98 to 101, wherein X is CH and L³ is optionally substituted nitrene.

Embodiment 104. The compound of embodiment 80, or a pharmaceutically acceptable salt or solvate thereof, having the structure:

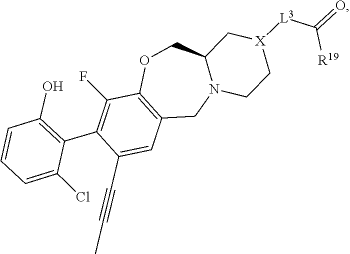

wherein X is selected from CH and N.

Embodiment 105. The compound, salt, or solvate of embodiment 104, wherein X is CH and L³ is —N(R¹²)—.

Embodiment 106. The compound, salt, or solvate of embodiment 104, wherein X is N and L³ is a bond.

Embodiment 107. A compound of Formula (XXI):

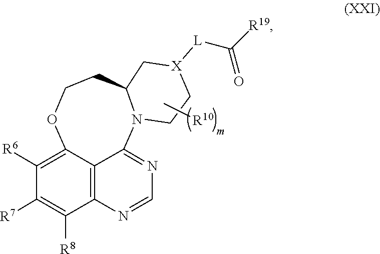

or a pharmaceutically acceptable salt or solvate thereof, wherein:
L is a bond and X is N; or L is —N(R¹²)— and X is CH;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R⁷ is selected from a phenyl and pyridyl; wherein the phenyl and pyridyl are optionally substituted;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3;
R⁶ is hydrogen, —CN, halogen, C₁₋₄ alkyl, C₁₋₄ haloalkyl, and C₁₋₄ alkoxyl;
R⁸ is hydrogen, halogen, C₁₋₄ alkyl, C₂₋₄ alkynyl, C₁₋₄ haloalkyl, and C₁₋₄ alkoxyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

Embodiment 108. The compound of embodiment 107, having the structure of Formula (XXII):

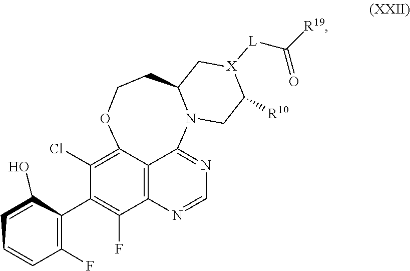

or a pharmaceutically acceptable salt or solvate thereof.

Embodiment 109. The compound, salt, or solvate of embodiment 107 or 108, wherein X is CH and L is —N(R¹²)—.

Embodiment 110. The compound, salt, or solvate of embodiment 107 or 108, wherein X is N and L is a bond.

Embodiment 111. The compound, salt, or solvate of any one of embodiments 107 to 110, wherein R¹⁰ is selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl).

Embodiment 112. The compound, salt, or solvate of any one of embodiments 107 to 110, wherein R¹⁰ is —CH₃.

Embodiment 113. The compound of embodiment 82, having the structure:

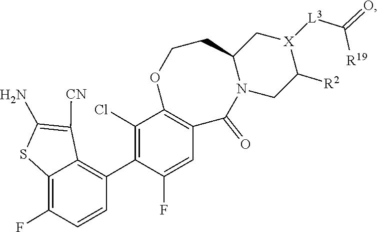

or a pharmaceutically acceptable salt or solvate thereof, wherein:
X is selected from CH and N; and
R² is selected from hydrogen, halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted.

Embodiment 114. The compound, salt, or solvate of embodiment 113, wherein R² is selected from hydrogen, halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl).

Embodiment 115. The compound, salt, or solvate of embodiment 113, wherein R² is selected from hydrogen, —CH₃, and —CH₂CN.

Embodiment 116. The compound, salt, or solvate of embodiment 113, wherein R² is hydrogen.

Embodiment 117. The compound, salt, or solvate of any one of embodiments 113 to 116, wherein X is N and L³ is a bond.

Embodiment 118. The compound, salt, or solvate of any one of embodiments 113 to 116, wherein X is CH and L³ is optionally substituted nitrene.

Embodiment 119. The compound, salt, or solvate of any one of embodiments 1 to 118, wherein R¹⁹ is selected from 1,2,3-triazol-1-yl, 1,2,3-triazol-2-yl, 1,2,4-triazol-1-yl, and 1,2,4-triazol-4-yl, each of which is optionally substituted.

Embodiment 120. The compound, salt, or solvate of any one of embodiments 1 to 118, wherein R¹⁹ is optionally substituted imidazol-1-yl.

Embodiment 121. The compound, salt, or solvate of any one of the preceding embodiments, wherein R¹⁹ is optionally substituted with one or two substituents independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents independently selected from halogen, —CN, C₁₋₆ alkyl, —O(C₁₋₆ alkyl), and —O(C₁₋₆ haloalkyl).

Embodiment 122. A pharmaceutical composition comprising a compound of any one of embodiments 1 to 121, or a pharmaceutically acceptable salt or solvate thereof, and a pharmaceutically acceptable excipient.

Embodiment 123. A method of modifying a Ras mutant protein, comprising contacting the Ras mutant protein with an effective amount of the compound, salt, or solvate of any one of embodiments 1 to 121.

Embodiment 124. The method of embodiment 123, wherein the modified Ras mutant protein exhibits a reduced Ras signaling output.

Embodiment 125. The method of embodiment 124, wherein the reduced Ras signaling output is evidenced by one or more output selected from (i) an increase in steady state level of GDP-bound modified protein; (ii) a reduction in steady state level of GTP-bound modified protein; (iii) a reduction of phosphorylated AKTs473; (iv) a reduction of phosphorylated ERK T202/Y204; (v) a reduction of phosphorylated S6 S235/236; (vi) a reduction of cell growth of a tumor cell expressing a Ras G12S mutant protein; and (vii) a reduction in Ras interaction with a Ras-pathway signaling protein.

Embodiment 126. The method of any one of embodiments 123 to 125, wherein the Ras mutant protein comprises an amino acid sequence in SEQ ID No. 4 having a serine residue corresponding to position 12 of SEQ ID No. 1.

Embodiment 127. The method of any one of embodiments 123 to 125, wherein the Ras mutant protein comprises an amino acid sequence of SEQ ID No. 4.

Embodiment 128. The method of any one of embodiments 123 to 125, wherein the modified Ras mutant protein comprises an amino acid sequence of SEQ ID No. 1, or a fragment thereof that comprises the serine residue corresponding to position 12 of SEQ ID No. 1, and wherein the compound selectively labels the serine residue as compared to (i) an aspartate residue of a K-Ras G12D mutant protein, said aspartate corresponding to position 12 of SEQ ID No. 2; (ii) a valine residue of a K-Ras G12V mutant protein, said valine corresponding to position 12 of SEQ ID No. 3; and/or (iii) a glycine residue of a K-Ras wildtype protein, said glycine corresponding to position 12 of SEQ ID No. 1.

Embodiment 129. The method of embodiment 128, wherein the compound selectively labels the serine residue by at least 2-fold when assayed under comparable conditions.

Embodiment 130. The method of embodiment 128, wherein the compound selectively labels the serine residue by at least 5-fold when assayed under comparable conditions.

Embodiment 131. The method of any one of embodiments 123 to 130, wherein the contacting occurs in vivo.

Embodiment 132. A method of treating cancer in a subject in need thereof, comprising administering to the subject a therapeutically effective amount of a compound of any one of embodiments 1 to 121, or a pharmaceutically acceptable salt or solvate thereof.

Embodiment 133. A method of treating cancer in a subject comprising a Ras mutant protein, the method comprising: inhibiting the Ras mutant protein of said subject by administering to said subject a compound of any one of embodiments 1 to 121, wherein the compound is characterized in that upon contacting the Ras mutant protein, said Ras mutant protein exhibits reduced Ras signaling output.

Embodiment 134. The method of embodiment 132 or 133, wherein the cancer is a solid tumor or a hematological cancer.

Embodiment 135. The method of any one of embodiments 132 to 134, wherein the cancer comprises a K-Ras G12S mutant protein.

Embodiment 136. A method of modulating signaling output of a Ras protein, comprising contacting a Ras protein with an effective amount of a compound of any one of embodiments 1 to 121, or a pharmaceutically acceptable salt or solvate thereof, thereby modulating the signaling output of the Ras protein.

Embodiment 137. A method of inhibiting cell growth, comprising administering an effective amount of a compound of any one of embodiments 1 to 121, or a pharmaceutically acceptable salt or solvate thereof, to a cell expressing a Ras protein, thereby inhibiting growth of said cells.

Embodiment 138. The method of any one of embodiments 123 to 137, comprising administering an additional agent.

### EXAMPLES

The following examples are provided for illustrative purposes only and not to limit the scope of the claims provided herein. Unless noted otherwise, all materials, such as reagents, starting materials and solvents, were purchased from commercial suppliers, such as Sigma-Aldrich, VWR, and the like, and were used without further purification. Reactions were run under nitrogen atmosphere, unless noted otherwise. The progress of reactions was monitored by thin layer chromatography (TLC), analytical high performance liquid chromatography (anal. HPLC), and mass spectrometry, the details of which may be provided in specific examples.

Reactions were worked up as described specifically in each preparation; commonly, reaction mixtures were purified by extraction and other purification methods such as temperature- and solvent-dependent crystallization, and precipitation. In addition, reaction mixtures were routinely purified by preparative HPLC, for example, using Microsorb C18 or Microsorb BDS column packings and conventional eluents. Progress of reactions was typically monitored by liquid chromatography mass spectrometry (LCMS). Characterization of isomers was typically done by Nuclear Overhauser effect spectroscopy (NOE). Characterization of reaction products was routinely carried out by mass spectrometry and/or ¹H-NMR spectroscopy. For NMR measurement, samples were dissolved in deuterated solvent (CD₃OD, CDCl₃, or DMSO-d₆).

### Example 1: Ras Sequences

| | Human K-Ras Wildtype sequence |
|---|---|
| | (SEQ ID NO. 1) |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| | Human K-Ras G12D |
| | (SEQ ID NO. 2) |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| | Human K-Ras G12V |
| | (SEQ ID NO. 3) |
| 1 | MTEYKLVVVG AVGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| | Human K-Ras G12S: |
| | (SEQ ID NO. 4) |
| 1 | MTEYKLVVVG ASGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| | Human N-Ras wildtype |
| | (SEQ ID NO. 5) |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNSKSF ADINLYREQI |
| 101 | KRVKDSDDVP MVLVGNKCDL PTRTVDTKQA HELAKSYGIP FIETSAKTRQ |
| 151 | GVEDAFYTLV REIRQYRMKK LNSSDDGTQG CMGLPCVVM |
| | Human H-Ras G12D |
| | (SEQ ID NO. 6) |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNTKSF EDIHQYREQI |
| 101 | KRVKDSDDVP MVLVGNKCDL AARTVESRQA QDLARSYGIP YIETSAKTRQ |
| 151 | GVEDAFYTLV REIRQHKLRK LNPPDESGPG CMSCKCVLS |
| | Human H-Ras wildtype |
| | (SEQ ID NO. 7) |
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHQYREQI |
| 101 | KRVKDSDDVP MVLVGNKCDL AARTVESRQA QDLARSYGIP YIETSAKTRQ |
| 151 | GVEDAFYTLV REIRQHKLRK LNPPDESGPG CMSCKCVLS |
| | Human N-Ras G12D |
| | (SEQ ID NO. 8) |
| 1 | MTEYKLVVVG ADGVGKSALT IQLIQNHFVD EYDPTIEDSY RKQVVIDGET |
| 51 | CLLDILDTAG QEEYSAMRDQ YMRTGEGFLC VFAINNSKSF ADINLYREQI |
| 101 | KRVKDSDDVP MVLVGNKCDL PTRTVDTKQA HELAKSYGIP FIETSAKTRQ |
| 151 | GVEDAFYTLV REIRQYRMKK LNSSDDGTQG CMGLPCVVM |
| | Human K-Ras G12C: |
| | (SEQ ID NO. 9) |
| 1 | MTEYKLVVVG ACGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |

### Example 2: Protein Expression

DNA expression constructs encoding one or more protein sequences of interest (e.g., KRAS fragments thereof, mutant variants thereof, etc.) and its corresponding DNA sequences are optimized for expression in E. coli and synthesized by, for example, the GeneArt Technology at Life Technologies. In some cases, the protein sequences of interest are fused with a tag (e.g., glutathione S-transferase (GST), histidine (His), or any other affinity tags) to facilitate recombinant expression and purification of the protein of interest. Such tag can be cleaved subsequent to purification. Alternatively, such tag may remain intact to the protein of interest and may not interfere with activities (e.g., target binding and/or phosphorylation) of the protein of interest

A resulting expression construct is additionally encoded with (i) att-site sequences at the 5′ and 3′ ends for subcloning into various destination vectors using, for example, the Gateway Technology, as well as (ii) a Tobacco Etch Virus (TEV) protease site for proteolytic cleavage of one or more tag sequences. The applied destination vectors can be a pET vector series from Novagen (e.g., with ampicillin resistance gene), which provides an N-terminal fusion of a GST-tag to the integrated gene of interest and/or a pET vector series (e.g., with ampicillin resistance gene), which provides an N-terminal fusion of a HIS-tag to the integrated gene. To generate the final expression vectors, the expression construct of the protein of interest is cloned into any of the applied destination vectors. The expression vectors are transformed into an E. coli strain, e.g., BL21 (DE3). Cultivation of the transformed strains for expression is performed in a 10 L or 1 L fermenter. The cultures are grown, for example, in Terrific Broth media (MP Biomedicals, Kat. #1 13045032) with 200 μg/mL ampicillin at a temperature of 37° C. to a density of 0.6 (OD600), shifted to a temperature of ~27° C. (for K-Ras expression vectors) induced for expression with 100 mM IPTG, and further cultivated for 24 hours. After cultivation, the transformed E. coli cells are harvested by centrifugation and the resulting pellet is suspended in a lysis buffer, as provided below, and lysed by passing three-times through a high-pressure device. The lysate is centrifuged (49000 g, 45 min, 4° C.) and the supernatant is used for further purification.

### Example 3: Ras Protein Purification

A Ras (e.g., K-Ras wildtype or a mutant such as K-Ras G12S, K-Ras G12D, K-Ras G12V or K-Ras G12C) construct or a variant thereof is tagged with GST. E. coli culture from a 10 L fermenter is lysed in lysis buffer (50 mM Tris HCl 7.5, 500 mM NaCl, 1 mM DTT, 0.5% CHAPS, Complete Protease Inhibitor Cocktail-(Roche)). As a first chromatography step, the centrifuged lysate is incubated with 50 mL Glutathione Agarose 4B (Macherey-Nagel; 745500.100) in a spinner flask (16 h, 10° C.). The Glutathione Agarose 4B loaded with protein is transferred to a chromatography column connected to a chromatography system, e.g., an Akta chromatography system. The column is washed with wash buffer (50 mM Tris HCl 7.5, 500 mM NaCl, 1 mM DTT) and the bound protein is eluted with elution buffer (50 mM Tris HCl 7.5, 500 mM NaCl, 1 mM DTT, 15 mM glutathione). The main fractions of the elution peak (monitored by OD280) are pooled. For further purification by size-exclusion chromatography, the above eluate volume is applied to a column Superdex 200 HR prep grade (GE Healthcare) and the resulting peak fractions of the eluted fusion protein is collected. Native mass spectrometry analyses of the final purified protein construct can be performed to assess its homogeneous load with GDP.

### Example 4: HTRF (Homogenous Time-Resolved Fluorescence) Resonance Energy Transfer Assay

The ability of a compound of the present disclosure to reduce Ras signaling output can be demonstrated by an HTRF assay. This assay can be also used to assess a selective inhibition or reduction of signaling output of a mutant Ras protein relative to a wildtype, or relative to a different mutant Ras protein. For example, the equilibrium interaction of wildtype KRAS or K-Ras mutant (e.g., wildtype or a mutant thereof) with SOS1 (e.g., hSOS1) can be assessed as a proxy or an indication for the ability of a subject compound to bind and inhibit Ras protein. The HTRF assay detects from (i) a fluorescence resonance energy transfer (FRET) donor (e.g., antiGST-Europium) that is bound to GST-tagged K-Ras mutant to (ii) a FRET acceptor (e.g., anti-6His-XL665) bound to a His-tagged hSOSL.

The assay buffer can contain ~5 mM HEPES pH 7.4, ~150 mM NaCl, ~1 mM DTT, 0.05% BSA and 0.0025% (v/v) Igepal. A Ras working solution is prepared in an assay buffer containing typically a suitable amount of the protein construct (e.g., GST-tagged K-Ras mutant) and the FRET donor (e.g., antiGST-Eu(K) from Cisbio, France). A SOS1 working solution is prepared in an assay buffer containing suitable amount of the protein construct (e.g., His-hSOS1) and the FRET acceptor (e.g., anti-6His-XL665 from Cisbio, France). A suitable amount of the protein construct will depend on the range of activity or range of IC50 values being detected or under investigation. For detecting an IC50 within a range of 500 nM, the protein constructs of the same range of molarity can be utilized. An inhibitor control solution is prepared in an assay buffer containing a comparable amount of the FRET acceptor without the SOS1 protein.

A fixed volume of DMSO with or without test compound is transferred into a 384-well plate. Ras working solution is added to all wells of the test plate. SOS1 working solution is added to all wells except for those that are subsequently filled with inhibitor control solution. Upon incubation for about 10 minutes or longer, the fluorescence is measured with a M1000Pro plate reader (Tecan) using HTRF detection (excitation 337 nm, emission 1: 620 nm, emission 2: 665 nm). Compounds are tested in duplicate at different concentrations (for example, 10 μM, 2.5 μM, 0.63 μM, 0.16 μM, 0.04 μM, 0.01 μM test compound). The ratiometric data (i.e., emission 2 divided by emission 1) is used to calculate IC50 values against Ras using GraphPad Prism (GraphPad software). Signaling output measured in terms of IC50 values can be obtained and a ratio of IC50 against one mutant relative to another mutant can be calculated. For instance, a selective reduction of K-Ras G12S signaling output can be evidenced by a ratio greater than one. In particular, a selective reduction of K-Ras G12S signaling relative to K-Ras WT signaling is evidenced if the ratio of IC50 (against K-Ras WT) to IC50 (against K-Ras G12S) is greater than 1. In some embodiments, one or more subject compounds disclosed herein are expected to exhibit selective inhibition of a Ras mutant (e.g., G12C, G12S, or G13C) over WT by at least 1-fold, and in some instances greater than 2-, 3-, 4- or 5-fold. In some embodiments, subject compounds are expected to exhibit an IC50 against KRas mutants (e.g., G12C or G12S) less than 500 nM, such as less than 100 nM, 50 nM, or even less.

### Example 5: GTPase Activity Assay

The ability of a compound of the present disclosure to inhibit Ras protein signaling can be demonstrated by a reduced GTPase activity. This assay can also be used to assess selective inhibition of a mutant Ras protein relative to a wildtype or different mutant Ras protein. For instance, the assay can be used to establish a subject compound's ability to selectively inhibit KRAS G12S relative to wildtype, KRAS G12S relative to KRAS G12V, KRAS G12S relative to KRAS G12D, KRAS G12C relative to KRAS G12D, or KRAS G12C relative to KRAS G12V or wildtype. In particular, intrinsic and GTPase-activating protein (GAP)-stimulated GTPase activity for a K-Ras construct or a mutant thereof can be measured using EnzCheck phosphate assay system (Life Technologies). For example, K-Ras WT, K-Ras D154Q mutant, K-Ras G12D mutant, K-Ras G12S mutant, and K-Ras G12D/D154Q mutant proteins (2.5 mg/mL) in buffer (20 mmol/L Tris, pH 8.0, 50 mM NaCl) are loaded with GTP at room temperature for 2 hours by exposing to exchange buffer containing EDTA. Proteins are buffer exchanged to assay buffer (30 mM Tris, pH 7.5, 1 mM DTT) and the concentration is adjusted to 2 mg/mL. GTP loading is verified by back extraction of nucleotide using 6M urea and evaluation of nucleotide peaks by HPLC using an ion-exchange column. The assay is performed in a clear 384-well plate (Costar) by combining GTP-loaded K-Ras proteins (50 mM final) with 2-amino-6-mercapto-7-methylpurine ribonucleoside (MESG) (200 mM final), and purine nucleotide phosphorylase (5 U/mL final). GTP hydrolysis is initiated by the addition of MgCl₂ at a working concentration of 40 mM. For GAP stimulation, Ras p21 protein activator 1 (P120GAP) can be included at 50 mM. Absorbance at 360 nm can be measured every 8 to 15 s for 1,000 s at 20° C. Samples are tested with or without a subject compound disclosed herein to assess the ability of each compound to inhibit signaling of a given Ras protein (e.g., a given mutant KRAS) of interest.

### Example 6: Nucleotide Exchange Assay

The ability of a compound of the present disclosure to inhibit Ras protein signaling can be demonstrated by reduced nucleotide exchange activity. This assay can be also used to assess selective inhibition of a mutant Ras protein relative to a wildtype or different mutant Ras protein. For example, 250 nM or 500 nM GDP-loaded K-Ras protein (e.g., wildtype or a mutant thereof, including those mentioned in Example 3) is incubated with different concentrations of compounds (for example ~60 μM, ~20 μM, ~6.7 μM, ~2.2 μM, ~0.7 μM, or ~0.2 μM subject compound). A control reaction without subject compound is also included. SOS1 (catalytic domain) protein is added to the K-Ras protein solution. The nucleotide exchange reaction is initiated by adding fluorescent labelled GDP (guanosine 5′-diphosphate, BODIPY™ FL 2′-(or-3′)-O—(N-(2-aminoethyl)urethane) to a final concentration of 0.36 μM. Fluorescence is measured every 30 s for 70 minutes at 490 nm/515 nm (excitation/emission) in a M1000Pro plate reader (Tecan). Data is exported and analyzed to calculate an IC50 using GraphPad Prism (GraphPad Software). Sample(s) can be tested with or without a subject compound disclosed herein to assess the ability of the compound to inhibit K-Ras signaling or its IC50 against a given Ras protein (e.g., a given mutant K-Ras) of interest.

### Example 7: Testing for Modification of Ras Protein Via Covalent Binding

Test compounds are prepared as 10 mM stock solutions in DMSO (Fisher cat #BP231-100). KRAS protein (His-tagged GDP-loaded wildtype 1-169, His-tagged GDP-loaded G12S 1-169 or His-tagged GDP-loaded G12D 1-169) is diluted to ~2 μM in appropriate buffer (e.g., a Hepes buffer at physiological conditions). For testing KRAS modification, compounds are diluted to 50× final test concentration in DMSO in 96-well storage plates. 2 μL of the diluted 50× compounds are added to appropriate wells in the PCR plate (Fisher cat #AB-0800). ~49 μL of the stock protein solution is added to each well of the 96-well PCR plate. Reactions are mixed carefully. The plate is sealed well with aluminum plate seal and stored in a drawer at room temperature for 24 hrs. 5 μL of 2% formic acid (Fisher cat #A117-50) in MilliQ H₂O is then added to each well followed by mixing with a pipette. The plate is then resealed with aluminum seal and stored until mass spectrometry analysis.

The extent of covalent modification of KRAS proteins can be determined by liquid chromatography electrospray mass spectrometry analysis of the intact proteins on a Thermo Q-Exactive Plus mass spectrometer. 20 μL of sample is injected onto a bioZen 3.6 μm Intact C4 column (Phenomenex cat #00α-4767-AN) placed in a column oven set to 40° C. and separated using a suitable LC gradient from ~20% to ~60% solvent B. Solvent A is 0.1% formic acid and solvent B is 0.1% formic acid in acetonitrile. HESI source settings are set to 40, 5 and 1 for the sheath, auxiliary and sweep gas flow, respectively. The spray voltage is 4 kV, and the capillary temperature is 320° C. S-lens RF level is 50 and auxiliary gas heater temperature is set to 200° C. The mass spectrometry is acquired using a scan range from 650 to 1750 m/z using positive polarity at a mass resolution of 70,000, AGC target of 1e6 ions and maximum injection time of 250 ms. The recorded protein mass spectrum is deconvoluted from the raw data file using Protein Deconvolution v4.0 (Thermo). The protein mass and adduct masses are exported with their peak intensities. The peak intensities for the unmodified and modified protein are used to calculate the percent covalent modification of the KRAS protein based on the following equation: % KRAS protein modification=((KRAS-compound)/(KRAS)+(KRAS-Compound))*100.

### Example 8: Ras Cellular Assay

The ability of a compound of the present disclosure to inhibit Ras protein signaling can be demonstrated by inhibiting growth of a given KRAS mutant cell line. For example, this assay can be also used to assess selective growth inhibition of a mutant Ras protein relative to a wildtype or different mutant Ras protein.

a. Growth of Cells with K-Ras G12C Mutation

MIA PaCa-2 (ATCC CRL-1420) and NCI-H1792 (ATCC CRL-5895) cell lines comprise a G12C mutation and can be used to assess Ras cellular signaling in vitro, e.g., in response to an inhibitor compound of the present disclosure. This cellular assay can also be used to discern selective inhibition of a subject compound against certain types of KRAS mutants, e.g., more potent inhibition against KRAS G12C relative to KRAS G12D mutant, by comparing inhibition of MIA PaCa-2 (G12C driven tumor cell line) to inhibition of GP2d (G12D driven tumor cell line). MIA PaCa-2 culture medium is prepared with DMEM/Ham's F12 (e.g., with stable glutamine, 10% FCS, and 2.5% horse serum. NCI-H1792 culture medium is prepared with RPMI 1640 (e.g., with stable glutamine) and 10% FCS.

On a first day (e.g., Day 1), Softagar (Select Agar, Invitrogen, 3% in ddH₂O autoclaved) is boiled and tempered at 48° C. Appropriate culture medium (i.e., medium) is tempered to 37° C. Agar (3%) is diluted 1:5 in medium (=0.6%) and plated into 96 well plates (Corning, #3904), then incubated at room temperature for agar solidification. A 3% agar is diluted to 0.25% in medium (1:12 dilution) and tempered at 42° C. Cells are trypsinized, counted, and tempered at 37° C. The cells (e.g., MIA PaCa-2 at about 125-150 cells, NCI-H1792 at about 1000 cells) are resuspended in 100 mL 0.25% Agar and plated, followed by incubation at room temperature for agar solidification. The wells are overlaid with 50 mL of the medium. Sister wells in a separate plate are plated for time zero determination. All plates are incubated overnight at 37° C. and 5% CO₂.

On a second day (e.g., Day 2), time zero values are measured. A 40 mL volume of Cell Titer 96 Aqueous Solution (Promega) is added to each well and incubated in the dark at 37° C. and 5% CO₂. Absorption can be measured at 490 nm and reference wavelength 660 nm. DMSO-prediluted test compounds are added to wells of interest, e.g., with HP Dispenser, to one or more desired concentrations (e.g., a final DMSO concentration of 0.3%).

On a tenth day (e.g., Day 10), absorption by wells treated with the test compounds and control wells are measured with, for example, Cell Titer 96 AQueous and analyzed in comparison to the time zero measurements. The IC50 values are determined using the four parameter fit. The resulting IC50 value is a measurement of the ability of the test compound to reduce cell growth of Ras-driven cells (e.g., tumor cell lines) in vitro and/or in vivo. One or more compounds disclosed herein is expected to exhibit an IC50 value less than 5 μM, 1 μM, 100 nM, or even less, against one or more KRAS G12C cell line (including MIA PaCa-2 and NCI-H1792).

b. Growth of Cells with K-Ras G12S Mutation

A549 (ATCC CRL-185) and LS123 (ATCC CRL-255) cell lines comprise a G12S mutation and can be used to assess Ras cellular signaling in vitro, e.g., in response to treatment with a compound described herein. A549 culture medium is prepared with RPMI-1640 and 10% heat-inactivated FBS. LS123 culture medium is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384WZ). The day after plating, cells are treated with a dilution series (e.g., a 10 point, 3-fold dilution series) of the compounds herein (e.g., approximately 40 μL final volume per well). Cell viability can be monitored (e.g., approximately 6 days later) according to the manufacturer's recommended instructions, where CellTiter-Glo reagent is added (e.g., approximately 10 μL), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the test compound to reduce cell growth of Ras-driven cells (e.g., tumor cell lines) in vitro and/or in vivo. One or more compounds disclosed herein is expected to exhibit an IC50 value less than 5 μM, 1 μM, 100 nM, or even less, against one or more KRAS G12S cell line (including A549 and LS123).

### Example 9: In Vivo Ras Inhibition

The in vivo reduction in Ras signaling output by a compound of the present disclosure is determined in a mouse tumor xenograft model, particularly by using a mutant K-Ras model including without limitation a K-Ras G12S model, a K-Ras G12C model, a K-Ras G12D model, a K-Ras G13D model, and a K-Ras G13C model. These models can be generated by the methods and procedures described below. In particular, the methods disclosed below involving the use of a K-Ras G12S mutant cell line for generating a K-Ras G12S xenograft model can be applied to other K-Ras mutant animal models using the respective K-Ras mutant cell lines described above.

Xenograft with K-Ras G12D, G12C, or G12S Mutation

Tumor xenografts are established by administration of tumor cells with a K-Ras G12D mutation (e.g., ASPC-1 cells), a K-Ras G12C mutation (e.g., MIA PaCa-2 cells), or a K-Ras G12S mutation (e.g., A549 or LS123 cells) into mice. Female 6- to 8-week-old athymic BALB/c nude (NCr) nu/nu mice are used for xenografts. The tumor cells (e.g., approximately 5×10⁶) are harvested on the day of use and injected in growth-factor-reduced Matrigel/PBS (e.g., 50% final concentration in 100 μL). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L×w²)/2, in which L and w refer to the length and width of the tumor, respectively. Percent tumor volume change can be calculated using the following formula: (V_{final}−Vᵢₙᵢₜₐₗ)/Vᵢₙᵢₜᵢₐₗ×100. Percent of tumor growth inhibition (% TGI) can be calculated using the following formula: % TGI=100×(1−(average V_{final}−Vᵢₙᵢₜᵢₐₗ of treatment group)/(average V_{final}−Vᵢₙᵢₜᵢₐₗ of control group). When tumors reach a threshold average size (e.g., approximately 200-400 mm³), mice are randomized into 3-10 mice per group and are treated with vehicle (e.g., 100% Labrasol®) or a compound disclosed herein, using, for example, a daily schedule by oral gavage. Results can be expressed as mean and standard deviation of the mean.

1-33. (canceled)

34. A compound of Formula (XVI): 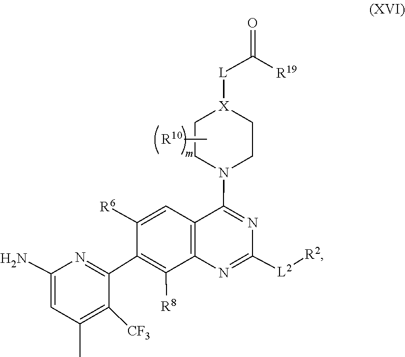
or a pharmaceutically acceptable salt or solvate thereof, wherein:
L is a bond and X is N; or L is —N(R¹²)— and X is CH;
R¹⁹ is selected from imidazol-1-yl, 1,2,3-triazol-1-yl, 1,2,3-triazol-2-yl, 1,2,4-triazol-1-yl, and 1,2,4-triazol-4-yl, each of which is optionally substituted;
R¹⁰ is independently selected from halogen, —CN, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), ═NR¹², ═C(R¹⁴)₂, ═O, —OR¹², —SR¹², —N(R¹²)(R¹³), —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹²; wherein two R¹⁰ and the atom(s) to which they are attached optionally form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
m is 0, 1, 2, or 3;
L² is selected from a bond, —O—, —N(R^{2a})—, and —S—;
R^{2a} is selected from hydrogen and C₁₋₃ alkyl, wherein C₁₋₃ alkyl is optionally substituted;
R² is selected from hydrogen, C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), halogen, —CN, —OR¹², —SR¹², —N(R¹²)(R¹³), C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, 2- to 6-membered heteroalkyl, 3- to 6-membered heteroalkenyl, 3- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted;
R⁶ and R⁸ are each independently selected from hydrogen, —F, —Cl, C₁₋₄ alkyl, C₁₋₄ haloalkyl, and C₁₋₄ alkoxyl;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted;
R¹³ is independently selected at each occurrence from hydrogen, C₁₋₆ alkyl, and C₁₋₆ haloalkyl; or R¹² and R¹³ attached to the same nitrogen atom form optionally substituted 3- to 10-membered heterocycle; and
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted.

35. The compound of claim 34, having the structure of Formula (XVII): 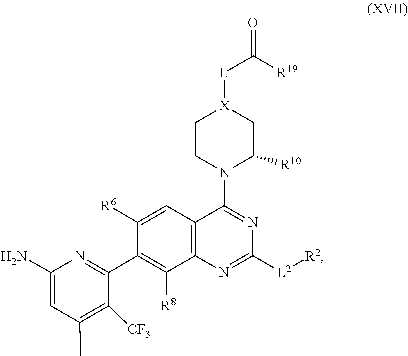
or a pharmaceutically acceptable salt or solvate thereof.

36. The compound, salt, or solvate of claim 34, wherein R⁶ is —Cl and R⁸ is —F.

37. The compound, salt, or solvate of claim 34, wherein L² is —O—.

38. The compound, salt, or solvate of claim 34, wherein R² is —C₀₋₆ alkyl-(3- to 12-membered heterocycle), wherein —C₀₋₆ alkyl-(3- to 12-membered heterocycle) is optionally substituted.

39. The compound, salt, or solvate of claim 34, wherein -L²-R² is

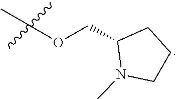 [SMILES: *OC[C@@H]1CCCN1C]

40. The compound, salt, or solvate of claim 34, wherein X is CH and L is —N(R¹²)—.

41. The compound, salt, or solvate of claim 34, wherein X is N and L is a bond.

42. The compound, salt, or solvate of claim 34, wherein R¹⁰ is independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and m is 1 or 2.

43. The compound, salt, or solvate of claim 34, wherein R¹⁰ is —CH₃.

44. The compound, salt, or solvate of claim 34, wherein R¹⁹ is selected from 1,2,3-triazol-1-yl, 1,2,3-triazol-2-yl, 1,2,4-triazol-1-yl, and 1,2,4-triazol-4-yl, each of which is optionally substituted.

45. The compound, salt, or solvate of claim 34, wherein R¹⁹ is optionally substituted imidazol-1-yl.

46. The compound, salt, or solvate of claim 34, wherein R¹⁹ is optionally substituted with one or two substituents independently selected from halogen, —CN, C₁₋₆ alkyl, and C₃₋₆ cycloalkyl, wherein C₁₋₆ alkyl and C₃₋₆ cycloalkyl are optionally substituted with one, two, or three substituents independently selected from halogen, —CN, C₁₋₆ alkyl, —O(C₁₋₆ alkyl), and —O(C₁₋₆ haloalkyl).

47. The compound of claim 34, wherein the compound is selected from: 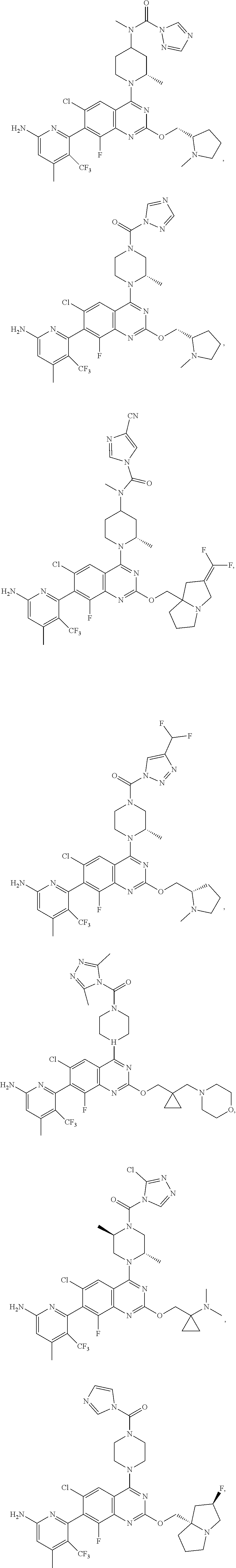 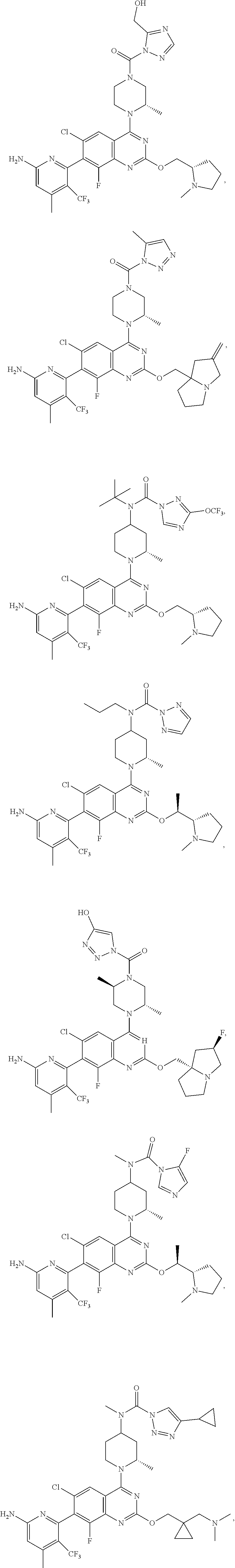 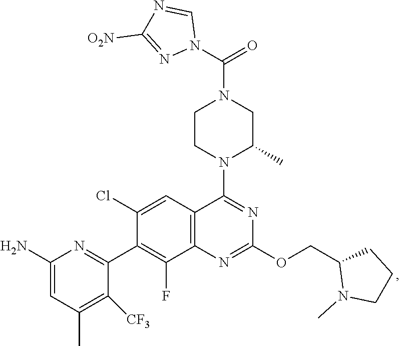 [SMILES: Cc1cc(N)nc(-c2c(Cl)cc3c(N4CCN(C(=O)n5cnc([N+](=O)[O-])n5)C[C@@H]4C)nc(OC[C@@H]4CCCN4C)nc3c2F)c1C(F)(F)F] 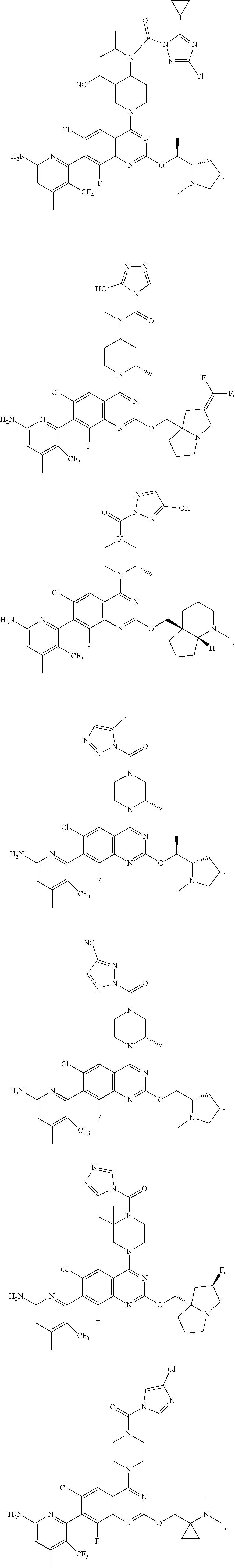 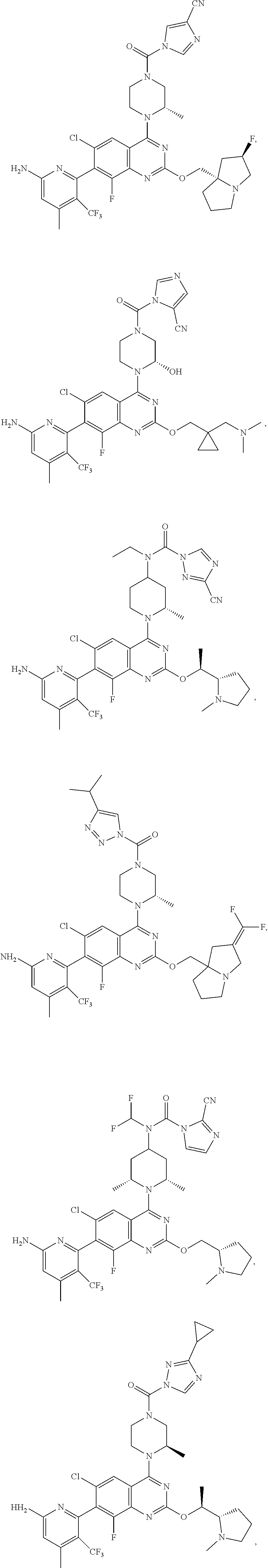 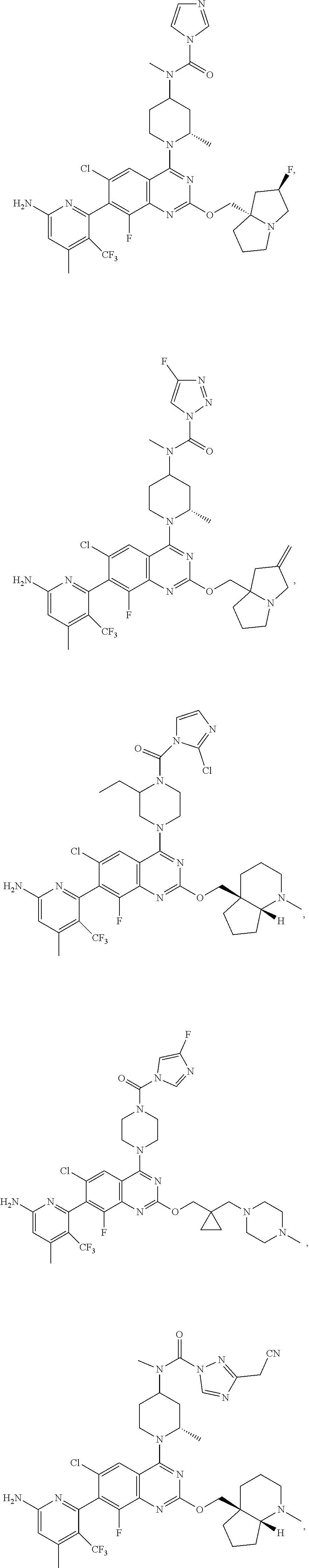 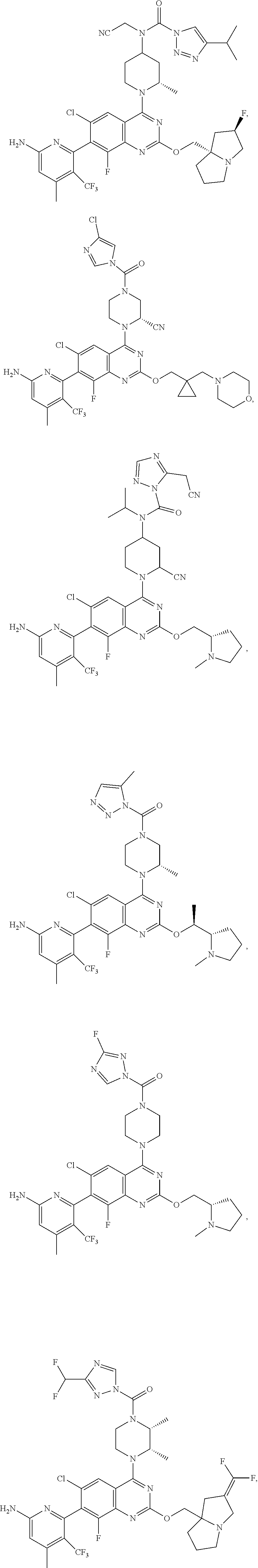 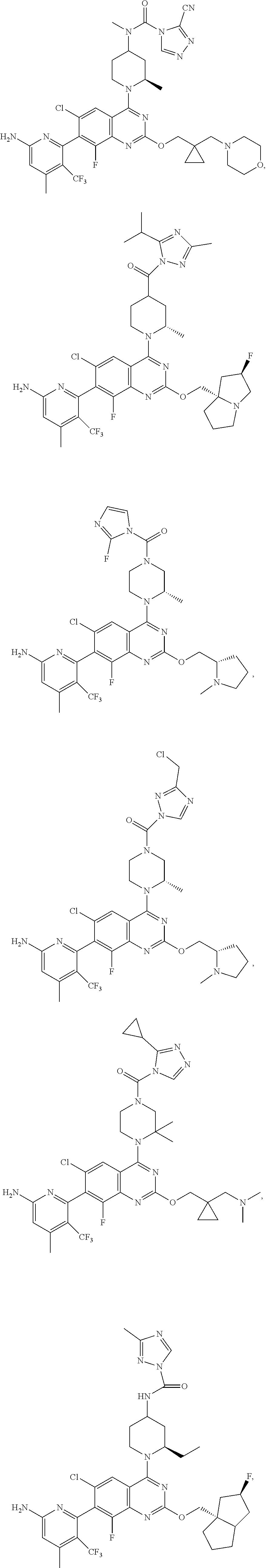 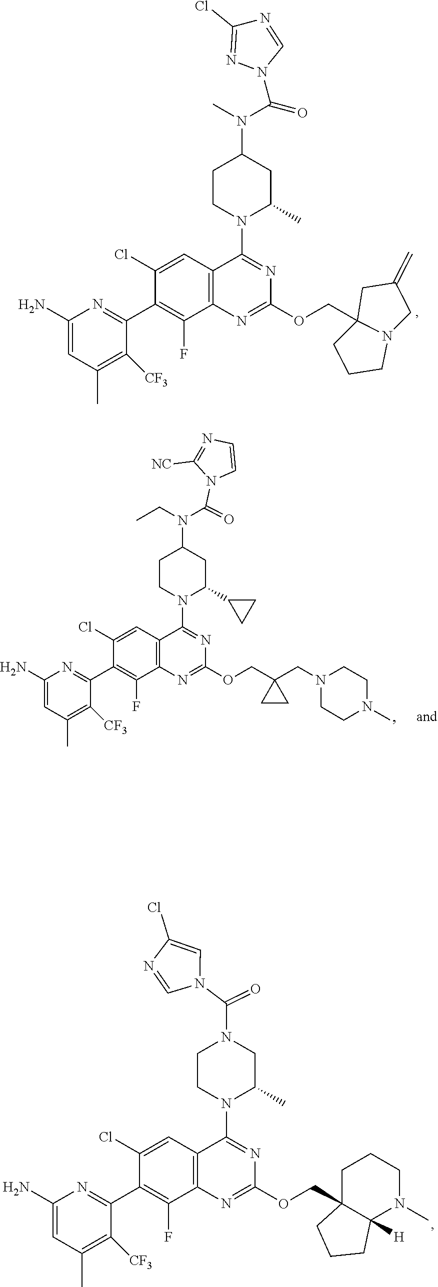 [SMILES: C=C1CN2CCCC2(COc2nc(N3CCC(N(C)C(=O)n4cnc(Cl)n4)C[C@@H]3C)c3cc(Cl)c(-c4nc(N)cc(C)c4C(F)(F)F)c(F)c3n2)C1 ; CCN(C(=O)n1ccnc1C#N)C1CCN(c2nc(OCC3(CN4CCN(C)CC4)CC3)nc3c(F)c(-c4nc(N)cc(C)c4C(F)(F)F)c(Cl)cc23)[C@@H](C2CC2)C1 ; Cc1cc(N)nc(-c2c(Cl)cc3c(N4CCN(C(=O)n5cnc(Cl)c5)C[C@@H]4C)nc(OC[C@]45CCC[C@H]4N(C)CCC5)nc3c2F)c1C(F)(F)F]
or a pharmaceutically acceptable salt or solvate thereof.

48. A pharmaceutical composition comprising a compound of claim 34, or a pharmaceutically acceptable salt or solvate thereof, and a pharmaceutically acceptable excipient.

49. A method of modifying a Ras mutant protein, comprising contacting the Ras mutant protein with an effective amount of the compound, salt, or solvate of claim 34.

50. A method of treating cancer in a subject comprising a Ras mutant protein, the method comprising: inhibiting the Ras mutant protein of said subject by administering to said subject a compound of claim 34, wherein the compound is characterized in that upon contacting the Ras mutant protein, said Ras mutant protein exhibits reduced Ras signaling output.

51. The method of claim 50, wherein the cancer is a solid tumor.

52. The method of claim 50, wherein the cancer comprises a K-Ras G12S mutant protein.

53. The method of claim 50, comprising administering an additional agent.
